(19) United States
(12) Patent Application Publication
(10) Pub. No.: US 2014/0031355 A1
(43) Pub. Date: Jan. 30, 2014

(54) HETEROCYCLIC COMPOUNDS AND THEIR USES

(71) Applicant: Amy Kaizerman, Salem, OR (US)

(72) Inventors: Benjamin Fisher, San Mateo, CA (US); Michael G. Johson, San Francisco, CA (US); Jacob Kaizerman, Salem, OR (US); Brian Lucas, San Francisco, CA (US); Youngsook Shin, Emeryville, CA (US)

(73) Assignee: Amgen Inc., Thousand Oaks, CA (US)

(21) Appl. No.: 13/880,448

(22) PCT Filed: Nov. 4, 2011
(86) PCT No.: PCT/US11/59309

§ 371 Date: Aug. 16, 2013

Related U.S. Application Data
(60) Provisional application No. 61/410,278, filed on Nov. 4, 2010.

(51) Int. Cl. C07D 239/48; C07D 401/14; C07D 473/34; C07D 401/12

(52) U.S. Cl. CPC C07D 239/48; C07D 401/12; C07D 401/14; C07D 473/34
USPC 514/248; 514/256; 544/328; 514/263.22; 544/277; 544/326; 514/269; 544/319; 544/235; 514/255.05; 544/295

(57) ABSTRACT

Substituted bicyclic heteroaryls and compositions containing them, for the treatment of general inflammation, arthritis, rheumatic diseases, osteoarthritis, inflammatory bowel disorders, inflammatory eye disorders, inflammatory or unstable bladder disorders, psoriasis, skin complaints with inflammatory components, chronic inflammatory conditions, including but not restricted to autoimmune diseases such as systemic lupus erythematosis (SLE), myestenia gravis, rheumatoid arthritis, acute disseminated encephalomyelitis, idiopathic thrombocytopenic purpura, multiples sclerosis, Sjoegren's syndrome and autoimmune hemolytic anemia, allergic conditions including all forms of hypersensitivity. The present invention also enables methods for treating cancers that are mediated, dependent on or associated with pi 105 activity, including but not restricted to leukemias, such as Acute Myeloid leukaemia (AML) Myelo-dysplastic syndrome (MDS) myelo-proliferative diseases (MPD) Chronic Myeloid Leukemia (CML) T-cell Acute Lymphoblastic leukaemia (T-ALL) B-cell Acute Lymphoblastic leukaemia (B-ALL) Non Hodgkins Lymphoma (NHL) B-cell lymphoma and solid tumors, such as breast cancer.

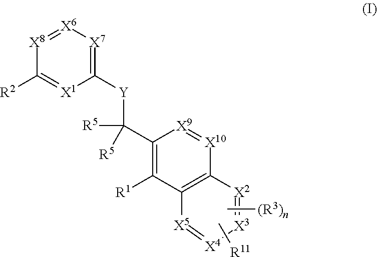

This application claims the benefit of U.S. Provisional Application No. 61/410,278 filed Nov. 4, 2011, which is hereby incorporated by reference.

The present invention relates generally to phosphatidylinositol 3-kinase (PI3K) enzymes, and more particularly to selective inhibitors of PI3K activity and to methods of using such materials.

### BACKGROUND OF THE INVENTION

Cell signaling via 3′-phosphorylated phosphoinositides has been implicated in a variety of cellular processes, e.g., malignant transformation, growth factor signaling, inflammation, and immunity (see Rameh et al., J. Biol Chem, 274:8347-8350 (1999) for a review). The enzyme responsible for generating these phosphorylated signaling products, phosphatidylinositol 3-kinase (PI 3-kinase; PI3K), was originally identified as an activity associated with viral oncoproteins and growth factor receptor tyrosine kinases that phosphorylates phosphatidylinositol (PI) and its phosphorylated derivatives at the 3′-hydroxyl of the inositol ring (Panayotou et al., Trends Cell Biol 2:358-60 (1992)).

The levels of phosphatidylinositol-3,4,5-triphosphate (PIP3), the primary product of PI 3-kinase activation, increase upon treatment of cells with a variety of stimuli. This includes signaling through receptors for the majority of growth factors and many inflammatory stimuli, hormones, neurotransmitters and antigens, and thus the activation of PI3Ks represents one, if not the most prevalent, signal transduction events associated with mammalian cell surface receptor activation (Cantley, Science 296:1655-1657 (2002); Vanhaesebroeck et al. Annu Rev. Biochem, 70: 535-602 (2001)). PI 3-kinase activation, therefore, is involved in a wide range of cellular responses including cell growth, migration, differentiation, and apoptosis (Parker et al., Current Biology, 5:577-99 (1995); Yao et al., Science, 267:2003-05 (1995)). Though the downstream targets of phosphorylated lipids generated following PI 3-kinase activation have not been fully characterized, it is known that pleckstrin-homology (PH) domain- and FYVE-finger domain-containing proteins are activated when binding to various phosphatidylinositol lipids (Sternmark et al., J Cell Sci, 112:4175-83 (1999); Lemmon et al., Trends Cell Biol, 7:237-42 (1997)). Two groups of PH-domain containing PI3K effectors have been studied in the context of immune cell signaling, members of the tyrosine kinase TEC family and the serine/threonine kinases of to AGC family. Members of the Tec family containing PH domains with apparent selectivity for PtdIns (3,4,5)P₃ include Tec, Btk, Itk and Etk. Binding of PH to PIP₃ is critical for tyrsosine kinase activity of the Tec family members (Schaeffer and Schwartzberg, Curr. Opin. Immunol. 12: 282-288 (2000)) AGC family members that are regulated by PI3K include the phosphoinositide-dependent kinase (PDK1), AKT (also termed PKB) and certain isoforms of protein kinase C (PKC) and S6 kinase. There are three isoforms of AKT and activation of AKT is strongly associated with PI3K-dependent proliferation and survival signals. Activation of AKT depends on phosphorylation by PDK1, which also has a 3-phosphoinositide-selective PH domain to recruit it to the membrane where it interacts with AKT. Other important PDK1 substrates are PKC and S6 kinase (Deane and Fruman, Annu Rev. Immunol. 22_{—}563-598 (2004)). In vitro, some isoforms of protein kinase C (PKC) are directly activated by PIP3. (Burgering et al., Nature, 376:599-602 (1995)).

Presently, the PI 3-kinase enzyme family has been divided into three classes based on their substrate specificities. Class I PI3Ks can phosphorylate phosphatidylinositol (PI), phosphatidylinositol-4-phosphate, and phosphatidyl-inositol-4,5-biphosphate (PIP2) to produce phosphatidylinositol-3-phosphate (PIP), phosphatidylinositol-3,4-biphosphate, and phosphatidylinositol-3,4,5-triphosphate, respectively. Class II PI3Ks phosphorylate PI and phosphatidyl-inositol-4-phosphate, whereas Class III PI3Ks can only phosphorylate PI.

The initial purification and molecular cloning of PI 3-kinase revealed that it was a heterodimer consisting of p85 and p110 subunits (Otsu et al., Cell, 65:91-104 (1991); Hiles et al., Cell, 70:419-29 (1992)). Since then, four distinct Class I PI3Ks have been identified, designated PI3K α, β, δ, and γ, each consisting of a distinct 110 kDa catalytic subunit and a regulatory subunit. More specifically, three of the catalytic subunits, i.e., p110α, p110β and p110δ, each interact with the same regulatory subunit, p85; whereas p110γ interacts with a distinct regulatory subunit, p101. As described below, the patterns of expression of each of these PI3Ks in human cells and tissues are also distinct. Though a wealth of information has been accumulated in recent past on the cellular functions of PI 3-kinases in general, the roles played by the individual isoforms are not fully understood.

Cloning of bovine p110α has been described. This protein was identified as related to the Saccharomyces cerevisiae protein: Vps34p, a protein involved in vacuolar protein processing. The recombinant p110α product was also shown to associate with p85α, to yield a PI3K activity in transfected COS-1 cells. See Hiles et al., Cell, 70, 419-29 (1992).

The cloning of a second human p110 isoform, designated p110β, is described in Hu et al., Mol Cell Biol, 13:7677-88 (1993). This isoform is said to associate with p85 in cells, and to be ubiquitously expressed, as p110β mRNA has been found in numerous human and mouse tissues as well as in human umbilical vein endothelial cells, Jurkat human leukemic T cells, 293 human embryonic kidney cells, mouse 3T3 fibroblasts, HeLa cells, and NBT2 rat bladder carcinoma cells. Such wide expression suggests that this isoform is broadly important in signaling pathways.

Identification of the p110δ isoform of PI 3-kinase is described in Chantry et al., J Biol Chem, 272:19236-41 (1997). It was observed that the human p110δ isoform is expressed in a tissue-restricted fashion. It is expressed at high levels in lymphocytes and lymphoid tissues and has been shown to play a key role in PI 3-kinase-mediated signaling in the immune system (Al-Alwan et al. JI 178: 2328-2335 (2007); Okkenhaug et al JI, 177: 5122-5128 (2006); Lee et al. PNAS, 103: 1289-1294 (2006)). P110δ has also been shown to be expressed at lower levels in breast cells, melanocytes and endothelial cells (Vogt et al. Virology, 344: 131-138 (2006) and has since been implicated in conferring selective migratory properties to breast cancer cells (Sawyer et al. Cancer Res. 63:1667-1675 (2003)). Details concerning the P110δ isoform also can be found in U.S. Pat. Nos. 5,858,753; 5,822,910; and 5,985,589. See also, Vanhaesebroeck et al., Proc Nat. Acad Sci USA, 94:4330-5 (1997), and international publication WO 97/46688.

In each of the PI3Kα, β, and δ subtypes, the p85 subunit acts to localize PI 3-kinase to the plasma membrane by the interaction of its SH2 domain with phosphorylated tyrosine residues (present in an appropriate sequence context) in target proteins (Rameh et al., Cell, 83:821-30 (1995)). Five isoforms of p85 have been identified (p85α, p85β, p55γ, p55α and p50α) encoded by three genes. Alternative transcripts of Pik3r1 gene encode the p85 α, p55 α and p50α proteins (Deane and Fruman, Annu Rev. Immunol. 22: 563-598 (2004)). p85α is ubiquitously expressed while p85β, is primarily found in the brain and lymphoid tissues (Volinia et al., Oncogene, 7:789-93 (1992)). Association of the p85 subunit to the PI 3-kinase p110α, β, or δ catalytic subunits appears to be required for the catalytic activity and stability of these enzymes. In addition, the binding of Ras proteins also upregulates PI 3-kinase activity.

The cloning of p110γ revealed still further complexity within the PI3K family of enzymes (Stoyanov et al., Science, 269:690-93 (1995)). The p110γ isoform is closely related to p110α and p110β (45-48% identity in the catalytic domain), but as noted does not make use of p85 as a targeting subunit. Instead, p110γ binds a p101 regulatory subunit that also binds to the βγ subunits of heterotrimeric G proteins. The p101 regulatory subunit for PI3 Kgamma was originally cloned in swine, and the human ortholog identified subsequently (Krugmann et al., J Biol Chem, 274:17152-8 (1999)). Interaction between the N-terminal region of p101 with the N-terminal region of p110γ is known to activate PI3Kγ through Gβγ. Recently, a p101-homologue has been identified, p84 or p87^{PIKAP} (PI3Kγ adapter protein of 87 kDa) that binds p110γ (Voigt et al. JBC, 281: 9977-9986 (2006), Suire et al. Curr. Biol. 15: 566-570 (2005)). p87^{PIKAP} is homologous to p101 in areas that bind p110γ and Gβγ and also mediates activation of p110γ downstream of G-protein-coupled receptors. Unlike p101, p87^{PIKAP} is highly expressed in the heart and may be crucial to PI3Kγ cardiac function.

A constitutively active PI3K polypeptide is described in international publication WO 96/25488. This publication discloses preparation of a chimeric fusion protein in which a 102-residue fragment of p85 known as the inter-SH2 (iSH2) region is fused through a linker region to the N-terminus of murine p110. The p85 iSH2 domain apparently is able to activate PI3K activity in a manner comparable to intact p85 (Klippel et al., Mol Cell Biol, 14:2675-85 (1994)).

Thus, PI 3-kinases can be defined by their amino acid identity or by their activity. Additional members of this growing gene family include more distantly related lipid and protein kinases including Vps34 TOR1, and TOR2 of Saccharomyces cerevisiae (and their mammalian homologs such as FRAP and mTOR), the ataxia telangiectasia gene product (ATR) and the catalytic subunit of DNA-dependent protein kinase (DNA-PK). See generally, Hunter, Cell, 83:1-4 (1995).

PI 3-kinase is also involved in a number of aspects of leukocyte activation. A p85-associated PI 3-kinase activity has been shown to physically associate with the cytoplasmic domain of CD28, which is an important costimulatory molecule for the activation of T-cells in response to antigen (Pages et al., Nature, 369:327-29 (1994); Rudd, Immunity, 4:527-34 (1996)). Activation of T cells through CD28 lowers the threshold for activation by antigen and increases the magnitude and duration of the proliferative response. These effects are linked to increases in the transcription of a number of genes including interleukin-2 (IL2), an important T cell growth factor (Fraser et al., Science, 251:313-16 (1991)). Mutation of CD28 such that it can no longer interact with PI 3-kinase leads to a failure to initiate IL2 production, suggesting a critical role for PI 3-kinase in T cell activation.

Specific inhibitors against individual members of a family of enzymes provide invaluable tools for deciphering functions of each enzyme. Two compounds, LY294002 and wortmannin, have been widely used as PI 3-kinase inhibitors. These compounds, however, are nonspecific PI3K inhibitors, as they do not distinguish among the four members of Class I PI 3-kinases. For example, the IC₅₀ values of wortmannin against each of the various Class I PI 3-kinases are in the range of 1-10 nM. Similarly, the IC₅₀ values for LY294002 against each of these PI 3-kinases is about 1 μM (Fruman et al., Ann Rev Biochem, 67:481-507 (1998)). Hence, the utility of these compounds in studying the roles of individual Class I PI 3-kinases is limited.

Based on studies using wortmannin, there is evidence that PI 3-kinase function also is required for some aspects of leukocyte signaling through G-protein coupled receptors (Thelen et al., Proc Natl Acad Sci USA, 91:4960-64 (1994)). Moreover, it has been shown that wortmannin and LY294002 block neutrophil migration and superoxide release. However, inasmuch as these compounds do not distinguish among the various isoforms of PI3K, it remains unclear from these studies which particular PI3K isoform or isoforms are involved in these phenomena and what functions the different Class I PI3K enzymes perform in both normal and diseased tissues in general. The co-expression of several PI3K isoforms in most tissues has confounded efforts to segregate the activities of each enzyme until recently.

The separation of the activities of the various PI3K isozymes has been advanced recently with the development of genetically manipulated mice that allowed the study of isoform-specific knock-out and kinase dead knock-in mice and the development of more selective inhibitors for some of the different isoforms. P110α and p110β knockout mice have been generated and are both embryonic lethal and little information can be obtained from these mice regarding the expression and function of p110 alpha and beta (Bi et al. Mamm. Genome, 13:169-172 (2002); Bi et al. J. Biol. Chem. 274:10963-10968 (1999)). More recently, p110α kinase dead knock in mice were generated with a single point mutation in the DFG motif of the ATP binding pocket (p110αD^{933A}) that impairs kinase activity but preserves mutant p110α kinase expression. In contrast to knock out mice, the knockin approach preserves signaling complex stoichiometry, scaffold functions and mimics small molecule approaches more realistically than knock out mice. Similar to the p110α KO mice, p110αD^{933A} homozygous mice are embryonic lethal. However, heterozygous mice are viable and fertile but display severely blunted signaling via insulin-receptor substrate (IRS) proteins, key mediators of insulin, insulin-like growth factor-1 and leptin action. Defective responsiveness to these hormones leads to hyperinsulinaemia, glucose intolerance, hyperphagia, increase adiposity and reduced overall growth in heterozygotes (Foukas, et al. Nature, 441: 366-370 (2006)). These studies revealed a defined, non-redundant role for p110α as an intermediate in IGF-1, insulin and leptin signaling that is not substituted for by other isoforms. We will have to await the description of the p110β kinase-dead knock in mice to further understand the function of this isoform (mice have been made but not yet published; Vanhaesebroeck).

P110γ knock out and kinase-dead knock in mice have both been generated and overall show similar and mild phenotypes with primary defects in migration of cells of the innate immune system and a defect in thymic development of T cells (Li et al. Science, 287: 1046-1049 (2000), Sasaki et al. Science, 287: 1040-1046 (2000), Patrucco et al. Cell, 118: 375-387 (2004)).

Similar to p110γ, PI3K delta knock out and kinase-dead knock-in mice have been made and are viable with mild and like phenotypes. The p110δ^{D910A} mutant knock in mice demonstrated an important role for delta in B cell development and function, with marginal zone B cells and CD5+ B1 cells nearly undetectable, and B- and T cell antigen receptor signaling (Clayton et al. J. Exp. Med. 196:753-763 (2002); Okkenhaug et al. Science, 297: 1031-1034 (2002)). The p110δ^{D910A} mice have been studied extensively and have elucidated the diverse role that delta plays in the immune system. T cell dependent and T cell independent immune responses are severely attenuated in p110δ^{D910A} and secretion of TH1 (INF-γ) and TH2 cytokine (IL-4, IL-5) are impaired (Okkenhaug et al. J. Immunol. 177: 5122-5128 (2006)). A human patient with a mutation in p110δ has also recently been described. A taiwanese boy with a primary B cell immunodeficiency and a gamma-hypoglobulinemia of previously unkown aetiology presented with a single base-pair substitution, m.3256G to A in codon 1021 in exon 24 of p110δ. This mutation resulted in a mis-sense amino acid substitution (E to K) at codon 1021, which is located in the highly conserved catalytic domain of p110δ protein. The patient has no other identified mutations and his phenotype is consistent with p110δ deficiency in mice as far as studied. (Jou et al. Int. J. Immunogenet. 33: 361-369 (2006)).

Isoform-selective small molecule compounds have been developed with varying success to all Class I PI3 kinase isoforms (Ito et al. J. Pharm. Exp. Therapeut., 321:1-8 (2007)). Inhibitors to alpha are desirable because mutations in p110α have been identified in several solid tumors; for example, an amplification mutation of alpha is associated with 50% of ovarian, cervical, lung and breast cancer and an activation mutation has been described in more than 50% of bowel and 25% of breast cancers (Hennessy et al. Nature Reviews, 4: 988-1004 (2005)). Yamanouchi has developed a compound YM-024 that inhibits alpha and delta equi-potently and is 8- and 28-fold selective over beta and gamma respectively (Ito et al. J. Pharm. Exp. Therapeut., 321:1-8 (2007)).

P110β is involved in thrombus formation (Jackson et al. Nature Med. 11: 507-514 (2005)) and small molecule inhibitors specific for this isoform are thought after for indication involving clotting disorders (TGX-221: 0.007 uM on beta; 14-fold selective over delta, and more than 500-fold selective over gamma and alpha) (Ito et al. J. Pharm. Exp. Therapeut., 321:1-8 (2007)).

Selective compounds to p110γ are being developed by several groups as immunosuppressive agents for autoimmune disease (Rueckle et al. Nature Reviews, 5: 903-918 (2006)). Of note, AS 605240 has been shown to be efficacious in a mouse model of rheumatoid arthritis (Camps et al. Nature Medicine, 11: 936-943 (2005)) and to delay onset of disease in a model of systemic lupus erythematosis (Barber et al. Nature Medicine, 11: 933-935 (205)).

Delta-selective inhibitors have also been described recently. The most selective compounds include the quinazolinone purine inhibitors (PIK39 and IC87114). IC87114 inhibits p110δ in the high nanomolar range (triple digit) and has greater than 100-fold selectivity against p110α, is 52 fold selective against p110β but lacks selectivity against p110γ (approx. 8-fold). It shows no activity against any protein kinases tested (Knight et al. Cell, 125: 733-747 (2006)). Using delta-selective compounds or genetically manipulated mice (p110δ^{D910A}) it was shown that in addition to playing a key role in B and T cell activation, delta is also partially involved in neutrophil migration and primed neutrophil respiratory burst and leads to a partial block of antigen-IgE mediated mast cell degranulation (Condliffe et al. Blood, 106: 1432-1440 (2005); Ali et al. Nature, 431: 1007-1011 (2002)). Hence p110δ is emerging as an important mediator of many key inflammatory responses that are also known to participate in aberrant inflammatory conditions, including but not limited to autoimmune disease and allergy. To support this notion, there is a growing body of p110δ target validation data derived from studies using both genetic tools and pharmacologic agents. Thus, using the delta-selective compound IC 87114 and the p110δ^{D910A} mice, Ali et al. (Nature, 431: 1007-1011 (2002)) have demonstrated that delta plays a critical role in a murine model of allergic disease. In the absence of functional delta, passive cutaneous anaphylaxis (PCA) is significantly reduced and can be attributed to a reduction in allergen-IgE induced mast cell activation and degranulation. In addition, inhibition of delta with IC 87114 has been shown to significantly ameliorate inflammation and disease in a murine model of asthma using ovalbumin-induced airway inflammation (Lee et al. FASEB, 20: 455-465 (2006). These data utilizing compound were corroborated in p110δ^{D910A} mutant mice using the same model of allergic airway inflammation by a different group (Nashed et al. Eur. J. Immunol. 37:416-424 (2007)).

There exists a need for further characterization of PI3Kδ function in inflammatory and auto-immune settings. Furthermore, our understanding of PI3Kδ requires further elaboration of the structural interactions of p110δ, both with its regulatory subunit and with other proteins in the cell. There also remains a need for more potent and selective or specific inhibitors of PI3K delta, in order to avoid potential toxicology associated with activity on isozymes p110 alpha (insulin signaling) and beta (platelet activation). In particular, selective or specific inhibitors of PI3Kδ are desirable for exploring the role of this isozyme further and for development of superior pharmaceuticals to modulate the activity of the isozyme.

### SUMMARY

The present invention comprises a new class of compounds having the general formula

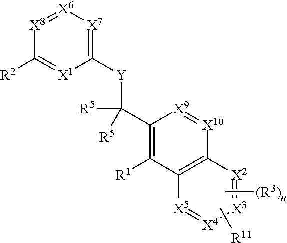

which are useful to inhibit the biological activity of human PI3Kδ. Another aspect of the invention is to provide compounds that inhibit PI3Kδ selectively while having relatively low inhibitory potency against the other PI3K isoforms. Another aspect of the invention is to provide methods of characterizing the function of human PI3Kδ. Another aspect of the invention is to provide methods of selectively modulating human PI3Kδ activity, and thereby promoting medical treatment of diseases mediated by PI3Kδ dysfunction. Other aspects and advantages of the invention will be readily apparent to the artisan having ordinary skill in the art.

### DETAILED DESCRIPTION

One aspect of the present invention relates to compounds having the structure:

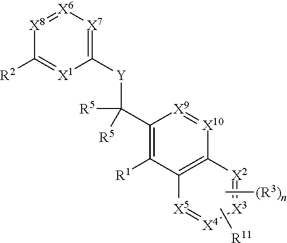

or any pharmaceutically-acceptable salt thereof, wherein:
X¹ is C(R¹⁰) or N;
X² is C or N;
X³ is C or N;
X⁴ is C or N;
X⁵ is C or N; wherein at least two of X², X³, X⁴ and X⁵ are C;
X⁶ is C(R⁶) or N;
X⁷ is C(R⁷) or N;
X⁸ is C(R¹⁰) or N; wherein no more than two of X¹, X⁶, X⁷ and X⁸ are N;
X⁹ is C(R⁴) or N;
X¹⁰ is C(R⁴) or N;
Y is N(R), O or S;
n is 0, 1, 2 or 3;
R¹ is selected from H, halo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, OC(═O)R^{a}, OC(═O)NR^{a}R^{a}, OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a}, —NR^{a}C₂₋₆alkOR^{a}, —NR^{a}C₂₋₆alkCO₂R^{a}, —NR^{a}C₂₋₆alkSO₂R^{b}, —CH₂C(═O)R^{a}, —CH₂C(═O)OR^{a}, —CH₂C(═O)NR^{a}R^{a}, —CH₂C(═NR^{a})NR^{a}R^{a}, —CH₂OR^{a}, —CH₂OC(═O)R^{a}, —CH₂C(═O)NR^{a}R^{a}, —CH₂C(═O)N(R^{a})S(═O)₂R^{a}, —CH₂OC₂₋₆alkNR^{a}R^{a}, —CH₂OC₂₋₆alkOR^{a}, —CH₂SR^{a}, —CH₂S(═O)R^{a}, —CH₂S(═O)₂R^{b}, —CH₂S(═O)₂NR^{a}R^{a}, —CH₂S(═O)₂NR^{a})C(═O)R^{a}, —CH₂S(═O)₂N(R^{a})C(═O)OR^{a}, —CH₂S(═O)₂N(R^{a})C(═O)NR^{a}R^{a}, —CH₂NR^{a}R^{a}, —CH₂N(R^{a})C(═O)R^{a}, —CH₂N(R^{a})C(═O)OR^{a}, —CH₂N(R^{a})C(═O)NR^{a}R^{a}, —CH₂N(R^{a})C(═NR^{a})NR^{a}R^{a}, —CH₂N(R^{a})S(═O)₂R^{a}, —CH₂N(R^{a})S(═O)₂NR^{a}R^{a}, —CH₂NR^{a}C₂₋₆alkNR^{a}R^{a}, —CH₂NR^{a}C₂₋₆alkOR^{a}, —CH₂NR^{a}C₂₋₆alkCO₂R^{a} and —CH₂NR^{a}C₂₋₆alkSO₂R^{b}; or R¹ is a direct-bonded, C₁₋₄alk-linked, OC₁₋₂alk-linked, C₁₋₂alkO-linked, N(R^{a})-linked or O-linked saturated, partially-saturated or unsaturated 3-, 4-, 5-, 6- or 7-membered monocyclic or 8-, 9-, 10- or 11-membered bicyclic ring containing 0, 1, 2, 3 or 4 atoms selected from N, O and S, but containing no more than one O or S atom, substituted by 0, 1, 2 or 3 substituents independently selected from halo, C₁₋₆alk, C₁₋₄haloalk, cyano, nitro, —OC(═O)NR^{a}R^{a}, OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}, wherein the available carbon atoms of the ring are additionally substituted by 0, 1 or 2 oxo or thioxo groups, and wherein the ring is additionally substituted by 0 or 1 directly bonded, SO₂ linked, C(═O) linked or CH₂ linked group selected from phenyl, pyridyl, pyrimidyl, morpholino, piperazinyl, piperadinyl, pyrrolidinyl, cyclopentyl, cyclohexyl all of which are further substituted by 0, 1, 2 or 3 groups selected from halo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —SR^{a}, —S(═O)R^{a}, —S(═O)₂R^{a}, —S(═O)₂NR^{a}R^{a}, —NR^{a}R^{a}, and —N(R^{a})C(═O)R^{a};
R² is selected from H, halo, C₁₋₆alk, C₁₋₄haloalk, cyano, nitro, OR^{a}, NR^{a}R^{a}, —C(═O)R^{a}, —C(═O)OR^{a}, —C(═O)NR^{a}R^{a}, —C(═NR^{a})NR^{a}R^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a} and —S(═O)₂N(R^{a})C(═O)NR^{a}R^{a};
R³ is, independently, in each instance, H, halo, nitro, cyano, C₁₋₄alk, OC₁₋₄alk, OC₁₋₄haloalk, NHC₁₋₄alk, N(C₁₋₄alk)C₁₋₄alk or C₁₋₄haloalk;
R⁴ is, independently, in each instance, H, halo, nitro, cyano, C₁₋₄alk, OC₁₋₄alk, OC₁₋₄haloalk, NHC₁₋₄alk, N(C₁₋₄alk)C₁₋₄alk, C₁₋₄haloalk or an unsaturated 5-, 6- or 7-membered monocyclic ring containing 0, 1, 2, 3 or 4 atoms selected from N, O and S, but containing no more than one O or S, the ring being substituted by 0, 1, 2 or 3 substituents selected from halo, C₁₋₄alk, C₁₋₃haloalk, —OC₁₋₄alk, —NH₂, —NHC₁₋₄alk, —N(C₁₋₄alk)C₁₋₄alk;
R⁵ is, independently, in each instance, H, halo, C₁₋₆alk, C₁₋₄haloalk, or C₁₋₆alk substituted by 1, 2 or 3 substituents selected from halo, cyano, OH, OC₁₋₄alk, C₁₋₄alk, C₁₋₃haloalk, OC₁₋₄alk, NH₂, NHC₁₋₄alk and N(C₁₋₄alk)C₁₋₄alk; or both R⁵ groups together form a C₃₋₆spiroalk substituted by 0, 1, 2 or 3 substituents selected from halo, cyano, OH, OC₁₋₄alk, C₁₋₄alk, C₁₋₃haloalk, OC₁₋₄alk, NH₂, NHC₁₋₄alk and N(C₁₋₄alk)C₁₋₄alk;
R⁶ is H, halo, NHR⁹ or OH, cyano, OC₁₋₄alk, C₁₋₄alk, C₁₋₃haloalk, OC₁₋₄alk, —C(═O)OR^{a}, —C(═O)N(R^{a})R^{a} or —N(R^{a})C(═O)R^{b};
R⁷ is selected from H, halo, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a}, —NR^{a}C₂₋₆alkOR^{a} and C₁₋₆alk, wherein the C₁₋₆alk is substituted by 0, 1, 2 or 3 substituents selected from halo, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, OC(═O)NR^{a}R^{a}, —OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}, and the C₁₋₆alk is additionally substituted by 0 or 1 saturated, partially-saturated or unsaturated 5-, 6- or 7-membered monocyclic rings containing 0, 1, 2, 3 or 4 atoms selected from N, O and S, but containing no more than one O or S, wherein the available carbon atoms of the ring are substituted by 0, 1 or 2 oxo or thioxo groups, wherein the ring is substituted by 0, 1, 2 or 3 substituents independently selected from halo, nitro, cyano, C₁₋₄alk, OC₁₋₄alk, OC₁₋₄haloalk, NHC₁₋₄alk, N(C₁₋₄alk)C₁₋₄alk and C₁₋₄haloalk; or R⁷ and R⁸ together form a —C═N— bridge wherein the carbon atom is substituted by H, halo, cyano, or a saturated, partially-saturated or unsaturated 5-, 6- or 7-membered monocyclic ring containing 0, 1, 2, 3 or 4 atoms selected from N, O and S, but containing no more than one O or S, wherein the available carbon atoms of the ring are substituted by 0, 1 or 2 oxo or thioxo groups, wherein the ring is substituted by 0, 1, 2, 3 or 4 substituents selected from halo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, OC(═O)R^{a}, OC(═O)NR^{a}R^{a}, OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}; or R⁷ and R⁹ together form a —N═C— bridge wherein the carbon atom is substituted by H, halo, C₁₋₆alk, C₁₋₄haloalk, cyano, nitro, OR^{a}, NR^{a}R^{a}, —C(═O)R^{a}, —C(═O)OR^{a}, —C(═O)NR^{a}R^{a}, —C(═NR^{a})NR^{a}R^{a}, —S(═O)R^{a}, —S(═O)₂R^{a} or —S(═O)₂NR^{a}R^{a};
R⁸ is H, C₁₋₆alk, C(═O)N(R^{a})R^{a}, C(═O)R^{b} or C₁₋₄haloalk;
R⁹ is H, C₁₋₆alk or C₁₋₄haloalk;
R¹⁰ is in each instance H, halo, C₁₋₃alk, C₁₋₃haloalk or cyano;
R¹¹ is selected from H, halo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{b}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a}, —NR^{a}C₂₋₆alkOR^{a}, —NR^{a}C₂₋₆alkCO₂R^{a}, —NR^{a}C₂₋₆alkSO₂R^{b}, —CH₂C(═O)R^{a}, —CH₂C(═O)OR^{a}, —CH₂C(═O)NR^{a}R^{a}, —CH₂C(═NR^{a})NR^{a}R^{a}, —CH₂OR^{a}, —CH₂C(═O)R^{a}, —CH₂C(═O)NR^{a}R^{a}, —CH₂C(═O)N(R^{a})S(═O)₂R^{a}, —CH₂OC₂₋₆alkNR^{a}R^{a}, —CH₂OC₂₋₆alkOR^{a}, —CH₂SR^{a}, —CH₂S(═O)R^{a}, —CH₂S(═O)₂R^{b}, —CH₂S(═O)₂NR^{a}R^{a}, —CH₂S(═O)₂N(R^{a})C(═O)R^{a}, —CH₂S(═O)₂N(R^{a})C(═O)OR^{a}, —CH₂S(═O)₂N(R^{a})C(═O)NR^{a}R^{a}, —CH₂NR^{a}R^{a}, —CH₂N(R^{a})C(═O)R^{a}, —CH₂N(R^{a})C(═O)OR^{a}, —CH₂N(R^{a})C(═O)NR^{a}R^{a}, —CH₂N(R^{a})C(═NR^{a})NR^{a}R^{a}, —CH₂N(R^{a})S(═O)₂R^{a}, —CH₂N(R^{a})S(═O)₂NR^{a}R^{a}, —CH₂NR^{a}C₂₋₆alkNR^{a}R^{a}, —CH₂NR^{a}C₂₋₆alkOR^{a}, —CH₂NR^{a}C₂₋₆alkCO₂R^{a}, —CH₂NR^{a}C₂₋₆alkSO₂R^{b}, —CH₂R^{c}, —C(═O)R^{c} and —C(═O)N(R^{a})R^{c};
R^{a} is independently, at each instance, H or R^{b};
R^{b} is independently, at each instance, phenyl, benzyl or C₁₋₆alk, the phenyl, benzyl and C₁₋₆alk being substituted by 0, 1, 2 or 3 substituents selected from halo, C₁₋₄alk, C₁₋₃haloalk, —OH, —OC₁₋₄alk, —NH₂, —NHC₁₋₄alk and —N(C₁₋₄alk)C₁₋₄alk; and
R^{c} is a saturated or partially-saturated 4-, 5- or 6-membered ring containing 1, 2 or 3 heteroatoms selected from N, O and S, the ring being substituted by 0, 1, 2 or 3 substituents selected from halo, C₁₋₄alk, C₁₋₃haloalk, —OC₁₋₄alk, —NH₂, —NHC₁₋₄alk and —N(C₁₋₄alk)C₁₋₄alk.

In another embodiment, in conjunction with any of the above or below embodiments, X⁹ is N and X¹⁰ is C(R⁴).

In another embodiment, in conjunction with any of the above or below embodiments, X⁹ is N and X¹⁰ is N.

In another embodiment, in conjunction with any of the above or below embodiments, X⁹ is C(R⁴) and X¹⁰ is N.

In another embodiment, in conjunction with any of the above or below embodiments, X⁹ is C(R⁴) and X¹⁰ is C(R⁴).

In another embodiment, in conjunction with any of the above or below embodiments, X¹ is N.

In another embodiment, in conjunction with any of the above or below embodiments, X¹ is C(R¹⁰).

In another embodiment, in conjunction with any of the above or below embodiments,
X² is C(R⁴);
X³ is C(R⁵);
X⁴ is C(R⁵); and
X⁵ is C(R⁴).

In another embodiment, in conjunction with any of the above or below embodiments,
X² is N;
X³ is C(R⁵);
X⁴ is C(R⁵); and
X⁵ is C(R⁴).

In another embodiment, in conjunction with any of the above or below embodiments,
X² is C(R⁴);
X³ is N;
X⁴ is C(R⁵); and
X⁵ is C(R⁴).

In another embodiment, in conjunction with any of the above or below embodiments,
X² is C(R⁴);
X³ is C(R⁵);
X⁴ is N; and
X⁵ is C(R⁴).

In another embodiment, in conjunction with any of the above or below embodiments,
X² is C(R⁴);
X³ is C(R⁵);
X⁴ is C(R⁵); and
X⁵ is N.

In another embodiment, in conjunction with any of the above or below embodiments, R¹ is selected from C₁₋₆alk and C₁₋₄haloalk.

In another embodiment, in conjunction with any of the above or below embodiments, R¹ is cyclopropyl.

In another embodiment, in conjunction with any of the above or below embodiments, R¹ is a direct-bonded unsaturated 5-, 6- or 7-membered monocyclic or 8-, 9-, 10- or 11-membered bicyclic ring containing 0, 1, 2, 3 or 4 atoms selected from N, O and S, but containing no more than one O or S atom, substituted by 0, 1, 2 or 3 substituents independently selected from halo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, OC(═O)R^{a}, OC(═O)NR^{a}R^{a}, OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}, wherein the available carbon atoms of the ring are additionally substituted by 0, 1 or 2 oxo or thioxo groups.

In another embodiment, in conjunction with any of the above or below embodiments, R¹ is a direct-bonded unsaturated 5-, 6- or 7-membered monocyclic ring containing 0, 1, 2, 3 or 4 atoms selected from N, O and S, but containing no more than one O or S atom, substituted by 0, 1, 2 or 3 substituents independently selected from halo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, OC(═O)R^{a}, OC(═O)NR^{a}R^{a}, OC(═O)N(R^{a})S(═O)₂R^{a}, —OC₂₋₆alkNR^{a}R^{a}, —OC₂₋₆alkOR^{a}, —SW, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}, wherein the available carbon atoms of the ring are additionally substituted by 0, 1 or 2 oxo or thioxo groups.

In another embodiment, in conjunction with any of the above or below embodiments, R¹ is phenyl or pyridine, both of which are substituted by 0, 1, 2 or 3 substituents independently selected from halo, C₁₋₆alk and C₁₋₄haloalk.

In another embodiment, in conjunction with any of the above or below embodiments, R¹ is a methylene-linked saturated, partially-saturated or unsaturated 5-, 6- or 7-membered monocyclic or 8-, 9-, 10- or 11-membered bicyclic ring containing 0, 1, 2, 3 or 4 atoms selected from N, O and S, but containing no more than one O or S atom, substituted by 0, 1, 2 or 3 substituents independently selected from halo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}, wherein the available carbon atoms of the ring are additionally substituted by 0, 1 or 2 oxo or thioxo groups.

In another embodiment, in conjunction with any of the above or below embodiments, R¹ is an ethylene-linked saturated, partially-saturated or unsaturated 5-, 6- or 7-membered monocyclic or 8-, 9-, 10- or 11-membered bicyclic ring containing 0, 1, 2, 3 or 4 atoms selected from N, O and S, but containing no more than one O or S atom, substituted by 0, 1, 2 or 3 substituents independently selected from halo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}, wherein the available carbon atoms of the ring are additionally substituted by 0, 1 or 2 oxo or thioxo groups.

In another embodiment, in conjunction with any of the above or below embodiments, R² is selected from halo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}.

In another embodiment, in conjunction with any of the above or below embodiments, R² is selected from halo, C₁₋₆alk and C₁₋₄haloalk.

In another embodiment, in conjunction with any of the above or below embodiments, R² is H.

In another embodiment, in conjunction with any of the above or below embodiments, R¹ and R² together form a saturated or partially-saturated 2-, 3-, 4- or 5-carbon bridge substituted by 0, 1, 2 or 3 substituents selected from halo, cyano, OH, OC₁₋₄alk, C₁₋₄alk, C₁₋₃haloalk, OC₁₋₄alk, NH₂, NHC₁₋₄alk and N(C₁₋₄alk)C₁₋₄alk.

In another embodiment, in conjunction with any of the above or below embodiments, R³ is selected from saturated, partially-saturated or unsaturated 5-, 6- or 7-membered monocyclic ring containing 0, 1, 2, 3 or 4 atoms selected from N, O and S, but containing no more than one O or S, wherein the available carbon atoms of the ring are substituted by 0, 1 or 2 oxo or thioxo groups, wherein the ring is additionally substituted by 0, 1, 2 or 3 substituents independently selected from halo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}.

In another embodiment, in conjunction with any of the above or below embodiments, R³ is selected from saturated 5-, 6- or 7-membered monocyclic ring containing 1, 2, 3 or 4 atoms selected from N, O and S, but containing no more than one O or S, wherein the available carbon atoms of the ring are substituted by 0, 1 or 2 oxo or thioxo groups, wherein the ring is additionally substituted by 0, 1, 2 or 3 substituents independently selected from halo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, OC(═O)R^{a}, OC(═O)NR^{a}R^{a}, OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}.

In another embodiment, in conjunction with any of the above or below embodiments, R³ is selected from saturated 5-, 6- or 7-membered monocyclic ring containing 1, 2, 3 or 4 atoms selected from N, O and S, but containing no more than one O or S, wherein the ring is substituted by 0, 1, 2 or 3 substituents independently selected from halo, C₁₋₆alk and C₁₋₄haloalk.

In another embodiment, in conjunction with any of the above or below embodiments, R³ is selected from saturated 6-membered monocyclic ring containing 1 or 2 atoms selected from N, O and S, but containing no more than one O or S, wherein the ring is substituted by 0, 1, 2 or 3 substituents independently selected from halo, C₁₋₆alk and C₁₋₄haloalk.

In another embodiment, in conjunction with any of the above or below embodiments, R³ is selected from saturated 6-membered monocyclic ring containing 1 or 2 atoms selected from N, O and S, but containing no more than one O or S.

In another embodiment, in conjunction with any of the above or below embodiments, R³ is selected from halo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, OC(═O)R^{a}, OC(═O)NR^{a}R^{a}, OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}.

In another embodiment, in conjunction with any of the above or below embodiments, R⁸ is selected from saturated, partially-saturated or unsaturated 5-, 6- or 7-membered monocyclic or 8-, 9-, 10- or 11-membered bicyclic ring containing 0, 1, 2, 3 or 4 atoms selected from N, O and S, but containing no more than one O or S, wherein the available carbon atoms of the ring are substituted by 0, 1 or 2 oxo or thioxo groups, wherein the ring is substituted by 0 or 1 R² substituents, and the ring is additionally substituted by 0, 1, 2 or 3 substituents independently selected from halo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, OC(═O)R^{a}, OC(═O)NR^{a}R^{a}, —OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}.

In another embodiment, in conjunction with any of the above or below embodiments, R⁸ is selected from saturated, partially-saturated or unsaturated 5-, 6- or 7-membered monocyclic ring containing 0, 1, 2, 3 or 4 atoms selected from N, O and S, but containing no more than one O or S, wherein the available carbon atoms of the ring are substituted by 0, 1 or 2 oxo or thioxo groups, wherein the ring is substituted by 0, 1, 2 or 3 substituents independently selected from halo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, OC(═O)R^{a}, —OC(═O)NR^{a}R^{a}, —OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}.

In another embodiment, in conjunction with any of the above or below embodiments, R⁸ is selected from saturated 5-, 6- or 7-membered monocyclic ring containing 1 or 2 atoms selected from N, O and S, but containing no more than one O or S, wherein the ring is substituted by 0, 1, 2 or 3 substituents independently selected from halo, C₁₋₆alk and C₁₋₄haloalk.

In another embodiment, in conjunction with any of the above or below embodiments, R⁸ is selected from halo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, OC(═O)R^{a}, OC(═O)NR^{a}R^{a}, OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}.

In another embodiment, in conjunction with any of the above or below embodiments, R⁸ is cyano.

Another aspect of the invention relates to a method of treating PI3K-mediated conditions or disorders.

In certain embodiments, the PI3K-mediated condition or disorder is selected from rheumatoid arthritis, ankylosing spondylitis, osteoarthritis, psoriatic arthritis, psoriasis, inflammatory diseases, and autoimmune diseases. In other embodiments, the PI3K-mediated condition or disorder is selected from cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease. In still other embodiments, the PI3K-mediated condition or disorder is selected from cancer, colon cancer, glioblastoma, endometrial carcinoma, hepatocellular cancer, lung cancer, melanoma, renal cell carcinoma, thyroid carcinoma, cell lymphoma, lymphoproliferative disorders, small cell lung cancer, squamous cell lung carcinoma, glioma, breast cancer, prostate cancer, ovarian cancer, cervical cancer, and leukemia. In yet another embodiment, the PI3K-mediated condition or disorder is selected from type II diabetes. In still other embodiments, the PI3K-mediated condition or disorder is selected from respiratory diseases, bronchitis, asthma, and chronic obstructive pulmonary disease. In certain embodiments, the subject is a human.

Another aspect of the invention relates to the treatment of rheumatoid arthritis, ankylosing spondylitis, osteoarthritis, psoriatic arthritis, psoriasis, inflammatory diseases or autoimmune diseases comprising the step of administering a compound according to any of the above embodiments.

Another aspect of the invention relates to the treatment of rheumatoid arthritis, ankylosing spondylitis, osteoarthritis, psoriatic arthritis, psoriasis, inflammatory diseases and autoimmune diseases, inflammatory bowel disorders, inflammatory eye disorders, inflammatory or unstable bladder disorders, skin complaints with inflammatory components, chronic inflammatory conditions, autoimmune diseases, systemic lupus erythematosis (SLE), myestenia gravis, rheumatoid arthritis, acute disseminated encephalomyelitis, idiopathic thrombocytopenic purpura, multiples sclerosis, Sjoegren's syndrome and autoimmune hemolytic anemia, allergic conditions and hypersensitivity, comprising the step of administering a compound according to any of the above or below embodiments.

Another aspect of the invention relates to the treatment of cancers that are mediated, dependent on or associated with p110δ activity, comprising the step of administering a compound according to any of the above or below embodiments.

Another aspect of the invention relates to the treatment of cancers are selected from acute myeloid leukaemia, myelo-dysplastic syndrome, myelo-proliferative diseases, chronic myeloid leukaemia, T-cell acute lymphoblastic leukaemia, B-cell acute lymphoblastic leukaemia, non-hodgkins lymphoma, B-cell lymphoma, solid tumors and breast cancer, comprising the step of administering a compound according to any of the above or below embodiments.

Another aspect of the invention relates to a pharmaceutical composition comprising a compound according to any of the above embodiments and a pharmaceutically-acceptable diluent or carrier.

Another aspect of the invention relates to the use of a compound according to any of the above embodiments as a medicament.

Another aspect of the invention relates to the use of a compound according to any of the above embodiments in the manufacture of a medicament for the treatment of rheumatoid arthritis, ankylosing spondylitis, osteoarthritis, psoriatic arthritis, psoriasis, inflammatory diseases, and autoimmune diseases.

The compounds of this invention may have in general several asymmetric centers and are typically depicted in the form of racemic mixtures. This invention is intended to encompass racemic mixtures, partially racemic mixtures and separate enantiomers and diasteromers.

Unless otherwise specified, the following definitions apply to terms found in the specification and claims:

“C_{α-β}alk” means an alkyl group comprising a minimum of α and a maximum of β carbon atoms in a branched, cyclical or linear relationship or any combination of the three, wherein α and β represent integers. The alkyl groups described in this section may also contain one or two double or triple bonds. Examples of C₁₋₆alk include, but are not limited to the following:

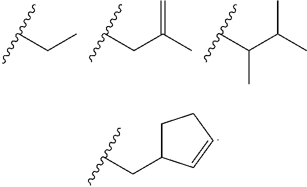

“Benzo group”, alone or in combination, means the divalent radical C₄H₄═, one representation of which is —CH═CH—CH═CH—, that when vicinally attached to another ring forms a benzene-like ring—for example tetrahydronaphthylene, indole and the like.

The terms “oxo” and “thioxo” represent the groups ═O (as in carbonyl) and ═S (as in thiocarbonyl), respectively.

“Halo” or “halogen” means a halogen atoms selected from F, Cl, Br and I.

“C_{V-W}haloalk” means an alk group, as described above, wherein any number—at least one—of the hydrogen atoms attached to the alkyl chain are replaced by F, Cl, Br or I.

“Heterocycle” means a ring comprising at least one carbon atom and at least one other atom selected from N, O and S. Examples of heterocycles that may be found in the claims include, but are not limited to, the following:

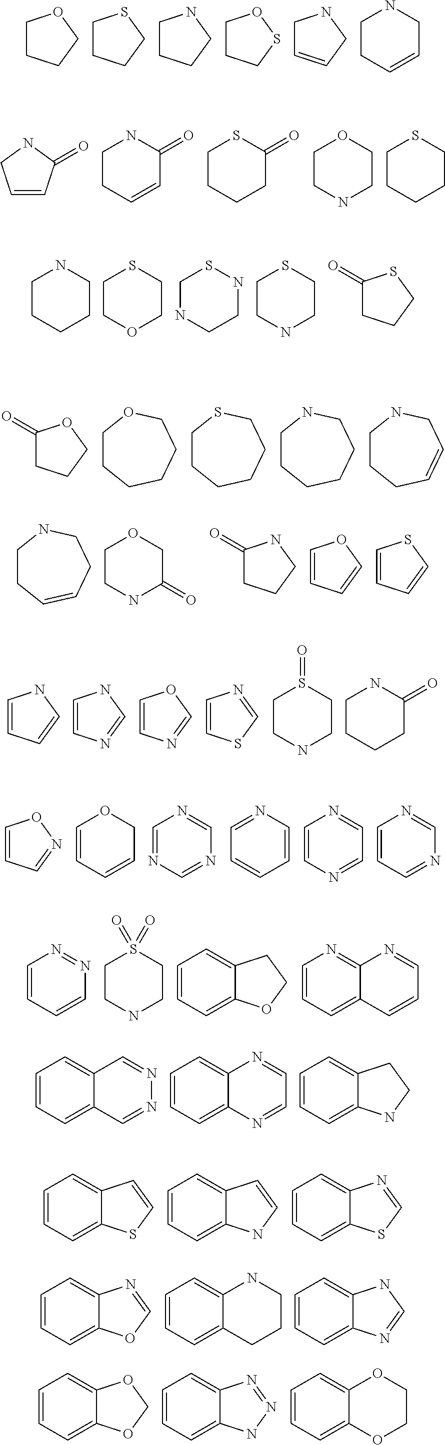

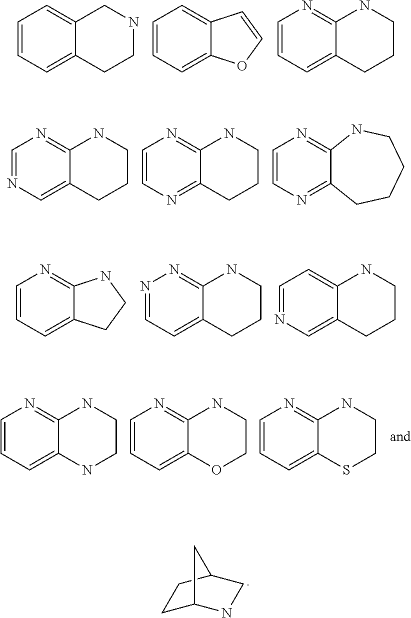

“Available nitrogen atoms” are those nitrogen atoms that are part of a heterocycle and are joined by two single bonds (e.g. piperidine), leaving an external bond available for substitution by, for example, H or CH₃.

“Pharmaceutically-acceptable salt” means a salt prepared by conventional means, and are well known by those skilled in the art. The “pharmacologically acceptable salts” include basic salts of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. When compounds of the invention include an acidic function such as a carboxy group, then suitable pharmaceutically acceptable cation pairs for the carboxy group are well known to those skilled in the art and include alkaline, alkaline earth, ammonium, quaternary ammonium cations and the like. For additional examples of “pharmacologically acceptable salts,” see infra and Berge et al., J. Pharm. Sci. 66:1 (1977).

“Saturated, partially saturated or unsaturated” includes substituents saturated with hydrogens, substituents completely unsaturated with hydrogens and substituents partially saturated with hydrogens.

“Leaving group” generally refers to groups readily displaceable by a nucleophile, such as an amine, a thiol or an alcohol nucleophile. Such leaving groups are well known in the art. Examples of such leaving groups include, but are not limited to, N-hydroxysuccinimide, N-hydroxybenzotriazole, halides, triflates, tosylates and the like. Preferred leaving groups are indicated herein where appropriate. “Protecting group” generally refers to groups well known in the art which are used to prevent selected reactive groups, such as carboxy, amino, hydroxy, mercapto and the like, from undergoing undesired reactions, such as nucleophilic, electrophilic, oxidation, reduction and the like. Preferred protecting groups are indicated herein where appropriate. Examples of amino protecting groups include, but are not limited to, aralkyl, substituted aralkyl, cycloalkenylalkyl and substituted cycloalkenyl alkyl, allyl, substituted allyl, acyl, alkoxycarbonyl, aralkoxycarbonyl, silyl and the like. Examples of aralkyl include, but are not limited to, benzyl, ortho-methylbenzyl, trityl and benzhydryl, which can be optionally substituted with halogen, alkyl, alkoxy, hydroxy, nitro, acylamino, acyl and the like, and salts, such as phosphonium and ammonium salts. Examples of aryl groups include phenyl, naphthyl, indanyl, anthracenyl, 9-(9-phenylfluorenyl), phenanthrenyl, durenyl and the like. Examples of cycloalkenylalkyl or substituted cycloalkylenylalkyl radicals, preferably have 6-10 carbon atoms, include, but are not limited to, cyclohexenyl methyl and the like. Suitable acyl, alkoxycarbonyl and aralkoxycarbonyl groups include benzyloxycarbonyl, t-butoxycarbonyl, iso-butoxycarbonyl, benzoyl, substituted benzoyl, butyryl, acetyl, trifluoroacetyl, trichloro acetyl, phthaloyl and the like. A mixture of protecting groups can be used to protect the same amino group, such as a primary amino group can be protected by both an aralkyl group and an aralkoxycarbonyl group. Amino protecting groups can also form a heterocyclic ring with the nitrogen to which they are attached, for example, 1,2-bis(methylene)benzene, phthalimidyl, succinimidyl, maleimidyl and the like and where these heterocyclic groups can further include adjoining aryl and cycloalkyl rings. In addition, the heterocyclic groups can be mono-, di- or tri-substituted, such as nitrophthalimidyl. Amino groups may also be protected against undesired reactions, such as oxidation, through the formation of an addition salt, such as hydrochloride, toluenesulfonic acid, trifluoroacetic acid and the like. Many of the amino protecting groups are also suitable for protecting carboxy, hydroxy and mercapto groups. For example, aralkyl groups. Alkyl groups are also suitable groups for protecting hydroxy and mercapto groups, such as tert-butyl. Silyl protecting groups are silicon atoms optionally substituted by one or more alkyl, aryl and aralkyl groups. Suitable silyl protecting groups include, but are not limited to, trimethylsilyl, triethylsilyl, triisopropylsilyl, tert-butyldimethylsilyl, dimethylphenylsilyl, 1,2-bis(dimethylsilyl)benzene, 1,2-bis(dimethylsilyl)ethane and diphenylmethylsilyl. Silylation of an amino groups provide mono- or di-silylamino groups. Silylation of aminoalcohol compounds can lead to a N,N,O-trisilyl derivative. Removal of the silyl function from a silyl ether function is readily accomplished by treatment with, for example, a metal hydroxide or ammonium fluoride reagent, either as a discrete reaction step or in situ during a reaction with the alcohol group. Suitable silylating agents are, for example, trimethylsilyl chloride, tert-butyl-dimethylsilyl chloride, phenyldimethylsilyl chloride, diphenylmethyl silyl chloride or their combination products with imidazole or DMF. Methods for silylation of amines and removal of silyl protecting groups are well known to those skilled in the art. Methods of preparation of these amine derivatives from corresponding amino acids, amino acid amides or amino acid esters are also well known to those skilled in the art of organic chemistry including amino acid/amino acid ester or aminoalcohol chemistry.

Protecting groups are removed under conditions which will not affect the remaining portion of the molecule. These methods are well known in the art and include acid hydrolysis, hydrogenolysis and the like. A preferred method involves removal of a protecting group, such as removal of a benzyloxycarbonyl group by hydrogenolysis utilizing palladium on carbon in a suitable solvent system such as an alcohol, acetic acid, and the like or mixtures thereof. A t-butoxycarbonyl protecting group can be removed utilizing an inorganic or organic acid, such as HCl or trifluoroacetic acid, in a suitable solvent system, such as dioxane or methylene chloride. The resulting amino salt can readily be neutralized to yield the free amine. Carboxy protecting group, such as methyl, ethyl, benzyl, tert-butyl, 4-methoxyphenylmethyl and the like, can be removed under hydrolysis and hydrogenolysis conditions well known to those skilled in the art.

It should be noted that compounds of the invention may contain groups that may exist in tautomeric forms, such as cyclic and acyclic amidine and guanidine groups, heteroatom substituted heteroaryl groups (Y′═O, S, NR), and the like, which are illustrated in the following examples:

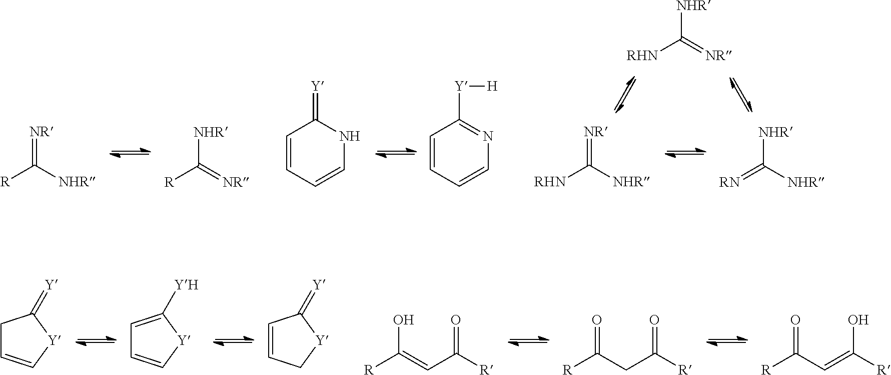

and though one form is named, described, displayed and/or claimed herein, all the tautomeric forms are intended to be inherently included in such name, description, display and/or claim.

Prodrugs of the compounds of this invention are also contemplated by this invention. A prodrug is an active or inactive compound that is modified chemically through in vivo physiological action, such as hydrolysis, metabolism and the like, into a compound of this invention following administration of the prodrug to a patient. The suitability and techniques involved in making and using prodrugs are well known by those skilled in the art. For a general discussion of prodrugs involving esters see Svensson and Tunek Drug Metabolism Reviews 165 (1988) and Bundgaard Design of Prodrugs, Elsevier (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bungaard J. Med. Chem. 2503 (1989)). Also, drugs containing an acidic NH group, such as imidazole, imide, indole and the like, have been masked with N-acyloxymethyl groups (Bundgaard Design of Prodrugs, Elsevier (1985)). Hydroxy groups have been masked as esters and ethers. EP 039,051 (Sloan and Little, Apr. 11, 1981) discloses Mannich-base hydroxamic acid prodrugs, their preparation and use.

The specification and claims contain listing of species using the language “selected from . . . and . . . ” and “is . . . or . . . ” (sometimes referred to as Markush groups). When this language is used in this application, unless otherwise stated it is meant to include the group as a whole, or any single members thereof, or any subgroups thereof. The use of this language is merely for shorthand purposes and is not meant in any way to limit the removal of individual elements or subgroups as needed.

### EXPERIMENTAL

The following abbreviations are used:
aq.—aqueous
BINAP—2,2′-bis(diphenylphosphino)-1,1′-binaphthyl
concd—concentrated
DCM—dichloromethane
DMF—N, N-dimethylformamide
DMSO—dimethylsulfoxide
Et₂O—diethyl ether
EtOAc—ethyl acetate
EtOH—ethyl alcohol
h—hour(s)
min—minutes
MeOH—methyl alcohol
NMP—1-methyl-2-pyrrolidinone
rt—room temperature
satd—saturated
TFA—trifluoroacetic acid
THF—tetrahydrofuran
X-Phos—2-dicyclohexylphosphino-2′,4′,6′-tri-isopropyl-1,1′-biphenyl

### General

Reagents and solvents used below can be obtained from commercial sources. ¹HNMR spectra were recorded on a Bruker 400 MHz and 500 MHz NMR spectrometer. Significant peaks are tabulated in the order: number of protons, multiplicity (s, singlet; d, doublet; t, triplet; q, quartet; m, multiplet; br s, broad singlet), and coupling constant(s) in Hertz (Hz). Mass spectrometry results are reported as the ratio of mass over charge, followed by the relative abundance of each ion (in parentheses electrospray ionization (ESI) mass spectrometry analysis was conducted on an Agilent 1100 series LC/MSD electrospray mass spectrometer. All compounds could be analyzed in the positive ESI mode using acetonitrile:water with 0.1% formic acid as the delivery solvent. Reverse phase analytical HPLC was carried out using an Agilent 1200 series on an Agilent Eclipse XDB-C18 5 μm column (4.6×150 mm) as the stationary phase and eluting with acetonitrile:H₂O with 0.1% TFA. Reverse phase semi-prep HPLC was carried out using an Agilent 1100 Series on a Phenomenex Gemini™ 10 μm C18 column (250×21.20 mm) as the stationary phase and eluting with acetonitrile:H₂O with 0.1% TFA.

### General Methods:

### General Method A0:

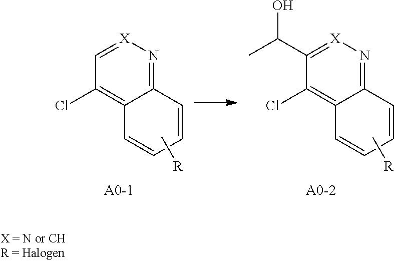

Intermediates of the type A0-2 can be synthesized as follows:

To a solution of A0-1 in THF at −78° C. was added freshly prepared 1M lithium diisopropylamide (LDA). After stirring for 20 min, acetaldehyde was added and the reaction was stirred at −78° C. for 1 h. The reaction was quenched with 50% sat NH₄Cl, warmed to rt and diluted with ethyl acetate. The layers were separated and the organic layer was washed with brine, dried over MgSO₄, filtered, and concentrated to afford A0-2. Compounds A0-2 were purified by column chromatography as necessary.

### General Method A1

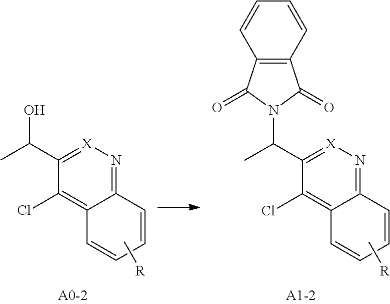

Intermediates of the type A1-2 can be synthesized as follows:

A solution of A0-2, triphenylphosphine and pthalimide in THF at 0° C. was treated with diisopropylazodicaroxylate (DIAD). The reaction was allowed to stir overnight and then was diluted with ethyl acetate, washed with NaHCO₃, brine, and dried over MgSO₄, filtered, and concentrated. Purification by column chromatography or crystallization from isopropanol afforded A1-2.

### General Method A2:

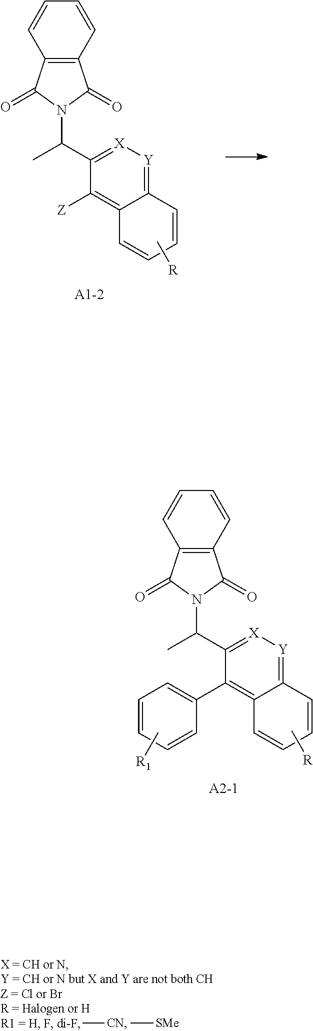

Intermediates of the type A2-1 can be synthesized as follows:

A reaction vessel containing K₃PO₄, palladium(II) acetate, A1-2, SPhos, and a phenylboronic acid was sealed and purged with argon. The reaction was diluted with toluene and heated to 90° C. After the reaction was judged to be complete, the reaction was cooled to rt and diluted with ethyl acetate. The organic layer was washed with brine, dried over MgSO₄, filtered, and concentrated. The residue was purified by column chromatography to afford A2-1.

### General Method A3:

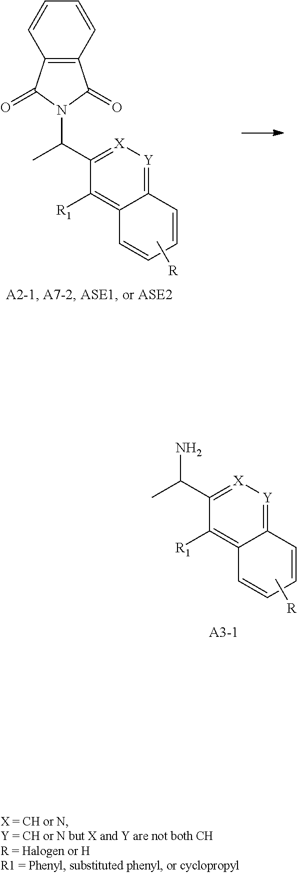

Intermediates of the type A3-1 can be synthesized as follows: A slurry of A2-1, A7-2, ASE1 or ASE2 in ethanol was treated with hydrazine hydrate and heated to 80° C. After the reaction was judged to be complete, the reaction was cooled to rt and diluted with ethyl acetate, filtered, and concentrated. The residue was redissolved in ethyl acetate and washed with water and brine, dried over MgSO₄, filtered and concentrated to afford A3-1.

### General Method A4:

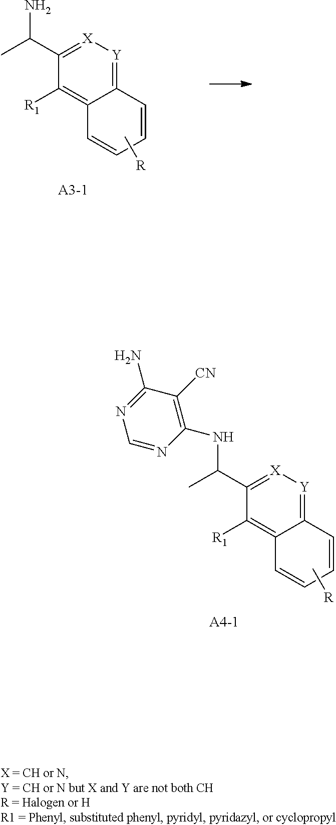

Compounds of the type A4-1 can be synthesized as follows:

A reaction flask containing 4-amino-6-chloropyrimidine-5-carbonitrile, A3-1, and DIEA in 1-butanol was heated to 120° C. After the reaction was judged to be complete by LC/MS, the mixture was cooled to rt and filtered. The resulting solid was washed with ethanol to afford A4-1. Further purification by recrystallization or chromatography was performed when necessary.

### General Method A5:

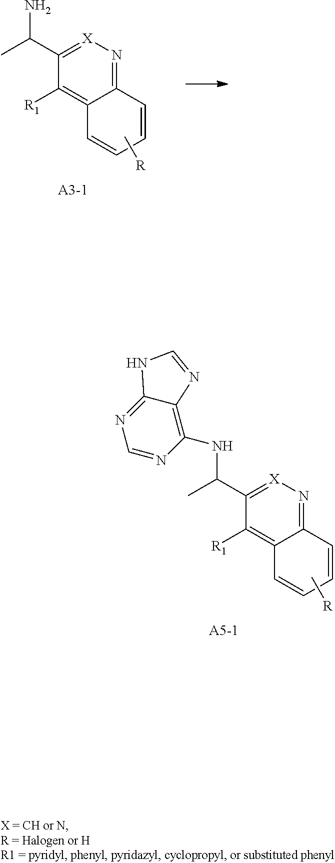

Compounds of the type A5-1 can be synthesized as follows: A reaction flask containing DIEA, 6-chloro-9H-purine, and A3-1 in 1-butanol was heated at 120° C. After the reaction was judged to be complete, the reaction was cooled to rt and the solvent was removed in vacuo. The residue was dissolved in DCM and washed with water and brine, dried over MgSO₄, filtered, and concentrated. Purification by column chromatography afforded A5-1.

### General Method A6:

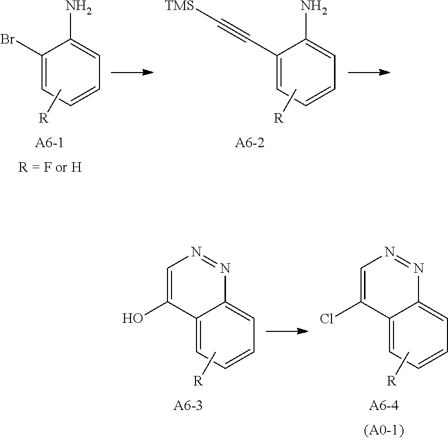

Intermediates of the type A6-1 can be synthesized as follows:

Copper(I) iodide, triethylamine, ethynyltrimethylsilane, bromoaniline A6-1, and palladium triphenylphosphine dichloride were combined and purged with nitrogen. DMF was added and the reaction was heated to 50° C. for 4 h or until the reaction was judged to be sufficiently complete. The reaction was cooled to rt and concentrated in vacuo. The residue was partitioned between water and DCM.

The organic phase was dried over MgSO₄, filtered, and concentrated. Purification by column chromatography afforded A6-2. To a solution of A6-2 in water was added 6N HCl. To the resulting mixture was added sodium nitrite dropwise as a solution in water. After 30 min, the reaction was heated to 100° C. for 3 h, then cooled to rt and quenched with sat NaHCO₃. The mixture was further cooled to 0° C., filtered, and washed with water and DCM. The solid was air dried to afford A6-3. To a solution of A6-3 in chlorobenzene was added POCl₃ and pyridine (0.237 mL, 2.92 mmol). The reaction was heated to 140° C. After the reaction was judged to be complete, the solution was cooled to rt and cautiously quenched with sat K₂CO₃. The product was extracted with DCM and filtered. Purification by column chromatography afforded A6-4, a subclass of compounds A0-1.

### General Method A7:

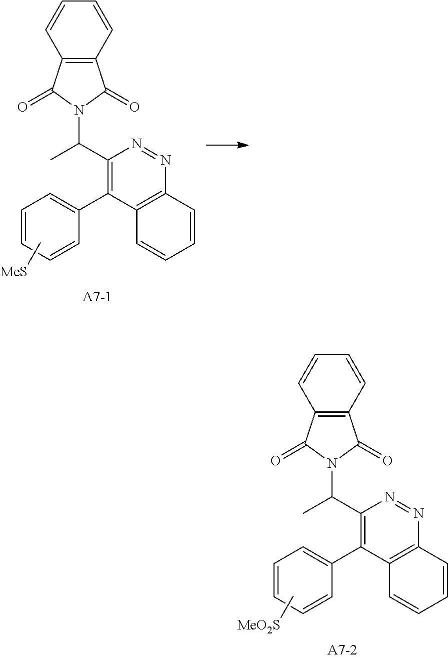

Intermediates of the type A7-2 can be synthesized as follows:

A solution A7-1 (a subclass of A2-1) in DCM was treated with oxone and montmorillonite K-10 clay (wetted with ˜18% water) in DCM. The reaction was allowed to stir overnight. The reaction was filtered and washed with sat sodium bicarbonate, extracted with ethyl acetate, washed with brine, dried over MgSO₄, filtered and concentrated. The residue was treated with titanium trichloride (30 wt % in 2 N HCl) and after workup, 4,5-dichloro-3,6-dioxocyclohexa-1,4-diene-1,2-dicarbonitrile (DDQ) in THF to afford the desired product. The solvent was removed and the residue was redissolved in DCM and filtered through celite. The organic phase was washed twice with sat NaHCO₃ and once with brine. The DCM layer was then dried over MgSO₄, filtered, and concentrated. Purification by column chromatography afforded A7-2.

### General Method A8:

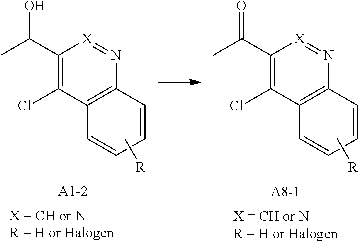

Intermediates of the type A8-1 can be synthesized as follows:

To a slurry of A0-2 in toluene was added manganese dioxide. The reaction was heated to 100° C. for 3 h, cooled to rt, and filtered through Celite™. The filter cake was washed with toluene and the filtrates were concentrated. Purification by column chromatography afforded A8-1.

### General Method A9:

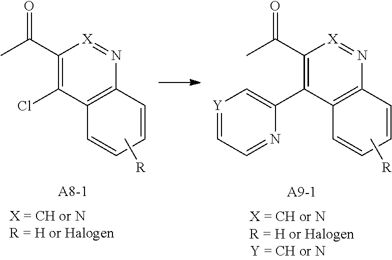

Intermediates of the type A9-1 can be synthesized as follows:

To a reaction vessel containing A8-1, Pd(ddpf)Cl₂, an aryltributylstannane, and 1,4-dioxane. The reaction was heated to 90° C. overnight, then cooled to rt and diluted with ethyl acetate. The organic phase was washed with NaHCO₃ and brine, dried over MgSO₄, filtered and concentrated. Purification by column chromatography afforded A9-1.

### General Method A10:

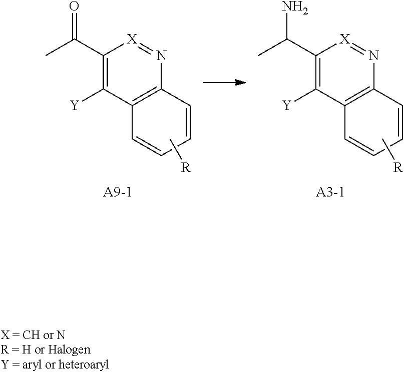

Intermediates of the type A3-1 can be synthesized as follows:

A mixture of titanium(IV) isopropoxide, ammonia (˜7M in methanol) and A9-1 were stirred overnight under an inert atmosphere overnight. The mixture was then treated with NaBH₄ (99 mg, 2.63 mmol). After the reaction was judged to be complete, it was worked up by addition of NH₄OH. The resulting solids were filtered off and the filtrate was concentrated and purified by column chromatography to afford A3-1.

### General Method A11:

Intermediates of the type A0-2 can be synthesized as follows:

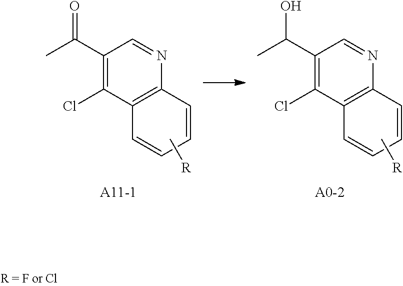

To a solution of A1′-1 in methanol at ˜10° C. was added sodium borohydride. The reaction was cooled to 0° C. and stirred for 30 min. The solvent was removed and the residue was redissolved in DCM/water. The layers were separated and the aqueous layer was extracted with DCM. The combined organic layers were washed with brine and dried over MgSO₄, filtered, and concentrated to afford A0-2.

### General Method B4:

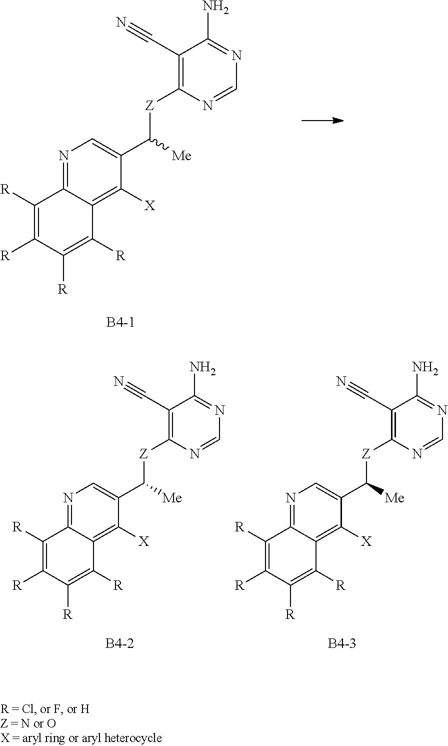

The enantiomers of B4-1 were separated on a chiral SFC column. The fractions containing the first peak to elute were combined and concentrated under vacuum to provide B4-2 (the stereochemistry is arbitrarily assigned). The fractions containing the second peak to elute were combined and concentrated under vacuum to provide B4-3 (The stereochemistry is arbitrarily assigned).

### General Method B13:

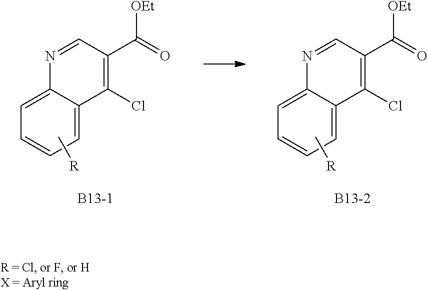

B13-1, an aryl boronic acid, and potassium carbonate were combined in DMF.

The solution was sparged with N₂ before adding PdCl₂(dppf)₂CH₂Cl₂ and then it was heated to 110° C. overnight. The next day the solution was concentrated under vacuum and the residue obtained was purified by column chromatography. The fractions containing the product were combined and concentrated under vacuum to provide B13-2.

### General Method B12:

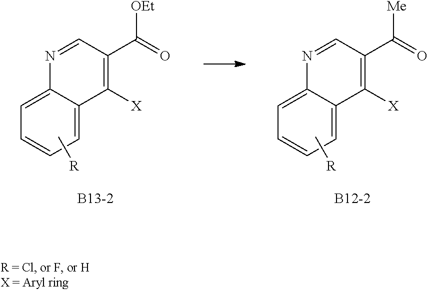

A suspension of B13-2 and N,O-dimethylhydroxylamine hydrochloride in anhydrous THF under an atmosphere of N₂ was cooled in a ice bath. To this was added slowly methylmagnesium bromide over a period of 10 min. The solution was allowed to warm to rt and then stirred for 3 h. The solution was poured into ice/sat NH₄Cl and then the product was extracted with DCM. The organics were dried over Na₂SO₄ and then concentrated under vacuum. The residue obtained was purified by column chromatography. The fractions containing the product were combined and concentrated under vacuum to give B12-2.

### General Method B11:

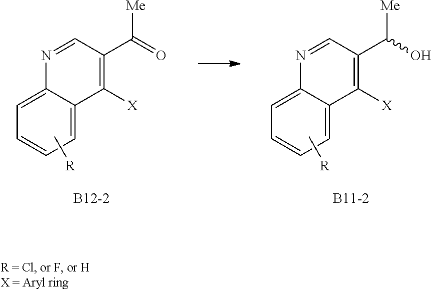

At 0° C. and under an atm. of N₂ was dissolved B12-2 in Methanol. To this was added sodium borohydride and the yellow solution was left to warm to rt. After 1 h the solution was concentrated under vacuum and then diluted with sat NaHCO₃. The product was extracted with Ethyl acetate and the organics were dried over MgSO₄ before being concentrated under vacuum. The residue obtained was purified by column chromatography. The fractions containing the product were combined and concentrated under vacuum to provide B11-2.

### General Method B10:

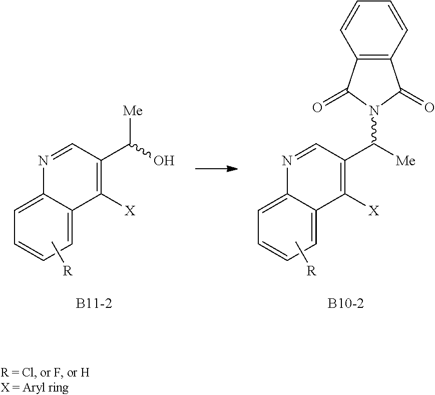

Combined isoindoline-1,3-dione, triphenylphosphine, and B11-2 in anhydrous THF. The solution was then cooled in a ice bath before adding diisopropylazodicarboxylate (DIAD). The solution was then left to warm to rt and stirred overnight. The solution was then concentrated under vacuum and then diluted with ethyl acetate. The organics were washed in succession with H₂O and brine, before being dried over MgSO₄ and then concentrated under vacuum. The yellow oil obtained was purified by column chromatography. The fractions containing the product were combined and concentrated under vacuum to provide B10-2.

### General Method B14:

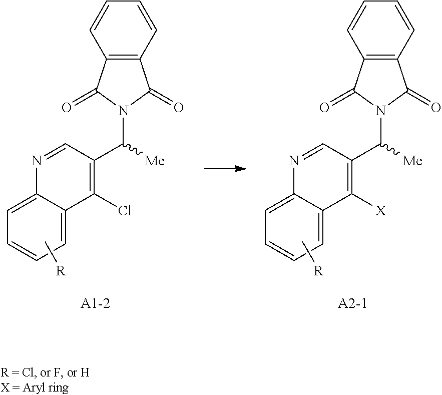

A1-2, potassium phosphate, and arylboronic acid were combined in t-amyl alcohol and 1,4-1,4-dioxane. The suspension was briefly sparged with N₂ before adding Pd(dba)₂ and dicyclohexyl(2′,4′,6′-triisopropylbiphenyl-2-yl)phosphine. The suspension was heated to 95° C. and monitored by LCMS for the absence of the starting material. The suspension was cooled to rt and then diluted into H₂O. The product was extracted with DCM. The organics were dried over MgSO₄ and then concentrated under vacuum. The residue obtained was purified by column chromatography. The fractions containing the product were combined and concentrated under vacuum to provide A2-1.

### General Method B5:

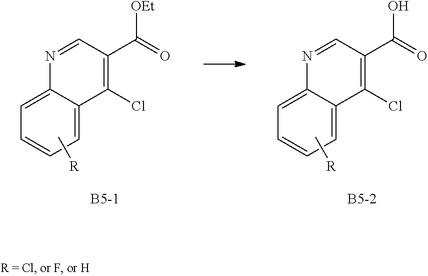

Compound B5-1 was dissolved in acetonitrile and then cooled in an ice bath. To this was added a solution of LiOH.2H₂O dissolved in water. The reaction mixture was stirred at rt for 6 h. The mixture was concentrated to half the volume under vacuum. The pH of the mixture was adjusted to ˜8-9 with 5N HCl. The solids were filtered off through a Buchner funnel and then washed with water followed by diethyl ether to provide B5-2.

### General Method B6:

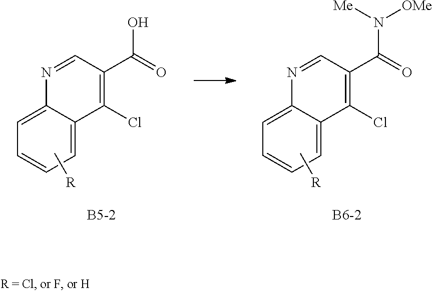

To a suspension of B5-2 in toluene at 0° C. was added SOCl₂. The mixture was refluxed at 110° C. for 12 h under N₂, after which time the mixture was evaporated under high vacuum. The residue obtained was dissolved in DCM and the solution was cooled in a ice bath before adding triethyl amine followed by N,O-dimethyl hydroxyl-amine hydrochloride. The mixture was stirred at 0° C. for 1 h, then diluted with water and the product was extracted with DCM. The organic layer was dried over Na₂SO₄ and concentrated under vacuum to provide B6-2.

### General Method B8:

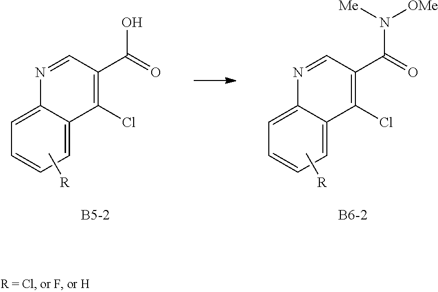

To a solution of B5-2 in DMF, HATU and DIPEA were added. The reaction mixture was stirred for 10 min, before N,O-dimethyl Hydroxyl-amine hydrochloride was added. The mixture was stirred overnight and then diluted with water. The product was extracted with ethyl acetate. The organics were dried over Na₂SO₄ and then concentrated under vacuum. The residue obtained was purified by column chromatography to provide B6-2.

### General Method B7:

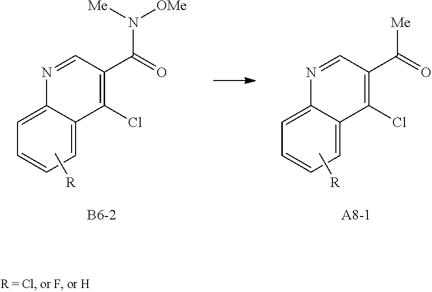

A solution of B6-2 in THF was cooled to −70° C. To this was added methyl lithium dropwise. The temperature of the reaction mixture was raised to −20° C. from −70° C. within one hour. The reaction mixture was quenched with sat NH₄Cl and the product was extracted with ethyl acetate. The organic layer was dried over Na₂SO₄ and then concentrated under vacuum. The residue obtained was purified by column chromatography to provide A8-1.

### Specific Examples

### Specific Example of General Method A1

### 1-(4-Chloroquinolin-3-yl)ethanol

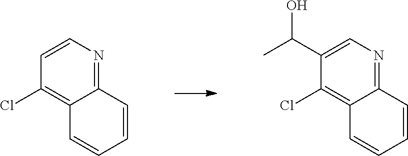

To a solution of 4-chloroquinoline (1.636 g, 10.00 mmol) in THF (100 mL) at −78° C. was added freshly prepared 1M lithium diisopropylamide (11 mL, 11 mmol, 1.1 eq). After stirring for 20 min, acetaldehyde (1.694 mL, 30.0 mmol) was added and the reaction was stirred at −78° C. for 1 h. The reaction was quenched with 50% sat NH₄Cl, warmed to rt and diluted with ethyl acetate. The layers were separated and the organic layer was washed with brine, dried over MgSO₄, filtered, and concentrated to afford 1-(4-chloroquinolin-3-yl)ethanol. ¹H NMR (500 MHz, CDCl₃) δ ppm 9.10 (s, 1H), 8.22 (d, J=8.6 Hz, 1H), 8.10 (d, J=8.3 Hz, 1H), 7.73 (ddd, J=8.3, 7.1, 1.5 Hz, 1H), 7.64 (ddd, J=8.1, 6.8, 1.0 Hz, 1H), 5.56 (q, J=6.6 Hz, 1H), 2.79 (br s, 1H), 1.62 (d, J=6.6 Hz, 3H).

### 2-(1-(4-Chloroquinolin-3-yl)ethyl)isoindoline-1,3-dione

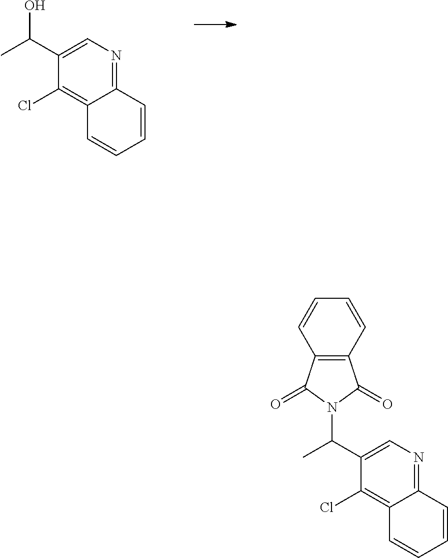 [SMILES: CC(O)c1cnc2ccccc2c1Cl ; CC(c1cnc2ccccc2c1Cl)N1C(=O)c2ccccc2C1=O]

To a solution of phthalimide (0.527 g, 3.58 mmol), triphenylphosphine (0.940 g, 3.58 mmol), and 1-(4-chloroquinolin-3-yl)ethanol (0.62 g, 2.99 mmol) in THF (29.9 mL) at 0° C. was added diisopropylazodicaroxylate (DIAD) (0.697 mL, 3.58 mmol). The reaction was allowed to stir overnight and then was diluted with ethyl acetate, washed with NaHCO₃, brine, and dried over MgSO₄, filtered, and concentrated. Purification by column chromatography afforded 2-(1-(4-chloroquinolin-3-yl)ethyl)isoindoline-1,3-dione. ¹H NMR (500 MHz, CDCl₃) δ ppm 9.33 (s, 1H), 8.22 (d, J=8.6 Hz, 1H), 8.12 (d, J=8.3 Hz, 1H), 7.82 (m, 2H), 7.76 (ddd, J=8.1, 6.9, 1.2 Hz, 1H), 7.71 (m, 2H), 6.10 (q, J=7.1 Hz, 1H), 2.05 (d, J=7.3 Hz, 3H). Mass Spectrum (ESI) m/e=337.2 (M+1).

### Specific Example of General Method A2

### 2-(1-(4-(4-Fluorophenyl)quinolin-3-yl)ethyl)isoindoline-1,3-dione

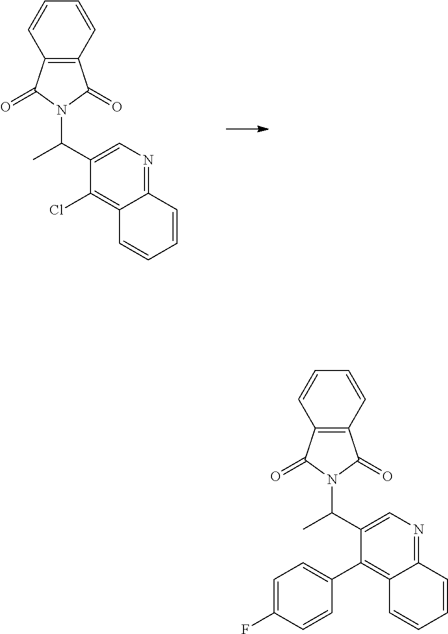 [SMILES: CC(c1cnc2ccccc2c1Cl)N1C(=O)c2ccccc2C1=O ; CC(c1cnc2ccccc2c1-c1ccc(F)cc1)N1C(=O)c2ccccc2C1=O]

A reaction vessel containing K₃PO₄ (126 mg, 0.594 mmol), palladium(II) acetate (2.67 mg, 0.012 mmol), 2-(1-(4-chloroquinolin-3-yl)ethyl)isoindoline-1,3-dione (200 mg, 0.594 mmol), 4-fluorophenylboronic acid (125 mg, 0.891 mmol), and SPhos (12.17 mg, 0.030 mmol) was sealed and purged with argon. To the reaction was diluted with 3 mL toluene and heated to 90° C. After 2 h, the reaction was cooled to rt and diluted with ethyl acetate. The organic layer was washed with brine, dried over MgSO₄, filtered, and concentrated. The residue was purified using 20-40% ethyl acetate in hexane to afford 2-(1-(4-(4-fluorophenyl)quinolin-3-yl)ethyl)isoindoline-1,3-dione. ¹H NMR (500 MHz, CDCl₃) δ ppm 9.44 (s, 1H), 8.15 (d, J=8.1 Hz, 1H), 7.75 (m, 2H), 7.69 (m, 3H), 7.42 (ddd, J=8.3, 6.9, 1.0 Hz, 1H), 7.27 (m, 1H), 7.15 (m, 1H), 7.08 (tt, J=8.5, 1.7, 1H), 5.52 (q, J=7.3 Hz, 1H), 1.93 (d, J=7.3 Hz, 3H). Mass Spectrum (ESI) m/e=397.2 (M+1).

### Specific Examples of General Method A3

### 1-(4-(4-Fluorophenyl)quinolin-3-yl)ethanamine

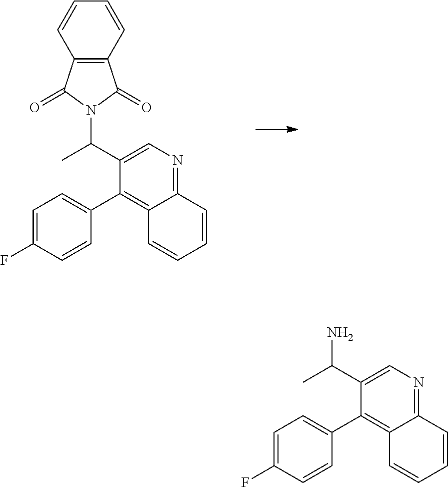 [SMILES: CC(c1cnc2ccccc2c1-c1ccc(F)cc1)N1C(=O)c2ccccc2C1=O ; CC(N)c1cnc2ccccc2c1-c1ccc(F)cc1]

A slurry of 1-(4-(4-fluorophenyl)quinolin-3-yl)ethanamine in ethanol (5045 μL) was treated with hydrazine hydrate (247 μL, 5.05 mmol) and heated to 80° C. After 1 h, the reaction was cooled to rt and diluted with ethyl acetate, filtered, and concentrated. The residue was redissolved in ethyl acetate and washed with water and brine, dried over MgSO₄, filtered, and concentrated to afford 1-(4-(4-fluorophenyl)quinolin-3-yl)ethanamine. ¹H NMR (500 MHz, CDCl₃) δ ppm 9.23 (s, 1H), 8.14 (d, J=8.3 Hz, 1H), 7.68 (m, 1H), 7.43 (m, 1H), 7.34 (m, 1H), 7.30-7.22 (series of m, 4H), 4.15 (q, J=6.6 Hz, 1H), 1.42 (d, J=6.6 Hz, 3H). Mass Spectrum (ESI) m/e=267.2 (M+1).

### 1-(4-Phenylquinolin-3-yl)ethanamine

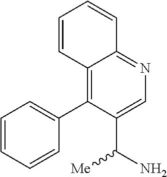 [SMILES: CC(N)c1cnc2ccccc2c1-c1ccccc1]

2-(1-(4-Phenylquinolin-3-yl)ethyl)isoindoline-1,3-dione (0.160 g, 0.423 mmol) and hydrazine hydrate (0.205 mL, 4.23 mmol) were combined in 10 mL of ethanol. The solution was heated at 60° C. for 3 h and then cooled to rt. The suspension obtained was diluted with ethyl acetate and then filtered through Celite™. The filtrates were washed with H₂O followed by brine and then dried over MgSO₄ before being concentrated under vacuum to provide 1-(4-phenylquinolin-3-yl)ethanamine (100 mg, crude) as a brown film which was carried on without further purification. Mass Spectrum (ESI) m/e=249.1 (M+1).

### Specific Examples of General Method A4

### Example 1

### 4-Amino-6-((1-(4-(4-fluorophenyl)-3-quinolinyl)ethyl)amino)-5-pyrimidinecarbonitrile

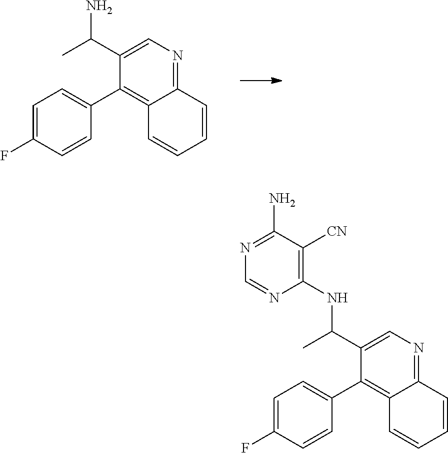 [SMILES: CC(N)c1cnc2ccccc2c1-c1ccc(F)cc1 ; CC(Nc1ncnc(N)c1C#N)c1cnc2ccccc2c1-c1ccc(F)cc1]

A reaction flask containing 4-amino-6-chloropyrimidine-5-carbonitrile (83 mg, 0.537 mmol), 1-(4-(4-fluorophenyl)quinolin-3-yl)ethanamine (135 mg, 0.507 mmol), and DIEA (177 μL, 1.014 mmol) in 1-butanol (5069 μL) was heated to 120° C. After the reaction was judged to be complete by LC/MS, the mixture was cooled to rt and filtered. The resulting solid was washed with ethanol to afford 4-amino-6-((1-(4-(4-fluorophenyl)-3-quinolinyl)ethyl)amino)-5-pyrimidinecarbonitrile. ¹H NMR (500 MHz, CDCl₃) δ ppm 9.01 (s, 1H), 8.13 (d, J=8.3 Hz, 1H), 8.00 (s, 1H), 7.69 (ddd J=8.1, 6.4, 1.2 Hz, 1H), 7.58 (m, 1H), 7.45 (m, 1H), 7.38 (m, 1H), 7.30-7.22 (series of m, 3H), 5.54 (d, J=6.6 Hz, 1H), 5.35-5.25 (series of m, 3H), 1.53 (d, J=7.1 Hz, 3H). Mass Spectrum (ESI) m/e=385.1 (M+1). The individual enantiomers were obtained according to the methods described in General Method B4 to give 4-amino-6-(((1S)-1-(4-(4-fluorophenyl)-3-quinolinyl)ethyl)amino)-5-pyrimidinecarbonitrile and 4-amino-6-(((1R)-1-(4-(4-fluorophenyl)-3-quinolinyl)ethyl)amino)-5-pyrimidinecarbonitrile and the spectral data of each chiral enantiomer was consistent with that of racemic 4-amino-6-((1-(4-(4-fluorophenyl)-3-quinolinyl)ethyl)amino)-5-pyrimidinecarbonitrile.

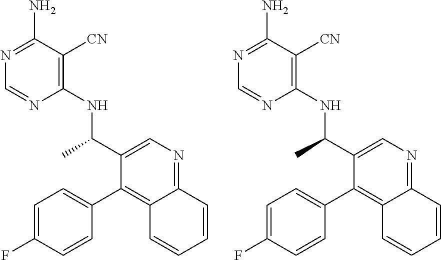

### Example 2

### 4-Amino-6-((1-(8-chloro-6-fluoro-4-(2-pyridinyl)-3-quinolinyl)-ethyl)amino)-5-pyrimidinecarbonitrile

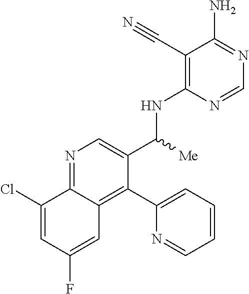 [SMILES: CC(Nc1ncnc(N)c1C#N)c1cnc2c(Cl)cc(F)cc2c1-c1ccccn1]

1-(8-Chloro-6-fluoro-4-(pyridin-2-yl)quinolin-3-yl)ethanamine (0.12 g, 0.398 mmol), N-ethyl-N-isopropylpropan-2-amine (0.514 g, 3.98 mmol), and 4-amino-6-chloropyrimidine-5-carbonitrile (0.074 g, 0.477 mmol) were combined in 4 mL of 1-butanol and then heated under N₂ to 110° C. for 1 h. The solvents were removed under vacuum and the residue obtained was purified by column chromatography using a gradient of 60% ethyl acetate/hexane to 100% ethyl acetate. The fractions containing the product were combined and concentrated under vacuum to provide 4-amino-6-((1-(8-chloro-6-fluoro-4-(2-pyridinyl)-3-quinolinyl)ethyl)amino)-5-pyrimidinecarbonitrile as a light yellow solid. A mixture of isomers was observed in the proton NMR trace. ¹H NMR (500 MHz, DMSO-d₆) δ ppm 9.31 (1H, br. s.), 8.80 (1H, d, J=3.4 Hz), 7.97-8.12 (2.7H, m), 7.85 (0.8H, br. s.), 7.75 (0.8H, d, J=7.6 Hz), 7.65 (0.4H, br. s.), 7.48-7.61 (1.2H, m), 7.08-7.36 (2H, m), 6.89 (1H, d, J=9.0 Hz), 5.40 (0.2H, br. s.), 4.96-5.19 (0.8H, m), 1.59 (0.6H, br. s.), 1.48 (2.3H, d, J=6.4 Hz). Mass Spectrum (ESI) m/e=420.1 (M+1) and 418.1 (M−1). The individual enantiomers were obtained according to the methods described in General Method B4 to give 4-1mino-6-(((1S)-1-(8-chloro-6-fluoro-4-(2-pyridinyl)-3-quinolinyl)ethyl)amino)-5-pyrimidinecarbonitrile and 4-1mino-6-(((1R)-1-(8-chloro-6-fluoro-4-(2-pyridin-yl)-3-quinolinyl)ethyl)amino)-5-pyrimidinecarbonitrile and the spectral data of each chiral enantiomer was consistent with that of racemic 4-1mino-6-((1-(8-chloro-6-fluoro-4-(2-pyridinyl)-3-quinolinyl)ethyl)amino)-5-pyrimidinecarbonitrile.

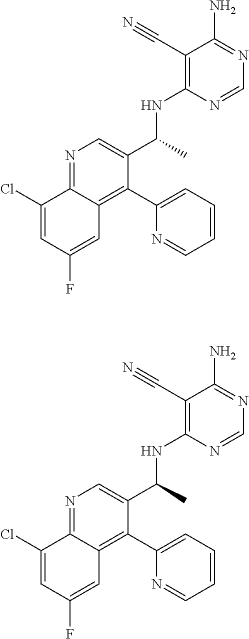 [SMILES: C[C@@H](Nc1ncnc(N)c1C#N)c1cnc2c(Cl)cc(F)cc2c1-c1ccccn1 ; C[C@H](Nc1ncnc(N)c1C#N)c1cnc2c(Cl)cc(F)cc2c1-c1ccccn1]

### Specific Example of General Method A5

### Example 3

### N-(1-(6-Fluoro-4-phenyl-3-quinolinyl)ethyl)-9H-purin-6-amine

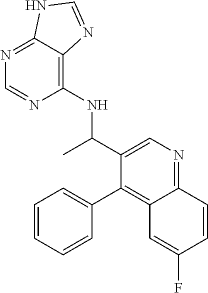 [SMILES: CC(Nc1ncnc2[nH]cnc12)c1cnc2ccc(F)cc2c1-c1ccccc1]

A reaction flask containing DIEA (39.3 μL, 0.225 mmol), 6-chloro-9H-purine (25.5 mg, 0.165 mmol), and 1-(6-fluoro-4-phenylquinolin-3-yl)ethanamine (40 mg, 0.150 mmol) in 1-butanol was heated at 120° C. After 14 h, the reaction was cooled to rt and the solvent was removed in vacuo. The residue was dissolved in DCM and washed with water and brine, dried over MgSO₄, filtered and concentrated. Purification by column chromatography using 0-80% (90:10:1 DCM:Methanol:NH₄OH) in DCM afforded N-(1-(6-fluoro-4-phenyl-3-quinolinyl)ethyl)-9H-purin-6-amine. The individual enantiomers were obtained by chiral SFC purification. ¹H NMR (500 MHz, CDCl₃) δ ppm 9.07 (s, 1H), 8.27 (s, 1H), 8.07 (dd, J=9.05, 5.4 Hz, 1H), 7.93 (s, 1H), 7.72 (d, J=7.3 Hz, 1H), 7.55 (m, 3H), 7.42 (td, J=8.1, 2.7 Hz, 1H), 7.27 (m, 1H), 6.34 (d, J=5.9 Hz, 1H), 5.45 (br s, 1H), 1.80 (d, J=6.8 Hz, 3H). Mass Spectrum (ESI) m/e=385.2 (M+1). The individual enantiomers were obtained according to the methods described in General Method B4 to give N-((1S)-1-(6-fluoro-4-phenyl-3-quinolinyl)ethyl)-9H-purin-6-amine and N-((1R)-1-(6-fluoro-4-phenyl-3-quinolinyl)ethyl)-9H-purin-6-amine and the spectral data of each chiral enantiomer was consistent with that of racemic N-(1-(6-fluoro-4-phenyl-3-quinolinyl)ethyl)-9H-purin-6-amine.

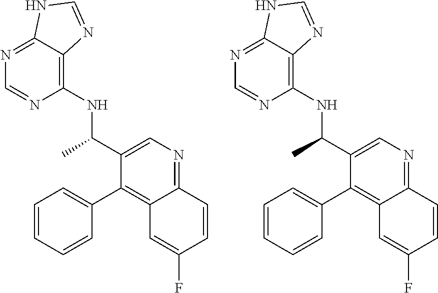 [SMILES: C[C@H](Nc1ncnc2[nH]cnc12)c1cnc2ccc(F)cc2c1-c1ccccc1 ; C[C@@H](Nc1ncnc2[nH]cnc12)c1cnc2ccc(F)cc2c1-c1ccccc1]

### Specific Example of General Method A6

### 4-Fluoro-2-((trimethylsilyl)ethynyl)aniline

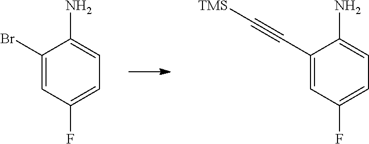 [SMILES: Nc1ccc(F)cc1Br ; C[Si](C)(C)C#Cc1cc(F)ccc1N]

Copper(I) iodide (0.188 g, 0.987 mmol), triethylamine (22.01 mL, 158 mmol), ethynyltrimethylsilane (16.61 mL, 118 mmol), 2-bromo-4-fluoroaniline (15 g, 79 mmol), palladium triphenylphosphine dichloride (3.11 g, 3.95 mmol) were combined and purged with nitrogen. DMF (200 mL) was added and the reaction was heated to 50° C. for 4 h. The reaction was cooled to rt and concentrated in vacuo. The residue was partitioned between water and DCM. The organic phase was dried over MgSO₄, filtered, and concentrated. Purification by column chromatography using 1-40% ethyl acetate in hexane afforded 4-fluoro-2-((trimethylsilyl)ethynyl)aniline. ¹H NMR (500 MHz, CDCl₃) δ ppm 7.00 (dd, J=9.1, 3.2 Hz, 1H), 6.85 (td, J=8.6, 2.9 Hz, 1H), 6.63 (dd, J=8.8, 4.7 Hz, 1H), 0.27 (s, 9H).

### 6-Fluorocinnolin-4-ol

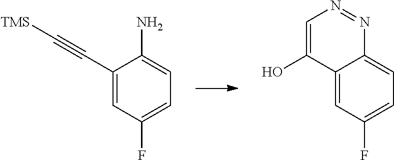 [SMILES: C[Si](C)(C)C#Cc1cc(F)ccc1N ; Oc1cnnc2ccc(F)cc12]

To a solution of 4-fluoro-2-((trimethylsilyl)ethynyl)aniline (6.5 g, 31.4 mmol) in water (62.7 mL) was added 55 mL of 6N HCl. To the resulting mixture was added sodium nitrite (3.24 g, 47.0 mmol) dropwise as a solution in 15 mL water. After 30 min, the reaction was heated to 100° C. for 3 h, then cooled to rt and quenched with sat NaHCO₃. The mixture was further cooled to 0° C., filtered, and washed with water and DCM. The solid was air dried to afford 6-fluorocinnolin-4-ol. ¹H NMR (500 MHz, DMSO-d₆) δ ppm 13.67 (br s, 1H), 7.73 (m, 4H). Mass Spectrum (ESI) m/e=165.2 (M+1).

### 4-Chloro-6-fluorocinnoline

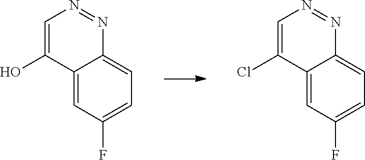 [SMILES: Oc1cnnc2ccc(F)cc12 ; Fc1ccc2nncc(Cl)c2c1]

To a solution of 6-fluorocinnolin-4-ol (1.6 g, 9.75 mmol) in chlorobenzene (32.7 mL, 322 mmol) was added POCl₃ (1.363 mL, 14.62 mmol), and pyridine (0.237 mL, 2.92 mmol). The reaction was heated to 140° C. After the reaction was judged to be complete, the solution was cooled to rt and cautiously quenched with sat K₂CO₃. The product was extracted with DCM and filtered. Purification by column chromatography afforded 4-chloro-6-fluorocinnoline. ¹H NMR (500 MHz, CDCl₃) δ ppm 9.34 (s, 1H), 8.62 (dd, J=8.8, 4.9 Hz, 1H), 7.80 (dd, J=8.8, 2.7 Hz, 1H), 7.70 (ddd, J=10.8, 8.1, 2.7 Hz, 1H). Mass Spectrum (ESI) m/e=183.2 (M+1).

### Specific Example of General Method A7

### 2-(1-(4-(4-(Methylsulfonyl)phenyl)cinnolin-3-yl)ethyl)isoindoline-1,3-dione

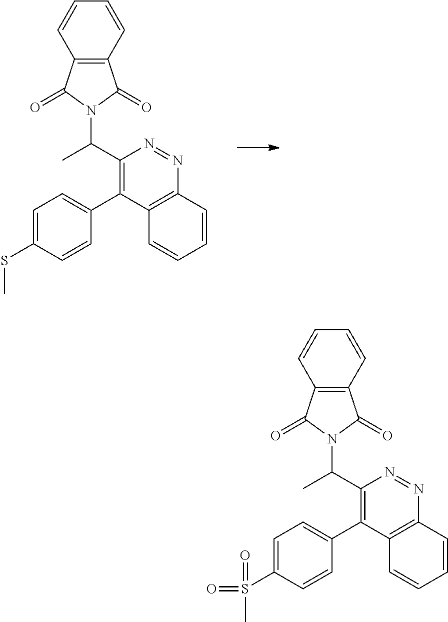 [SMILES: CSc1ccc(-c2c(C(C)N3C(=O)c4ccccc4C3=O)nnc3ccccc23)cc1 ; CC(c1nnc2ccccc2c1-c1ccc(S(C)(=O)=O)cc1)N1C(=O)c2ccccc2C1=O]

A solution 2-(1-(4-(4-(methylsulfonyl)phenyl)cinnolin-3-yl)ethyl)isoindoline-1,3-dione (210 mg, 0.459 mmol) in 5 mL DCM was treated with oxone (705 mg, 1.148 mmol) and 600 mg montmorillonite K-10 clay (wetted with ˜18% water) in 5 mL DCM. The reaction was allowed to stir overnight. LC/MS indicated that some over-oxidation had occurred. The reaction was filtered and washed with sat sodium bicarbonate, extracted with ethyl acetate, washed with brine, dried over MgSO₄, filtered, and concentrated. The residue was treated with titanium trichloride (30 wt % in 2N HCl) (1.18 g, 2.30 mmol) and after workup, 4,5-dichloro-3,6-dioxocyclohexa-1,4-diene-1,2-dicarbonitrile (208 mg, 0.918 mmol) in THF to afford the desired product. The solvent was removed and the residue was redissolved in DCM and filtered through celite. The organic phased was washed twice with sat NaHCO₃ and once with brine. The DCM layer was then dried over MgSO₄, filtered, and concentrated. Purification by column chromatography (50-60% ethyl acetate in hexane) afforded 2-(1-(4-(4-(methylsulfonyl)phenyl)cinnolin-3-yl)ethyl)isoindoline-1,3-dione. ¹H NMR (500 MHz, CDCl₃) δ ppm 8.63 (d, J=8.8 Hz, 1H), 8.16 (dd, J=7.8, 2.0 Hz, 1H), 7.86 (m, 1H), 7.79 (dd, J=8.1, 2.0 Hz, 1H), 7.69 (s, 4H), 7.66 (m, 1H), 7.59 (dd, J=7.8, 1.7 Hz, 1H), 7.40 (dd, J=8.1, 1.7, 1H), 7.30 (d, J=8.6 Hz, 1H), 5.80 (q, J=7.3 Hz, 1H), 3.15 (s, 3H), 2.10 (d, J=7.1 Hz, 3H). Mass Spectrum (ESI) m/e=458.2 (M+1).

### Specific Example of General Method A8

### 1-(4-Chloro-6-fluorocinnolin-3-yl)ethanone

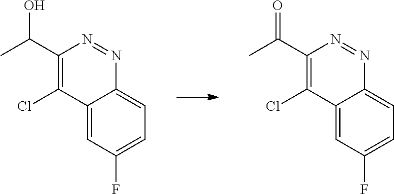

To a slurry of 1-(4-chloro-6-fluorocinnolin-3-yl)ethanol (565 mg, 2.493 mmol) in 25 mL toluene was added manganese dioxide (1734 mg, 19.94 mmol). The reaction was heated to 100° C. for 3 h, cooled to rt, and filtered through Celite™ The filter cake was washed with toluene and the filtrates were concentrated. Purification by column chromatography using 10-30% ethyl acetate in hexane afforded 1-(4-chloro-6-fluorocinnolin-3-yl)ethanone. ¹H NMR (500 MHz, CDCl₃) δ ppm 8.68 (dd, J=9.3 Hz, 5.1 Hz 1H), 8.02 (dd, J=8.8 Hz, 2.7 Hz, 1H), 7.77 (ddd, J=10.5, 7.8, 2.7 Hz, 1H), 3.02 (s, 3H). Mass Spectrum (ESI) m/e=225.1 (M+1).

### Specific Examples of General Method A9

### 1-(8-Chloro-6-fluoro-4-(pyridin-2-yl)quinolin-3-yl)ethanone

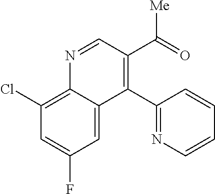 [SMILES: CC(=O)c1cnc2c(Cl)cc(F)cc2c1-c1ccccn1]

1-(4,8-Dichloro-6-fluoroquinolin-3-yl)ethanone (0.314 g, 1.217 mmol), and 2-(tributylstannyl)pyridine (0.476 mL, 1.460 mmol) were combined in 12 mL of anhydrous 1,4-dioxane. The solution was sparged with N₂ before adding PdCl₂(dppf)CH₂Cl₂ (0.099 g, 0.122 mmol). The solution was then heated at 90° C. for 2 h. The solution was cooled to rt and then loaded on to silica gel and then purified by column chromatography using a gradient of 20% ethyl acetate/hexane to 60% ethyl acetate/hexane. The fractions containing the product were combined and concentrated under vacuum to provide 1-(8-chloro-6-fluoro-4-(pyridin-2-yl)-quinolin-3-yl)ethanone as a brown solid. ¹H NMR (500 MHz, CHLOROFORM-d) δ ppm 9.22 (1H, s), 8.84 (1H, dt, J=4.9, 0.7 Hz), 7.92 (1H, td, J=7.7, 1.7 Hz), 7.75 (1H, dd, J=8.1, 2.7 Hz), 7.50 (1H, ddd, J=7.6, 4.9, 1.0 Hz), 7.46 (1H, dd, J=7.8, 0.7 Hz), 7.24 (1H, dd, J=9.3, 2.7 Hz), 2.19 (3H, s). Mass Spectrum (ESI) m/e=301.0 (M+1).

### 1-(6-Fluoro-4-(pyridin-2-yl)quinolin-3-yl)ethanone

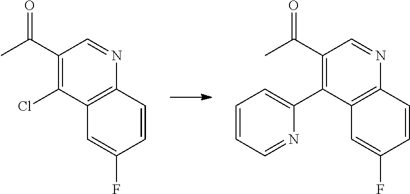 [SMILES: CC(=O)c1cnc2ccc(F)cc2c1Cl ; CC(=O)c1cnc2ccc(F)cc2c1-c1ccccn1]

To a reaction vessel containing Pd(ddpf)Cl₂ (163 mg, 0.2 mmol), 2-(tributyl-stannyl)pyridine (734 mg, 2.0 mmol) and 1-(4-chloro-6-fluoroquinolin-3-yl)-ethanone (446 mg, 2.0 mmol) was added 1,4-dioxane (12 mL). The reaction was heated to 90° C. overnight, then cooled to rt and diluted with 80 mL ethyl acetate. The organic phase was washed with 10 mL NaHCO₃ and 10 mL brine, dried over MgSO₄, filtered and concentrated. Purification by column chromatography using 50-70% ethyl acetate in hexane afforded 1-(6-fluoro-4-(pyridin-2-yl)quinolin-3-yl)ethanone. ¹H NMR (500 MHz, CDCl₃) δ ppm 9.13 (s, 1H), 8.85 (ddd, J=4.9, 1.7, 1.2 Hz, 1H), 8.23 (dd, J=9.3, 5.6 Hz, 1H), 7.93 (td, J=7.6, 1.7 Hz, 1H), 7.57 (ddd, J=10.8, 7.8, 2.9 Hz, 1H), 7.51-7.47 (series of m, 2H), 7.29 (dd, J=10.0, 2.7 Hz, 1H), 2.20 (s, 3H). Mass Spectrum (ESI) m/e=267.1 (M+1).

### Specific Examples of General Method A10

### 1-(8-Chloro-6-fluoro-4-(pyridin-2-yl)quinolin-3-yl)ethanamine

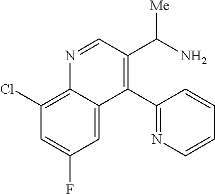 [SMILES: CC(N)c1cnc2c(Cl)cc(F)cc2c1-c1ccccn1]

1-(8-Chloro-6-fluoro-4-(pyridin-2-yl)quinolin-3-yl)ethanone (0.276 g, 0.918 mmol) was dissolved in ammonia 7M in methanol (5.00 mL, 35.0 mmol) and then to this was added titanium (IV) isopropoxide (0.538 mL, 1.836 mmol). The solution was then stirred at rt overnight. The next day the solution was cooled in an ice bath before adding sodium borohydride (0.069 g, 1.836 mmol). After 20 min, water was added to the suspension followed by DCM. The suspension was stirred vigorously and then filtered through filter paper. The solids were washed thoroughly with DCM and H₂O. The filtrates were partitioned and the aqueous layer was washed with DCM. The organics were dried over MgSO₄ and then concentrated under vacuum to provide 1-(8-chloro-6-fluoro-4-(pyridin-2-yl)-quinolin-3-yl)ethanone (230 mg) as a yellow foam which was carried on without further purification. Mass Spectrum (ESI) m/e=302.2 (M+1).

### 1-(6-Fluoro-4-(pyridin-2-yl)quinolin-3-yl)ethanamine

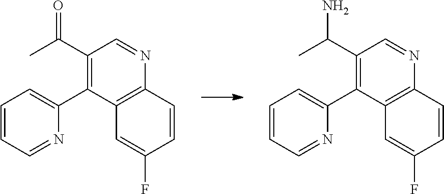 [SMILES: CC(=O)c1cnc2ccc(F)cc2c1-c1ccccn1 ; CC(N)c1cnc2ccc(F)cc2c1-c1ccccn1]

A mixture of titanium(IV) isopropoxide (770 μL, 2.63 mmol), ammonia (˜7M in methanol, 939 μL, 6.57 mmol) and 1-(6-fluoro-4-(pyridin-2-yl)quinolin-3-yl)-ethanone (350 mg, 1.314 mmol) were stirred overnight under an inert atmosphere. The mixture was then treated with NaBH₄ (99 mg, 2.63 mmol). After the reaction was judged to be complete, it was worked up by addition of NH₄OH. The resulting solids were filtered off and the filtrate was concentrated and purified using 0-100% (90:10:1 DCM:Methanol:NH₄OH) in DCM to afford 1-(6-fluoro-4-(pyridin-2-yl)quinolin-3-yl)ethanamine. ¹H NMR (500 MHz, CDCl₃) δ ppm 9.21 (s, 1H), 8.83 (ddd, J=5.1, 2.0, 1.0 1H), 8.15 (dd, J=9.1, 5.4, 1H), 7.91 (td, J=7.6, 1.7, 1H), 7.49-7.36 (series of m, 3H), 6.91 (m, 1H), 4.05 (m, 1H), 1.43 (m, 3H).

### Specific Example of General Method A11

### 1-(4-Chloro-6-fluoroquinolin-3-yl)ethanol

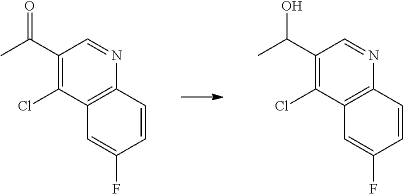

To a solution of 1-(4-chloro-6-fluoroquinolin-3-yl)ethanone (1 g, 4.47 mmol) in 20 mL of in methanol at ˜10° C. was added sodium borohydride (0.169 g, 4.47 mmol). The reaction was cooled to 0° C. and stirred for 30 min. The solvent was removed and the residue was redissolved in DCM/water. The layers were separated and the aqueous layer was extracted with DCM. The combined organic layers were washed with brine and dried over MgSO₄, filtered and concentrated to afford 1-(4-chloro-6-fluoroquinolin-3-yl)ethanol. ¹H NMR (500 MHz, CDCl₃) δ ppm 9.08 (s, 1H), 8.11 (dd, J=9.2, 5.3 Hz, 1H), 7.84 (ddd, J=9.6, 2.7, 0.4 Hz, 1H), 7.51 (ddd, J=10.8, 7.8, 2.7 Hz, 1H), 5.55 (qd, J=6.5, 3.7 Hz, 1H), 2.49 (d, J=3.7 Hz, 1H), 1.62 (d, J=6.5 Hz, 3H).

### Specific Example of General Method B4

### Example 4

### 4-Amino-6-(((1S)-1-(8-chloro-6-fluoro-4-(2-pyridinyl)-3-quinolinyl)ethyl)amino)-5-pyrimidinecarbonitrile and 4-Amino-6-(((1R)-1-(8-chloro-6-fluoro-4-(2-pyridinyl)-3-quinolinyl)ethyl)amino)-5-pyrimidinecarbonitrile

### 4-Amino-6-(((15)-1-(8-chloro-6-fluoro-4-(2-pyridinyl)-3-quinolinyl)ethyl)-amino)-5-pyrimidinecarbonitrile

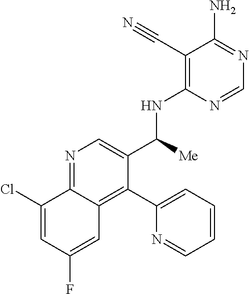

The enantiomers of 4-amino-6-(1-(8-chloro-6-fluoro-4-(pyridin-2-yl)quinolin-3-yl)ethylamino)pyrimidine-5-carbonitrile were separated on a OJ-H chiral SFC column (3×15 cm) eluting with 25% methanol (20 mM NH₄)/CO₂, 100 Bar. The fractions containing the first peak to elute were combined and concentrated under vacuum to provide 4-amino-6-(((1S)-1-(8-chloro-6-fluoro-4-(2-pyridinyl)-3-quinolinyl)ethyl)amino)-5-pyrimidinecarbonitrile as a white solid. The stereochemistry is arbitrarily assigned. A mixture of isomers was observed in the proton NMR trace. ¹H NMR (400 MHz, DMSO-d₆) δ ppm 9.30 (1H, s), 8.79 (1 H, d, J=4.7 Hz), 7.95-8.13 (2.7H, m), 7.84 (0.8H, br. s.), 7.74 (0.8H, d, J=8.0 Hz), 7.64 (0.3H, br. s.), 7.57 (1.2H, t, J=6.7 Hz), 7.22 (2H, br. s.), 6.89 (1H, d, J=9.6 Hz), 5.31-5.51 (0.2H, m), 5.05 (0.8H, m, J=13.0, 6.4, 6.4 Hz), 1.57 (0.5H, br. s.), 1.47 (2.4H, d, J=6.1 Hz). Mass Spectrum (ESI) m/e=420.1 (M+1). EE>99%.

### 4-Amino-6-(((1R)-1-(8-chloro-6-fluoro-4-(2-pyridinyl)-3-quinolinyl)ethyl)-amino)-5-pyrimidinecarbonitrile

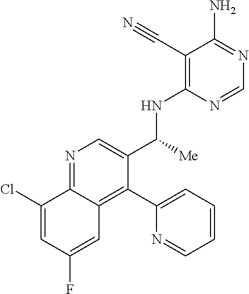 [SMILES: C[C@@H](Nc1ncnc(N)c1C#N)c1cnc2c(Cl)cc(F)cc2c1-c1ccccn1]

The enantiomers of 4-amino-6-(1-(8-chloro-6-fluoro-4-(pyridin-2-yl)quinolin-3-yl)ethylamino)pyrimidine-5-carbonitrile were separated on a OJ-H chiral SFC column (3×15 cm) eluting with 25% methanol (20 mM NH₄)/CO₂, 100 Bar. The fractions containing the second peak to elute were combined and concentrated under vacuum to provide 4-amino-6-(((1R)-1-(8-chloro-6-fluoro-4-(2-pyridinyl)-3-quinolinyl)ethyl)amino)-5-pyrimidinecarbonitrile as a white solid. The stereochemistry is arbitrarily assigned. A mixture of isomers was observed in the proton NMR trace. ¹H NMR (400 MHz, DMSO-d₆) δ ppm 9.30 (1H, s), 8.79 (1 H, d, J=4.7 Hz), 7.95-8.13 (2.7H, m), 7.84 (0.8H, br. s.), 7.74 (0.8H, d, J=8.0 Hz), 7.64 (0.3H, br. s.), 7.57 (1.2H, t, J=6.7 Hz), 7.22 (2H, br. s.), 6.89 (1H, d, J=9.6 Hz), 5.31-5.51 (0.2H, m), 5.05 (0.8H, m, J=13.0, 6.4, 6.4 Hz), 1.57 (0.5H, br. s.), 1.47 (2.4H, d, J=6.1 Hz). Mass Spectrum (ESI) m/e=420.1 (M+1). EE>99%.

### Specific Example of General Method B11

### 1-(4-Phenylquinolin-3-yl)ethanol

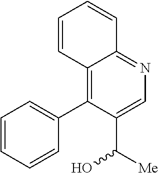 [SMILES: CC(O)c1cnc2ccccc2c1-c1ccccc1]

At 0° C. and under an atmosphere of N₂ was dissolved 1-(4-phenylquinolin-3-yl)-ethanone (0.229 g, 0.926 mmol) in 7 mL of methanol. To this solution was added sodium borohydride (0.042 mL, 1.204 mmol). The yellow solution was left to warm to rt. After 1 h the solution was concentrated under vacuum and then diluted with sat NaHCO₃. The product was extracted with ethyl acetate and the organics were dried over MgSO₄ before being concentrated under vacuum. The residue obtained was purified by column chromatography using a gradient of 40% ethyl acetate/hexane to 60% ethyl acetate/hexane. The fractions containing the product were combined and concentrated under vacuum to provide 1-(4-phenylquinolin-3-yl)ethanol as a light yellowish/white foam. ¹H NMR (500 MHz, DMSO-d₆) δ ppm 9.15 (1H, s), 8.06 (1H, d, J=8.1 Hz), 7.72 (1H, ddd, J=8.4, 6.9, 1.3 Hz), 7.47-7.62 (4H, m), 7.33-7.38 (1H, m), 7.26-7.33 (2H, m), 5.35 (1H, d, J=3.7 Hz), 4.63 (1H, qd, J=6.4, 3.9 Hz), 1.32 (3H, d, J=6.4 Hz). TLC (30% ethyl acetate/hexane, product's R_{f}=0.31).

### Specific Example of General Method B10

### 2-(1-(4-Phenylquinolin-3-yl)ethyl)isoindoline-1,3-dione

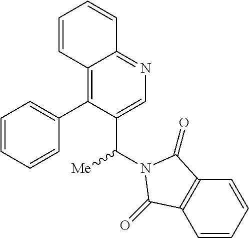 [SMILES: CC(c1cnc2ccccc2c1-c1ccccc1)N1C(=O)c2ccccc2C1=O]

Combined isoindoline-1,3-dione (0.110 g, 0.746 mmol), triphenylphosphine (0.196 g, 0.746 mmol), and 1-(4-phenylquinolin-3-yl)ethanol (0.155 g, 0.622 mmol) in 8 mL of anhydrous THF. The solution was then cooled in an ice bath before adding diisopropylazodicarboxylate (DIAD) (0.147 mL, 0.746 mmol). The solution was then left to warm to rt and stirred over the weekend. The solution was then concentrated under vacuum and then diluted with ethyl acetate. The organics were washed in succession with H₂O and brine, before being dried over MgSO₄ and then concentrated under vacuum. The yellow oil obtained was purified by column chromatography using a gradient of 10% ethyl acetate/hexane to 50% ethyl acetate/hexane. The fractions containing the product were combined and concentrated under vacuum to provide 2-(1-(4-phenylquinolin-3-yl)ethyl)-isoindoline-1,3-dione (169 mg, crude) as a light yellow solid which was carried on without further purification. Mass Spectrum (ESI) m/e=379.1 (M+1).

### Specific Example of General Method B5

### 4,7-Dichloro-quinoline-3-carboxylic acid

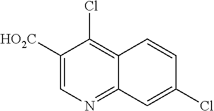 [SMILES: O=C(O)c1cnc2cc(Cl)ccc2c1Cl]

4,7-Dichloro-quinoline-3-carboxylic acid ethyl ester (Journal of Medicinal Chemistry, 2006, vol. 49, #21 p. 6351-6363) (35 g, 129.62 mmol) was dissolved in acetonitrile (175 mL) and then cooled in a ice bath. To this was added a solution of LiOH.2H₂O (8.16 g, 194.28 mmol) dissolved in water (150 mL). The reaction mixture was stirred at rt for 6 h. The mixture was concentrated to half the volume under vacuum. The pH of the mixture was adjusted to ˜8-9 with 5N HCl. The solids were filtered off through a Buchner funnel and then washed with water followed by diethyl ether to provide 4,7-dichloro-quinoline-3-carboxylic acid as a solid. Mass Spectrum (ESI) m/e=241.99 (M+2). TLC (50% ethyl acetate in hexane, product's R_{f}=0.2).

### Specific Example of General Method B6

### 4,7-Dichloro-N-methoxy-N-methylquinoline-3-carboxamide

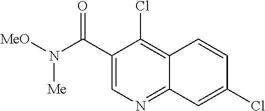 [SMILES: CON(C)C(=O)c1cnc2cc(Cl)ccc2c1Cl]

To a suspension of 4,7-dichloro-quinoline-3-carboxylic acid (8 g) in toluene (100 mL) at 0° C. was added SOCl₂ (100 mL). The mixture was refluxed at 110° C. for 12 h. The mixture was evaporated under high vacuum. Under an atmosphere of N₂, the residue obtained was combined with DCM (70 mL). The solution was cooled in a ice bath before adding triethyl amine (18.48 mL, 132.84 mmol) followed by N,O-dimethyl hydroxyl-amine hydrochloride (2.9 g, 29.736 mmol). The mixture was stirred at 0° C. for 1 h. The mixture was diluted with water and the product was extracted with DCM. The organic layer was dried over Na₂SO₄ and concentrated under vacuum to provide 4,7-dichloro-N-methoxy-N-methylquinoline-3-carboxamide as a brown solid, the material was carried on without further purification. Mass Spectrum (ESI) m/e=285.0 (M+1). TLC (30% ethyl acetate in hexane, product's R_{f}=0.7).

### Specific Example of General Method B7

### 1-(4,7-Dichloro-quinolin-3-yl)-ethanone

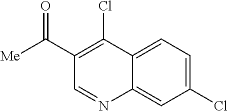 [SMILES: CC(=O)c1cnc2cc(Cl)ccc2c1Cl]

A solution of 4,7-dichloro-N-methoxy-N-methylquinoline-3-carboxamide (7 g, 24.64 mmol) in THF (70 mL) was cooled to −70° C. To this was added methyl lithium (1.5M in THF, 18 mL) dropwise. The temperature of the reaction mixture was raised to −20° C. from −70° C. within 1 h. The reaction mixture was quenched with sat NH₄Cl and the product was extracted with ethyl acetate. The organic layer was dried over Na₂SO₄ and then concentrated under vacuum. The residue obtained was purified by column chromatography using 5% ethyl acetate in hexane as eluent to provide 1-(4,7-dichloro-quinolin-3-yl)-ethanone as a solid. ¹HNMR: (400 MHz, CDCl₃) δ ppm 8.99 (s, 1H), 8.321 (d, J=8.8 Hz, 1H), 8.148 (d, J=2 Hz, 1H), 7.672 (dd, J=8.8 Hz, 2 Hz, 1H), 2.801 (s, 3H). Mass Spectrum (ESI) m/e=240.06 (M+1). TLC (30% ethyl acetate in hexane, product's R_{f}=0.8).

### Specific Example of General Method B8

### 4,6-Dichloro-N-methoxy-N-methylquinoline-3-carboxamide

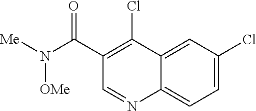 [SMILES: CON(C)C(=O)c1cnc2ccc(Cl)cc2c1Cl]

To a solution of 4,6-dichloro-quinoline-3-carboxylic acid (prepared as in General Method B5 from ethyl 4,6-dichloroquinoline-3-carboxylate (Journal of Medicinal Chemistry, 1993, vol. 36, #11, p. 1669-1673.) (20 g, 0.0826 mol) in DMF (100 mL), HATU (47 g, 0.123 mol) and DIPEA (26.6 g, 0.2066 mol) were added. The reaction mixture was stirred for 10 min, before N,O-dimethyl hydroxyl-amine hydrochloride (9.6 g, 0.099 mol) was added. The mixture was stirred overnight and then diluted with water. The product was extracted with ethyl acetate. The organic phase was dried over Na₂SO₄ and then concentrated under vacuum. The residue obtained was purified by column chromatography using 10% ethyl acetate in hexane as eluent to obtain 4,6-dichloro-quinoline-3-carboxylic acid methoxy-methyl-amide as a solid. TLC (40% ethyl acetate in hexane, product's R_{f}=0.6).

### Specific Example of General Method B12

### 1-(4-Phenylquinolin-3-yl)ethanone

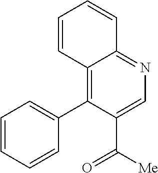 [SMILES: CC(=O)c1cnc2ccccc2c1-c1ccccc1]

Following a similar protocol as described in Tetrahedron Letters, 36(31), 5461-4; 1995, a suspension of ethyl 4-phenylquinoline-3-carboxylate (0.414 g, 1.493 mmol) and N,O-dimethylhydroxylamine hydrochloride (0.146 g, 1.493 mmol) in 20 mL of anhydrous THF under an atmosphere of N₂ was cooled in a ice bath. To this was added slowly methylmagnesium bromide 3.0 M in Et₂O (3.98 mL, 11.94 mmol) over a period of 10 min. The solution was allowed to warm to rt overnight. The next day 20 mL of 2N HCl was added and then the solution was stirred at 35° C. for 2 h. The pH of the solution was adjusted to ˜9 with sat NaHCO₃ and then the product was extracted with DCM. The organics were dried over Na₂SO₄ and then concentrated under vacuum. An orange oil was obtained and purified by column chromatography using a gradient of 20% ethyl acetate/hexane to ethyl acetate. The fractions containing the product were combined and concentrated under vacuum to give 1-(4-phenylquinolin-3-yl)-ethanone 229 mg of a yellow oil, the material was carried on without further purification. Mass Spectrum (ESI) m/e=248.1 (M+1).

### Specific Example of General Method B13

### Ethyl 4-phenylquinoline-3-carboxylate

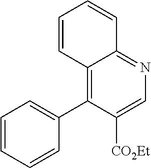 [SMILES: CCOC(=O)c1cnc2ccccc2c1-c1ccccc1]

Ethyl 4-chloroquinoline-3-carboxylate (Journal of Medicinal Chemistry, 2006, vol. 49, #21, p. 6351-6363) (0.443 g, 1.880 mmol), phenylboronic acid (0.344, 2.82 mmol), and potassium carbonate (0.779 g, 5.64 mmol) were combined in 18 mL of DMF. The solution was sparged with N₂ before adding PdCl₂(dppf)2-CH2Cl2 (0.154 g, 0.188 mmol) and then it was heated to 110° C. overnight. The next day the solution was concentrated under vacuum and the residue obtained was purified by column chromatography using a gradient of 15% ethyl acetate/hexane to 60% ethyl acetate/hexane. The fractions containing the product were combined and concentrated under vacuum to provide ethyl 4-phenyl-quinoline-3-carboxylate as clear oil. ¹H NMR (500 MHz, CHLOROFORM-d) δ ppm 9.36 (1H, s), 8.22 (1H, d, J=8.6 Hz), 7.81 (1H, ddd, J=8.4, 6.8, 1.3 Hz), 7.62 (1H, d, J=7.6 Hz), 7.49-7.55 (4H, m), 7.29-7.34 (2H, m), 4.13 (2H, q, J=7.3 Hz), 1.02 (3H, t, J=7.2 Hz). Mass Spectrum (ESI) m/e=278.0 (M+1).

### Specific Example of General Method B14

### 2-(1-(8-Fluoro-4-(3-fluorophenyl)quinolin-3-yl)ethyl)isoindoline-1,3-dione

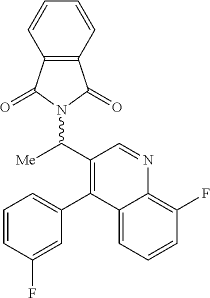 [SMILES: CC(c1cnc2c(F)cccc2c1-c1cccc(F)c1)N1C(=O)c2ccccc2C1=O]

2-(1-(4-Chloro-8-fluoroquinolin-3-yl)ethyl)isoindoline-1,3-dione (0.117 g, 0.330 mmol), potassium phosphate (0.210 g, 0.989 mmol), and 3-fluorophenylboronic acid (0.092 g, 0.660 mmol) were combined in 5 mL of t-amyl alcohol and 5 mL of 1,4-dioxane. The suspension was briefly sparged with N₂ before adding Pd(dba)₂ (0.013 g, 0.022 mmol) and dicyclohexyl(2′,4′,6′-triisopropylbiphenyl-2-yl)-phosphine (0.021 g, 0.044 mmol). The suspension was heated to 95° C. and monitored by LC/MS positive for the absence of the starting material. After 4 h the suspension was cooled to rt and then diluted into H₂O. The product was extracted with DCM. The organics were dried over MgSO₄ and then concentrated under vacuum. The residue obtained was purified by column chromatography using a gradient of 20% ethyl acetate/hexane to 100% ethyl acetate. The fractions containing the product were combined and concentrated under vacuum to provide 2-(1-(4-(3,5-difluorophenyl)-8-fluoroquinolin-3-yl)ethyl)isoindoline-1,3-dione as a pink solid. Mass Spectrum (ESI) m/e=415.2 (M+1).

### Additional Specific Examples

### 2-(1-(4-Cyclopropyl-6-fluoroquinolin-3-yl)ethyl)isoindoline-1,3-dione

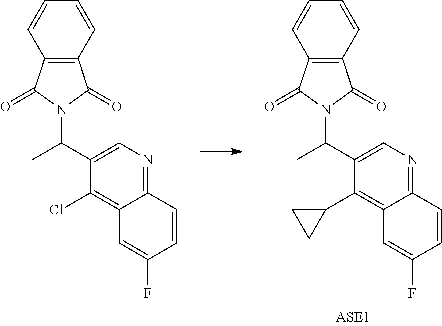 [SMILES: CC(c1cnc2ccc(F)cc2c1Cl)N1C(=O)c2ccccc2C1=O ; CC(c1cnc2ccc(F)cc2c1C1CC1)N1C(=O)c2ccccc2C1=O]

A reaction vessel was charged with tetrakistriphenylphosphine palladium (0) (65.1 mg, 0.056 mmol), 2-(1-(4-chloro-6-fluoroquinolin-3-yl)ethyl)isoindoline-1,3-dione (200 mg, 0.564 mmol) and toluene (4 mL). The vessel was purged with argon and treated with 0.5 M cyclopropylzinc(II) bromide in THF (1691 μL, 0.846 mmol) and heated to 90° C. The reaction was monitored by LC/MS and an additional 1.5 eq of cyclopropylzinc(II) bromide was added to progress the reaction to near completion. The reaction was cooled to rt and quenched with 50% sat NH₄Cl and diluted with ethyl acetate. The layers were separated and the organic layer was washed with brine, dried over MgSO₄, filtered, and concentrated. Purification using 15-20% ethyl acetate in hexane afforded 2-(1-(4-cyclopropyl-6-fluoroquinolin-3-yl)ethyl)isoindoline-1,3-dione. ¹H NMR (500 MHz, CDCl₃) δ ppm 9.33 (s, 1H), 8.10 (dd, J=11.0, 2.9 Hz, 1H), 8.06 (dd, J=9.2, 5.7 Hz, 1H), 7.80 (m, 2H), 7.70 (m, 2H), 7.43 (ddd, J=9.4, 8.2, 2.7 Hz, 1H), 6.57 (q, J=7.3 Hz, 1H), 2.12 (tt, J=8.4, 5.9 Hz, 1H), 2.07 (d, J=7.6 Hz, 3H), 1.47 (tdd, J=9.4, 5.9, 4.7 Hz, 1H), 1.40 (tt, J=9.6, 4.7 Hz, 1H), 0.89 (m, 1H), 0.76 (tt, J=9.6, 5.6 Hz, 1H). Mass Spectrum (ESI) m/e=361.2 (M+1).

### 4-(2-Formylphenyl)but-3-yn-2-yl acetate

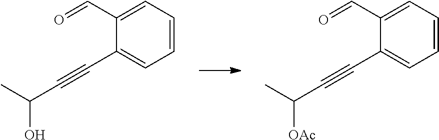 [SMILES: CC(O)C#Cc1ccccc1C=O ; CC(=O)OC(C)C#Cc1ccccc1C=O]

To a solution of 2-(3-hydroxybut-1-ynyl)benzaldehyde (1 g, 5.74 mmol) (Shu, Xing-Zhong; Zhao, Shu-Chun; Ji, Ke-Gong; Zheng, Zhao-Jing; Liu, Xue-Yuan; Liang, Yong-Min Eur. J. Org. Chem., 2009, 1, 117) and triethylamine (1.600 mL, 11.48 mmol) in 20 mL of anhydrous DCM was added acetyl chloride (1 M solution in DCM, 7.46 mL, 7.46 mmol). After 1 h, an additional charge of 2 mL of 1M acetyl chloride was added. The reaction was stirred for 1 h and quenched with sat NaHCO₃. The layers were separated and the organic layer was washed with brine. Concentration and purification by column chromatography using 10-20% ethyl acetate in hexane afforded 4-(2-formylphenyl)but-3-yn-2-yl acetate. ¹H NMR (500 MHz, CDCl₃) δ ppm 10.49 (s, 1H), 7.93 (d, J=7.6 Hz, 1H), 7.56 (m, 2H), 7.47 (m, 1H), 5.71 (q, J=6.6 Hz, 1H), 2.13 (s, 3H), 1.63 (d, J=6.9 Hz, 3H). Mass Spectrum (ESI) m/e=239.2 (M+23).

### (Z)-4-(2-((Hydroxyimino)methyl)phenyl)but-3-yn-2-yl acetate

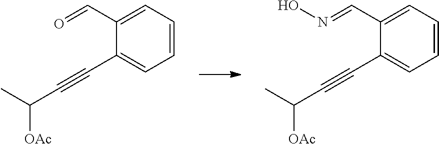 [SMILES: CC(=O)OC(C)C#Cc1ccccc1C=O ; CC(=O)OC(C)C#Cc1ccccc1/C=N/O]

To a solution of 4-(2-formylphenyl)but-3-yn-2-yl acetate (0.65 g, 3.01 mmol) and pyridine (0.485 mL, 6.01 mmol) in ethanol (30.1 mL) was added hydroxylamine hydrochloride (0.418 g, 6.01 mmol). After 30 min, LC/MS and NMR showed the reaction was complete. The solvent was removed in vacuo and the residue was redissolved in ethyl acetate and washed with sat CuSO₄, water and brine. The organic phase was dried over MgSO₄, filtered and concentrated to afford (Z)-4-(2-((hydroxyimino)methyl)phenyl)but-3-yn-2-yl acetate. The oxime geometry was not confirmed. ¹H NMR (500 MHz, CDCl₃) δ ppm 8.59 (br s, 1H), 7.85 (m, 1H), 7.47 (m, 1H), 7.34 (m, 2H), 5.70 (q, J=6.6 Hz, 1H), 2.13 (s, 3H), 1.62 (d, J=6.9 Hz, 3H). Mass Spectrum (ESI) m/e=232.2 (M+1).

### 3-(1-Acetoxyethyl)-4-bromoisoquinoline 2-oxide

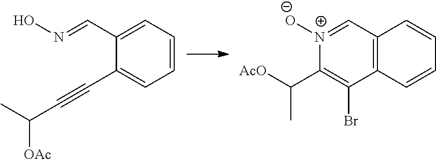 [SMILES: CC(=O)OC(C)C#Cc1ccccc1/C=N/O ; CC(=O)OC(C)c1c(Br)c2ccccc2c[n+]1[O-]]

To a solution of (Z)-4-(2-((hydroxyimino)methyl)phenyl)but-3-yn-2-yl acetate (924 mg, 4 mmol) in 40 mL DCM at 0° C. was added N-bromosuccinimide-(NBS, 800 mg, 4.4 mmol) in 40 mL anhydrous DCM. After 1 h, the reaction was treated with 0.1 M Na₂S₂O₃. The layers were separated and the organic phase was washed with sat NaHCO₃ and brine, dried over MgSO₄, filtered, and concentrated. Purification using 0-95% ethyl acetate in hexane afforded 3-(1-acetoxyethyl)-4-bromoisoquinoline 2-oxide. ¹H NMR (500 MHz, CDCl₃) δ ppm 8.77 (s, 1H), 8.16 (d, J=83 Hz, 1H), 7.61 (m, 3H), 6.92 (q, J=7.1 Hz, 1H), 2.16 (s, 3H), 1.82 (d, J=5.9 Hz, 3H) ppm. Mass Spectrum (ESI) m/e=310.0 (M+1).

### 4-Bromo-3-(1-hydroxyethyl)isoquinoline 2-oxide

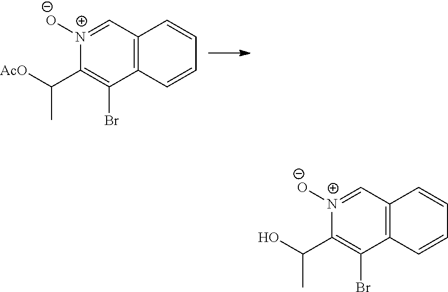 [SMILES: CC(=O)OC(C)c1c(Br)c2ccccc2c[n+]1[O-] ; CC(O)c1c(Br)c2ccccc2c[n+]1[O-]]

To a solution of 3-(1-acetoxyethyl)-4-bromoisoquinoline 2-oxide (780 mg, 2.51 mmol) in 20 mL methanol as added aqueous potassium carbonate (1 M, 5533 μL, 5.53 mmol). After 30 min, the solvent was removed and the residue was redissolved in ethyl acetate and washed with water and brine. The organic phase was dried over MgSO₄, filtered, and concentrated to afford 4-bromo-3-(1-hydroxyethyl)isoquinoline 2-oxide. ¹H NMR (500 MHz, CDCl₃) δ ppm 8.80 (s, 1H), 8.21 (d, J=8.8 Hz, 1H), 7.76 (m, 2H), 7.68 (ddd, J=8.1, 7.2, 1.2 Hz, 1H), 5.72 (q, J=5.9 Hz, 1H), 1.74 (j=6.9 Hz, 3H).

### 4-Bromo-3-(1-(1,3-dioxoisoindolin-2-yl)ethyl)isoquinoline 2-oxide

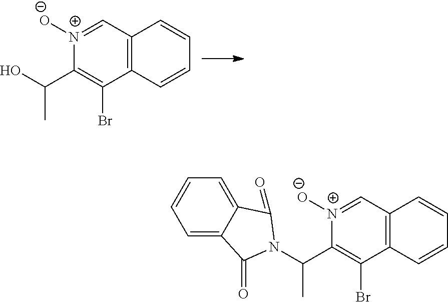 [SMILES: CC(O)c1c(Br)c2ccccc2c[n+]1[O-] ; CC(c1c(Br)c2ccccc2c[n+]1[O-])N1C(=O)c2ccccc2C1=O]

To a solution of triphenylphosphine (411 mg, 1.567 mmol), phthalimide (230 mg, 1.567 mmol) and 4-bromo-3-(1-hydroxyethyl)isoquinoline 2-oxide (350 mg, 1.305 mmol) in 13 mL of anhydrous THF was added DIAD (305 μL, 1.567 mmol) dropwise. After 2 h, the solvent was removed in vacuo. The residue was treated with 8 mL of isopropanol and sonicated in an ultrasound bath until a precipitate formed. The mixture was stirred for 1 h, filtered, and washed with isopropanol to afford 4-bromo-3-(1-(1,3-dioxoisoindolin-2-yl)ethyl)isoquinoline 2-oxide as a 1:1 solvate with isopropanol. ¹H NMR (500 MHz, CDCl₃) δ ppm 8.79 (s, 1H), 8.23 (d, J=8.8 Hz, 1H), 7.81 (m, 2H), 7.70 (m, 4H), 7.65 (m, 1H), 6.47 (q, J=7.3 Hz, 1H), 4.05 (septet, J=6.1 Hz, 1H) (isopropanol), 2.25 (d, J=7.6 Hz, 3H), 1.23 (d, J=6.1 Hz, 6H) (isopropanol). Mass Spectrum (ESI) m/e=397.0 (M+1).

### 2-(1-(4-Bromoisoquinolin-3-yl)ethyl)isoindoline-1,3-dione

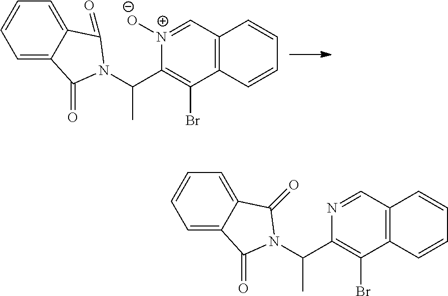 [SMILES: CC(c1c(Br)c2ccccc2c[n+]1[O-])N1C(=O)c2ccccc2C1=O ; CC(c1ncc2ccccc2c1Br)N1C(=O)c2ccccc2C1=O]

To a solution of 4-bromo-3-(1-(1,3-dioxoisoindolin-2-yl)ethyl)isoquinoline 2-oxide (450 mg, 1.133 mmol) in THF (10 mL) was added titanium(III) chloride (30 wt % in 2N HCl, 1281 mg, 2.492 mmol) dropwise. After 10 min, added an additional 300 mg of TiCl₃ solution was added. The reaction was quenched with sat NaHCO₃ solution. The aqueous solution was extracted with ethyl acetate. The organic phase was washed with brine, dried over MgSO₄, filtered, and concentrated. Purification by column chromatography (10-20% ethyl acetate in hexane) afforded 2-(1-(4-bromoisoquinolin-3-yl)ethyl)isoindoline-1,3-dione. ¹H NMR (500 MHz, CDCl₃) δ ppm 9.22 (s, 1H), 8.23 (d, J=8.6 Hz, 1H), 7.97 (d, J=8.3 Hz, 1H), 7.85-7.78 (series of m, 3H), 7.71 (m, 2H), 7.67 (ddd, J=8.1, 7.1, 1.0 Hz, 1H), 6.07 (q, J=7.1 Hz, 1H), 2.06 (d, J=7.3 Hz, 3H). Mass Spectrum (ESI) m/e=381.1 (M+1).

### 2-(1-(4-Phenylisoquinolin-3-yl)ethyl)isoindoline-1,3-dione (ASE2)

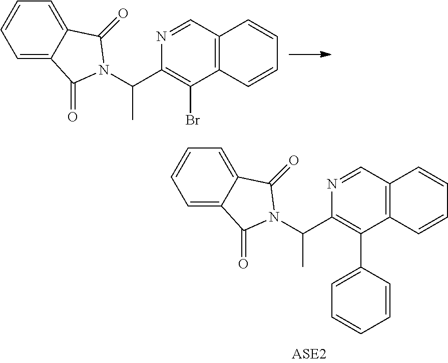

In a reaction vessel was combined potassium phosphate (55.6 mg, 0.262 mmol), phenylboronic acid (23.99 mg, 0.197 mmol), palladium (II) acetate (0.589 mg, 2.62 μmmol), 2-dicyclohexylphosphino-2,6-dimethoxybiphenyl (2.69 mg, 6.56 μmol) and 2-(1-(4-bromoisoquinolin-3-yl)ethyl)isoindoline-1,3-dione (50 mg, 0.131 mmol). The mixture was purged with argon, diluted with toluene (2 mL) and heated at 100° C. overnight. The reaction was repeated on a 2× scale and the reactions were combined for workup. Purification by column chromatography using 10-20% ethyl acetate in hexane afforded 2-(1-(4-phenylisoquinolin-3-yl)-ethyl)isoindoline-1,3-dione. ¹H NMR (500 MHz, CDCl₃) δ ppm 9.33 (1H), 8.01 (m, 1H), 7.74 (m, 2H), 7.68 (m, 2H), 7.57 (m, 3H), 7.43 (tt, J=7.3, 1.2 Hz, 1H), 7.35 (m, 2H), 7.30 (m, 1H), 7.25 (m, 1H), 5.67 (q, J=7.1 Hz, 1H), 1.91 (d, J=7.3 Hz, 3H). Mass Spectrum (ESI) m/e=379.2 (M+1).

### (E)-N-((1-Bromonaphthalen-2-yl)methylene)-2-methylpropane-2-sulfinamide

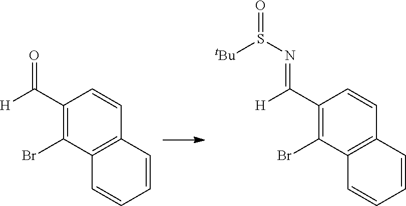 [SMILES: O=Cc1ccc2ccccc2c1Br ; CC(C)(C)S(=O)/N=C/c1ccc2ccccc2c1Br]

To a solution of 2-methyl-2-propane-sulfinamide (88 mg, 0.723 mmol) and 1-bromo-2-naphthaldehyde (170 mg, 0.723 mmol) dissolved in tetrahydrofuran (5 mL) was added titanium (iv) ethoxide (0.299 mL, 1.446 mmol). The resulting solution was heated to 75° C. overnight. After sixteen hours thin layer chromatography indicated very little starting material remained. The reaction was equilibrated to rt then poured into 50 mL brine. The resulting precipitate was removed by filtration, rinsing with 50 mL ethyl acetate. The organic separation was stirred over anhydrous magnesium sulfate, filtered and the filtrate concentrated under reduced pressure to afford a yellow, crystalline solid. ¹H NMR (400 MHz, CHLOROFORM-d) δ ppm 9.17 (1H, s), 8.21-8.34 (1H, m), 7.95 (1H, d, J=8.4 Hz), 7.62-7.76 (2H, m), 7.39-7.56 (2H, m), 1.20 (9H, s).

### N-(1-(1-Bromonaphthalen-2-yl)ethyl)-2-methylpropane-2-sulfinamide

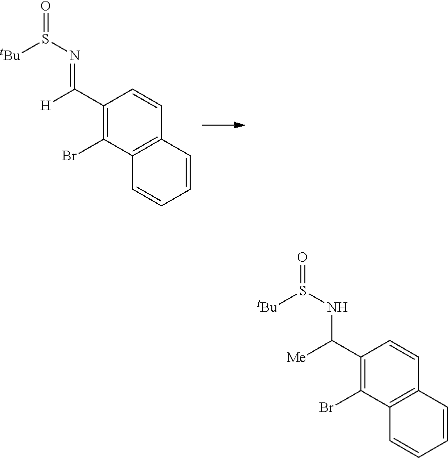 [SMILES: CC(C)(C)S(=O)/N=C/c1ccc2ccccc2c1Br ; CC(NS(=O)C(C)(C)C)c1ccc2ccccc2c1Br]

To a solution of (E)-N-((1-bromonaphthalen-2-yl)methylene)-2-methylpropane-2-sulfinamide (240 mg, 0.710 mmol) dissolved in tetrahydrofuran (7 mL) cooled by an acetone dry ice bath was added 3.0M methylmagnesium bromide in diethyl ether (0.710 mL, 2.129 mmol). After 15 min the cold bath was removed and the reaction stirred to rt overnight. After sixteen hours the reaction was poured into 25 mL sat aqueous ammonium chloride solution and extracted with 2×25 mL ethyl acetate. The combined organic extracts were stirred over anhydrous magnesium sulfate, filtered and the filtrate concentrated under reduced pressure to afford a colorless foamy solid. Mass Spectrum (ESI) m/e=354.0 and 356.0 (M+1).

### N-(1-(1-(3,5-Difluorophenyl)naphthalen-2-yl)ethyl)-2-methylpropane-2-sulfinamide

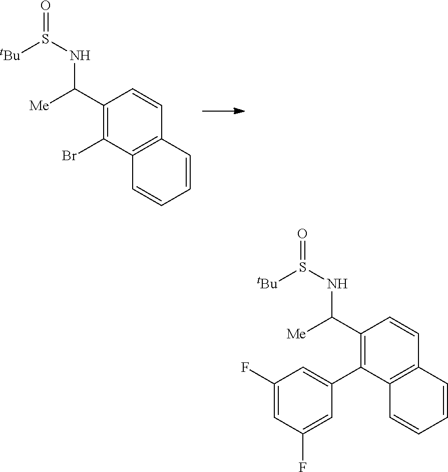 [SMILES: CC(NS(=O)C(C)(C)C)c1ccc2ccccc2c1Br ; CC(NS(=O)C(C)(C)C)c1ccc2ccccc2c1-c1cc(F)cc(F)c1]

A mixture of 3,5-difluorophenylboronic acid (167 mg, 1.058 mmol), palladium (II) acetate (15.84 mg, 0.071 mmol), 2-dicyclohexylphosphino-2′,6′-dimethoxy-1,1′-biphenyl (72.4 mg, 0.176 mmol), potassium phosphate (0.117 mL, 1.411 mmol) and N-(1-(1-bromonaphthalen-2-yl)ethyl)-2-methylpropane-2-sulfinamide (250 mg, 0.706 mmol) in toluene (9 mL) was purged with nitrogen then heated to 100° C. overnight. After 20 h, the toluene was removed under reduced pressure and the concentrated partitioned between 30 mL each water and ethyl acetate. The organic separation was stirred over MgSO₄, filtered and the filtrate concentrated under reduced pressure to afford a yellow oil. The product was isolated by chromatography on silica gel (40 g RediSep™ Rf Gold cartridge) eluting with 20-60% ethyl acetate in hexane to afford product as a colorless oil. Mass Spectrum (ESI) m/e=388.2 (M+1).

### 1-(1-(3,5-Difluorophenyl)naphthalen-2-yl)ethanamine

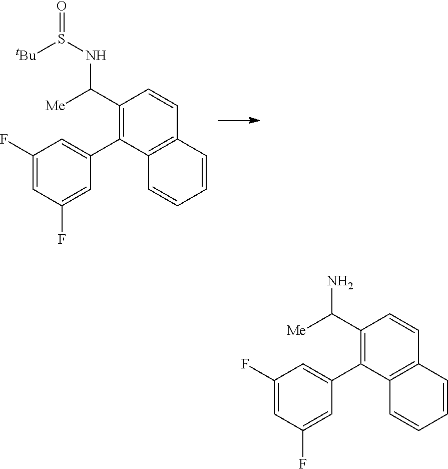 [SMILES: CC(NS(=O)C(C)(C)C)c1ccc2ccccc2c1-c1cc(F)cc(F)c1 ; CC(N)c1ccc2ccccc2c1-c1cc(F)cc(F)c1]

To a rt solution of N-(1-(1-(3,5-difluorophenyl)naphthalen-2-yl)ethyl)-2-methylpropane-2-sulfinamide (170 mg, 0.439 mmol) dissolved in tetrahydrofuran (5 mL) was added concentrated HCl (0.20 mL, 6.58 mmol) all in one portion. The reaction was stirred at ambient temperature for 5 minutes, after which time LC/MS indicated no starting material remained. The reaction was partitioned between 25 mL sat aqueous sodium bicarbonate and 30 mL ethyl acetate. The organic separation was stirred over anhydrous magnesium sulfate, filtered and the filtrate concentrated under reduced pressure to afford a foamy solid. Mass Spectrum (ESI) m/e=267.0 (M-NH₂).

### Example 5

### 4-Amino-6-((1-(1-(3,5-difluorophenyl)-2-naphthalenyl)ethyl)-amino)-5-pyrimidinecarbonitrile

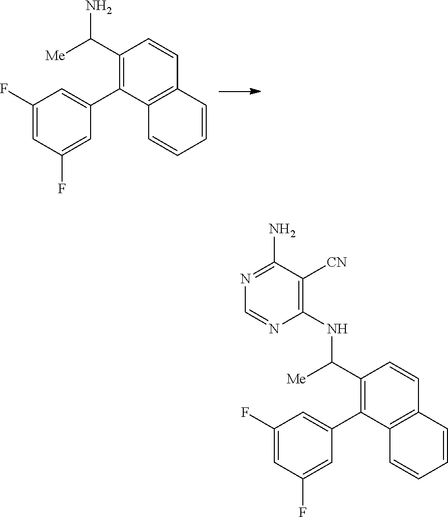 [SMILES: CC(N)c1ccc2ccccc2c1-c1cc(F)cc(F)c1 ; CC(Nc1ncnc(N)c1C#N)c1ccc2ccccc2c1-c1cc(F)cc(F)c1]

A mixture of 1-(1-(3,5-difluorophenyl)naphthalen-2-yl)ethanamine (124 mg, 0.438 mmol), 4-amino-6-chloropyrimidine-5-carbonitrile (71.0 mg, 0.460 mmol) and DIEA (0.114 mL, 0.657 mmol) in 1-butanol (3 mL) was heated to 100° C. overnight. After 16 h, the reaction was removed from heat. A precipitate formed upon cooling and was collected by filtration, rinsing with cold 1-butanol to afford 4-amino-6-((1-(1-(3,5-difluorophenyl)-2-naphthalenyl)ethyl)amino)-5-pyrimidinecarbonitrile as a colorless solid. ¹H NMR (400 MHz, DMSO-d₆) δ ppm 8.00 (1H, d, J=8.8 Hz), 7.90-7.97 (1H, m), 7.82-7.90 (2H, m), 7.75 (1H, d, J=7.2 Hz), 7.40-7.56 (2H, m), 7.36 (1H, m), 7.24 (3H, d, J=8.4 Hz), 7.11 (2H, d, J=8.4 Hz), 5.09 (1H, quin, J=7.1 Hz), 1.43 (3H, d, J=7.2 Hz). Mass Spectrum (ESI) m/e=402.0 (M+1).

### Methyl 8-hydroxyquinoline-7-carboxylate

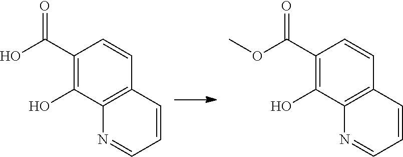

A 500 mL flask was charged with 8-hydroxyquinoline-7-carboxylic acid (10.0 g, 52.9 mmol) and methanol (300 mL). Concentrated sulfuric acid (5 mL) was added and the flask fitted with a Dean-Stark trap and water cooled condenser. The reaction was heated such that distillation occurred at a rate of about 10 mL/h. After 14 h the reaction was concentrated and dissolved in 400 mL ethyl acetate. This solution was washed twice with 100 mL sat NaHCO₃ and once with 100 mL sat NaCl, then dried over MgSO₄. Removal of solvent gave a white solid. ¹H NMR (500 MHz, DMSO-d₆): δ ppm 3.94 (s, 3H), 7.42 (d, J=8.8 Hz, 1H), 7.69 (dd, J=8.3, 4.2 Hz, 1H), 7.85 (d, J=8.8 Hz, 1H), 8.38 (dd, J=8.3, 2.0 Hz, 1H), 8.95 (dd, J=4.2, 1.7 Hz, 1H), 11.27 (br.s, 1H). Mass Spectrum (ESI) m/e=204.1 (M+1).

### Methyl 8-(trifluoromethylsulfonyloxy)quinoline-7-carboxylate

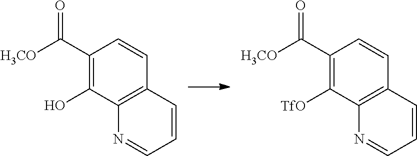 [SMILES: COC(=O)c1ccc2cccnc2c1O ; COC(=O)c1ccc2cccnc2c1OS(=O)(=O)C(F)(F)F]

Methyl 8-hydroxyquinoline-7-carboxylate (2.50 g, 12.30 mmol) and 4-(dimethyl-amino)-pyridine (0.075 g, 0.615 mmol) were dissolved in DCM (41.0 mL) and triethylamine (3.42 mL, 24.61 mmol). N-phenyltrifluoromethanesulfonimide (4.83 g, 13.53 mmol) was added in portions over 3 min and the reaction stirred at rt for 16 h. The reaction was added to sat NaHCO₃ (125 mL) and extracted three times with 100 mL DCM. The combined extracts were dried over magnesium sulfate and evaporated to give a white solid. ¹H NMR (500 MHz, DMSO-d₆) δ ppm 3.97 (s, 3H), 7.84 (dd, J=8.4, 4.3 Hz, 1H), 8.08 (d, J=8.6 Hz, 1H), 8.26 (d, J=8.8 Hz, 1H), 8.64 (dd, J=8.6, 1.7 Hz, 1H), 9.16 (dd, J=4.2, 1.7 Hz, 1H). Mass Spectrum (ESI) m/e=336.1 (M+1).

### Methyl 8-(3,5-difluorophenyl)quinoline-7-carboxylate

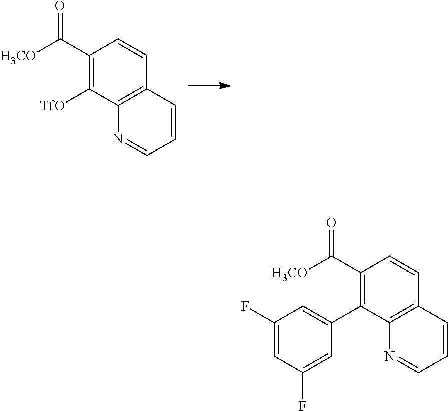 [SMILES: COC(=O)c1ccc2cccnc2c1OS(=O)(=O)C(F)(F)F ; COC(=O)c1ccc2cccnc2c1-c1cc(F)cc(F)c1]

A 100 mL flask was charged with methyl 8-(trifluoromethylsulfonyloxy)-quinoline-7-carboxylate (1.00 g, 2.98 mmol), potassium phosphate (1.27 g, 5.97 mmol), 2-dicyclohexylphosphino-2′,6′-dimethoxy-1,1′-biphenyl (0.184 g, 0.447 mmol), tris(dibenzylideneacetone)dipalladium (0.205 g, 0.224 mmol), 3,5-difluorophenylboronic acid (0.707 g, 4.47 mmol), and 1,4-dioxane (25 mL). The flask was evacuated and backfilled with argon six times, then heated in a 100° C. bath for 5.5 h. The reaction was added to 10% potassium carbonate solution (125 mL) and extracted three times with 100 mL DCM. The combined extracts were dried over MgSO₄ and evaporated. The resulting residue was chromatographed over silica gel using a gradient of hexane/0-30% ethyl acetate to give a pale yellow solid. ¹H NMR (500 MHz, CDCl₃) δ ppm 3.71 (s, 3H), 6.91 (m, 3H), 7.52 (dd, J=8.3, 4.2 Hz, 1H), 7.97 (m, 2H), 8.27 (dd, J=8.3, 2.0 Hz, 1H), 9.00 (dd, J=4.2, 1.7 Hz, 1H). Mass Spectrum (ESI) m/e=300.1 (M+1).

### (8-(3,5-Difluorophenyl)quinolin-7-yl)methanol

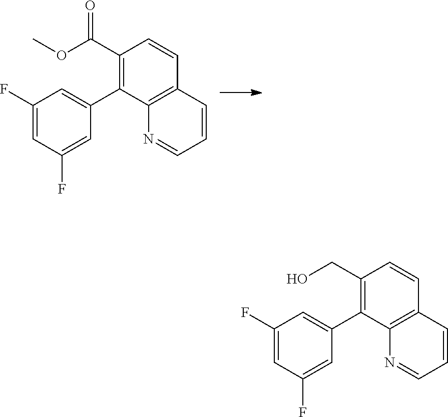 [SMILES: COC(=O)c1ccc2cccnc2c1-c1cc(F)cc(F)c1 ; OCc1ccc2cccnc2c1-c1cc(F)cc(F)c1]

Methyl 8-(3,5-difluorophenyl)quinoline-7-carboxylate (735 mg, 2.456 mmol) was suspended in dry THF (20 mL) under argon. The flask was cooled to 0° C. and lithium aluminum hydride, 1.0M solution in diethyl ether (2.70 mL, 2.70 mmol) was added over 1 minute. The reaction was allowed to warm to rt over 2.5 h. 0.5 mL water was added, followed by 0.5 mL 5N NaOH and then 1.5 mL water. The resulting suspension was stirred for 30 min and added to 75 mL 10% K₂CO₃, then extracted three times with DCM. The combined organics were dried over magnesium sulfate and evaporated to give a yellow foam. Chromatography over silica gel with a gradient of hexane/0-30% ethyl acetate gave a white solid. ¹H NMR (500 MHz, CDCl₃) δ ppm 1.72 (t, J=5.7 Hz×2, 1H), 4.67 (d, J=5.1 Hz, 2H), 6.91 (m, 3H), 7.43 (dd, J=8.3, 4.2 Hz, 1H), 7.85 (d, J=8.6 Hz, 1H), 7.94 (d, J=8.6 Hz, 1H), 8.22 (dd, J=8.2, 1.8 Hz, 1H), 8.91 (dd, J=4.2, 2.0 Hz, 1H). Mass Spectrum (ESI) m/e=272.1 (M+1).

### 8-(3,5-Difluorophenyl)_{q} uinoline-7-carbaldehyde

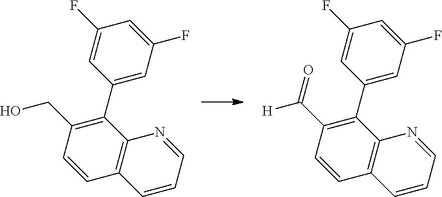 [SMILES: OCc1ccc2cccnc2c1-c1cc(F)cc(F)c1 ; O=Cc1ccc2cccnc2c1-c1cc(F)cc(F)c1]

A 50 mL flask was charged with (8-(3,5-difluorophenyl)quinolin-7-yl)methanol (478 mg, 1.762 mmol), 2-iodoxybenzoic acid, stabilized (45 wt %) (1316 mg, 2.115 mmol), and DMSO (8 mL). The solution was stirred at rt for 18 h, then added to ethyl acetate (75 mL). The resulting solution was washed successively with 75 mL 10% K₂CO₃, 75 mL water, and 75 mL sat NaCl. The organic phase was dried over MgSO₄ and evaporated to give a white solid. ¹H NMR (500 MHz, CDCl₃) δ ppm 7.01 (m, 3H), 7.58 (dd, J=8.3, 4.2 Hz, 1H), 8.00 (d, J=8.6 Hz, 1H), 8.17 (d, J=8.6 Hz, 1H), 8.29 (dd, J=8.2, 1.8 Hz, 1H), 9.02 (dd, J=4.2, 2.0 Hz, 1H), 10.02 (s, 1H). Mass Spectrum (ESI) m/e=270.1 (M+1)

### (E)-N-((8-(3,5-Difluorophenyl)quinolin-7-yl)methylene)-2-methylpropane-2-sulfinamide

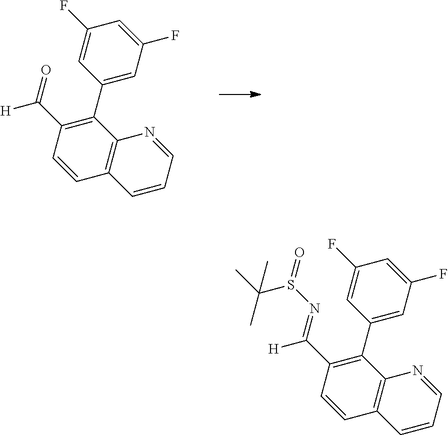 [SMILES: O=Cc1ccc2cccnc2c1-c1cc(F)cc(F)c1 ; CC(C)(C)S(=O)/N=C/c1ccc2cccnc2c1-c1cc(F)cc(F)c1]

A 100 mL flask was charged with 8-(3,5-difluorophenyl)quinoline-7-carbaldehyde (450 mg, 1.67 mmol), titanium (IV) ethoxide (0.692 mL, 3.34 mmol), 2-methyl-2-propanesulfinamide (203 mg, 1.671 mmol), and dry THF (5 mL). An argon atmosphere was introduced to the flask and the reaction heated at 65° C. for 16 h. The resulting solution was added to 25 mL ethyl acetate and 25 mL sat NaCl, then filtered through Celite™. The layers were separated and the aqueous phase extracted two times with 75 mL ethyl acetate. The combined organics were dried over MgSO₄ and evaporated to give a pale yellow tar. This residue was chromatographed over silica gel with a gradient of hexane/0-30% ethyl acetate to give a pale yellow solid. ¹H NMR (500 MHz, CDCl₃) δ ppm 1.27 (s, 9H), 6.95 (m, 3H), 7.51 (dd, J=8.3, 4.3 Hz, 1H), 7.95 (d, J=8.6 Hz, 1H), 8.25 (dd, J=8.3, 2.0 Hz, 1H), 8.30 (d, J=8.6 Hz, 1H), 8.57 (s, 1H), 8.97 (dd, J=4.2, 2.0 Hz, 1H). Mass Spectrum (ESI) m/e=373.1 (M+1).

### N-(1-(8-(3,5-Difluorophenyl)quinolin-7-yl)ethyl)-2-methylpropane-2-sulfinamide

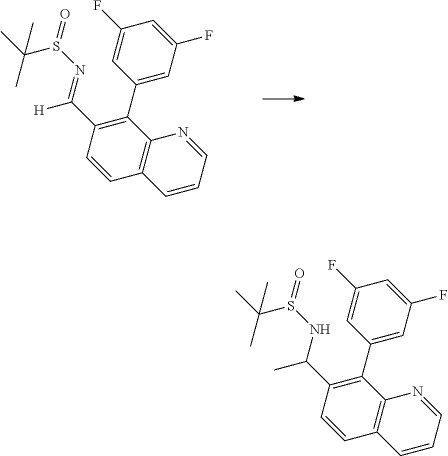 [SMILES: CC(C)(C)S(=O)/N=C/c1ccc2cccnc2c1-c1cc(F)cc(F)c1 ; CC(NS(=O)C(C)(C)C)c1ccc2cccnc2c1-c1cc(F)cc(F)c1]

A 50 mL flask was charged with (E)-N-((8-(3,5-difluorophenyl)quinolin-7-yl)-methylene)-2-methylpropane-2-sulfinamide (419 mg, 1.125 mmol) and dry THF (8 mL) under argon. The flask was cooled in a dry ice/acetone bath and methylmagnesium bromide, 3.0M in diethyl ether (2.250 mL, 6.75 mmol) was added over 1 min. The reaction was allowed to stir at rt for 2.5 h, then 5 mL sat NH₄Cl was added slowly. 25 mL water was added and the resulting mixture extracted three times with 30 mL DCM. The combined organics were dried over magnesium sulfate and evaporated to give a pale yellow solid. Mass Spectrum (ESI) m/e=389.1 (M+1).

### 1-(8-(3,5-Difluorophenyl)quinolin-7-ethanamine

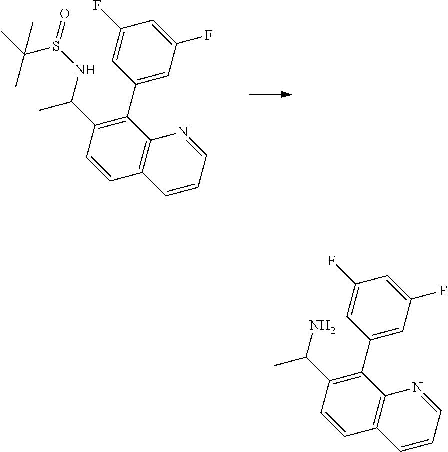 [SMILES: CC(NS(=O)C(C)(C)C)c1ccc2cccnc2c1-c1cc(F)cc(F)c1 ; CC(N)c1ccc2cccnc2c1-c1cc(F)cc(F)c1]

N-(1-(8-(3,5-Difluorophenyl)quinolin-7-yl)ethyl)-2-methylpropane-2-sulfinamide (440 mg, 1.133 mmol) was dissolved in THF (8 mL). Concentrated hydrochloric acid (0.40 mL, 13.16 mmol) was added and the reaction stirred at rt for 1.5 h. The solution was added to 75 mL 10% K₂CO₃ and extracted three times with 75 mL DCM. The combined organics were dried over magnesium sulfate and evaporated to give a pale yellow solid. ¹H NMR (500 MHz, CDCl₃) δ ppm 1.26 (d, J=6.1 Hz, 3H), 4.22 (br. S, 1H), 6.86 (m, 3H), 7.38 (dd, J=8.3, 4.2 Hz, 1H), 7.90 (m, 2H), 8.16 (d, J=8.3, 1H), 8.87 (dd, J=4.4, 2.0 Hz, 1H). Mass Spectrum (ESI) m/e=285.1 (M+1).

### Example 6

### 4-Amino-6-((1-(8-(3,5-difluorophenyl)-7-quinolinyl)ethyl)amino)-5-pyrimidinecarbonitrile

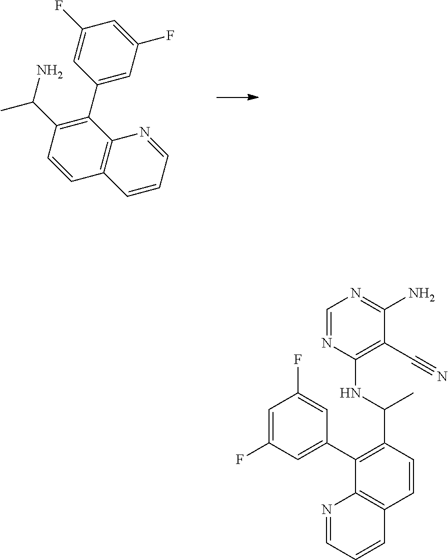 [SMILES: CC(N)c1ccc2cccnc2c1-c1cc(F)cc(F)c1 ; CC(Nc1ncnc(N)c1C#N)c1ccc2cccnc2c1-c1cc(F)cc(F)c1]

A small vial was charged with 1-(8-(3,5-difluorophenyl)quinolin-7-yl)ethanamine (120 mg, 0.422 mmol), 4-amino-6-chloropyrimidine-5-carbonitrile (71.8 mg, 0.464 mmol), DIEA (0.147 mL, 0.844 mmol), and 1-butanol (2.5 mL). The reaction was heated at 110° C. for 19 h, then allowed to cool and added to 30 mL 10% aq. K₂CO₃. This mixture was extracted three times with 30 mL DCM and the combined organics were dried over magnesium sulfate and evaporated to give a pale yellow solid. Preparative HPLC using a gradient of 10-60% acetonitrile over 35 min gave 4-amino-6-((1-(8-(3,5-difluorophenyl)-7-quinolinyl)ethyl)-amino)-5-pyrimidinecarbonitrile as a white solid. ¹H NMR (500 MHz, DMSO-d₆) δ 1.42 (d, J=7.1 Hz, 3H), 5.17 (m, 1H), 7.03 (d, J=9.0 Hz, 1H), 7.24 (m, 3H), 7.51 (dd, J=8.2, 4.3 Hz, 1H), 7.87 (m, 2H), 7.92 (d, J=8.6 Hz, 1H), 8.04 (d, J=8.6 Hz, 1H), 8.36 (d, J=8.0 Hz, 1H), 8.79 (dd, J=4.2, 1.7 Hz, 1H). Mass Spectrum (ESI) m/e=403.1 (M+1).

### Example 7

### N-(1-(8-(3,5-Difluorophenyl)quinolin-7-yl)ethyl)-9H-purin-6-amine

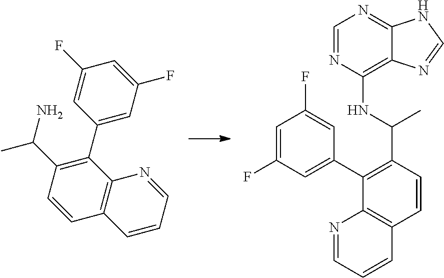 [SMILES: CC(N)c1ccc2cccnc2c1-c1cc(F)cc(F)c1 ; CC(Nc1ncnc2[nH]cnc12)c1ccc2cccnc2c1-c1cc(F)cc(F)c1]

A small vial was charged with 1-(8-(3,5-difluorophenyl)quinolin-7-yl)ethanamine (120 mg, 0.422 mmol), 6-chloro-9H-purine (71.8 mg, 0.464 mmol), DIEA (0.147 mL, 0.844 mmol), and 1-butanol (2.5 mL). The reaction was heated at 110° C. for 19 h, then allowed to cool and added to 30 mL 10% aq. K₂CO₃. This mixture was extracted three times with 30 mL DCM and the combined organics were dried over MgSO₄ and evaporated to give a pale yellow solid. Preparative HPLC using a gradient of 10-60% acetonitrile over 35 min gave the product as a white solid. ¹H NMR (500 MHz, DMSO-d₆) δ ppm 1.47 (d, J=7.1 Hz, 3H), 5.76 (m, 1H), 7.06 (m, 1H), 7.28 (m, 1H), 7.41 (d, J=9.7 Hz, 1H), 7.49 (dd, J=8.1, 4.2 Hz, 1H), 7.98 (m, 2H), 8.03 (s, 1H), 8.11 (br. S, 1H), 8.32 (dd, J=8.2, 1.8 Hz, 1H), 8.79 (dd, J=4.2, 1.7 Hz, 1H), 12.89 (s, 1H). Mass Spectrum (ESI) m/e=403.1 (M+1).

### 2-Chloro-4-fluoro-6-((trimethylsilyl)ethynyl)aniline

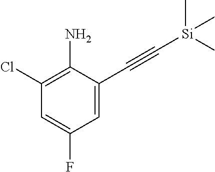

2-Bromo-6-chloro-4-fluoroaniline (20 g, 89 mmol) was added to 380 mL of diisopropylamine. The solution was sparged with N₂ before adding (trimethyl-silyl)acetylene (38 mL, 267 mmol) PdCl₂(PPh₃)₂CH₂Cl₂ (2.8 g, 3.56 mmol), and copper(I) iodide (0.339 g, 1.782 mmol). The suspension was then heated to 70° C. under an atmosphere of N₂. After 3 h the suspension was cooled to rt and then transferred to a 500 mL round-bottomed flask with ethyl acetate. The solvents were removed under vacuum and the residue obtained was partially dissolved in Et₂O and H₂O. The suspension was filtered and the filtrates were partitioned. The organic phase was washed with H₂O followed by brine. After the organics were dried over MgSO₄ they were concentrated under vacuum to give a brown/black liquid. The liquid was purified by column chromatography using a gradient of 100% hexane to 5% ethyl acetate/hexane. The fractions containing the product were combined and concentrated under vacuum to provide 2-chloro-4-fluoro-6-((trimethylsilyl)ethynyl)aniline as a orange/brown liquid. ¹H NMR (500 MHz, CHLOROFORM-d) δ ppm 7.02 (1H, dd, J=8.1, 2.9 Hz), 6.97 (1H, dd, J=8.6, 2.9 Hz), 4.46 (2H, br. s.), 0.28 (9H, s). Mass Spectrum (ESI) m/e=242.1 (M+1).

### 1-(2-Amino-3-chloro-5-fluorophenyl)ethanone

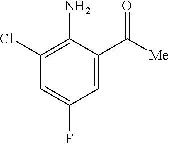 [SMILES: CC(=O)c1cc(F)cc(Cl)c1N]

2-Chloro-4-fluoro-6-((trimethylsilyl)ethynyl)aniline (12.19 g, 50.4 mmol) and sulfuric acid (2.016 mL, 37.8 mmol) were combined in methanol (200 mL). The solution was then heated to a gentle reflux for 3 h. The solution was cooled to rt and then most of the solvents were removed under vacuum. The residue obtained was diluted with ethyl acetate and then washed with sat NaHCO₃. The organics were dried over Na₂SO₄ and then concentrated under vacuum, to give brown oil. The oil was purified by column chromatography using a gradient of 100% hexane to 10% ethyl acetate/hexane. The fractions containing the pure product were combined and concentrated under vacuum to provide 1-(2-amino-3-chloro-5-fluorophenyl)ethanone as a yellow solid. ¹H NMR (500 MHz, CHLOROFORM-d) δ ppm 7.39 (1H, dd, J=9.3, 2.9 Hz), 7.27 (observed under the chloroform peak) (1H, dd, J=7.6, 2.9 Hz), 6.65 (2H, br. s.), 2.59 (3H, s). Mass Spectrum (ESI) m/e=188.1 (M+1).

### 4,8-Dichloro-6-fluoroquinoline-3-carbaldehyde

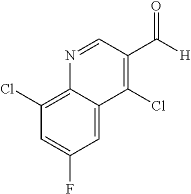 [SMILES: O=Cc1cnc2c(Cl)cc(F)cc2c1Cl]

Following a similar protocol as described in Indian Journal of Chemistry, Vol 36B, July 1997, pp 541-44: 1-(2-amino-3-chloro-5-fluorophenyl)ethanone (1.66 g, 8.85 mmol) was dissolved in 11 mL of anhydrous DMF under an atmosphere of N₂. The solution was cooled in an ice bath before slowly adding phosphorus oxychloride (3.30 mL, 35.4 mmol) over a period of 15 min. The solution was then allowed to warm to rt. After 30 min the solution was heated to 75° C. for 1.5 h. After cooling the solution to rt, it was cooled in a ice bath and then quenched with ice (˜90 mL). The solution was stirred until most of the ice dissolved and then the solids were filtered off and washed with H₂O. The solids were dissolved in DCM and then dried over Na₂SO₄, before being concentrated under vacuum to provide 4,8-dichloro-6-fluoroquinoline-3-carbaldehyde as a yellow solid. ¹H NMR (500 MHz, CHLOROFORM-d) δ ppm 10.72 (1H, s), 9.34 (1H, s), 8.01 (1H, dd, J=8.8, 2.7 Hz), 7.87 (1H, dd, J=7.9, 2.8 Hz). Mass Spectrum (ESI) m/e=244.0 (M+1).

### 1-(4,8-Dichloro-6-fluoroquinolin-3-yl)ethanol

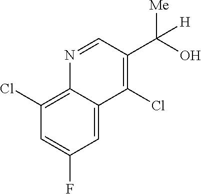 [SMILES: CC(O)c1cnc2c(Cl)cc(F)cc2c1Cl]

4,8-Dichloro-6-fluoroquinoline-3-carbaldehyde (0.059 g, 0.242 mmol) was dissolved in 2 mL of anhydrous THF and then cooled in a dry ice/acetone bath. After 5 min methylmagnesium bromide 2.83 M in Et₂O (0.094 mL, 0.266 mmol) was slowly added and the solution was stirred in the dry ice acetone bath for 30 min before being allowed to warm to rt. After 10 min the reaction was quenched with sat NaHCO₃ and then the product was extracted with DCM. The organics were dried over Na₂SO₄ and then concentrated under vacuum to give the crude product as a yellow oil. The oil was purified by column chromatography using a gradient of 20% ethyl acetate/hexane to 40% ethyl acetate/hexane. The fractions containing the product were combined and concentrated under vacuum to provide 1-(4,8-dichloro-6-fluoroquinolin-3-yl)ethanol as a light yellow solid. ¹H NMR (500 MHz, CHLOROFORM-d) δ ppm 9.16 (1H, s), 7.75 (1H, dd, J=9.3, 2.7 Hz), 7.66 (1H, dd, J=8.1, 2.7 Hz), 5.51 (1H, qd, J=6.4, 3.2 Hz), 2.81 (1H, d, J=2.9 Hz), 1.59 (3H, d, J=6.6 Hz). Mass Spectrum (ESI) m/e=259.9 (M+1).

### 1-(4,8-Dichloro-6-fluoroquinolin-3-yl)ethanone

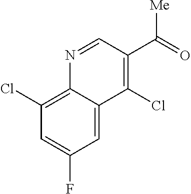 [SMILES: CC(=O)c1cnc2c(Cl)cc(F)cc2c1Cl]

1-(4,8-Dichloro-6-fluoroquinolin-3-yl)ethanol (1.050 g, 4.04 mmol) and manganese(IV) oxide (2.81 g, 32.3 mmol) were combined in 50 mL of anhydrous toluene and heated at 110° C. overnight. The next day the suspension was cooled to rt and then diluted with DCM. After the suspension was filtered through a pad of celite, the solids were washed with DCM and the filtrate was concentrated under vacuum to give 1-(4,8-dichloro-6-fluoroquinolin-3-yl)ethanone as a greenish/white solid. ¹H NMR (500 MHz, CHLOROFORM-d) δ ppm 9.03 (1H, s), 7.97 (1H, dd, J=9.0, 2.7 Hz), 7.80 (1H, dd, J=8.1, 2.7 Hz), 2.81 (3H, s). Mass Spectrum (ESI) m/e=258.0 (M+1).

### 4-Chloro-8-fluoro-N-methoxy-N-methylquinoline-3-carboxamide

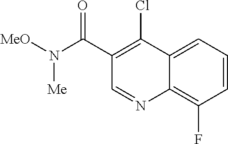 [SMILES: CON(C)C(=O)c1cnc2c(F)cccc2c1Cl]

To a solution of 4-chloro-8-fluoroquinoline-3-carboxylic acid [prepared as in General Method B5, from ethyl 4-chloro-8-fluoroquinoline-3-carboxylate (BIOLIPDX AB Patent: WO2007/51982 A1, 2007)](1 g, 4.41 mmol) in DMF (20 mL) was added N,O-Dimethyl Hydroxyl-amine hydrochloride (0.5 g, 5.29 mmol), EDC (0.845 g, 5.29 mmol), HOBT (0.74 g, 4.85 mmol) and triethylamine (1.3 g, 13.23 mmol). The reaction mixture was stirred at rt overnight, diluted with water, and the product was extracted with diethyl ether. The organic phase was dried over Na₂SO₄, the solids were filtered off and the filtrate was concentrated under vacuum. The residue obtained was washed with diethyl ether followed by pentane to obtain 4-chloro-8-fluoroquinoline-3-carboxylic acid methoxy-methyl-amide as a solid. TLC (50% ethyl acetate in hexane, product's R_{f}=0.5).

### 1-(4-Chloro-7-fluoroquinolin-3-yl)ethanone

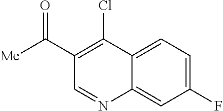 [SMILES: CC(=O)c1cnc2cc(F)ccc2c1Cl]

1-(4-Chloro-7-fluoroquinolin-3-yl)ethanone was prepared according to the methods described in General Methods B5, B6, and B7 starting from ethyl 4-chloro-7-fluoroquinoline-3-carboxylate. ¹HNMR (400 MHz, CDCl₃) δ ppm 9.00 (s, 1H), 8.425-8.388 (m, 1H), 7.794-7.764 (m, 1H), 7.530-7.481 (m, 1H), 2.802 (s, 3H). Mass Spectrum (ESI) m/e=224.08 (M+1).

### 1-(4-Chloro-8-fluoroquinolin-3-yl)ethanone

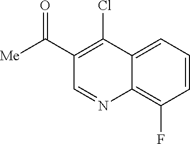 [SMILES: CC(=O)c1cnc2c(F)cccc2c1Cl]

1-(4-Chloro-8-fluoroquinolin-3-yl)ethanone was prepared according to the methods described in General Method B7 from 4-chloro-8-fluoroquinoline-3-carboxylic acid methoxy-methyl-amide. ¹HNMR: (400 MHz, CDCl₃) δ ppm 9.109 (s, 1H), 8.207-8.164 (m, 1H), 7.873-7.807 (m, 2H), 2.765 (s, 3H). Mass Spectrum (ESI) m/e=224.06. (M+1).

### 1-(4,6-Dichloroquinolin-3-yl)ethanone

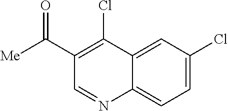 [SMILES: CC(=O)c1cnc2ccc(Cl)cc2c1Cl]

1-(4,6-Dichloroquinolin-3-yl)ethanone was prepared according to the methods described in General Method B7 from 4,6-dichloro-N-methoxy-N-methyl-quinoline-3-carboxamide. ¹HNMR: (400 MHz, CDCl₃) δ ppm 9.095 (s, 1H), 8.354 (d, J=2.4 Hz, 1H), 8.177 (d, J=8.8 Hz, 1H), 7.998 (dd, J=8.8 Hz, 2.4 Hz, 1H), 2.756 (s, 3H). Mass Spectrum (ESI) m/e=240.13 (M+1).

### 1-(4-Chloro-6-fluoroquinolin-3-yl)ethanone

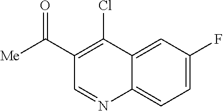 [SMILES: CC(=O)c1cnc2ccc(F)cc2c1Cl]

1-(4-Chloro-6-fluoroquinolin-3-yl)ethanone was prepared according to the methods described in General Methods B5, B8 and B7 starting from ethyl 4-chloro-6-fluoroquinoline-3-carboxylate (Journal of Medicinal Chemistry, 2006, vol. 49, #21, p. 6351-6363). ¹HNMR (400 MHz, CDCl₃) δ ppm 9.059 (s, 1H), 8.257-8.220 (m, 1H), 8.086-8.054 (m, 1H), 7.933-7.882 (m, 1H), 3.325 (s, 3H). Mass Spectrum (ESI) m/e=224 (M+1).

### 1-(4,8-Dichloroquinolin-3-yl)ethanone

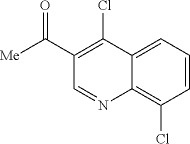 [SMILES: CC(=O)c1cnc2c(Cl)cccc2c1Cl]

1-(4,8-Dichloroquinolin-3-yl)ethanone was prepared according to the methods described in General Methods B5, B8 and B7 starting from ethyl 4,8-dichloroquinoline-3-carboxylate. ¹HNMR (400 MHz, CDCl₃) δ ppm 9.176 (s, 1H), 8.367-8.342 (m, 1H), 8.182-8.160 (m, 2H), 2.765 (s, 3H). Mass Spectrum (ESI) m/e=240 (M+1).

### 4-Chloro-N-methoxy-N-methylquinoline-3-carboxamide

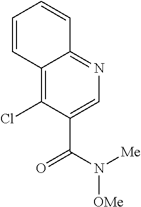 [SMILES: CON(C)C(=O)c1cnc2ccccc2c1Cl]

To a slurry of ethyl 4-chloroquinoline-3-carboxylate (Journal of Medicinal Chemistry, 2006, vol. 49, #21, p. 6351-6363) (0.696 g, 2.95 mmol), and N,O-dimethylhydroxylamine hydrochloride (0.432 g, 4.43 mmol) in 10 mL of anhydrous THF cooled in a brine/ice bath under an atmosphere of N₂ was added isopropylmagnesium chloride 2.0M in Et₂O (3.69 mL, 7.38 mmol) dropwise over a period of 10 min. The solution was then stirred in the brine/ice bath for 20 min before it was quenched with sat NH₄Cl. The product was extracted with ethyl acetate and the organics were dried over MgSO₄ before being concentrated under vacuum. The yellow solids obtained were purified by column chromatography using a gradient of 50% ethyl acetate/hexane to 100% ethyl acetate. The fractions containing the product were combined and concentrated under vacuum to give 4-chloro-N-methoxy-N-methylquinoline-3-carboxamide. ¹H NMR (500 MHz, CHLOROFORM-d) δ ppm 8.82 (1H, s), 8.33 (1H, d, J=7.8 Hz), 8.18 (1H, d, J=8.3 Hz), 7.85 (1H, td, J=7.7, 1.2 Hz), 7.70-7.76 (1H, m), 3.45-3.58 (6H, br m). Mass Spectrum (ESI) m/e=251.1 (M+1). TLC (50% ethyl acetate/hexane, product's R_{f}=0.24).

### 1-(4-Chloroquinolin-3-yl)ethanone

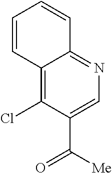 [SMILES: CC(=O)c1cnc2ccccc2c1Cl]

To a solution of 4-chloro-N-methoxy-N-methylquinoline-3-carboxamide (0.350 g, 1.396 mmol) in 10 mL of anhydrous THF cooled in a brine/ice under an atmosphere of N₂ was slowly added methylmagnesium bromide 3.0M in Et₂O (0.512 mL, 1.536 mmol) over a period of 2 min. The solution (with solids present) was then allowed to warm to rt and left overnight. The next day LCMS shows ˜20% of the starting material present. An additional charge of 0.2 mL of methyl magnesium bromide 3.0M in Et₂O was added and the suspension was stirred at rt for 2 h. The reaction was quenched with the addition of sat NH₄Cl and the product was extracted with DCM. The organics were dried over Na₂SO₄ before being concentrated under vacuum. The brownish oil obtained was purified by column chromatography using a gradient of 15% ethyl acetate/hexane to 40% ethyl acetate/hexane. The fractions containing the product were combined and concentrated under vacuum to provide 1-(4-chloroquinolin-3-yl)ethanone as a off white solid. ¹H-NMR (500 MHz, CHLOROFORM-d) δ ppm 8.96 (1H, s), 8.31-8.35 (1H, m), 8.10-8.13 (1H, m), 7.82 (1H, ddd, J=8.4, 6.9, 1.3 Hz), 7.69 (1H, ddd, J=8.4, 7.0, 1.2 Hz), 2.78 (3H, s). Mass Spectrum (ESI) m/e=206.1 (M+1).

### 1-(4-(Pyridin-2-yl)quinolin-3-yl)ethanone

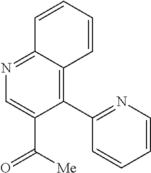 [SMILES: CC(=O)c1cnc2ccccc2c1-c1ccccn1]

1-(4-(Pyridin-2-yl)quinolin-3-yl)ethanone was prepared according to the methods described in General Method A9 from 1-(4-chloroquinolin-3-yl)ethanone. ¹H NMR (500 MHz, CHLOROFORM-d) δ ppm 9.20 (1H, s), 8.83 (1H, br. s.), 8.21 (1H, d, J=8.6 Hz), 7.91 (1H, t, J=7.5 Hz), 7.81 (1H, ddd, J=8.4, 6.9, 1.3 Hz), 7.65 (1H, d, J=8.3 Hz), 7.55 (1H, t, J=7.7 Hz), 7.45-7.52 (2H, m), 2.18 (3H, s). Mass Spectrum (ESI) m/e=249.2 (M+1). TLC (100% ethyl acetate, product's R_{f}=0.59).

### 1-(4-(Pyridin-2-yl)quinolin-3-yl)ethanamine and 1-(4-(pyridin-2-yl)quinolin-3-yl)ethanol

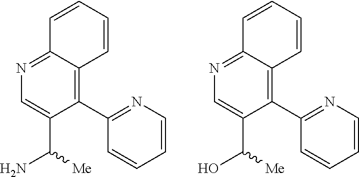

1-(4-(Pyridin-2-yl)quinolin-3-yl)ethanone (0.160 g, 0.644 mmol), and ammonia 7M in methanol (0.460 mL, 3.22 mmol) were combined in 2 mL of anhydrous methanol under N₂. Titanium(IV) isopropoxide (0.378 mL, 1.289 mmol) was then added and the solution was left to stir at rt for 6 h. Sodium borohydride (0.037 g, 0.967 mmol) was then added and the suspension was stirred at rt overnight. The reaction was quenched with sat NH₄Cl and the solution was filtered through filter paper and washed with DCM. The filtrates were partitioned and the aqueous layer was washed with DCM. The combined organics were dried over Na₂SO₄ and then concentrated under vacuum. The yellow oil obtained was purified by column chromatography using a gradient of DCM to 10% methanol/0.5% NH₄OH (˜28% in water)/DCM. The fractions 35-37 were combined and concentrated under vacuum to give 1-(4-(pyridin-2-yl)quinolin-3-yl)ethanamine as a light yellowish oil. The fractions 27-29 were combined and concentrated under vacuum to give 1-(4-(pyridin-2-yl)quinolin-3-yl)ethanol a light yellowish oil.

### Example 8

### 4-Amino-6-(1-(4-(pyridin-2-yl)quinolin-3-yl)ethoxy)pyrimidine-5-carbonitrile

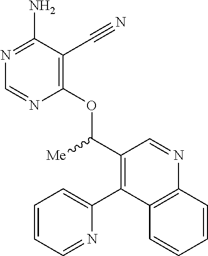 [SMILES: CC(Oc1ncnc(N)c1C#N)c1cnc2ccccc2c1-c1ccccn1]

1-(4-(Pyridin-2-yl)quinolin-3-yl)ethanol (0.061 g, 0.244 mmol), and 4-amino-6-chloropyrimidine-5-carbonitrile (0.066 g, 0.427 mmol) were dissolved in 3 mL of anhydrous DMF under an atmosphere of N₂. Sodium hydride 60% in mineral oil (0.029 g, 0.731 mmol) was then added and the suspension was stirred at rt overnight. The next day sat NH₄Cl was added and the product was extracted with DCM. The organics were dried over MgSO₄ and then concentrated under vacuum. The residue obtained was purified by column chromatography using a gradient of DCM to 10% methanol/0.5% NH4OH (˜28% in water)/DCM. The fractions containing the product were combined and concentrated under vacuum to give 4-amino-6-(1-(4-(pyridin-2-yl)quinolin-3-yl)ethoxy)pyrimidine-5-carbonitrile as a clear glass. A mixture of isomers was observed in the proton NMR trace. ¹H NMR (500 MHz, CHLOROFORM-d) δ ppm 9.21 (1H, br. s.), 8.84 (1H, d, J=4.2 Hz), 8.13-8.20 (1H, m), 8.03 (1H, br. s.), 7.92 (1H, td, J=7.7, 1.5 Hz), 7.70 (1H, ddd, J=8.4, 6.9, 1.3 Hz), 7.60 (0.8H, br. s.), 7.46 (2.2H, t, J=7.6 Hz), 7.36-7.42 (1H, m), 6.39 (0.2H, br. s.), 6.10 (0.8H, br. s.), 5.81 (2.3H, br. s.), 1.56-1.89 (0.7H, m). Mass Spectrum (ESI) m/e=369.1 (M+1) and 367.0 (M−1). The individual enantiomers were obtained by chiral SFC purification.

### 4-Amino-6-((1S)-1-(4-(2-pyridinyl)-3-quinolinyl)ethoxy)-5-pyrimidinecarbonitrile

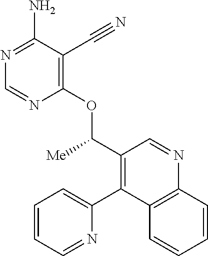 [SMILES: C[C@H](Oc1ncnc(N)c1C#N)c1cnc2ccccc2c1-c1ccccn1]

4-Amino-((1S)-1-(4-(2-pyridinyl)-3-quinolinyl)ethoxy)-5-pyrimidinecarbonitrile was prepared according to the methods described in General Method B4 starting from 4-amino-6-(1-(4-(pyridin-2-yl)quinolin-3-yl)ethoxy)pyrimidine-5-carbonitrile. The stereochemistry is arbitrarily assigned. A mixture of isomers was observed in the proton NMR trace. ¹H NMR (500 MHz, CHLOROFORM-d) δ ppm 9.21 (1H, br. s.), 8.85 (1H, d, J=4.2 Hz), 8.17 (1H, d, J=8.6 Hz), 8.05 (1 H, br. s.), 7.93 (1H, td, J=7.6, 1.3 Hz), 7.69-7.76 (1H, m), 7.56-7.64 (0.75H, m), 7.47 (2H, t, J=7.1 Hz), 7.36-7.43 (1H, m), 6.41 (0.23H, br. s.), 6.11 (0.71H, br. s.), 5.52 (2H, br. s.), 1.78 (2.3H, br. s.), 1.67 (1H, br. s.). Mass Spectrum (ESI) m/e=369.1 (M+1) and 367.1 (M−1). EE>99%.

### 4-Amino-6-((1R)-1-(4-(2-pyridinyl)-3-quinolinyl)ethoxy)-5-pyrimidinecarbonitrile

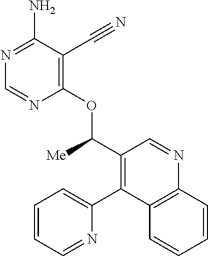 [SMILES: C[C@@H](Oc1ncnc(N)c1C#N)c1cnc2ccccc2c1-c1ccccn1]

4-Amino-((1R)-1-(4-(2-pyridinyl)-3-quinolinyl)ethoxy)-5-pyrimidinecarbonitrile was prepared according to the methods described in General Method B4 starting from 4-amino-6-(1-(4-(pyridin-2-yl)quinolin-3-yl)ethoxy)pyrimidine-5-carbonitrile. The stereochemistry is arbitrarily assigned. A mixture of isomers was observed in the proton NMR trace. ¹H NMR (500 MHz, CHLOROFORM-d) δ ppm 9.21 (1H, br. s.), 8.85 (1H, d, J=4.2 Hz), 8.17 (1H, d, J=8.6 Hz), 8.05 (1 H, br. s.), 7.93 (1H, td, J=7.6, 1.3 Hz), 7.69-7.76 (1H, m), 7.56-7.64 (0.75H, m), 7.47 (2H, t, J=7.1 Hz), 7.36-7.43 (1H, m), 6.41 (0.23H, br. s.), 6.11 (0.71H, br. s.), 5.52 (2H, br. s.), 1.78 (2.3H, br. s.), 1.67 (1H, br. s.). Mass Spectrum (ESI) m/e=369.1 (M+1) and 367.1 (M−1). EE>99%.

### Additional Compounds Made Via General Methods:

The following compounds were made via general methods A0, A1, A2, A3, and A4 as described above.

### Example 9

### 4-Amino-6-((1-(4-(3,5-difluorophenyl)-3-quinolinyl)ethyl)amino)-5-pyrimidinecarbonitrile

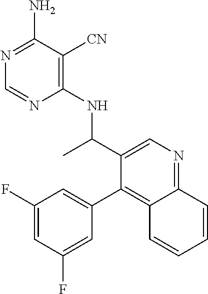 [SMILES: CC(Nc1ncnc(N)c1C#N)c1cnc2ccccc2c1-c1cc(F)cc(F)c1]

¹H NMR (500 MHz, CDCl₃) δ ppm 9.01 (s, 1H), 8.14 (d, J=8.1 Hz, 1H), 8.02 (s, 1H), 7.72 (ddd, J=8.3, 6.9, 1.5 Hz 1H), 7.48 (ddd, J=8.6, 1.5, 0.5 Hz, 1H), 7.38 (ddd, J=8.6, 1.5, 0.5 Hz, 1H), 7.22 (ddt, J=8.8, 2.2, 1.2 Hz, 1H), 6.99 (tt, J=9.0, 1.5 Hz, 1H), 6.81 (ddt, J=8.6, 2.2, 1.2 Hz, 1H), 5.55 (d, J=6.4 Hz, 1H), 5.31 (br s, 2H), 5.25 (dq, J=7.1, 7.1 Hz, 1H), 1.56 (d, J=7.1 Hz, 3H). Mass Spectrum (ESI) m/e=403.1 (M+1). The individual enantiomers were obtained according to the methods described in General Method B4 to give 4-amino-6-(((1S)-1-(4-(3,5-difluorophenyl)-3-quinolinyl)ethyl)amino)-5-pyrimidinecarbonitrile and 4-amino-6-(((1R)-1-(4-(3,5-difluorophenyl)-3-quinolinyl)ethyl)-amino)-5-pyrimidinecarbonitrile and the spectral data of each chiral enantiomer was consistent with that of racemic 4-amino-6-((1-(4-(3,5-difluorophenyl)-3-quinolinyl)ethyl)amino)-5-pyrimidinecarbonitrile.

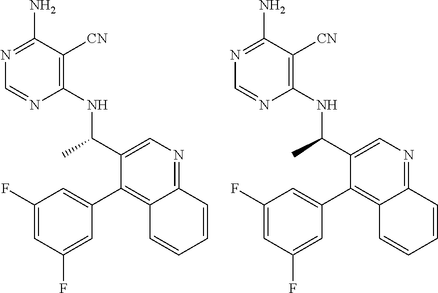

The following compounds were made via general methods A6, A0, A1, A2. A3, and A4 as described above.

### Example 10

### 4-Amino-6-((1-(4-phenyl-3-cinnolinyl)ethyl)amino)-5-pyrimidinecarbonitrile

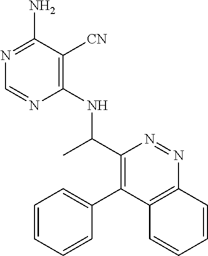 [SMILES: CC(Nc1ncnc(N)c1C#N)c1nnc2ccccc2c1-c1ccccc1]

¹H NMR (500 MHz, DMSO-d₆) δ ppm 8.52 (ddd, J=8.4, 1.2, 0.6 Hz, 1H), 7.95 (ddd, J=8.0, 6.7, 1.2 Hz, 1H), 7.89 (s, 1H), 7.80 (ddd, J=8.2, 6.7, 1.2 Hz, 1H), 7.59 (m, 5H), 7.45 (m, 1H), 7.41 (ddd, J=8.4, 1.2, 0.6 Hz, 1H), 7.25 (br s, 2H), 5.46 (quintet, J=7.0 hz, 1H), 1.52 (d, J=6.9 Hz, 3H). Mass Spectrum (ESI) m/e=368.2 (M+1). The individual enantiomers were obtained according to the methods described in General Method B4 to give 4-amino-6-(((1R)-1-(4-phenyl-3-cinnolinyl)ethyl)amino)-5-pyrimidinecarbonitrile and 4-amino-6-(((1S)-1-(4-phenyl-3-cinnolinyl)ethyl)amino)-5-pyrimidinecarbonitrile and the spectral data of each chiral enantiomer was consistent with that of racemic 4-amino-6-((1-(4-phenyl-3-cinnolinyl)ethyl)amino)-5-pyrimidinecarbonitrile.

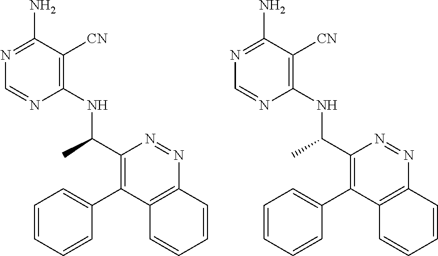 [SMILES: C[C@@H](Nc1ncnc(N)c1C#N)c1nnc2ccccc2c1-c1ccccc1 ; C[C@H](Nc1ncnc(N)c1C#N)c1nnc2ccccc2c1-c1ccccc1]

### Example 11

### 4-Amino-6-((1-(4-(3-fluorophenyl)-3-cinnolinyl)ethyl)amino)-5-pyrimidinecarbonitrile

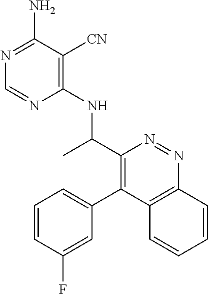 [SMILES: CC(Nc1ncnc(N)c1C#N)c1nnc2ccccc2c1-c1cccc(F)c1]

The rt ¹H-NMR reflects a roughly 1:1 mixture of isomers. ¹H NMR (500 MHz, DMSO-d₆) δ ppm 8.54 (m, 1H), 7.96 (m, 1H), 7.85 (m, 1H), 7.63 (m, 2H), 7.45-7.12 (series of m, 6H), 5.44 (m, 1H), 1.57 (m, 3H). Mass Spectrum (ESI) m/e=386.2 (M+1). The individual enantiomers were obtained according to the methods described in General Method B4 to give 4-amino-6-(((1R)-1-(4-(3-fluorophenyl)-3-cinnolinyl)ethyl)amino)-5-pyrimidinecarbonitrile and 4-amino-6-(((1S)-1-(4-(3-fluorophenyl)-3-cinnolinyl)ethyl)amino)-5-pyrimidinecarbonitrile and the spectral data of each chiral enantiomer was consistent with that of racemic 4-amino-6-((1-(4-(3-fluorophenyl)-3-cinnolinyl)ethyl)amino)-5-pyrimidinecarbonitrile.

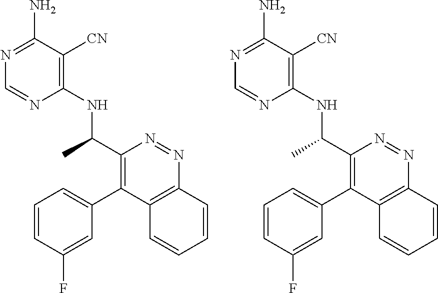 [SMILES: C[C@@H](Nc1ncnc(N)c1C#N)c1nnc2ccccc2c1-c1cccc(F)c1 ; C[C@H](Nc1ncnc(N)c1C#N)c1nnc2ccccc2c1-c1cccc(F)c1]

### Example 12

### 4-Amino-6-((1-(4-(3,5-difluorophenyl)-3-cinnolinyl)ethyl)-amino)-5-pyrimidinecarbonitrile

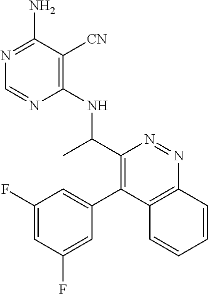 [SMILES: CC(Nc1ncnc(N)c1C#N)c1nnc2ccccc2c1-c1cc(F)cc(F)c1]

¹H NMR (500 MHz, DMSO-d₆) δ ppm 8.54 (d, J=9.3 Hz, 1H), 7.97 (ddd, J=8.1, 6.6, 1.2 Hz, 1H), 7.87 (s, 1H), 7.83 (ddd, J=9.8, 6.8, 1.2 Hz, 1H), 7.62 (d, J=7.1 Hz, 1H), 7.47 (d, J=8.3 Hz, 1H), 7.44 (m, 1H), 7.35-7.15 (series of m, 4H), 5.45 (quintet, J=6.85 Hz, 1H), 1.60 (d, J=6.9 Hz, 3H). Mass Spectrum (ESI) m/e=404.2 (M+1). The individual enantiomers were obtained according to the methods described in General Method B4 to give 4-amino-6-(((1R)-1-(4-(3,5-difluorophenyl)-3-cinnolinyl)ethyl)amino)-5-pyrimidinecarbonitrile and 4-amino-6-(((1S)-1-(4-(3,5-difluorophenyl)-3-cinnolinyl)ethyl)amino)-5-pyrimidinecarbonitrile and the spectral data of each chiral enantiomer was consistent with that of racemic 4-amino-6-((1-(4-(3,5-difluorophenyl)-3-cinnolinyl)ethyl)amino)-5-pyrimidinecarbonitrile.

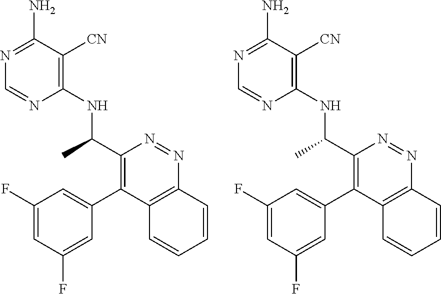

### Example 13

### 4-Amino-6-((1-(6-fluoro-4-phenyl-3-cinnolinyl)ethyl)amino)-5-pyrimidinecarbonitrile

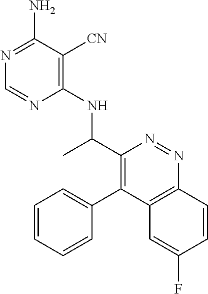 [SMILES: CC(Nc1ncnc(N)c1C#N)c1nnc2ccc(F)cc2c1-c1ccccc1]

¹H NMR (500 MHz, DMSO-d₆) δ ppm 8.65 (dd, J=9.3, 5.4 Hz, 1H), 7.87 (m, 2H), 7.60 (m, 5H), 7.45 (m, 1H), 7.25 (br s, 2H), 6.97 (dd, J=9.5, 2.7 Hz, 1H), 5.42 (J=6.7 Hz, 1H), 1.52 (d, J=6.9 Hz, 3H). Mass Spectrum (ESI) m/e=384.1 (M+1). The individual enantiomers were obtained according to the methods described in General Method B4 to give 4-amino-6-(((1R)-1-(6-fluoro-4-phenyl-3-cinnolinyl)ethyl)amino)-5-pyrimidinecarbonitrile and 4-amino-6-(((1S)-1-(6-fluoro-4-phenyl-3-cinnolinyl)ethyl)amino)-5-pyrimidinecarbonitrile and the spectral data of each chiral enantiomer was consistent with that of racemic 4-amino-6-((1-(6-fluoro-4-phenyl-3-cinnolinyl)ethyl)amino)-5-pyrimidinecarbonitrile.

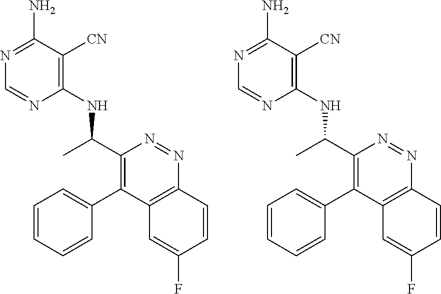 [SMILES: C[C@@H](Nc1ncnc(N)c1C#N)c1nnc2ccc(F)cc2c1-c1ccccc1 ; C[C@H](Nc1ncnc(N)c1C#N)c1nnc2ccc(F)cc2c1-c1ccccc1]

### Example 14

### 4-Amino-6-((1-(6-fluoro-4-(3-fluorophenyl)-3-cinnolinyl)ethyl)-amino)-5-pyrimidinecarbonitrile

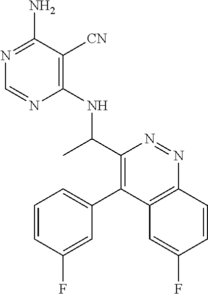 [SMILES: CC(Nc1ncnc(N)c1C#N)c1nnc2ccc(F)cc2c1-c1cccc(F)c1]

The rt ¹H-NMR reflects a roughly 1:1 mixture of isomers. ¹H NMR (500 MHz, DMSO-d₆) δ ppm 8.65 (m, 1H), 7.89 (m, 2H), 7.62 (m, 2H), 7.45-7.10 (series of m, 5H), 7.03 (m, 1H), 5.43 (m, 1H), 1.55 (m, 3H). Mass Spectrum (ESI) m/e=404.2 (M+1).

The following compound was made via general methods A6, A0, A1, A2. A3, and A5 as described above:

### Example 15

### N-(-1-(4-Phenyl-3-cinnolinyl)ethyl)-9H-purin-6-amine

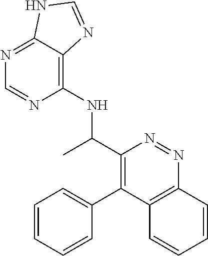 [SMILES: CC(Nc1ncnc2[nH]cnc12)c1nnc2ccccc2c1-c1ccccc1]

¹H NMR (500 MHz, DMSO-d₆) δ ppm 13.0-12.1 (br m, 1H), 8.50 (d, J=8.3 Hz, 1H), 8.14 (br s, 1H), 8.08 (s, 1H), 7.93 (ddd, J=8.3, 6.9, 1.2 Hz, 1H), 7.92 (br s, 1H), 7.80 (ddd, J=8.3, 6.8, 1.2 Hz, 1H), 7.63 (m, 4H), 7.47 (m, 1H), 7.42 (d, J=8.6 Hz, 1H), 5.55 (br s, 1H), 1.61 (d J=6.9 Hz, 3H). Mass Spectrum (ESI) m/e=368.2 (M+1). The individual enantiomers were obtained according to the methods described in General Method B4 to give N-((1R)-1-(4-phenyl-3-cinnolinyl)ethyl)-9H-purin-6-amine and N-((1S)-1-(4-phenyl-3-cinnolinyl)ethyl)-9H-purin-6-amine and the spectral data of each chiral enantiomer was consistent with that of racemic N-(-1-(4-phenyl-3-cinnolinyl)ethyl)-9H-purin-6-amine.

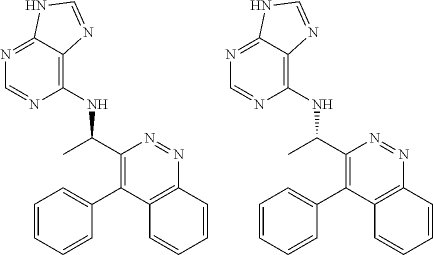 [SMILES: C[C@@H](Nc1ncnc2[nH]cnc12)c1nnc2ccccc2c1-c1ccccc1 ; C[C@H](Nc1ncnc2[nH]cnc12)c1nnc2ccccc2c1-c1ccccc1]

The following compounds were made via general methods All, A1, A2, A3, A4 starting from 1-(4-chloro-6-fluoroquinolin-3-yl)ethanone (synthesis according to general methods B5, B8 and B7):

### Example 16

### 4-Amino-6-((1-(6-fluoro-4-phenyl-3-quinolinyl)ethyl)amino)-5-pyrimidinecarbonitrile

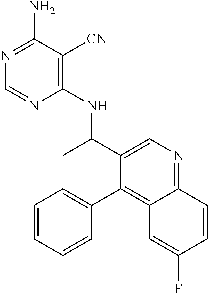 [SMILES: CC(Nc1ncnc(N)c1C#N)c1cnc2ccc(F)cc2c1-c1ccccc1]

¹H NMR (500 MHz, DMSO-d₆) δ ppm 9.17 (s, 1H), 8.12 (dd, J=9.3, 5.6 Hz, 1H), 7.87 (d, J=7.1 Hz, 1H), 7.85 (s, 1H), 7.67-7.52 (series of m, 5H), 7.33 (d, J=7.1 Hz, 1H), 7.20 (br s, 2H), 6.83 (dd, J=10.3, 2.1 Hz, 1H), 5.11 (quintet, J=7.1 Hz, 1H), 1.46 (d, J=7.1 Hz, 3H). Mass Spectrum (ESI) m/e=385.2 (M+1). The individual enantiomers were obtained according to the methods described in General Method B4 to give 4-amino-6-(((1S)-1-(6-fluoro-4-phenyl-3-quinolinyl)ethyl)amino)-5-pyrimidinecarbonitrile and 4-amino-6-(((1R)-1-(6-fluoro-4-phenyl-3-quinolinyl)ethyl)amino)-5-pyrimidinecarbonitrile and the spectral data of each chiral enantiomer was consistent with that of racemic 4-amino-6-((1-(6-fluoro-4-phenyl-3-quinolinyl)ethyl)amino)-5-pyrimidinecarbonitrile.

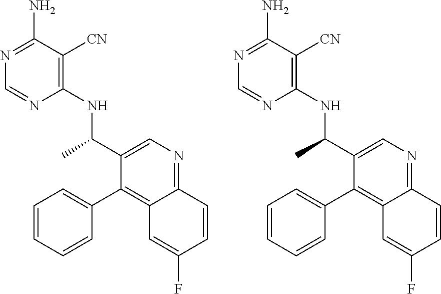 [SMILES: C[C@H](Nc1ncnc(N)c1C#N)c1cnc2ccc(F)cc2c1-c1ccccc1 ; C[C@@H](Nc1ncnc(N)c1C#N)c1cnc2ccc(F)cc2c1-c1ccccc1]

### Example 17

### 4-Amino-6-((1-(4-(3-cyanophenyl)-6-fluoro-3-quinolinyl)ethyl)-amino)-5-pyrimidinecarbonitrile

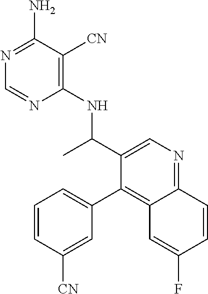 [SMILES: CC(Nc1ncnc(N)c1C#N)c1cnc2ccc(F)cc2c1-c1cccc(C#N)c1]

The rt ¹H-NMR reflects a roughly 1:1 mixture of isomers.

¹H NMR (500 MHz, DMSO-d₆) δ ppm 9.25 (s, 0.5H), 9.21 (s, 0.5H), 8.14 (m, 1H), 8.03 (m, 0.5H), 8.01 (m, 1H), 7.97 (d, J=7.6 Hz, 0.5H), 7.92 (m, 1H), 7.88 (dt, J=7.87, 1.2 Hz, 0.5H), 7.85 (m, 1H), 7.79 (m, 1H), 7.72 (dt, J=7.8, 1.5 Hz, 0.5H), 7.67 (m, 1H), 7.22 (br m, 2H), 6.88 (dd, J=10.3, 3.0 Hz, 0.5H), 6.84 (dd, J=10.3, 2.9 Hz, 0.5H), 4.98 (m, 1H), 1.52 (d, J=7.3 Hz, 1.5H), 1.47 (d, J=7.1 Hz, 1.5H). Mass Spectrum (ESI) m/e=410.2 (M+1). The individual enantiomers were obtained according to the methods described in General Method B4 to give 4-amino-6-(((1S)-1-(4-(3-cyanophenyl)-6-fluoro-3-quinolinyl)ethyl)amino)-5-pyrimidinecarbonitrile and 4-amino-6-(((1R)-1-(4-(3-cyanophenyl)-6-fluoro-3-quinolinyl)ethyl)amino)-5-pyrimidinecarbonitrile and the spectral data of each chiral enantiomer was consistent with that of racemic 4-amino-6-((1-(4-(3-cyanophenyl)-6-fluoro-3-quinolinyl)ethyl)amino)-5-pyrimidinecarbonitrile.

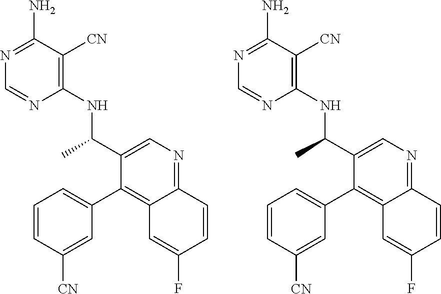 [SMILES: C[C@H](Nc1ncnc(N)c1C#N)c1cnc2ccc(F)cc2c1-c1cccc(C#N)c1 ; C[C@@H](Nc1ncnc(N)c1C#N)c1cnc2ccc(F)cc2c1-c1cccc(C#N)c1]

### Example 18

### 4-Amino-6-((1-(4-(4-cyanophenyl)-6-fluoro-3-quinolinyl)ethyl)-amino)-5-pyrimidinecarbonitrile

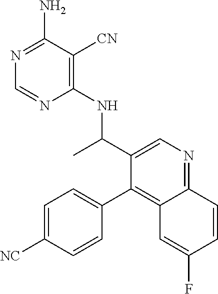 [SMILES: CC(Nc1ncnc(N)c1C#N)c1cnc2ccc(F)cc2c1-c1ccc(C#N)cc1]

¹H NMR (500 MHz, DMSO-d₆) δ ppm 9.22 (s, 1H) 8.13 (dd, J=9.3, 5.6 Hz, 1H) 8.07 (dd, J=7.8, 1.7 Hz, 1H) 8.04 (dd, J=7.9, 1.6 Hz, 1H) 7.92 (d, J=7.1 Hz, 1H) 7.86 (s, 1H) 7.76 (dd, J=7.9, 1.6 Hz, 1H) 7.66 (td, J=8.7, 2.8 Hz, 1H) 7.59 (dd, J=7.9, 1.6 Hz, 1H) 7.22 (br. s., 2H) 6.83 (dd, J=10.1, 2.8 Hz, 1H) 4.97 (quintet, J=7.1 Hz, 1H), 1.48 (d, J=7.3 Hz, 3H). Mass Spectrum (ESI) m/e=410.2 (M+1).

### Example 19

### 4-Amino-6-((1-(6-fluoro-4-(3-fluorophenyl)-3-quinolinyl)ethyl)-amino)-5-pyrimidinecarbonitrile

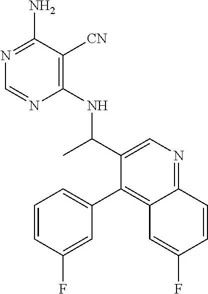 [SMILES: CC(Nc1ncnc(N)c1C#N)c1cnc2ccc(F)cc2c1-c1cccc(F)c1]

The rt ¹H-NMR reflects a roughly 1:1 mixture of isomers.

¹H NMR (500 MHz, DMSO-d₆) δ ppm 9.21 (s, 0.5H), 9.19 (s, 0.5H), 8.13 (m, 1H), 7.93 (d, J=7.3 Hz, 0.5H), 7.90 (d, J=7.3 Hz, 0.5H), 7.85 (m, 1H), 7.65 (m, 2H), 7.40 (m, 2H), 7.30-7.11 (series of m, 3H), 6.89 (m, 1H), 5.10 (m, 1H), 1.51 (d, J=7.3 Hz, 1.5H), 1.47 (d, J=7.3 Hz, 1.5H). Mass Spectrum (ESI) m/e=403.2 (M+1). The individual enantiomers were obtained according to the methods described in General Method B4 to give 4-amino-6-(((1S)-1-(6-fluoro-4-(3-fluorophenyl)-3-quinolinyl)ethyl)amino)-5-pyrimidinecarbonitrile and 4-amino-6-(((1R)-1-(6-fluoro-4-(3-fluorophenyl)-3-quinolinyl)ethyl)amino)-5-pyrimidinecarbonitrile and the spectral data of each chiral enantiomer was consistent with that of racemic 4-amino-6-((1-(6-fluoro-4-(3-fluorophenyl)-3-quinolinyl)ethyl)amino)-5-pyrimidinecarbonitrile.

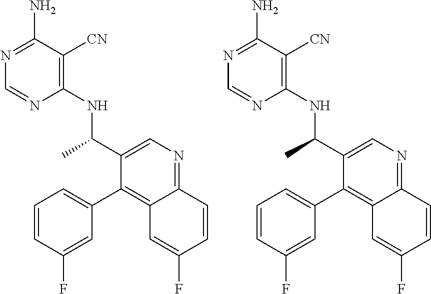 [SMILES: C[C@H](Nc1ncnc(N)c1C#N)c1cnc2ccc(F)cc2c1-c1cccc(F)c1 ; C[C@@H](Nc1ncnc(N)c1C#N)c1cnc2ccc(F)cc2c1-c1cccc(F)c1]

### Example 20

### 4-Amino-6-((1-(4-(3,5-difluorophenyl)-6-fluoro-3-quinolinyl)-ethyl)amino)-5-pyrimidinecarbonitrile

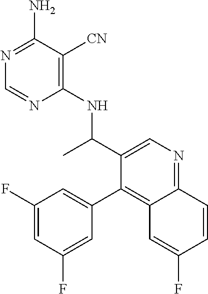 [SMILES: CC(Nc1ncnc(N)c1C#N)c1cnc2ccc(F)cc2c1-c1cc(F)cc(F)c1]

¹H NMR (500 MHz, DMSO-d₆) δ ppm 9.20 (s, 1H), 8.13 (dd, J=9.0, 5.6 Hz, 1H), 7.91 (d, J=7.3 Hz, 1H), 7.84 (s, 1H), 7.67 (td, J=8.7, 2.8 Hz, 1H), 7.42 (tt, J=9.4, 2.3 Hz, 1H), 7.25-7.33 (m, 1H), 7.12-7.25 (m, 2H), 6.96 (dd, J=10.1, 2.8 Hz, 1H), 5.08 (quintet, J=7.2 Hz, 1H), 1.50 (d, J=7.1 Hz, 3H). Mass Spectrum (ESI) m/e=421.2 (M+1). The individual enantiomers were obtained according to the methods described in General Method B4 to give 4-amino-6-(((1S)-1-(4-(3,5-difluorophenyl)-6-fluoro-3-quinolinyl)ethyl)amino)-5-pyrimidinecarbonitrile and 4-amino-6-(((1R)-1-(4-(3,5-difluorophenyl)-6-fluoro-3-quinolinyl)ethyl)amino)-5-pyrimidinecarbonitrile and the spectral data of each chiral enantiomer was consistent with that of racemic 4-amino-6-((1-(4-(3,5-difluorophenyl)-6-fluoro-3-quinolinyl)ethyl)amino)-5-pyrimidinecarbonitrile.

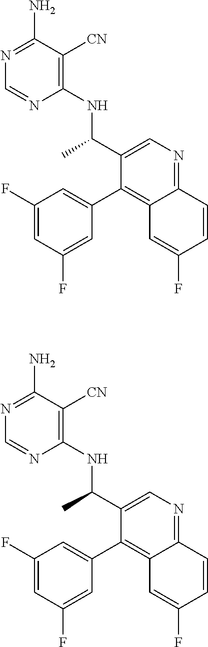 [SMILES: C[C@H](Nc1ncnc(N)c1C#N)c1cnc2ccc(F)cc2c1-c1cc(F)cc(F)c1 ; C[C@@H](Nc1ncnc(N)c1C#N)c1cnc2ccc(F)cc2c1-c1cc(F)cc(F)c1]

The following compound was made via general methods All, A1, A2, A3, A5 starting from 1-(4-chloro-6-fluoroquinolin-3-yl)ethanone (synthesis according to general methods B5, B8 and B7):

### Example 21

### N-(1-(6-Fluoro-4-(3-fluorophenyl)-3-quinolinyl)ethyl)-9H-purin-6-amine

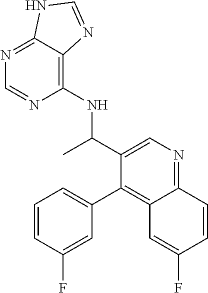 [SMILES: CC(Nc1ncnc2[nH]cnc12)c1cnc2ccc(F)cc2c1-c1cccc(F)c1]

The rt ¹H-NMR reflects a roughly 1:1 mixture of isomers. ¹H NMR (500 MHz, DMSO-d₆) δ ppm 12.9 (br s, 1H), 9.22 (m, 1H), 8.40 (br s, 1H), 8.10 (m, 3H), 7.64 (m, 3H), 7.56 (d, J=7.6 Hz, 0.5H), 7.40 (m, 1H), 7.32 (ddd, J=9.3, 2.5, 1.2 Hz, 0.5H), 7.23 (d, J=7.6 Hz, 0.5H), 6.90 (m, 1H), 5.24 (br s, 1H), 1.54 (d, J=7.1 Hz, 1.5H), 1.51 (d, J=7.1 Hz, 1.5H). Mass Spectrum (ESI) m/e=403.2 (M+1). The individual enantiomers were obtained according to the methods described in General Method B4 to give N-((1S)-1-(6-fluoro-4-(3-fluorophenyl)-3-quinolinyl)ethyl)-9H-purin-6-amine and N-((1S)-1-(6-fluoro-4-(3-fluorophenyl)-3-quinolinyl)ethyl)-9H-purin-6-amine and the spectral data of each chiral enantiomer was consistent with that of racemic N-(1-(6-fluoro-4-(3-fluorophenyl)-3-quinolinyl)ethyl)-9H-purin-6-amine.

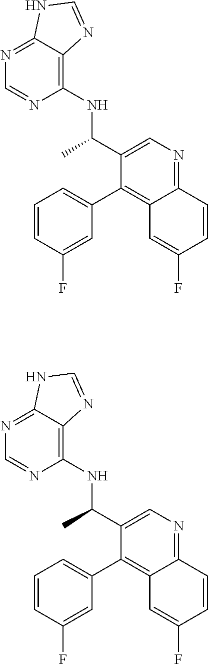 [SMILES: C[C@H](Nc1ncnc2[nH]cnc12)c1cnc2ccc(F)cc2c1-c1cccc(F)c1 ; C[C@@H](Nc1ncnc2[nH]cnc12)c1cnc2ccc(F)cc2c1-c1cccc(F)c1]

The following compounds were made via General Methods A6, A0, A1, A2, A7, A3, A4:

### Example 22

### 4-Amino-6-((1-(4-(4-(methylsulfonyl)phenyl)-3-cinnolinyl)ethyl)-amino)-5-pyrimidinecarbonitrile

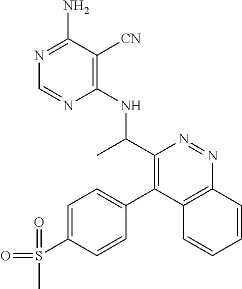 [SMILES: CC(Nc1ncnc(N)c1C#N)c1nnc2ccccc2c1-c1ccc(S(C)(=O)=O)cc1]

¹H NMR (500 MHz, DMSO-d₆) δ ppm 8.54 (d, J=8.2 Hz, 1H), 8.13 (m, 1H), 7.97 (ddd J=8.2, 6.8, 1.2 Hz, 1H), 7.88 (s, 1H), 7.83 (m, 2H), 7.75 (m, 1H), 7.67 (d, J=7.2 Hz, 1H), 7.36 (d, J=8.2 Hz, 1H), 7.21 (br s, 2H), 5.36 (quintet, J=6.7 Hz, 1H), 3.35 (s, 3H), 1.6 (d, J=7.0 Hz, 3H). Mass Spectrum (ESI) m/e=444.0 (M+1).

### Example 23

### 4-Amino-6-((1-(4-(3-(methylsulfonyl)phenyl)-3-cinnolinyl)ethyl)-amino)-5-pyrimidinecarbonitrile

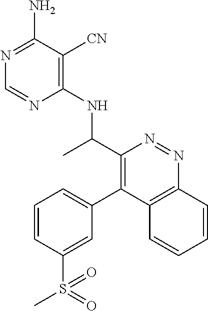 [SMILES: CC(Nc1ncnc(N)c1C#N)c1nnc2ccccc2c1-c1cccc(S(C)(=O)=O)c1]

The rt ¹H-NMR reflects a roughly 6:4 mixture of isomers. ¹H NMR (500 MHz, DMSO-d₆) δ ppm 8.56 (m, 1H), 8.24 (m, 0.6H), 8.16 (dt, J=7.2, 1.8 Hz, 0.6H), 8.13 (dt, J=7.2, 2.0 Hz, 0.4H), 8.04 (m, 0.4H), 7.94-7.82 (series of m, 4H), 7.75 (d, J=7.0 Hz, 0.6H), 7.70 (d, J=7.0 Hz, 0.4H), 7.41 (m, 1H), 7.24 (br s, 2H), 5.35 (m, 1H), 3.29 (m, 1.8H), 1.59 (m, 3H). Mass Spectrum (ESI) m/e=444.0 (M+1).

The following compound was made from 2-(1-(4-cyclopropyl-6-fluoroquinolin-3-yl)ethyl)isoindoline-1,3-dione (ASE1) via general methods A3, A4:

### Example 24

### 4-Amino-6-((1-(4-cyclopropyl-6-fluoro-3-quinolinyl)ethyl)-amino)-5-pyrimidinecarbonitrile

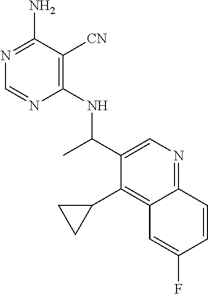 [SMILES: CC(Nc1ncnc(N)c1C#N)c1cnc2ccc(F)cc2c1C1CC1]

¹H NMR (500 MHz, DMSO-d₆) δ ppm 9.03 (s, 1H), 8.14 (dd J=11.0, 2.9 Hz, 1H), 7.91 (m, 2H), 7.60 (ddd, J=9.0, 8.3, 2.6 Hz, 1H), 7.20 (br s, 2H), 6.16 (quintet, J=7.1 Hz, 1H), 2.23 (tt, J=8.3, 6.1 Hz, 1H), 1.60 (d, J=7.1 Hz, 3H), 1.30 (m, 2H), 1.00 (m, 1H), 0.69 (m, 1H). Mass Spectrum (ESI) m/e=349.2 (M+1). The individual enantiomers were obtained according to the methods described in General Method B4 to give 4-amino-6-(((1S)-1-(4-cyclopropyl-6-fluoro-3-quinolinyl)ethyl)amino)-5-pyrimidinecarbonitrile and 4-amino-6-(((1R)-1-(4-cyclopropyl-6-fluoro-3-quinolinyl)ethyl)amino)-5-pyrimidinecarbonitrile and the spectral data of each chiral enantiomer was consistent with that of racemic 4-amino-6-((1-(4-cyclopropyl-6-fluoro-3-quinolinyl)ethyl)amino)-5-pyrimidinecarbonitrile.

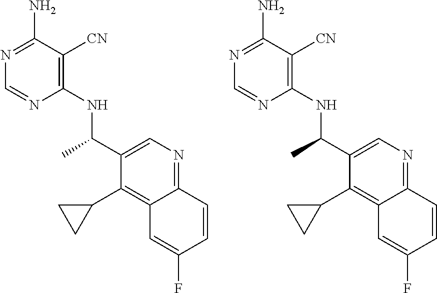 [SMILES: C[C@H](Nc1ncnc(N)c1C#N)c1cnc2ccc(F)cc2c1C1CC1 ; C[C@@H](Nc1ncnc(N)c1C#N)c1cnc2ccc(F)cc2c1C1CC1]

The following compounds were made via general methods A9, A10, A4 described above.

### Example 25

### 4-Amino-6-((1-(6-fluoro-4-(2-pyridinyl)-3-quinolinyl)ethyl)-amino)-5-pyrimidinecarbonitrile

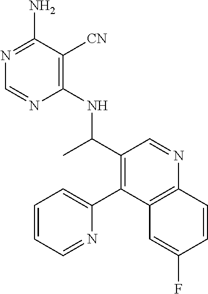 [SMILES: CC(Nc1ncnc(N)c1C#N)c1cnc2ccc(F)cc2c1-c1ccccn1]

The rt ¹H-NMR spectrum reflects a roughly 4:1 mixture of isomers.

¹H NMR (500 MHz, DMSO-d₆) δ ppm 9.25 (m, 1H), 8.80 (m, 1H), 8.07-7.50 (series of m, 6H), 7.45 (td, J=9.05, 2.2 Hz, 1H), 7.34 (dd, J=9.3, 6.3 Hz, 1H), 7.21 (br s, 2H), 5.43 (m, 0.2H), 5.10 (m, 0.8H), 1.57 (br s, 0.6H), 1.46 (br d, J=6.8 Hz, 2.4H). Mass Spectrum (ESI) m/e=386.2 (M+1). The individual enantiomers were obtained according to the methods described in General Method B4 to give 4-amino-6-(((1S)-1-(6-fluoro-4-(2-pyridinyl)-3-quinolinyl)-ethyl)amino)-5-pyrimidinecarbonitrile and 4-amino-6-(((1R)-1-(6-fluoro-4-(2-pyridinyl)-3-quinolinyl)ethyl)amino)-5-pyrimidinecarbonitrile and the spectral data of each chiral enantiomer was consistent with that of racemic 4-amino-6-((1-(6-fluoro-4-(2-pyridinyl)-3-quinolinyl)ethyl)amino)-5-pyrimidinecarbonitrile.

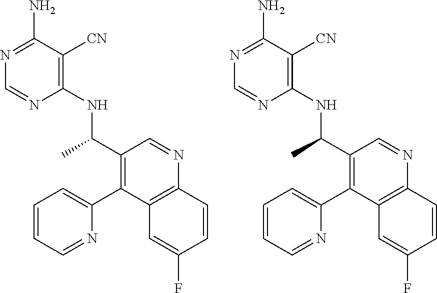 [SMILES: C[C@H](Nc1ncnc(N)c1C#N)c1cnc2ccc(F)cc2c1-c1ccccn1 ; C[C@@H](Nc1ncnc(N)c1C#N)c1cnc2ccc(F)cc2c1-c1ccccn1]

### Example 26

### 4-Amino-6-((1-(7-fluoro-4-(2-pyridinyl)-3-quinolinyl)ethyl)-amino)-5-pyrimidinecarbonitrile

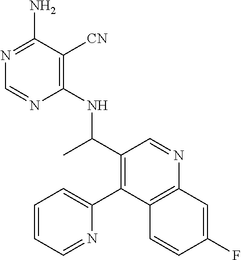 [SMILES: CC(Nc1ncnc(N)c1C#N)c1cnc2cc(F)ccc2c1-c1ccccn1]

The rt ¹H-NMR spectrum reflects a roughly 4:1 mixture of isomers.

¹H NMR (500 MHz, DMSO-d₆) δ ppm 9.24 (m, 1H), 8.80 (m, 1H), 8.08-7.48 (series of m, 6H), 7.45 (td, J=9.05, 2.7 Hz, 1H), 7.34 (dd, J=9.3, 6.3 Hz, 1H), 7.21 (br s, 2H), 5.43 (m, 0.2H), 5.09 (m, 0.8H), 1.57 (br s, 0.6H), 1.46 (br d, J=6.4 Hz, 2.4H). Mass Spectrum (ESI) m/e=386.2 (M+1). The individual enantiomers were obtained according to the methods described in General Method B4 to give 4-amino-6-(((1S)-1-(7-fluoro-4-(2-pyridinyl)-3-quinolinyl)-ethyl)amino)-5-pyrimidinecarbonitrile and 4-amino-6-(((1R)-1-(7-fluoro-4-(2-pyridinyl)-3-quinolinyl)ethyl)amino)-5-pyrimidinecarbonitrile and the spectral data of each chiral enantiomer was consistent with that of racemic 4-amino-6-((1-(7-fluoro-4-(2-pyridinyl)-3-quinolinyl)ethyl)amino)-5-pyrimidinecarbonitrile.

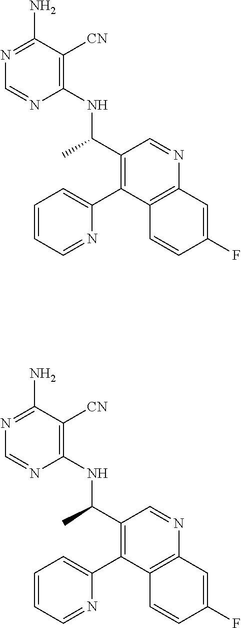 [SMILES: C[C@H](Nc1ncnc(N)c1C#N)c1cnc2cc(F)ccc2c1-c1ccccn1 ; C[C@@H](Nc1ncnc(N)c1C#N)c1cnc2cc(F)ccc2c1-c1ccccn1]

### Example 27

### 4-Amino-6-((1-(6-fluoro-4-(2-pyrazinyl)-3-quinolinyl)ethyl)-amino)-5-pyrimidinecarbonitrile

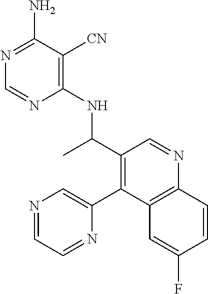 [SMILES: CC(Nc1ncnc(N)c1C#N)c1cnc2ccc(F)cc2c1-c1cnccn1]

The rt ¹H-NMR spectrum reflects a mixture of isomers. ¹H NMR (500 MHz, DMSO-d₆) δ ppm 9.28 (s, 1H), 9.00-8.70 (series of m, 3H), 8.17 (dd. J=9.3, 5.6 Hz, 1H), 8.07-7.55 (series of m, 3H), 7.21 (br s, 2H), 7.02 (dd, J=10.3, 3.0 Hz, 1H), 5.34 (br s, 0.25H), 4.95 (br s, 0.75H), 1.70-1.45 (m, 3H). Mass Spectrum (ESI) m/e=385.1 (M+1).

### Example 28

### 4-Amino-6-((1-(7-fluoro-4-(2-pyrazinyl)-3-quinolinyl)ethyl)-amino)-5-pyrimidinecarbonitrile

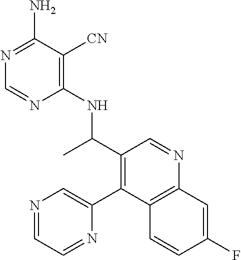 [SMILES: CC(Nc1ncnc(N)c1C#N)c1cnc2cc(F)ccc2c1-c1cnccn1]

The rt ¹H-NMR spectrum reflects a mixture of isomers. ¹H NMR (500 MHz, DMSO-d₆) δ ppm 9.35 (s, 1H), 8.86 (series of m, 3H), 7.99 (br s, 0.75H), 7.86 (dd, J=10.0, 2.9 Hz, 1H) 7.85 (br s, 1H), 7.62 (br s, 0.25H), 7.49 (td, J=10.5, 2.5 Hz, 1H), 7.40 (dd, J=9.3, 6.1 Hz, 1H), 7.21 (br s, 2H), 5.35 (br s, 0.35H), 4.96 (br s, 0.75H), 1.70-1.45 (m, 3H). Mass Spectrum (ESI) m/e=385.1 (M+1).

The following compounds were made via general methods A9, A10, A5 described above.

### Example 29

### N-(1-(6-Fluoro-4-(2-pyridinyl)-3-quinolinyl)ethyl)-9H-purin-6-amine

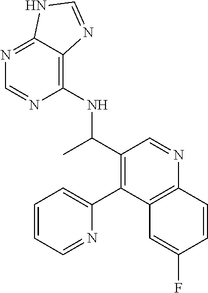 [SMILES: CC(Nc1ncnc2[nH]cnc12)c1cnc2ccc(F)cc2c1-c1ccccn1]

¹H NMR (500 MHz, DMSO-d₆) δ ppm 12.88 (br s, 0.85H), 11.97 (br s, 0.05H), 9.26 (br s, 1H), 8.82 (br d, J=3.4 Hz, 1H), 8.57-7.48 (series of m, 8H), 6.89 (br d, J=7.8 Hz, 1H), 5.60-5.50 (br m, 1H), 1.70-1.45 (br m, 1H). Mass Spectrum (ESI) m/e=386.2 (M+1).

### Example 30

### N-(1-(7-Fluoro-4-(2-pyridinyl)-3-quinolinyl)ethyl)-9H-purin-6-amine

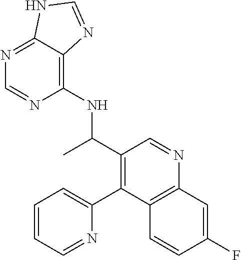 [SMILES: CC(Nc1ncnc2[nH]cnc12)c1cnc2cc(F)ccc2c1-c1ccccn1]

The rt ¹H-NMR spectrum reflects a mixture of isomers. ¹H NMR (500 MHz, DMSO-d₆) δ ppm 12.89 (br s, 1H), 11.97 (br s, 0.05H), 9.45-9.10 (series of m, 1H), 8.80 (br s, 1H), 8.55-7.50 (series of m, 8H), 7.45 (td, J=9.3, 2.7 Hz, 1H), 7.33 (m, 1H), 5.57-5.05 (series of m, 1H), 1.67-1.47 (series of m, 3H). Mass Spectrum (ESI) m/e=386.2 (M+1).

The following compounds were made via A6, A0, A10, A4 as described above:

### Example 31

### 4-Amino-6-((1-(4-(2-pyridinyl)-3-cinnolinyl)ethyl)amino)-5-pyrimidinecarbonitrile

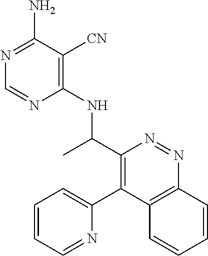 [SMILES: CC(Nc1ncnc(N)c1C#N)c1nnc2ccccc2c1-c1ccccn1]

¹H NMR (500 MHz, DMSO-d₆) δ ppm 8.82 (br d, J=4.9 Hz, 1H), 8.56 (br d, J=8.6 Hz, 1H), 8.06 (td, J=7.6, 1.5 Hz, 1H), 7.97 (ddd, J=8.1, 6.8, 1.0 Hz, 1H), 7.86 (s, 1H), 7.83 (ddd, J=8.1, 6.9, 1.0 Hz, 1H), 7.71 (br d, J=7.6 Hz, 1H), 7.64 (br m, 1H), 7.60 (dd, J=7.6, 4.8 Hz, 1 h), 7.47 (d, J=8.6 Hz, 1H), 7.25 (br s, 2H), 5.52 (br s, 1H), 1.55 (d, J=6.9 Hz, 3H). Mass Spectrum (ESI) m/e=369.2 (M+1). The individual enantiomers were obtained according to the methods described in General Method B4 to give 4-amino-6-(((1R)-1-(4-(2-pyridinyl)-3-cinnolinyl)ethyl)amino)-5-pyrimidinecarbonitrile and 4-amino-6-(((1S)-1-(4-(2-pyridinyl)-3-cinnolinyl)ethyl)amino)-5-pyrimidinecarbonitrile and the spectral data of each chiral enantiomer was consistent with that of racemic 4-amino-6-((1-(4-(2-pyridinyl)-3-cinnolinyl)ethyl)amino)-5-pyrimidinecarbonitrile.

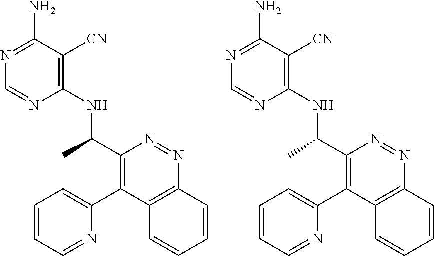 [SMILES: C[C@@H](Nc1ncnc(N)c1C#N)c1nnc2ccccc2c1-c1ccccn1 ; C[C@H](Nc1ncnc(N)c1C#N)c1nnc2ccccc2c1-c1ccccn1]

### Example 32

### 4-Amino-6-((1-(6-fluoro-4-(2-pyridinyl)-3-cinnolinyl)ethyl)-amino)-5-pyrimidinecarbonitrile

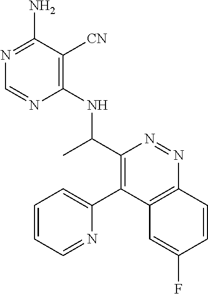 [SMILES: CC(Nc1ncnc(N)c1C#N)c1nnc2ccc(F)cc2c1-c1ccccn1]

¹H NMR (500 MHz, DMSO-d₆) δ ppm 8.82 (br d, J=4.9 Hz, 1H), 8.56 (dd, J=9.3, 5.6 Hz, 1H), 8.07 (td, J=7.8, 1.7 Hz, 1H), 7.91 (td, J=8.6, 2.7 Hz, 1H), 7.84 (s, 1H), 7.73 (d, J=7.8 Hz, 1H), 7.65 (br m, 1H), 7.60 (ddd, J=7.6, 4.9, 0.7 Hz, 1H), 7.24 (br s, 2H), 7.11 (dd, J=9.5, 2.7 Hz, 1H), 5.52 (br s, 1H), 1.56 (d, J=6.9 Hz, 3H). Mass Spectrum (ESI) m/e=387.2 (M+1). The individual enantiomers were obtained according to the methods described in General Method B4 to give 4-amino-6-(((1R)-1-(6-fluoro-4-(2-pyridinyl)-3-cinnolinyl)-ethyl)amino)-5-pyrimidinecarbonitrile and 4-amino-6-(((1S)-1-(6-fluoro-4-(2-pyridinyl)-3-cinnolinyl)ethyl)amino)-5-pyrimidinecarbonitrile and the spectral data of each chiral enantiomer was consistent with that of racemic 4-amino-6-((1-(6-fluoro-4-(2-pyridinyl)-3-cinnolinyl)ethyl)amino)-5-pyrimidinecarbonitrile.

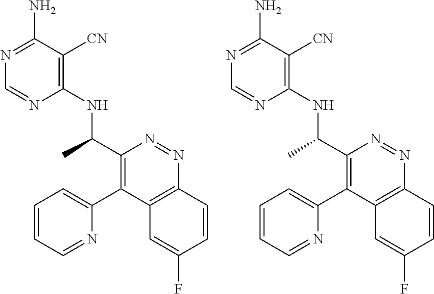 [SMILES: C[C@@H](Nc1ncnc(N)c1C#N)c1nnc2ccc(F)cc2c1-c1ccccn1 ; C[C@H](Nc1ncnc(N)c1C#N)c1nnc2ccc(F)cc2c1-c1ccccn1]

The following compounds were made via general methods A6, A0, A10, A5 as described above:

### Example 33

### N-(1-(6-Fluoro-4-(2-pyridinyl)-3-cinnolinyl)ethyl)-9H-purin-6-amine

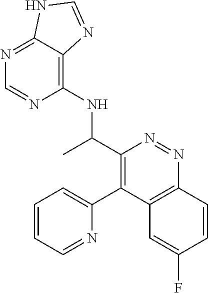 [SMILES: CC(Nc1ncnc2[nH]cnc12)c1nnc2ccc(F)cc2c1-c1ccccn1]

¹H NMR (500 MHz, DMSO-d₆) δ ppm 12.8 (br s, 1H), 8.84 (br d, J=3.7 Hz, 1H), 8.65 (dd, J=9.3, 5.6 Hz, 1H), 8.20-7.78 (series of m, 6H), 7.61 (m, 1H), 7.15 (dd, J=9.5, 2.7 Hz, 1H), 5.57 (br s, 1H), 1.66 (d, J=6.9 Hz, 3H). Mass Spectrum (ESI) m/e=387.2 (M+1).

The following compound was made by general methods A3, A4 from 2-(1-(4-phenylisoquinolin-3-yl)ethyl)isoindoline-1,3-dione (ASE2):

### Example 34

### 4-Amino-6-((1-(4-phenyl-3-isoquinolinyl)ethyl)amino)-5-pyrimidinecarbonitrile

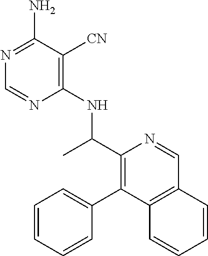 [SMILES: CC(Nc1ncnc(N)c1C#N)c1ncc2ccccc2c1-c1ccccc1]

¹H NMR (500 MHz, DMSO-d₆) δ ppm 9.46 (s, 1H), 8.20 (m, 1H), 7.94 (m, 1H), 7.70 (m, 2H), 7.62-7.52 (series of m, 3H), 7.40 (m, 2H), 7.36-7.24 (series of m, 3H), 7.11 (d, J=7.6 Hz, 1H), 5.27 (quintent, J=6.6 Hz, 1H), 1.34 (d, J=6.6 Hz, 3H). Mass Spectrum (ESI) m/e=367 (M+1). The individual enantiomers were obtained according to the methods described in General Method B4 to give 4-amino-6-(((1S)-1-(4-phenyl-3-isoquinolinyl)ethyl)amino)-5-pyrimidinecarbonitrile and 4-amino-6-(((1R)-1-(4-phenyl-3-isoquinolinyl)ethyl)amino)-5-pyrimidinecarbonitrile and the spectral data of each chiral enantiomer was consistent with that of racemic 4-amino-6-(((1R)-1-(4-phenyl-3-isoquinolinyl)ethyl)-amino)-5-pyrimidinecarbonitrile.

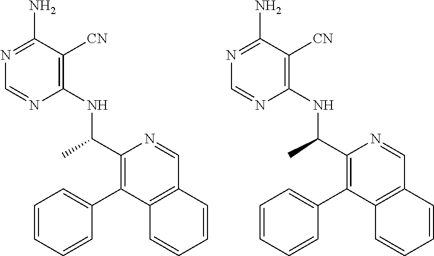 [SMILES: C[C@H](Nc1ncnc(N)c1C#N)c1ncc2ccccc2c1-c1ccccc1 ; C[C@@H](Nc1ncnc(N)c1C#N)c1ncc2ccccc2c1-c1ccccc1]

### Example 35

### 4-Amino-6-((1-(4-phenyl-3-quinolinyl)ethyl)amino)-5-pyrimidinecarbonitrile

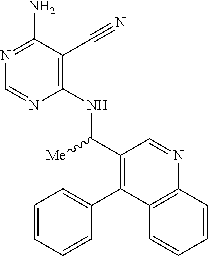 [SMILES: CC(Nc1ncnc(N)c1C#N)c1cnc2ccccc2c1-c1ccccc1]

4-Amino-6-(1-(4-phenylquinolin-3-yl)ethylamino)pyrimidine-5-carbonitrile was prepared according to the methods described in General Methods B13, B12, B11, B10, A3 and A4, starting from ethyl 4-chloroquinoline-3-carboxylate (Journal of Medicinal Chemistry, 2006, vol. 49, #21, p. 6351-6363). ¹H NMR (500 MHz, DMSO-d₆) δ ppm 9.19 (1H, s), 8.02 (1H, d, J=8.3 Hz), 7.88 (1H, d, J=7.3 Hz), 7.86 (1H, s), 7.70 (1H, ddd, J=8.3, 6.8, 1.5 Hz), 7.44-7.63 (5H, m), 7.25-7.34 (2H, m), 7.22 (2H, br. s.), 5.12 (1H, qd, J=7.1, 6.8 Hz), 1.46 (3H, d, J=7.1 Hz). Mass Spectrum (ESI) m/e=367.1 (M+1). The individual enantiomers were obtained by chiral SFC purification.

### 4-Amino-6-(((1R)-1-(4-phenyl-3-quinolinyl)ethyl)amino)-5-pyrimidinecarbonitrile

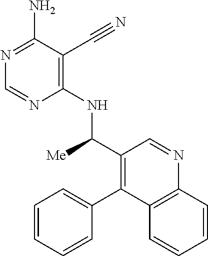 [SMILES: C[C@@H](Nc1ncnc(N)c1C#N)c1cnc2ccccc2c1-c1ccccc1]

4-Amino-6-(((1R)-1-(4-phenyl-3-quinolinyl)ethyl)amino)-5-pyrimidinecarbonitrile was prepared according to the methods described in General Method B4 starting from 4-amino-6-(1-(4-phenylquinolin-3-yl)ethylamino)pyrimidine-5-carbonitrile. The stereochemistry is arbitrarily assigned. ¹H NMR (500 MHz, DMSO-d₆) δ ppm 9.19 (1H, s), 8.02 (1H, d, J=7.8 Hz), 7.88 (1H, d, J=7.3 Hz), 7.86 (1H, s), 7.70 (1H, ddd, J=8.3, 6.8, 1.5 Hz), 7.51-7.62 (4H, m), 7.46-7.51 (1H, m), 7.31 (1H, d, J=7.6 Hz), 7.27 (1H, d, J=7.6 Hz), 7.18 (2H, br. s.), 5.12 (1H, quin, J=7.2 Hz), 1.46 (3H, d, J=7.1 Hz). Mass Spectrum (ESI) m/e=367.1 (M+1) and 365.0 (M−1). EE>99%.

### 4-Amino-6-(((1S)-1-(4-phenyl-3-quinolinyl)ethyl)amino)-5-pyrimidinecarbonitrile

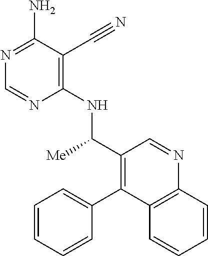 [SMILES: C[C@H](Nc1ncnc(N)c1C#N)c1cnc2ccccc2c1-c1ccccc1]

4-Amino-6-(((1S)-1-(4-phenyl-3-quinolinyl)ethyl)amino)-5-pyrimidinecarbonitrile was prepared according to the methods described in General Method B4 starting from 4-amino-6-(1-(4-phenylquinolin-3-yl)ethylamino)pyrimidine-5-carbonitrile. The stereochemistry is arbitrarily assigned. ¹H NMR (500 MHz, DMSO-d₆) δ ppm 9.19 (1H, s), 8.02 (1H, d, J=8.1 Hz), 7.88 (1H, d, J=7.3 Hz), 7.86 (1H, s), 7.70 (1H, ddd, J=8.4, 6.9, 1.3 Hz), 7.51-7.62 (4H, m), 7.46-7.51 (1H, m), 7.31 (1H, d, J=7.6 Hz), 7.27 (1H, d, J=7.3 Hz), 7.09-7.25 (2H, m), 5.12 (1H, quin, J=7.2 Hz), 1.46 (3H, d, J=7.1 Hz). Mass Spectrum (ESI) m/e=367.1 (M+1) and 365.0 (M−1). EE>99%.

### Example 36

### 4-Amino-6-((1-(5-fluoro-4-phenyl-3-quinolinyl)ethyl)amino)-5-pyrimidinecarbonitrile

### 1-(5-Fluoro-4-phenylquinolin-3-yl)ethanamine

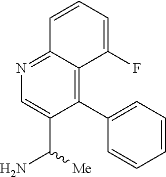 [SMILES: CC(N)c1cnc2cccc(F)c2c1-c1ccccc1]

1-(5-Fluoro-4-phenylquinolin-3-yl)ethanamine was prepared according to the methods described in General Methods B13, B12, B11, B10, and A3 from ethyl 4-chloro-5-fluoroquinoline-3-carboxylate (Bioorganic & Medicinal Chemistry, 2003, vol. 11, #23, p. 5259-5272). Mass Spectrum (ESI) m/e=267.1 (M+1).

### 4-Amino-6-((1-(5-fluoro-4-phenyl-3-quinolinyl)ethyl)amino)-5-pyrimidinecarbonitrile

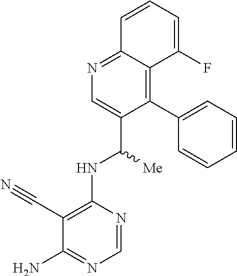 [SMILES: CC(Nc1ncnc(N)c1C#N)c1cnc2cccc(F)c2c1-c1ccccc1]

4-Amino-6-(1-(5-fluoro-4-phenylquinolin-3-yl)ethylamino)pyrimidine-5-carbonitrile was prepared according to the methods described in General Method A4 from 1-(5-fluoro-4-phenylquinolin-3-yl)ethanamine. ¹H NMR (400 MHz, DMSO-d₆) δ ppm 9.21 (1H, s), 7.83-7.96 (3H, m), 7.69 (1H, td, J=8.1, 5.4 Hz), 7.58 (1H, d, J=7.4 Hz), 7.39-7.53 (3H, m), 7.11-7.33 (4H, m), 5.01 (1H, quin, J=7.1 Hz), 1.41 (3H, d, J=7.2 Hz). Mass Spectrum (ESI) m/e=385.2 (M+1) and 383.2 (M−1). The individual enantiomers were obtained by chiral SFC purification.

### 4-Amino-6-(((1R)-1-(5-fluoro-4-phenyl-3-quinolinyl)ethyl)amino)-5-pyrimidinecarbonitrile

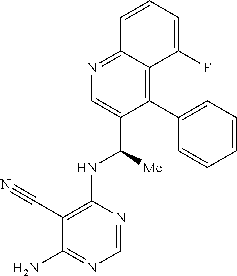 [SMILES: C[C@@H](Nc1ncnc(N)c1C#N)c1cnc2cccc(F)c2c1-c1ccccc1]

4-Amino-6-(((1R)-1-(5-fluoro-4-phenyl-3-quinolinyl)ethyl)amino)-5-pyrimidinecarbonitrile was prepared according to the methods described in General Method B4 starting from 4-amino-6-(1-(5-fluoro-4-phenylquinolin-3-yl)ethylamino)pyrimidine-5-carbonitrile. The stereochemistry is arbitrarily assigned. ¹H NMR (500 MHz, CHLOROFORM-d) δ ppm 9.01 (1H, br. s.), 8.02 (1H, s), 7.98 (1H, d, J=8.6 Hz), 7.62 (1H, td, J=8.1, 5.4 Hz), 7.57-7.60 (1H, m), 7.44-7.53 (3H, m), 7.22-7.26 (1H, m), 7.10 (1H, dd, J=12.1, 7.7 Hz), 5.51 (1H, d, J=6.4 Hz), 5.33 (2H, br. s.), 5.21 (1H, quin, J=6.8 Hz), 1.50 (3H, d, J=7.1 Hz). Mass Spectrum (ESI) m/e=385.1 (M+1) and 383.0 (M−1).

### 4-Amino-6-(((1S)-1-(5-fluoro-4-phenyl-3-quinolinyl)ethyl)amino)-5-pyrimidinecarbonitrile

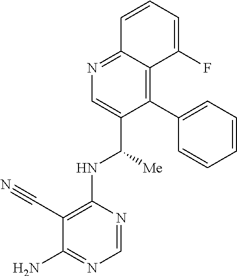 [SMILES: C[C@H](Nc1ncnc(N)c1C#N)c1cnc2cccc(F)c2c1-c1ccccc1]

4-Amino-6-(((1S)-1-(5-fluoro-4-phenyl-3-quinolinyl)ethyl)amino)-5-pyrimidinecarbonitrile was prepared according to the methods described in General Method B4 starting from 4-amino-6-(1-(5-fluoro-4-phenylquinolin-3-yl)ethylamino)pyrimidine-5-carbonitrile. The stereochemistry is arbitrarily assigned. ¹H NMR (500 MHz, CHLOROFORM-d) δ ppm 9.02 (1H, br. s.), 8.02 (1H, s), 7.97 (1H, d, J=8.3 Hz), 7.62 (1H, td, J=8.1, 5.4 Hz), 7.56-7.60 (1H, m), 7.45-7.53 (3H, m), 7.22-7.26 (1H, m), 7.10 (1H, dd, J=12.0, 7.8 Hz), 5.54 (1H, d, J=6.1 Hz), 5.37 (2H, br. s.), 5.21 (1H, quin, J=6.8 Hz), 1.50 (3H, d, J=6.8 Hz). Mass Spectrum (ESI) m/e=385.1 (M+1) and 383.0 (M−1).

### Example 37

### 4-Amino-6-((1-(4-(2-pyridinyl)-3-quinolinyl)ethyl)amino)-5-pyrimidinecarbonitrile

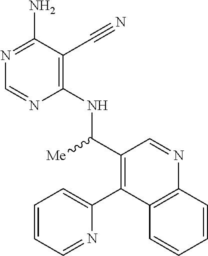 [SMILES: CC(Nc1ncnc(N)c1C#N)c1cnc2ccccc2c1-c1ccccn1]

4-Amino-6-((1-(4-(2-pyridinyl)-3-quinolinyl)ethyl)amino)-5-pyrimidinecarbonitrile was prepared according to the methods described in General Method A4 starting from 1-(4-(pyridin-2-yl)quinolin-3-yl)ethanamine. A mixture of isomers was observed in the proton NMR trace. ¹H NMR (500 MHz, CHLOROFORM-d) δ ppm 9.07 (1H, s), 8.99 (0.86H, d, J=4.2 Hz), 8.84 (0.16H, br. s.), 8.22 (1H, d, J=8.6 Hz), 8.10 (0.86H, s), 7.96 (1H, t, J=7.5 Hz), 7.89 (0.16H, br. s.), 7.69-7.79 (1H, m), 7.42-7.67 (4.75H, m), 7.34 (0.16H, br. s.), 5.61 (0.87H, t, J=7.2 Hz), 5.48 (0.19H, br. s.), 5.33-5.45 (2H, br. s.), 1.61-1.85 (0.38H, br. s.), 1.13-1.39 (2.66H, d, J=7.09 Hz). Mass Spectrum (ESI) m/e=368.0 (M+1) and 366.1 (M−1).

### 4-Amino-6-(((1S)-1-(4-(2-pyridinyl)-3-quinolinyl)ethyl)amino)-5-pyrimidinecarbonitrile

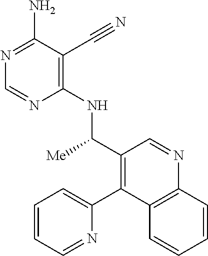 [SMILES: C[C@H](Nc1ncnc(N)c1C#N)c1cnc2ccccc2c1-c1ccccn1]

4-Amino-6-(((1S)-1-(4-(2-pyridinyl)-3-quinolinyl)ethyl)amino)-5-pyrimidinecarbonitrile was prepared according to the methods described in General Method B4 starting from 4-amino-6-((1-(4-(2-pyridinyl)-3-quinolinyl)ethyl)amino)-5-pyrimidinecarbonitrile. The stereochemistry is arbitrarily assigned. A mixture of isomers was observed in the proton NMR trace. ¹H NMR (500 MHz, DMSO-d₆) δ ppm 9.22 (1H, br. s), 8.79 (1H, d, J=3.4 Hz), 8.01-8.11 (2H, m), 7.98 (1H, d, J=6.6 Hz), 7.84 (0.8H, s), 7.73 (1H, ddd, J=8.4, 7.0, 1.2 Hz), 7.70 (1H, d, J=7.8 Hz), 7.45-7.59 (2.4H, m), 7.27 (1H, d, J=8.6 Hz), 7.20 (2H, br. s.), 5.42 (0.2H, br. s.), 4.98-5.19 (0.8H, m), 1.55 (0.58H, br. s.), 1.45 (2.5H, d, J=6.6 Hz). Mass Spectrum (ESI) m/e=368.0 (M+1) and 366.1 (M−1). EE>99%.

### 4-Amino-6-(((1R)-1-(4-(2-pyridinyl)-3-quinolinyl)ethyl)amino)-5-pyrimidinecarbonitrile

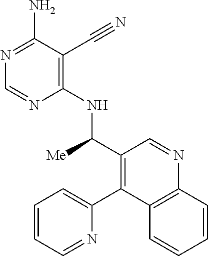 [SMILES: C[C@@H](Nc1ncnc(N)c1C#N)c1cnc2ccccc2c1-c1ccccn1]

4-Amino-6-(((1R)-1-(4-(2-pyridinyl)-3-quinolinyl)ethyl)amino)-5-pyrimidinecarbonitrile was prepared according to the methods described in General Method B4 starting from 4-amino-6-((1-(4-(2-pyridinyl)-3-quinolinyl)ethyl)amino)-5-pyrimidinecarbonitrile. The stereochemistry is arbitrarily assigned. A mixture of isomers was observed in the proton NMR trace. ¹H NMR (500 MHz, DMSO-d₆) δ ppm 9.22 (1H, br. s), 8.79 (1H, d, J=3.4 Hz), 8.01-8.11 (2H, m), 7.98 (1H, d, J=6.6 Hz), 7.84 (0.8H, s), 7.73 (1H, ddd, J=8.4, 7.0, 1.2 Hz), 7.70 (1H, d, J=7.8 Hz), 7.45-7.59 (2.4H, m), 7.27 (1H, d, J=8.6 Hz), 7.20 (2H, br. s.), 5.42 (0.2H, br. s.), 4.98-5.19 (0.8H, m), 1.55 (0.58H, br. s.), 1.45 (2.5H, d, J=6.6 Hz). Mass Spectrum (ESI) m/e=368.0 (M+1) and 366.1 (M−1). EE>99%.

### Example 38

### 4-Amino-6-((1-(8-chloro-4-(2-pyridinyl)-3-quinolinyl)ethyl)-amino)-5-pyrimidinecarbonitrile

### N-(1-(8-Chloro-4-(pyridin-2-yl)quinolin-3-yl)ethylidene)-2-methylpropane-2-<sulfinamide

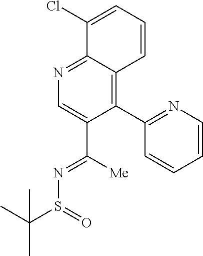 [SMILES: C/C(=N\S(=O)C(C)(C)C)c1cnc2c(Cl)cccc2c1-c1ccccn1]

Tetraethoxytitanium (0.314 mL, 1.514 mmol), 2-methylpropane-2-sulfinamide (0.096 g, 0.795 mmol), and 1-(8-chloro-4-(pyridin-2-yl)quinolin-3-yl)ethanone (0.214 g, 0.757 mmol) were combined in THF (3 mL) under an atmosphere of N₂. The solution was then heated at 60° C. overnight. The next day more tetraethoxytitanium (0.314 mL, 1.514 mmol) and 2-methylpropane-2-sulfinamide (0.096 g, 0.795 mmol) were added and the solution heated to a reflux for 4 h. The solution was poured into brine and ethyl acetate with stirring. The solids were filtered off through Celite™ and the filtrate was partitioned. The organic layer was washed with brine, dried over MgSO₄ and then concentrated under vacuum to give brownish oil. The brownish oil was purified by column chromatography. The fractions containing the product were combined and concentrated under vacuum to give yellow oil which was carried on without further purification. Mass Spectrum (ESI) m/e=386.2 (M+1).

### N-(1-(8-Chloro-4-(pyridin-2-yl)quinolin-3-yl)ethyl)-2-methylpropane-2-sulfinamide

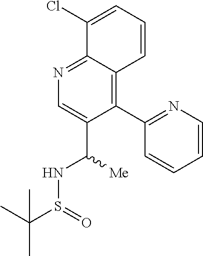 [SMILES: CC(NS(=O)C(C)(C)C)c1cnc2c(Cl)cccc2c1-c1ccccn1]

(E)-N-(1-(8-Chloro-4-(pyridin-2-yl)quinolin-3-yl)ethylidene)-2-methylpropane-2-sulfinamide (0.150 g, 0.389 mmol) was dissolved in THF (4 mL), and H₂O (0.065 mL) before being cooled in a brine dry ice bath under an atmosphere of N₂. Sodium tetrahydroborate (0.029 g, 0.777 mmol) was added and the solution was left to slowly warm to rt. Four days later methanol was added and then solution was concentrated under vacuum. The solids obtained were dissolved in methanol and concentrated under vacuum. The solids obtained were dissolved in ethyl acetate and washed with sat NaHCO₃ followed by brine. The organics were dried over MgSO₄ and then concentrated under vacuum. The residue obtained was carried on without further purification. Mass Spectrum (ESI) m/e=388.2 (M+1).

### 1-(8-Chloro-4-(pyridin-2-yl)quinolin-3-yl)ethanamine

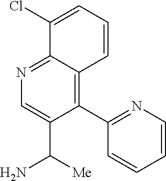 [SMILES: CC(N)c1cnc2c(Cl)cccc2c1-c1ccccn1]

N-(1-(8-Chloro-4-(pyridin-2-yl)quinolin-3-yl)ethyl)-2-methylpropane-2-sulfinamide (0.151 g, 0.389 mmol) was dissolved in THF (5 mL), before adding concentrated HCl (0.5 ml). The solution was stirred at rt. for 15 min and then made basic with 4 N NaOH, the pH was adjusted to ˜9 with sat NaHCO₃. The product was then extracted with ethyl acetate. The organic layer was dried over MgSO₄ and concentrated under vacuum to give a yellowish film. The yellowish film was purified by column chromatography using a gradient of 2% methanol/0.1% NH₄OH (˜28% in water)/DCM to 10% methanol/0.5% NH₄OH (˜28% in water)/DCM. The fractions containing the product were combined and concentrated under vacuum to give 1-(8-chloro-4-(pyridin-2-yl)-quinolin-3-yl)ethanamine as a light yellow film. Mass Spectrum (ESI) m/e=284.2 (M+1).

### 4-Amino-6-((1-(8-chloro-4-(2-pyridinyl)-3-quinolinyl)ethyl)amino)-5-pyrimidinecarbonitrile

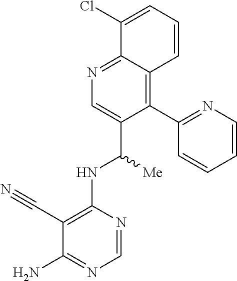 [SMILES: CC(Nc1ncnc(N)c1C#N)c1cnc2c(Cl)cccc2c1-c1ccccn1]

4-Amino-6-((1-(8-chloro-4-(2-pyridinyl)-3-quinolinyl)ethyl)amino)-5-pyrimidinecarbonitrile was prepared according to the methods described in General Method A4 from 1-(8-chloro-4-(pyridin-2-yl)quinolin-3-yl)ethanamine. A mixture of isomers was observed in the proton NMR trace. ¹H NMR (500 MHz, DMSO-d₆) δ ppm 9.32 (1H, br. s), 8.79 (0.85H, d, J=4.2 Hz), 8.75 (0.15H, br. s.), 8.02-8.10 (0.85H, m), 8.00 (1H, d, J=7.1 Hz), 7.93 (1H, dd, J=7.6, 1.0 Hz), 7.84 (0.8H, s), 7.71 (0.85H, d, J=7.1 Hz), 7.66 (0.2H, br. s.), 7.53-7.61 (1H, m), 7.49 (1.3H, t, J=7.9 Hz), 7.23 (3H, d, J=8.3 Hz), 5.41 (0.17H, br. s.), 5.06 (0.8H, quin, J=6.8 Hz), 1.57 (0.57H, br. s.), 1.48 (2.41H, d, J=6.8 Hz). Mass Spectrum (ESI) m/e=402.2 (M+1) and 400.2 (M−1).

### 4-Amino-6-(((1S)-1-(8-chloro-4-(2-pyridinyl)-3-quinolinyl)ethyl)amino)-5-pyrimidinecarbonitrile

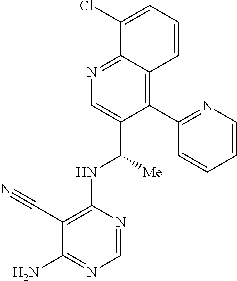 [SMILES: C[C@H](Nc1ncnc(N)c1C#N)c1cnc2c(Cl)cccc2c1-c1ccccn1]

4-Amino-6-(((1S)-1-(8-chloro-4-(2-pyridinyl)-3-quinolinyl)ethyl)amino)-5-pyrimidinecarbonitrile was prepared according to the methods described in General Method B4 starting from 4-amino-6-((1-(8-chloro-4-(2-pyridinyl)-3-quinolinyl)ethyl)amino)-5-pyrimidinecarbonitrile. The stereochemistry is arbitrarily assigned. A mixture of isomers was observed in the proton NMR trace. ¹H NMR (500 MHz, CHLOROFORM-d) δ ppm 9.10-9.29 (1H, m), 8.78-9.04 (1H, m), 7.79-8.20 (3H, m), 7.46-7.69 (3H, m), 7.34-7.45 (2H, m), 5.18-5.76 (3H, m), 1.11-1.81 (3H, m). Mass Spectrum (ESI) m/e=402.1 (M+1) and 400.0 (M−1).

### 4-Amino-6-(((1R)-1-(8-chloro-4-(2-pyridinyl)-3-quinolinyl)ethyl)amino)-5-pyrimidinecarbonitrile

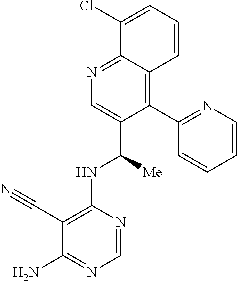 [SMILES: C[C@@H](Nc1ncnc(N)c1C#N)c1cnc2c(Cl)cccc2c1-c1ccccn1]

4-Amino-6-(((1R)-1-(8-chloro-4-(2-pyridinyl)-3-quinolinyl)ethyl)amino)-5-pyrimidinecarbonitrile was prepared according to the methods described in General Method B4 starting from 4-amino-6-((1-(8-chloro-4-(2-pyridinyl)-3-quinolinyl)ethyl)amino)-5-pyrimidinecarbonitrile. The stereochemistry is arbitrarily assigned. ¹H NMR (500 MHz, CHLOROFORM-d) δ ppm 9.12-9.24 (1H, m), 8.76-9.02 (1H, m), 7.78-8.21 (3H, m), 7.44-7.68 (3H, m), 7.32-7.43 (2H, m), 5.05-5.75 (3H, m), 1.03-1.79 (3H, m). Mass Spectrum (ESI) m/e=402.1 (M+1) and 400.0 (M−1).

### Example 39

### 4-Amino-6-((1-(8-fluoro-4-(2-pyridinyl)-3-quinolinyl)ethyl)-amino)-5-pyrimidinecarbonitrile

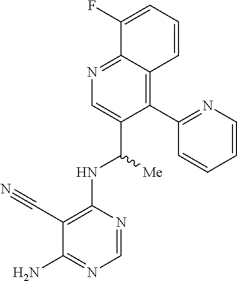 [SMILES: CC(Nc1ncnc(N)c1C#N)c1cnc2c(F)cccc2c1-c1ccccn1]

4-Amino-6-((1-(8-fluoro-4-(2-pyridinyl)-3-quinolinyl)ethyl)amino)-5-pyrimidinecarbonitrile was prepared according to the methods described in General Methods A9, A10, and A4 from 1-(4-chloro-8-fluoroquinolin-3-yl)ethanone. A mixture of isomers was observed in the proton NMR trace. ¹H NMR (500 MHz, DMSO-d₆) δ ppm 9.27 (1H, s), 8.67-8.86 (1H, m), 7.93-8.14 (2H, m), 7.84 (1H, s), 7.62-7.78 (1H, m), 7.38-7.62 (3H, m), 7.21 (2H, br. s.), 7.07 (1H, d, J=8.3 Hz), 4.93-5.50 (1H, m), 1.34-1.64 (3H, m). Mass Spectrum (ESI) m/e=386.0 (M+1) and 384.1 (M−1).

### 4-Amino-6-(((1S)-1-(8-fluoro-4-(2-pyridinyl)-3-quinolinyl)ethyl)amino)-5-pyrimidinecarbonitrile

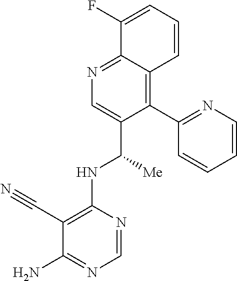 [SMILES: C[C@H](Nc1ncnc(N)c1C#N)c1cnc2c(F)cccc2c1-c1ccccn1]

4-Amino-6-(((1S)-1-(8-fluoro-4-(2-pyridinyl)-3-quinolinyl)ethyl)amino)-5-pyrimidinecarbonitrile was prepared according to the methods described in General Method B4 starting from 4-amino-6-((1-(8-fluoro-4-(2-pyridinyl)-3-quinolinyl)ethyl)amino)-5-pyrimidinecarbonitrile. The stereochemistry is arbitrarily assigned. A mixture of isomers was observed in the proton NMR trace. ¹H NMR (500 MHz, CHLOROFORM-d) δ ppm 9.10 (1H, s), 8.77-9.01 (1H, m), 7.83-8.16 (2H, m), 7.32-7.66 (5H, m), 7.02-7.26 (1H, m), 5.59 (1H, quin, J=7.2 Hz), 5.02-5.35 (2H, m), 1.70 (0.46H, br. s.), 1.19-1.34 (2.59H, m). Mass Spectrum (ESI) m/e=386.0 (M+1).

### 4-Amino-6-(((1R)-1-(8-fluoro-4-(2-pyridinyl)-3-quinolinyl)ethyl)amino)-5-pyrimidinecarbonitrile

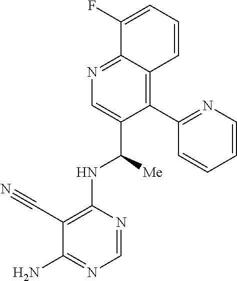 [SMILES: C[C@@H](Nc1ncnc(N)c1C#N)c1cnc2c(F)cccc2c1-c1ccccn1]

4-Amino-6-(((1R)-1-(8-fluoro-4-(2-pyridinyl)-3-quinolinyl)ethyl)amino)-5-pyrimidinecarbonitrile was prepared according to the methods described in General Method B4 starting from 4-amino-6-((1-(8-fluoro-4-(2-pyridinyl)-3-quinolinyl)ethyl)amino)-5-pyrimidinecarbonitrile. The stereochemistry is arbitrarily assigned. A mixture of isomers was observed in the proton NMR trace. ¹H NMR (500 MHz, CHLOROFORM-d) δ ppm 9.10 (1H, s), 8.75-9.02 (1H, m), 7.80-8.17 (2H, m), 7.35-7.68 (5H, m), 7.02-7.25 (1H, m), 5.43-5.67 (1H, m), 5.00-5.37 (2H, m), 1.69 (0.36H, br. s.), 1.19-1.34 (2.7H, m). Mass Spectrum (ESI) m/e=386.0 (M+1).

### Example 40

### 4-Amino-6-((1-(7-chloro-4-(2-pyridinyl)-3-quinolinyl)ethyl)-amino)-5-pyrimidinecarbonitrile

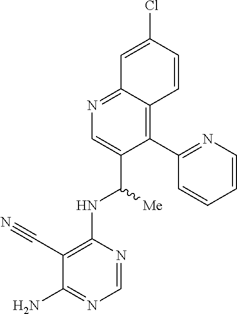 [SMILES: CC(Nc1ncnc(N)c1C#N)c1cnc2cc(Cl)ccc2c1-c1ccccn1]

4-Amino-6-((1-(7-chloro-4-(2-pyridinyl)-3-quinolinyl)ethyl)amino)-5-pyrimidinecarbonitrile was prepared according to the methods described in General Methods A9, A10, and A4 from 1-(4,7-dichloro-quinolin-3-yl)-ethanone. A mixture of isomers was observed in the proton NMR trace. ¹H NMR (500 MHz, DMSO-d₆) δ ppm 9.26 (1H, br. s.), 8.78 (1H, br. s.), 8.12 (1H, d, J=2.2 Hz), 7.42-8.08 (6H, m), 7.00-7.34 (3H, m), 4.97-5.50 (1H, m), 1.36-1.65 (3H, m). Mass Spectrum (ESI) m/e=402.1 (M+1) and 400.0 (M−1).

### 4-Amino-6-(((1S)-1-(7-chloro-4-(2-pyridinyl)-3-quinolinyl)ethyl)amino)-5-pyrimidinecarbonitrile

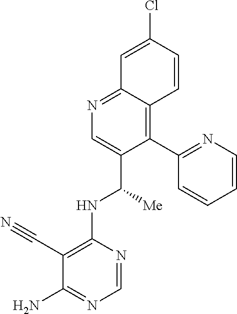 [SMILES: C[C@H](Nc1ncnc(N)c1C#N)c1cnc2cc(Cl)ccc2c1-c1ccccn1]

4-Amino-6-(((1S)-1-(7-chloro-4-(2-pyridinyl)-3-quinolinyl)ethyl)amino)-5-pyrimidinecarbonitrile was prepared according to the methods described in General Method B4 starting from 4-amino-6-((1-(7-chloro-4-(2-pyridinyl)-3-quinolinyl)ethyl)amino)-5-pyrimidinecarbonitrile. The stereochemistry is arbitrarily assigned. A mixture of isomers was observed in the proton NMR trace. ¹H NMR (500 MHz, CHLOROFORM-d) δ ppm 9.04 (1H, s), 8.79-9.01 (1H, m), 8.15 (1H, s), 7.85-8.13 (2H, m), 7.36-7.64 (4.5H, m), 7.20-7.26 (0.3H, m), 5.43-5.64 (1H, m), 5.03-5.34 (2H, m), 1.69 (0.4H, br. s.), 1.17-1.29 (2.6H, m). Mass Spectrum (ESI) m/e=402.1 (M+1).

### 4-Amino-6-(((1R)-1-(7-chloro-4-(2-pyridinyl)-3-quinolinyl)ethyl)amino)-5-pyrimidinecarbonitrile

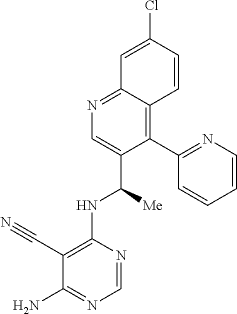 [SMILES: C[C@@H](Nc1ncnc(N)c1C#N)c1cnc2cc(Cl)ccc2c1-c1ccccn1]

4-Amino-6-(((1R)-1-(7-chloro-4-(2-pyridinyl)-3-quinolinyl)ethyl)amino)-5-pyrimidinecarbonitrile was prepared according to the methods described in General Method B4 starting from 4-amino-6-((1-(7-chloro-4-(2-pyridinyl)-3-quinolinyl)ethyl)amino)-5-pyrimidinecarbonitrile. The stereochemistry is arbitrarily assigned. A mixture of isomers was observed in the proton NMR trace. ¹H NMR (500 MHz, CHLOROFORM-d) δ ppm 9.04 (1H, s), 8.80-9.01 (1H, m), 8.15 (1H, s), 7.82-8.12 (2H, m), 7.37-7.64 (4.6H, m), 7.17-7.26 (0.2H, m), 5.44-5.67 (1H, m), 5.04-5.32 (2H, m), 1.69 (0.46H, br. s.), 1.16-1.29 (2.64H, m). Mass Spectrum (ESI) m/e=402.1 (M+1).

### Example 41

### 4-Amino-6-((1-(6-chloro-4-(2-pyridinyl)-3-quinolinyl)ethyl)-amino)-5-pyrimidinecarbonitrile

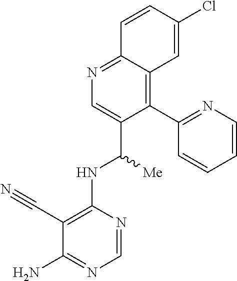 [SMILES: CC(Nc1ncnc(N)c1C#N)c1cnc2ccc(Cl)cc2c1-c1ccccn1]

4-Amino-6-((1-(6-chloro-4-(2-pyridinyl)-3-quinolinyl)ethyl)amino)-5-pyrimidinecarbonitrile was prepared according to the methods described in General Methods A9, A10, and A4 from 1-(4,6-dichloroquinolin-3-yl)ethanone. A mixture of isomers was observed in the proton NMR trace. ¹H NMR (500 MHz, DMSO-d₆) δ ppm 9.25 (1H, br. s.), 8.80 (1H, d, J=3.7 Hz), 7.93-8.20 (3 H, m), 7.43-7.90 (4H, m), 7.20 (4H, br. s.), 4.87-5.49 (1H, m), 1.38-1.67 (3 H, m). Mass Spectrum (ESI) m/e=402.1 (M+1) and 400.0 (M−1).

### 4-Amino-6-(((1S)-1-(6-chloro-4-(2-pyridinyl)-3-quinolinyl)ethyl)amino)-5-pyrimidinecarbonitrile

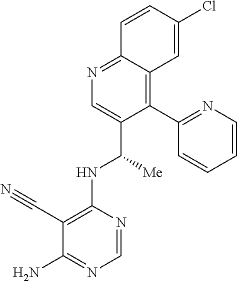 [SMILES: C[C@H](Nc1ncnc(N)c1C#N)c1cnc2ccc(Cl)cc2c1-c1ccccn1]

4-Amino-6-(((1S)-1-(6-chloro-4-(2-pyridinyl)-3-quinolinyl)ethyl)amino)-5-pyrimidinecarbonitrile was prepared according to the methods described in General Method B4 starting from 4-amino-6-((1-(6-chloro-4-(2-pyridinyl)-3-quinolinyl)ethyl)amino)-5-pyrimidinecarbonitrile. The stereochemistry is arbitrarily assigned. A mixture of isomers was observed in the proton NMR trace. ¹H NMR (500 MHz, CHLOROFORM-d) δ ppm 9.02 (1H, s), 8.82-9.01 (1H, m), 7.30-8.18 (8H, m), 5.41-5.65 (1H, m), 5.02-5.33 (2H, m), 1.69 (0.4H, br. s.), 1.23-1.35 (2.6H, m). Mass Spectrum (ESI) m/e=402.1 (M+1).

### 4-Amino-6-(((1R)-1-(6-chloro-4-(2-pyridinyl)-3-quinolinyl)ethyl)amino)-5-pyrimidinecarbonitrile

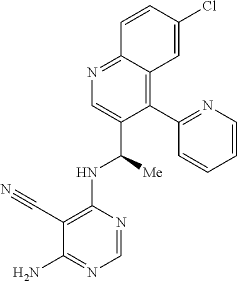 [SMILES: C[C@@H](Nc1ncnc(N)c1C#N)c1cnc2ccc(Cl)cc2c1-c1ccccn1]

4-Amino-6-(((1R)-1-(6-chloro-4-(2-pyridinyl)-3-quinolinyl)ethyl)amino)-5-pyrimidinecarbonitrile was prepared according to the methods described in General Method B4 starting from 4-amino-6-((1-(6-chloro-4-(2-pyridinyl)-3-quinolinyl)ethyl)amino)-5-pyrimidinecarbonitrile. The stereochemistry is arbitrarily assigned. A mixture of isomers was observed in the proton NMR trace. ¹H NMR (500 MHz, CHLOROFORM-d) δ ppm 9.02 (1H, s), 8.79-9.01 (1H, m), 7.29-8.15 (8H, m), 5.40-5.64 (1H, m), 5.00-5.33 (2H, m), 1.69 (0.5H, br. s.), 1.23-1.39 (2.7H, m). Mass Spectrum (ESI) m/e=402.1 (M+1).

### Example 42

### 4-Amino-6-((1-(8-chloro-4-(3-fluorophenyl)-3-quinolinyl)ethyl)-amino)-5-pyrimidinecarbonitrile

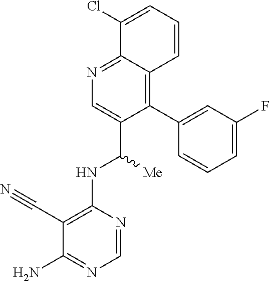 [SMILES: CC(Nc1ncnc(N)c1C#N)c1cnc2c(Cl)cccc2c1-c1cccc(F)c1]

4-Amino-6-((1-(8-chloro-4-(3-fluorophenyl)-3-quinolinyl)ethyl)amino)-5-pyrimidinecarbonitrile was prepared according to the methods described in General Methods B11, B10, B14, A3, and A4 from 1-(4,8-dichloroquinolin-3-yl)ethanone. A mixture of isomers was observed in the proton NMR trace. ¹H NMR (400 MHz, CD₃OD) δ ppm 9.09-9.18 (1H, m), 7.83-7.92 (2H, m), 7.57-7.65 (1H, m), 7.24-7.50 (4H, m), 7.08-7.18 (1H, m), 5.28 (1H, dq, J=14.5, 7.2 Hz), 1.52-1.61 (3H, m). Mass Spectrum (ESI) m/e=419.0 (M+1) and 417.1 (M−1).

### 4-Amino-6-(((1S)-1-(8-chloro-4-(3-fluorophenyl)-3-quinolinyl)ethyl)amino)-5-pyrimidinecarbonitrile

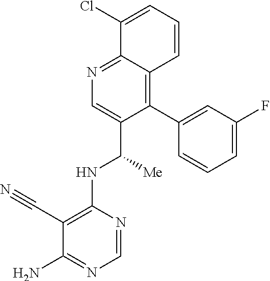 [SMILES: C[C@H](Nc1ncnc(N)c1C#N)c1cnc2c(Cl)cccc2c1-c1cccc(F)c1]

4-Amino-6-(((1S)-1-(8-chloro-4-(3-fluorophenyl)-3-quinolinyl)ethyl)amino)-5-pyrimidinecarbonitrile was prepared according to the methods described in General B4 starting from 4-amino-6-((1-(8-chloro-4-(3-fluorophenyl)-3-quinolinyl)ethyl)amino)-5-pyrimidinecarbonitrile. The stereochemistry is arbitrarily assigned. A mixture of isomers was observed in the proton NMR trace. ¹H NMR (500 MHz, CHLOROFORM-d) δ ppm 9.15 (1H, d, J=1.5 Hz), 7.95-8.06 (1H, m), 7.82 (1H, d, J=7.1 Hz), 7.48-7.61 (1H, m), 7.29-7.43 (3H, m), 7.21-7.26 (1H, m), 6.92-7.09 (1H, m), 5.59-5.75 (1H, m), 5.45 (2H, br. s.), 5.19-5.30 (1H, m), 1.49-1.60 (3H, m). Mass Spectrum (ESI) m/e=419.0 (M+1) and 417.0 (M−1).

### 4-Amino-6-(((1R)-1-(8-chloro-4-(3-fluorophenyl)-3-quinolinyl)ethyl)amino)-5-pyrimidinecarbonitrile

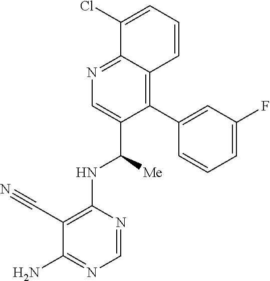 [SMILES: C[C@@H](Nc1ncnc(N)c1C#N)c1cnc2c(Cl)cccc2c1-c1cccc(F)c1]

4-Amino-6-(((1R)-1-(8-chloro-4-(3-fluorophenyl)-3-quinolinyl)ethyl)amino)-5-pyrimidinecarbonitrile was prepared according to the methods described in General B4 starting from 4-amino-6-((1-(8-chloro-4-(3-fluorophenyl)-3-quinolinyl)ethyl)amino)-5-pyrimidinecarbonitrile. The stereochemistry is arbitrarily assigned. A mixture of isomers was observed in the proton NMR trace. ¹H NMR (500 MHz, CHLOROFORM-d) δ ppm 9.15 (1H, d, J=1.5 Hz), 7.93-8.08 (1H, m), 7.82 (1H, d, J=7.1 Hz), 7.49-7.61 (1H, m), 7.29-7.43 (3H, m), 7.20-7.27 (1H, m), 6.91-7.08 (1H, m), 5.55-5.67 (1H, m), 5.34-5.46 (2H, m), 5.18-5.30 (1H, m), 1.48-1.60 (3H, m). Mass Spectrum (ESI) m/e=419.0 (M+1) and 417.1 (M−1).

### 1-(4-Chloro-8-fluoroquinolin-3-yl)ethanol

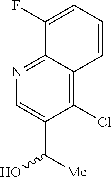 [SMILES: CC(O)c1cnc2c(F)cccc2c1Cl]

1-(4-Chloro-8-fluoroquinolin-3-yl)ethanol was prepared according to the methods described in General Method B11 from 1-(4-chloro-8-fluoroquinolin-3-yl)-ethanone. ¹H NMR (400 MHz, CHLOROFORM-d) δ ppm 9.19 (1H, s), 8.04 (1H, d, J=8.6 Hz), 7.60 (1H, td, J=8.2, 5.1 Hz), 7.46 (1H, ddd, J=9.9, 7.9, 0.8 Hz), 5.58 (1H, q, J=6.6 Hz), 1.64 (3H, d, J=6.5 Hz). Mass Spectrum (ESI) m/e=226.2 (M+1).

### 2-(1-(4-Chloro-8-fluoroquinolin-3-yl)ethyl)isoindoline-1,3-dione

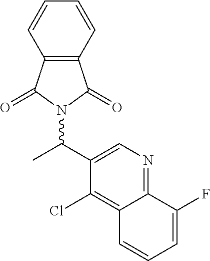 [SMILES: CC(c1cnc2c(F)cccc2c1Cl)N1C(=O)c2ccccc2C1=O]

2-(1-(4-Chloro-8-fluoroquinolin-3-yl)ethyl)isoindoline-1,3-dione was prepared according to the methods described in General Method B10 from 1-(4-chloro-8-fluoroquinolin-3-yl)ethanol. Mass Spectrum (ESI) m/e=355.2 (M+1).

### Example 43

### 4-Amino-6-((1-(8-fluoro-4-phenyl-3-quinolinyl)ethyl)amino)-5-pyrimidinecarbonitrile

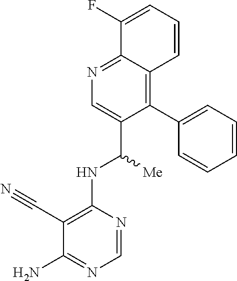 [SMILES: CC(Nc1ncnc(N)c1C#N)c1cnc2c(F)cccc2c1-c1ccccc1]

4-Amino-6-((1-(8-fluoro-4-phenyl-3-quinolinyl)ethyl)amino)-5-pyrimidinecarbonitrile was prepared according to the methods described in General Methods B11, B10, B14, A3, and A4 from 1-(4-chloro-8-fluoroquinolin-3-yl)ethanone. ¹H NMR (500 MHz, DMSO-d₆) δ ppm 9.24 (1H, s), 7.92 (1H, s), 7.86 (1H, s), 7.51-7.64 (5H, m), 7.47 (1H, td, J=8.1, 5.4 Hz), 7.32 (1H, d, J=7.1 Hz), 7.21 (2H, br. s.), 7.08 (1H, d, J=8.6 Hz), 5.10 (1H, quin, J=7.0 Hz), 1.47 (3H, d, J=7.1 Hz). Mass Spectrum (ESI) m/e=385.1 (M+1) and 383.0 (M−1).

### 4-Amino-6-(((1R)-1-(8-fluoro-4-phenyl-3-quinolinyl)ethyl)amino)-5-pyrimidinecarbonitrile

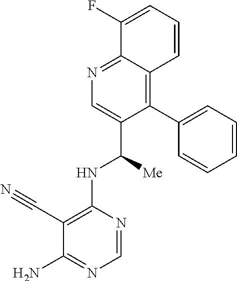 [SMILES: C[C@@H](Nc1ncnc(N)c1C#N)c1cnc2c(F)cccc2c1-c1ccccc1]

4-Amino-6-(((1R)-1-(8-fluoro-4-phenyl-3-quinolinyl)ethyl)amino)-5-pyrimidinecarbonitrile (was prepared according to the methods described in General Method B4 starting from 4-amino-6-((1-(8-fluoro-4-phenyl-3-quinolinyl)ethyl)amino)-5-pyrimidinecarbonitrile. The stereochemistry is arbitrarily assigned. ¹H NMR (500 MHz, CHLOROFORM-d) δ ppm 9.09 (1H, br. s.), 8.00 (1H, s), 7.48-7.63 (4H, m), 7.32-7.45 (2H, m), 7.22-7.26 (1H, m), 7.18 (1H, d, J=7.6 Hz), 5.67 (1H, d, J=6.4 Hz), 5.53 (2H, br. s.), 5.32 (1H, quin, J=6.9 Hz), 1.56 (3H, d, J=7.1 Hz). Mass Spectrum (ESI) m/e=385.1 (M+1) and 383.0 (M−1).

### 4-Amino-6-(((1S)-1-(8-fluoro-4-phenyl-3-quinolinyl)ethyl)amino)-5-pyrimidinecarbonitrile

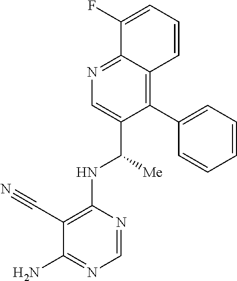 [SMILES: C[C@H](Nc1ncnc(N)c1C#N)c1cnc2c(F)cccc2c1-c1ccccc1]

4-Amino-6-(((1S)-1-(8-fluoro-4-phenyl-3-quinolinyl)ethyl)amino)-5-pyrimidinecarbonitrile was prepared according to the methods described in General Method B4 starting from 4-amino-6-((1-(8-fluoro-4-phenyl-3-quinolinyl)ethyl)amino)-5-pyrimidinecarbonitrile. The stereochemistry is arbitrarily assigned. ¹H NMR (500 MHz, CHLOROFORM-d) δ ppm 9.10 (1H, s), 8.00 (1H, s), 7.49-7.60 (4H, m), 7.31-7.44 (2H, m), 7.22-7.26 (1H, m), 7.18 (1H, d, J=7.8 Hz), 5.74 (1H, d, J=6.4 Hz), 5.63 (2H, br. s.), 5.33 (1H, quin, J=7.0 Hz), 1.56 (3H, d, J=7.1 Hz). Mass Spectrum (ESI) m/e=385.1 (M+1) and 383.0 (M−1).

### Example 44

### 4-Amino-6-((1-(4-(3,5-difluorophenyl)-8-fluoro-3-quinolinyl)-ethyl)amino)-5-pyrimidinecarbonitrile

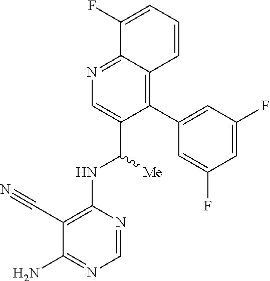 [SMILES: CC(Nc1ncnc(N)c1C#N)c1cnc2c(F)cccc2c1-c1cc(F)cc(F)c1]

4-Amino-6-((1-(4-(3,5-difluorophenyl)-8-fluoro-3-quinolinyl)ethyl)amino)-5-pyrimidinecarbonitrile was prepared according to the methods described in General Methods B11, B10, B14, A3, and A4 from 1-(4-chloro-8-fluoroquinolin-3-yl)ethanone. ¹H NMR (400 MHz, DMSO-d₆) δ ppm 9.27 (1H, s), 7.94 (1H, d, J=7.0 Hz), 7.85 (1H, s), 7.49-7.64 (2H, m), 7.43 (1H, tt, J=9.4, 2.2 Hz), 7.27-7.32 (1H, m), 7.17-7.27 (3H, m), 7.14 (1H, d, J=8.2 Hz), 5.09 (1H, quin, J=7.1 Hz), 1.52 (3H, d, J=7.0 Hz). Mass Spectrum (ESI) m/e=421.1 (M+1) and 419.0 (M−1).

### 4-Amino-6-(((1S)-1-(4-(3,5-difluorophenyl)-8-fluoro-3-quinolinyl)ethyl)-amino)-5-pyrimidinecarbonitrile

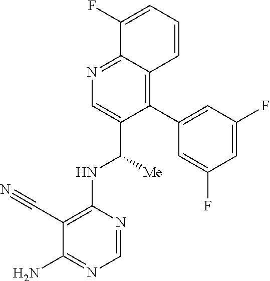 [SMILES: C[C@H](Nc1ncnc(N)c1C#N)c1cnc2c(F)cccc2c1-c1cc(F)cc(F)c1]

4-Amino-6-(((1S)-1-(4-(3,5-difluorophenyl)-8-fluoro-3-quinolinyl)ethyl)amino)-5-pyrimidinecarbonitrile was prepared according to the methods described in General Method B4 starting from 4-amino-6-((1-(4-(3,5-difluorophenyl)-8-fluoro-3-quinolinyl)ethyl)amino)-5-pyrimidinecarbonitrile. The stereochemistry is arbitrarily assigned. ¹H NMR (500 MHz, CHLOROFORM-d) δ ppm 9.07 (1H, s), 8.02 (1H, s), 7.36-7.46 (2H, m), 7.20-7.25 (1H, m), 7.14-7.19 (1H, m), 7.00 (1H, tt, J=9.0, 2.3 Hz), 6.78-6.84 (1H, m), 5.62 (1H, d, J=6.1 Hz), 5.38 (2H, br. s), 5.23 (1H, quin, J=6.8 Hz), 1.57 (3H, d, J=7.1 Hz). Mass Spectrum (ESI) m/e=421.1 (M+1) and 419.0 (M−1).

### 4-Amino-6-(((1R)-1-(4-(3,5-difluorophenyl)-8-fluoro-3-quinolinyl)ethyl)-amino)-5-pyrimidinecarbonitrile

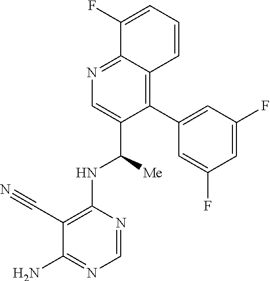 [SMILES: C[C@@H](Nc1ncnc(N)c1C#N)c1cnc2c(F)cccc2c1-c1cc(F)cc(F)c1]

4-Amino-6-(((1R)-1-(4-(3,5-difluorophenyl)-8-fluoro-3-quinolinyl)ethyl)amino)-5-pyrimidinecarbonitrile was prepared according to the methods described in General Method B4 starting from 4-amino-6-((1-(4-(3,5-difluorophenyl)-8-fluoro-3-quinolinyl)ethyl)amino)-5-pyrimidinecarbonitrile. The stereochemistry is arbitrarily assigned. ¹H NMR (500 MHz, CHLOROFORM-d) δ ppm 9.07 (1H, s), 8.02 (1H, s), 7.36-7.46 (2H, m), 7.23 (1H, dt, J=8.6, 0.9 Hz), 7.15-7.19 (1 H, m), 7.00 (1H, tt, J=8.9, 2.3 Hz), 6.81 (1H, dt, J=8.3, 1.0 Hz), 5.61 (1H, d, J=6.4 Hz), 5.37 (2H, br. s), 5.23 (1H, quin, J=6.8 Hz), 1.57 (3H, d, J=7.1 Hz). Mass Spectrum (ESI) m/e=421.1 (M+1) and 419.0 (M−1).

### Example 45

### 4-Amino-6-((1-(8-fluoro-4-(3-fluorophenyl)-3-quinolinyl)ethyl)-amino)-5-pyrimidinecarbonitrile

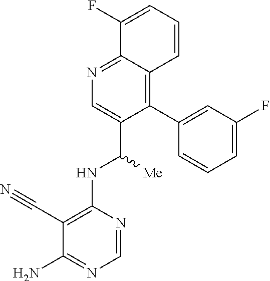 [SMILES: CC(Nc1ncnc(N)c1C#N)c1cnc2c(F)cccc2c1-c1cccc(F)c1]

4-Amino-6-((1-(8-fluoro-4-(3-fluorophenyl)-3-quinolinyl)ethyl)amino)-5-pyrimidinecarbonitrile was prepared according to the methods described in General Methods B11, B10, B14, A3, and A4 from 1-(4-chloro-8-fluoroquinolin-3-yl)ethanone. A mixture of isomers was observed in the proton NMR trace. ¹H NMR (400 MHz, DMSO-d₆) δ ppm 9.26 (1H, d, J=6.5 Hz), 7.93 (1H, dd, J=14.2, 7.1 Hz), 7.85 (1H, d, J=7.0 Hz), 7.45-7.68 (3H, m), 7.32-7.45 (2 H, m), 7.14-7.32 (3H, m), 7.09 (1H, t, J=7.4 Hz), 5.08 (1H, sxt, J=6.9 Hz), 1.49 (3H, m). Mass Spectrum (ESI) m/e=403.1 (M+1) and 401.0 (M−1).

### 4-Amino-6-(((1S)-1-(8-fluoro-4-(3-fluorophenyl)-3-quinolinyl)ethyl)amino)-5-pyrimidinecarbonitrile

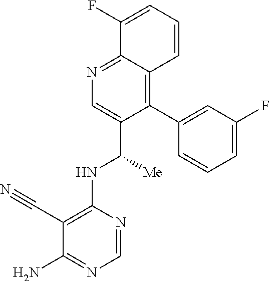 [SMILES: C[C@H](Nc1ncnc(N)c1C#N)c1cnc2c(F)cccc2c1-c1cccc(F)c1]

4-Amino-6-(((1S)-1-(8-fluoro-4-(3-fluorophenyl)-3-quinolinyl)ethyl)amino)-5-pyrimidinecarbonitrile was prepared according to the methods described in General Method B4 starting from 4-amino-6-((1-(8-fluoro-4-(3-fluorophenyl)-3-quinolinyl)ethyl)amino)-5-pyrimidinecarbonitrile. The stereochemistry is arbitrarily assigned. A mixture of isomers was observed in the proton NMR trace. ¹H NMR (500 MHz, DMSO-d₆) δ ppm 9.25 (1H, d, J=8.1 Hz), 7.93 (1H, dd, J=17.7, 7.2 Hz), 7.85 (1H, d, J=8.8 Hz), 7.53-7.68 (2H, m), 7.46-7.52 (1H, m), 7.33-7.44 (2H, m), 7.27-7.31 (1H, m), 7.17-7.26 (2H, m), 7.09 (1H, t, J=8.3 Hz), 5.08 (1H, sxt, J=7.2 Hz), 1.49 (1.5H, d, J=7.09 Hz), 1.48 (1.5H, d, J=7.34 Hz). Mass Spectrum (ESI) m/e=403.1 (M+1).

### 4-Amino-6-(((1R)-1-(8-fluoro-4-(3-fluorophenyl)-3-quinolinyl)ethyl)amino)-5-pyrimidinecarbonitrile

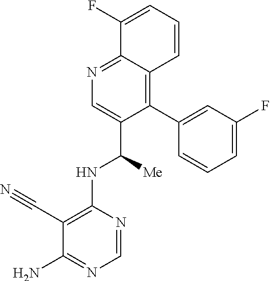 [SMILES: C[C@@H](Nc1ncnc(N)c1C#N)c1cnc2c(F)cccc2c1-c1cccc(F)c1]

4-Amino-6-(((1R)-1-(8-fluoro-4-(3-fluorophenyl)-3-quinolinyl)ethyl)amino)-5-pyrimidinecarbonitrile was prepared according to the methods described in General Method B4 starting from 4-amino-6-((1-(8-fluoro-4-(3-fluorophenyl)-3-quinolinyl)ethyl)amino)-5-pyrimidinecarbonitrile. The stereochemistry is arbitrarily assigned. A mixture of isomers was observed in the proton NMR trace. ¹H NMR (500 MHz, DMSO-d₆) δ ppm 9.25 (1H, d, J=8.1 Hz), 7.93 (1H, dd, J=17.9, 7.3 Hz), 7.85 (1H, d, J=8.8 Hz), 7.53-7.67 (2H, m), 7.46-7.53 (1H, m), 7.33-7.44 (2H, m), 7.27-7.31 (1H, m), 7.20 (2H, d, J=7.6 Hz), 7.09 (1H, t, J=8.3 Hz), 5.08 (1H, sxt, J=7.2 Hz), 1.50 (1.5H, d, J=7.1 Hz), 1.48 (1.5H, d, J=7.3 Hz). Mass Spectrum (ESI) m/e=403.1 (M+1).

### Example 46

### 4-Amino-6-((1-(8-chloro-4-(1H-pyrazol-5-yl)-3-quinolinyl)-ethyl)amino)-5-pyrimidinecarbonitrile

### 1-(8-Chloro-4-(1H-pyrazol-5-yl)quinolin-3-yl)ethanone

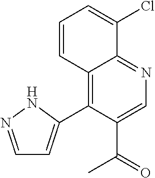 [SMILES: CC(=O)c1cnc2c(Cl)cccc2c1-c1ccn[nH]1]

1-(4,8-Dichloroquinolin-3-yl)ethanone (0.1 g, 0.417 mmol), potassium carbonate (0.173 g, 1.250 mmol), 1H-pyrazol-5-ylboronic acid (0.070 g, 0.625 mmol), and PdCl₂(dppf)₂CH₂Cl₂ (0.034 g, 0.042 mmol) were combined in 3 mL of anhydrous DMF under an atmosphere of N₂. The solution was heated to 80° C. overnight and then cooled to rt and diluted with ethyl acetate. The organic phase was washed with, brine, H₂O, then with brine again. The organic phase was dried over Na₂SO₄, filtered, and concentrated under vacuum. The residue thus obtained was purified by column chromatography using a gradient of 10% ethyl acetate/hexane to 60% ethyl acetate/hexane. The fractions containing the product were combined and concentrated under vacuum to give 1-(8-chloro-4-(1H-pyrazol-5-yl)quinolin-3-yl)ethanone as a clear film. ¹H NMR (500 MHz, CHLOROFORM-d) δ ppm 9.24 (1H, s), 8.00 (1H, dd, J=7.3, 1.2 Hz), 7.96 (1H, d, J=2.0 Hz), 7.81 (1H, d, J=2.4 Hz), 7.70 (1H, dd, J=8.6, 1.2 Hz), 7.57 (1H, dd, J=8.6, 7.6 Hz), 6.71 (1H, t, J=2.2 Hz), 1.97 (3H, s). Mass Spectrum (ESI) m/e=272.0 (M+1).

### 1-(8-Chloro-4-(1H-pyrazol-5-yl)quinolin-3-yl)ethanamine

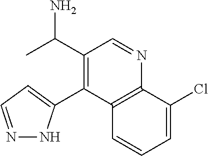 [SMILES: CC(N)c1cnc2c(Cl)cccc2c1-c1ccn[nH]1]

1-(8-Chloro-4-(1H-pyrazol-5-yl)quinolin-3-yl)ethanamine was prepared according to the methods described in General Method A10 from 1-(8-chloro-4-(1H-pyrazol-5-yl)quinolin-3-yl)ethanone. Mass Spectrum (ESI) m/e=273.1 (M+1).

### 4-Amino-6-((1-(8-chloro-4-(1H-pyrazol-5-yl)-3-quinolinyl)ethyl)amino)-5-pyrimidinecarbonitrile

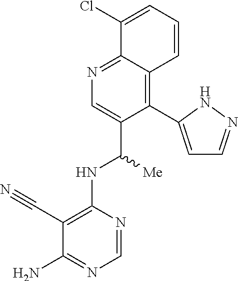 [SMILES: CC(Nc1ncnc(N)c1C#N)c1cnc2c(Cl)cccc2c1-c1ccn[nH]1]

4-Amino-6-((1-(8-chloro-4-(1H-pyrazol-5-yl)-3-quinolinyl)ethyl)amino)-5-pyrimidinecarbonitrile was prepared according to the methods described in General Method A4 from 1-(8-chloro-4-(1H-pyrazol-5-yl)quinolin-3-yl)-ethanamine. ¹H NMR (500 MHz, DMSO-d₆) δ ppm 9.38 (1H, s), 8.29 (1H, d, J=1.5 Hz), 8.00 (1H, dd, J=7.6, 1.2 Hz), 7.96 (1H, br. s.), 7.93 (1H, d, J=1.7 Hz), 7.86 (1H, br. s), 7.60 (1H, t, J=8.1 Hz), 7.25 (2H, br. s.), 7.14 (1H, dd, J=8.4, 1.1 Hz), 6.70 (1H, t, J=2.1 Hz), 5.05 (1H, br. s.), 1.52 (3H, d, J=7.3 Hz). Mass Spectrum (ESI) m/e=391.0 (M+1).

### Example 47

### 3-(1-((6-Amino-5-cyano-4-pyrimidinyl)amino)ethyl)-4-(2-pyridinyl)-8-quinolinecarbonitrile

### 2-(1-(8-Chloro-4-(pyridin-2-yl)quinolin-3-yl)ethyl)isoindoline-1,3-dione

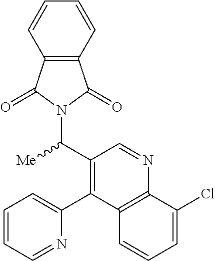 [SMILES: CC(c1cnc2c(Cl)cccc2c1-c1ccccn1)N1C(=O)c2ccccc2C1=O]

Isobenzofuran-1,3-dione (0.058 g, 0.395 mmol), N-ethyl-N-isopropylpropan-2-amine (0.068 mL, 0.395 mmol), and 1-(8-chloro-4-(pyridin-2-yl)quinolin-3-yl)-ethanamine (prepared from 1-(4,8-dichloroquinolin-3-yl)ethanone using General Method A10) (0.112 g, 0.395 mmol) were combined in 8 mL of anhydrous toluene. The flask was equipped with a dean stark trap and the solution was heated to a vigorous reflux for 24 h. After cooling the solution to rt it was concentrated under vacuum. The residue obtained was dissolved in DCM. The organic layer was washed with sat NaHCO₃ and dried over MgSO₄ before being concentrated under vacuum. The residue obtained was purified by column chromatography using a gradient of 40% ethyl acetate/hexane to 60% ethyl acetate/hexane. The fractions containing the product were combined and concentrated under vacuum to provide 2-(1-(8-chloro-4-(pyridin-2-yl)quinolin-3-yl)ethyl)isoindoline-1,3-dione as a off white solid. A mixture of isomers was observed in the proton NMR trace. ¹H NMR (500 MHz, CHLOROFORM-d) δ ppm 9.51-9.73 (1H, m), 8.39-8.98 (1H, m), 7.61-8.00 (6H, m), 7.28-7.51 (2.42H, m), 7.04-7.26 (1.65H, m), 5.41-5.65 (1H, m), 1.90-2.02 (3H, m). Mass Spectrum (ESI) m/e=414.2 (M+1).

### 3-(1-(1,3-Dioxoisoindolin-2-yl)ethyl)-4-(pyridin-2-yl)quinoline-8-carbonitrile

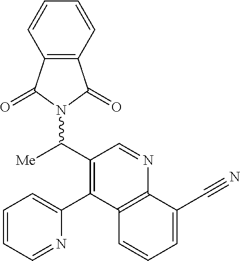 [SMILES: CC(c1cnc2c(C#N)cccc2c1-c1ccccn1)N1C(=O)c2ccccc2C1=O]

XPhos precatalyst (dicyclohexyl(2′,4′,6′-triisopropylbiphenyl-2-yl)phosphine palladium(II) phenethylamine chloride, see Briscoe, M. R.; Fors, B. P.; Buchwald, S. L. J. Am. Chem. Soc. 2008, 130, 6686) (0.110 g, 0.145 mmol) was combined with 0.5 mL of NMP under an atmosphere of N₂. The suspension was then cooled in an ice bath before adding LiHMDS 1M in THF (0.116 mL, 0.116 mmol). The solids went into the solution with the addition of the base. To this was then added 2-(1-(8-chloro-4-(pyridin-2-yl)quinolin-3-yl)ethyl)isoindoline-1,3-dione (0.120 g, 0.290 mmol) dissolved in 0.3 mL of NMP, rinsed with NMP and added (2×0.2 mL). The solution was heated to 100° C. and then a solution of tributylstannane-carbonitrile (0.092 g, 0.290 mmol) dissolved in 0.5 mL of NMP was slowly added over a period of 30 min followed by NMP (0.3 mL). The solution was heated at 100° C. for 4 h, cooled to rt, and diluted with ethyl acetate. The organics were then washed in succession with sat NH₄Cl, sat KF, H₂O, and brine. The organic phase was dried over MgSO₄ and concentrated under vacuum to give brown oil. The oil was purified by column chromatography using a gradient of 50% ethyl acetate/hexane to 100% ethyl acetate. The fractions containing the product were combined and concentrated under vacuum to provide 3-(1-(1,3-dioxoisoindolin-2-yl)ethyl)-4-(pyridin-2-yl)quinoline-8-carbonitrile as a light brownish solid. A mixture of isomers was observed in the proton NMR trace. ¹H NMR (500 MHz, CHLOROFORM-d) δ ppm 9.60-9.77 (1H, m), 8.36-8.97 (1H, m), 7.36-8.15 (10H, m), 7.26 (0H, s), 5.46-5.67 (1H, m), 1.93-2.01 (3H, m). Mass Spectrum (ESI) m/e=405.1 (M+1).

### 3-(1-((6-Amino-5-cyano-4-pyrimidinyl)amino)ethyl)-4-(2-pyridinyl)-8-quinolinecarbonitrile

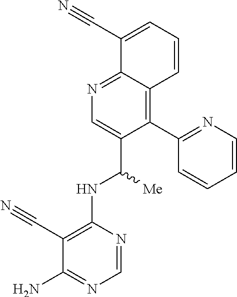 [SMILES: CC(Nc1ncnc(N)c1C#N)c1cnc2c(C#N)cccc2c1-c1ccccn1]

3-(1-((6-Amino-5-cyano-4-pyrimidinyl)amino)ethyl)-4-(2-pyridinyl)-8-quinolinecarbonitrile was prepared according to the methods described in General Methods A3 and A4 from 3-(1-(1,3-dioxoisoindolin-2-yl)ethyl)-4-(pyridin-2-yl)-quinoline-8-carbonitrile. The stereochemistry is arbitrarily assigned. A mixture of isomers was observed in the proton NMR trace. ¹H NMR (500 MHz, DMSO-d₆) δ ppm 9.40 (1H, br. s.), 8.80 (1H, br. s.), 8.27-8.46 (1H, m), 7.48-8.11 (7 H, m), 7.22 (2H, br. s.), 4.95-5.52 (1H, m), 1.39-1.69 (3H, m). Mass Spectrum (ESI) m/e=393.1 (M+1).

The following compounds were made via general methods All, A1, A2, A3, A4 starting from 1-(4-chloro-7-fluoroquinolin-3-yl)ethanone (synthesis according to general methods B5, B8 and B7):

### Example 48

### 4-Amino-6-((1-(7-fluoro-4-phenylquinolin-3-yl)ethyl)amino)-pyrimidine-5-carbonitrile

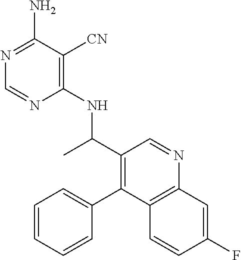 [SMILES: CC(Nc1ncnc(N)c1C#N)c1cnc2cc(F)ccc2c1-c1ccccc1]

¹H NMR (500 MHz, DMSO-d₆) δ 9.22 (s, 1H), 7.88 (d, J=7.1 Hz, 1H), 7.85 (s, 1H), 7.77 (dd, J=10.0, 2.5 Hz, 1H), 7.61-7.50 (series of m, 4H), 7.44 (td, J=9.0, 2.7 Hz, 1H), 7.32 (m, 2H), 7.19 (br s, 2H), 5.10 (quintet, J=7.1 Hz, 1H), 1.46 (d, J=7.1 Hz, 3H) ppm. Mass Spectrum (ESI) m/e=385.2 (M+1). The individual enantiomers were obtained according to the methods described in General Method B4 to give (R)-4-amino-6-((1-(7-fluoro-4-phenylquinolin-3-yl)ethyl)-amino)pyrimidine-5-carbonitrile and (S)-4-amino-6-((1-(7-fluoro-4-phenylquinolin-3-yl)ethyl)amino)pyrimidine-5-carbonitrile. The spectra obtained for the individual enantiomers was consistent with that obtained for the racemate.

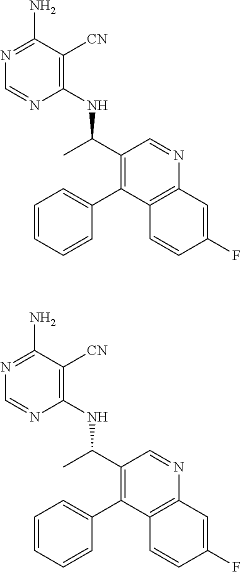 [SMILES: C[C@@H](Nc1ncnc(N)c1C#N)c1cnc2cc(F)ccc2c1-c1ccccc1 ; C[C@H](Nc1ncnc(N)c1C#N)c1cnc2cc(F)ccc2c1-c1ccccc1]

### Example 49

### 4-Amino-6-((1-(7-fluoro-4-(3-fluorophenyl)quinolin-3-yl)ethyl)amino)pyrimidine-5-carbonitrile

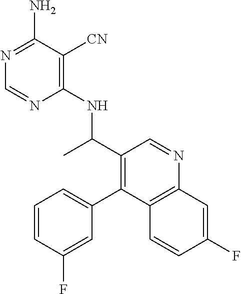 [SMILES: CC(Nc1ncnc(N)c1C#N)c1cnc2cc(F)ccc2c1-c1cccc(F)c1]

The NMR spectrum reflects a roughly 1:1 mixture of isomers at room temperature. ¹H NMR (500 MHz, DMSO-d₆) δ 9.24 (s, 0.5H), 9.22 (s, 0.5H), 7.91 (d, J=7.3 Hz, 0.5H), 7.87 (d, J=7.3 Hz, 0.5H), 7.85 (s, 0.5H), 7.84 (s, 0.5H), 7.79 (m, 1H), 7.61 (m, 1H), 7.50-7.10 (series of m, 7H), 5.08 (m, 1H), 1.49 (d, J=7.1 Hz, 1.5H), 1.47 (d, J=7.1 Hz, 1.5H) ppm. Mass Spectrum (ESI) m/e=403.2 (M+1). The individual enantiomers were obtained according to the methods described in General Method B4 to give (R)-4-amino-6-((1-(7-fluoro-4-(3-fluorophenyl)quinolin-3-yl)ethyl)amino)pyrimidine-5-carbonitrile and (S)-4-amino-6-((1-(7-fluoro-4-(3-fluorophenyl)quinolin-3-yl)ethyl)amino)pyrimidine-5-carbonitrile. The spectra obtained for the individual enantiomers was consistent with that obtained for the racemate.

The following compounds were made via general methods A9, A10, A4 starting from 1-(4-chloro-7-fluoroquinolin-3-yl)ethanone (synthesis according to general methods B5, B8 and B7):

### Example 50

### 4-Amino-6-((1-(7-fluoro-4-(pyridin-3-yl)quinolin-3-yl)ethyl)-amino)pyrimidine-5-carbonitrile

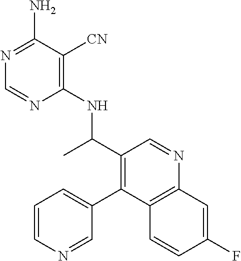 [SMILES: CC(Nc1ncnc(N)c1C#N)c1cnc2cc(F)ccc2c1-c1cccnc1]

The NMR spectrum reflects a roughly 1:1 mixture of isomers at room temperature. ¹H NMR (500 MHz, DMSO-d₆) δ 9.28 (s, 0.5H), 9.26 (s, 0.5H), 8.74 (dd, J=4.9, 1.5 Hz, 0.5H), 8.73 (dd, J=4.9, 1.7 Hz, 0.5H), 8.71 (d, J=2.0 Hz, 0.5H), 8.56 (d, J=2.0 Hz, 0.5H), 8.00 (dt, J=7.8, 1.7 Hz, 0.5H), 7.94 (m, 1H), 7.86-7.77 (series of m, 2.5H), 7.64 (dd, J=7.6, 4.9 Hz, 0.5H), 7.60 (dd, J=7.6, 4.9 Hz, 0.5H), 7.47 (m, 1H), 7.32 (d, J=6.1 Hz, 0.5H), 7.31 (d, J=6.1 Hz, 0.5H), 7.20 (br s, 2H), 4.99 (m, 1H), 1.52 (d, J=7.1 Hz, 1.5H), 1.49 (d, J=7.1 Hz, 1.5H) ppm. Mass Spectrum (ESI) m/e=386.2 (M+1). The individual enantiomers were obtained according to the methods described in General Method B4 to give (R)-4-amino-6-((1-(7-fluoro-4-(pyridin-3-yl)quinolin-3-yl)ethyl)amino)pyrimidine-5-carbonitrile and (S)-4-amino-6-((1-(7-fluoro-4-(pyridin-3-yl)quinolin-3-yl)ethyl)amino)pyrimidine-5-carbonitrile. The spectra obtained for the individual enantiomers was consistent with that obtained for the racemate.

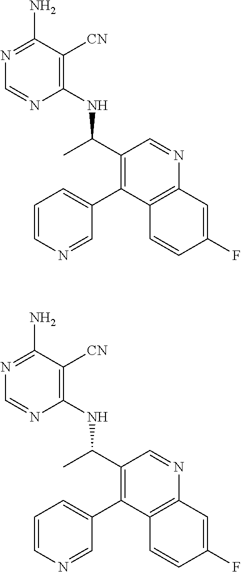 [SMILES: C[C@@H](Nc1ncnc(N)c1C#N)c1cnc2cc(F)ccc2c1-c1cccnc1 ; C[C@H](Nc1ncnc(N)c1C#N)c1cnc2cc(F)ccc2c1-c1cccnc1]

### Example 51

### 1-(7-Fluoro-4-(5-fluoropyridin-3-yl)quinolin-3-yl)ethanone

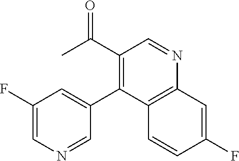 [SMILES: CC(=O)c1cnc2cc(F)ccc2c1-c1cncc(F)c1]

To a reaction vessel was added K₃PO₄ (634 mg, 2.99 mmol), 2-(dicyclohexylphosphino)-2′,4′,6′-tri-1-propyl-1,1′-biphenyl(X-Phos) (47.5 mg, 0.100 mmol), bis(dibenzylideneacetone)palladium (28.7 mg, 0.050 mmol), 5-fluoropyridin-3-ylboronic acid (211 mg, 1.496 mmol), 1-(4-chloro-7-fluoroquinolin-3-yl)ethanone (223 mg, 0.997 mmol) in 2-methyl-2-butanol (4986 μL) and dioxane (4986 μL) under argon. The reaction was heated to 100° C. overnight, then cooled to rt and filtered through celite. The celite pad was rinsed with DCM and concentrated. The crude residue was purified by column chromatography (silica gel, eluting with 20-40% ea in hexanes) to afford 1-(7-fluoro-4-(5-fluoropyridin-3-yl)quinolin-3-yl)ethanone. ¹H NMR (500 MHz, CDCl₃) δ9.27 (s, 1H), 8.67 (d, J=2.5 Hz, 1H), 8.38 (s, 1H), 7.89 (dd, 9.3, 2.5 Hz, 1H), 7.55 (dd, J=9.3, 5.9 Hz, 1H), 7.49 (ddd, J=8.3, 2.5, 2.0 Hz, 1H), 7.36 (ddd, J=9.3, 7.8, 2.5 Hz, 1H), 2.39 (s, 3H) ppm.

The following compounds were made from 1-(7-fluoro-4-(5-fluoropyridin-3-yl)quinolin-3-yl)ethanone according to General Methods A10, A4.

### Example 52

### 4-Amino-6-((1-(7-fluoro-4-(5-fluoropyridin-3-yl)quinolin-3-yl)ethyl)amino)pyrimidine-5-carbonitrile

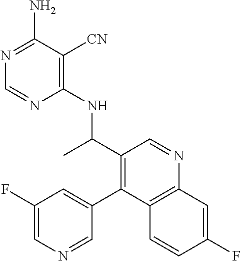 [SMILES: CC(Nc1ncnc(N)c1C#N)c1cnc2cc(F)ccc2c1-c1cncc(F)c1]

The NMR spectrum reflects a roughly 1:1 mixture of isomers at room temperature. ¹H NMR (500 MHz, DMSO-d₆) δ 9.30 (s, 0.5H), 9.27 (s, 0.5H), 8.77 (d, J=2.7H, 0.5H), 8.73 (d, J=2.7 Hz, 0.5H), 8.56 (br s, 0.5H), 8.46 (br s, 0.5H), 7.96 (m, 1.5H), 7.92 (d, 7.3 Hz, 0.5H), 7.82 (m, 2H), 7.48 (m, 1H), 7.28 (m, 1H), 7.22 (br s, 2H), 5.01 (quintet, J=7.1 Hz, 0.5H), 4.96 (quintet, J=7.1 Hz, 0.5H), 1.53 (d, J=6.9 hz, 1.5H), 1.52 (d, J=6.9 hz, 1.5H) ppm. Mass Spectrum (ESI) m/e=404.2 (M+1). The individual enantiomers were obtained according to the methods described in General Method B4 to give (R)-4-amino-6-((1-(7-fluoro-4-(5-fluoropyridin-3-yl)quinolin-3-yl)ethyl)amino)pyrimidine-5-carbonitrile and (S)-4-amino-6-((1-(7-fluoro-4-(5-fluoropyridin-3-yl)quinolin-3-yl)ethyl)amino)pyrimidine-5-carbonitrile.

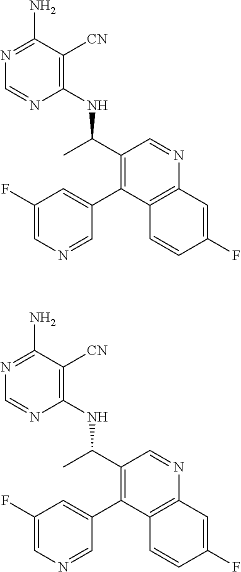 [SMILES: C[C@@H](Nc1ncnc(N)c1C#N)c1cnc2cc(F)ccc2c1-c1cncc(F)c1 ; C[C@H](Nc1ncnc(N)c1C#N)c1cnc2cc(F)ccc2c1-c1cncc(F)c1]

### Example 53

### 4-Amino-6-((1-(6-fluoro-4-phenylisoquinolin-3-yl)ethyl)amino)-pyrimidine-5-carbonitrile

### 4-(5-fluoro-2-formylphenyl)but-3-yn-2-yl acetate

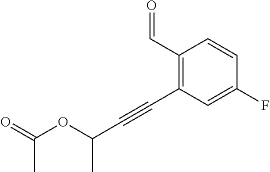 [SMILES: CC(=O)OC(C)C#Cc1cc(F)ccc1C=O]

A reaction vessel was charged with PdCl₂(PPh₃)₂ (1.383 g, 1.970 mmol), triethylamine (206 mL, 1478 mmol), but-3-yn-2-yl acetate (8.28 g, 73.9 mmol) and 2-bromo-4-fluorobenzaldehyde (10 g, 49.3 mmol). The mixture was sparged with nitrogen. To this mixture was added copper(I) iodide (0.188 g, 0.985 mmol). The reaction was stirred at rt for 1 h, then at 40° C. for 2 h. The reaction was cooled to rt and the solvent was removed in vacuo. Purification using 9:1 hexanes:ethyl acetate chromatography afforded 4-(5-fluoro-2-formylphenyl)but-3-yn-2-yl acetate. ¹H NMR (500 MHz, DMSO-d₆) δ 10.26 (s, 1H), 7.92 (dd, J=8.6, 5.9 Hz, 1H), 7.52 (dd, J=9.3, 2.7 Hz, 1H), 7.47 (td, J=8.6, 2.5 Hz, 1H), 5.66 (q, J=6.9 Hz, 1H), 2.08 (s, 3H), 1.56 (d, J=6.9 Hz, 3H) ppm. Mass Spectrum (ESI) m/e=257.2 (M+23).

### (E)-4-(5-fluoro-2-((hydroxyimino)methyl)phenyl)but-3-yn-2-yl acetate

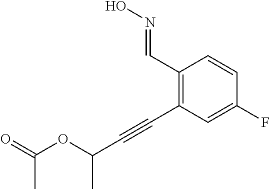 [SMILES: CC(=O)OC(C)C#Cc1cc(F)ccc1/C=N/O]

To a reaction vessel was added hydroxyl ammonium chloride (1.492 mL, 35.9 mmol), pyridine (3.38 mL, 41.8 mmol), 4-(5-fluoro-2-formylphenyl)but-3-yn-2-yl acetate (7.0 g, 29.9 mmol) in ethanol (299 mL). The reaction was stirred at rt for 2 h, and the solvent was removed in vacuo. The residue was redissolved in ethyl acetate and washed with CuSO₄ solution, water, sat. NaHCO₃ and brine. The organic phase was dried over MgSO₄, filtered and concentrated. Isolated (E)-4-(5-fluoro-2-((hydroxyimino)methyl)phenyl)but-3-yn-2-yl acetate. ¹H NMR (500 MHz, DMSO-d₆) δ 11.60 (s, 1H), 8.33 (s, 1H), 7.85 (dd, J=9.0, 5.9 Hz, 1H), 7.36 (dd, J=9.3, 2.7 Hz, 1H), 7.30 (td, J=8.6, 2.7 Hz, 1H), 5.63 (q, J=6.6 Hz, 1H), 2.08 (s, 3H), 1.53 (d, J=6.6 Hz, 3H) ppm.

### 3-(1-acetoxyethyl)-4-bromo-6-fluoroisoquinoline 2-oxide

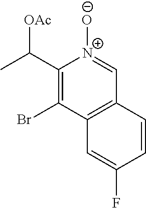 [SMILES: CC(=O)OC(C)c1c(Br)c2cc(F)ccc2c[n+]1[O-]]

(E)-4-(5-fluoro-2-((hydroxyimino)methyl)phenyl)but-3-yn-2-yl acetate (1250 mg, 5.02 mmol) was dissolved in 20 mL of anhydrous DCM. The solution was added via cannula to a solution of NBS in 20 mL DCM at 0° C. After 45 min, the reaction was quenched with 100 mL of sat NaHCO₃ solution. The layers were separated and the organic phase was washed with brine. The organic phase was dried over MgSO₄, filtered and concentrated. Purification by column chromatography with 50-80% ethyl acetate in hexanes afforded 3-(1-acetoxyethyl)-4-bromo-6-fluoroisoquinoline 2-oxide. ¹H NMR (500 MHz, DMSO-d₆) δ 9.17 (s, 1H), 8.07 (dd, J=9.0, 5.6 Hz, 1H), 7.85 (dd, J=10.5, 2.2 Hz, 1H), 7.70 (td, J=8.8, 2.5 Hz, 1H), 6.72 (q, J=6.85 Hz, 1H), 2.06 (s, 3H), 1.65 (d, J=7.1 Hz, 3H) ppm.

### 4-bromo-6-fluoro-3-(1-hydroxyethyl)isoquinoline 2-oxide

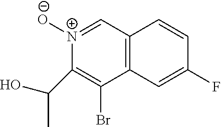 [SMILES: CC(O)c1c(Br)c2cc(F)ccc2c[n+]1[O-]]

To a solution of 3-(1-acetoxyethyl)-4-bromo-6-fluoroisoquinoline 2-oxide (1.5 g, 4.57 mmol) in 75 mL of methanol was added potassium carbonate (10.06 mL, 10.06 mmol) as a 1 M solution in water. The reaction was stirred at rt for 30 min. The solvent was removed in vacuo and the residue was partitioned between ethyl acetate and water. The layers were separated and the organic phase was washed with brine, dried over MgSO₄, filtered and concentrated to afford 4-bromo-6-fluoro-3-(1-hydroxyethyl)isoquinoline-2-oxide. ¹H NMR (500 MHz, DMSO-d₆) δ 9.28 (s, 1H), 8.15 (dd, J=9.0, 5.6 Hz, 1 h), 7.86 (dd, J=10.3, 2.5 Hz, 1H), 7.74 (td, J=8.8, 2.5 Hz, 1H), 6.97 (d, J=10.3 Hz, 1H), 5.57 (dq, J=10.0, 6.9 Hz, 1H), 1.56 (d, J=6.9 Hz, 3H) ppm.

### 4-bromo-3-(1-(1,3-dioxoisoindolin-2-yl)ethyl)-6-fluoroisoquinoline 2-oxide

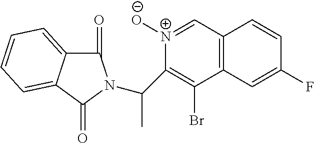 [SMILES: CC(c1c(Br)c2cc(F)ccc2c[n+]1[O-])N1C(=O)c2ccccc2C1=O]

To a solution of isoindoline-1,3-dione (0.741 g, 5.03 mmol), triphenylphosphine (1.320 g, 5.03 mmol), and 4-bromo-6-fluoro-3-(1-hydroxyethyl)isoquinoline 2-oxide (1.2 g, 4.19 mmol) in THF (41.9 mL) at 0° C. was added DIAD (0.991 mL, 5.03 mmol) dropwise. The reaction was warmed to rt and stirred overnight. The solvent was removed in vacuo and the resultant product was slurried in IPA (˜10 mL) to afford a white solid. The solid was filtered and washed with IPA to afford 4-bromo-3-(1-(1,3-dioxoisoindolin-2-yl)ethyl)-6-fluoroisoquinoline 2-oxide. ¹H NMR (500 MHz, DMSO-d₆) δ 9.09 (s, 1H), 8.05 (dd, J=9.0, 5.6 Hz, 1H), 7.81 (m, 5H), 7.69 (td, J=8.8, 2.5 Hz, 1H), 6.27 (q, J=7.3 Hz, 1H), 2.03 (d, J=7.6 Hz, 3H) ppm.

### 2-(1-(4-bromo-6-fluoroisoquinolin-3-yl)ethyl)isoindoline-1,3-dione

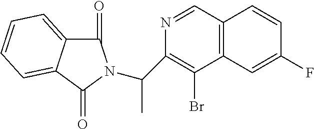 [SMILES: CC(c1ncc2ccc(F)cc2c1Br)N1C(=O)c2ccccc2C1=O]

To a solution of 4-bromo-3-(1-(1,3-dioxoisoindolin-2-yl)ethyl)-6-fluoroisoquinoline 2-oxide (1.0 g, 2.408 mmol) in THF (24.08 mL) was added titanium(III) chloride (2.72 g, 5.30 mmol) (30 wt % solution in 2N HCl). After 30 min, the reaction was quenched with sat, NaHCO₃ solution. The reaction was extracted with ethyl acetate and the organic phase was washed with brine, dried over MgSO₄, filtered and concentrated. Purification by column chromatography afforded 2-(1-(4-bromo-6-fluoroisoquinolin-3-yl)ethyl)isoindoline-1,3-dione. ¹H NMR (500 MHz, DMSO-d₆) δ 9.32 (s, 1H), 8.33 (d, J=8.8, 5.6 Hz, 1H), 7.85 (s, 4H), 7.82 (dd, J=10.7, 2.2 Hz, 1H), 7.72 (td, J=8.8, 2.5 Hz, 1H), 5.86 (q, J=7.1 Hz, 1H), 1.92 (d, J=7.1 Hz, 3H) ppm.

The following compound was made from 2-(1-(4-bromo-6-fluoroisoquinolin-3-yl)ethyl)isoindoline-1,3-dione (above) according to General Methods A2, A3, A4:

### 4-amino-6-((1-(6-fluoro-4-phenylisoquinolin-3-yl)ethyl)amino)pyrimidine-5-carbonitrile

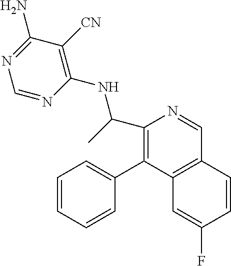 [SMILES: CC(Nc1ncnc(N)c1C#N)c1ncc2ccc(F)cc2c1-c1ccccc1]

¹H NMR (500 MHz, DMSO-d₆) δ 9.47 (s, 1H), 8.35 (dd, J=9.0, 5.9 Hz, 1H), 7.94 (s, 1H), 7.60 (m, 4H), 7.42 (m, 2H), 7.29 (br s, 2H), 7.10 (d, J=7.6 Hz, 1H), 6.83 (dd, J=10.5, 2.2 Hz, 1H), 5.26 (quintet, J=6.6 Hz, 1H), 1.33 (d, J=6.8 Hz, 3H) ppm. Mass Spectrum (ESI) m/e=385.2 (M+1).

### Example 54

### 4-Amino-6-((1-(6-fluoro-8-methyl-4-(pyridin-2-yl)quinolin-3-yl)ethyl)amino)pyrimidine-5-carbonitrile

### (E)-N-(1-(8-chloro-6-fluoro-4-(pyridin-2-yl)quinolin-3-yl)ethylidene)-2-methylpropane-2-sulfinamide

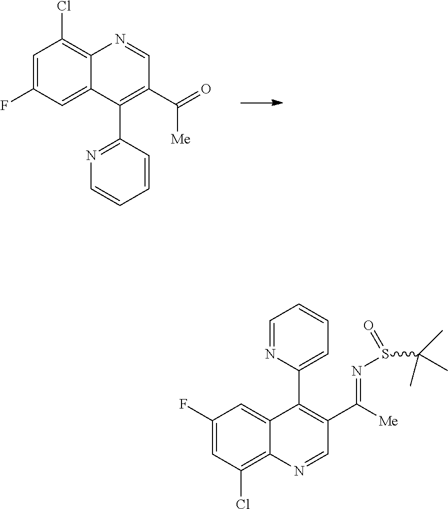 [SMILES: CC(=O)c1cnc2c(Cl)cc(F)cc2c1-c1ccccn1 ; C/C(=N\S(=O)C(C)(C)C)c1cnc2c(Cl)cc(F)cc2c1-c1ccccn1]

Tetraisopropoxytitanium (2.023 mL, 6.83 mmol), 2-methylpropane-2-sulfinamide (0.473 g, 3.90 mmol), and 1-(8-chloro-6-fluoro-4-(pyridin-2-yl)quinolin-3-yl)-ethanone (0.587 g, 1.952 mmol) were combined in 10 ml of anhydrous toluene. The solution was heated to 110° C. for 3 h and then at 75° C. overnight. The next day the solution was cooled to rt and then diluted with DCM before it was filtered through a plug of celite. The solids were washed with DCM and then the filtrates were concentrated under vacuum. The residue obtained was partially dissolved in acetone/H₂O and then filtered through a plug of silica gel. The silica gel was washed with acetone to isolate the product. The filtrates were concentrated under vacuum and the residue obtained was chromatographed over silica gel eluting with 4% MeOH/DCM. The fractions containing the product were combined and concentrated under vacuum to provide (E)-N-(1-(8-chloro-6-fluoro-4-(pyridin-2-yl)quinolin-3-yl)ethylidene)-2-methylpropane-2-sulfinamide as a brownish film which was carried on without further purification. Mass Spectrum (ESI) m/e=404.0 (M+1).

### N-(1-(8-chloro-6-fluoro-4-(pyridin-2-yl)quinolin-3-yl)ethyl)-2-methylpropane-2-sulfinamide

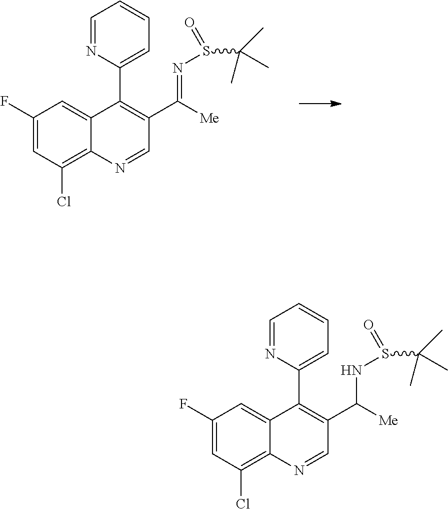 [SMILES: C/C(=N\S(=O)C(C)(C)C)c1cnc2c(Cl)cc(F)cc2c1-c1ccccn1 ; CC(NS(=O)C(C)(C)C)c1cnc2c(Cl)cc(F)cc2c1-c1ccccn1]

(E)-N-(1-(8-chloro-6-fluoro-4-(pyridin-2-yl)quinolin-3-yl)ethylidene)-2-methylpropane-2-sulfinamide (0.464 g, 1.15 mmol) was dissolved in THF (9.15 mL) and water (0.187 mL) and then cooled under an atmosphere of N₂ in a dry ice/brine bath. To this was added NaBH₄ (0.112 g, 2.97 mmol) and solution allowed to warm to rt overnight. The next day the solution was diluted with MeOH and concentrated under vacuum. The residue obtained was diluted with ethyl acetate and washed with sat. NaHCO₃ followed by brine. The organic layer was dried over MgSO₄ and concentrated under vacuum. The residue obtained was chromatographed over silica gel eluting with a gradient of 20% acetone/hexane to 40% acetone/hexane. The fractions containing product were combined and concentrated under vacuum to give N-(1-(8-chloro-6-fluoro-4-(pyridin-2-yl)quinolin-3-yl)ethyl)-2-methylpropane-2-sulfinamide as a brown oil (697 mg) which was carried on without further purification. Mass Spectrum (ESI) m/e=406.0 (M+1).

### N-(1-(6-fluoro-8-methyl-4-(pyridin-2-yl)quinolin-3-yl)ethyl)-2-methylpropane-2-sulfinamide

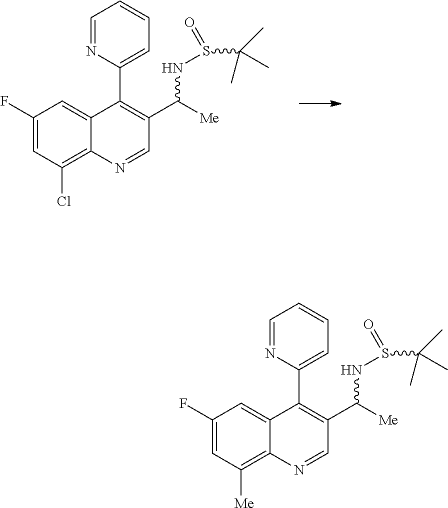 [SMILES: CC(NS(=O)C(C)(C)C)c1cnc2c(Cl)cc(F)cc2c1-c1ccccn1 ; Cc1cc(F)cc2c(-c3ccccn3)c(C(C)NS(=O)C(C)(C)C)cnc12]

N-(1-(8-chloro-6-fluoro-4-(pyridin-2-yl)quinolin-3-yl)ethyl)-2-methylpropane-2-sulfinamide (0.697 g, 1.78 mmol), potassium phosphate (1.82 g, 8.59 mmol), and 2,6-dimethyl-1,3,6,2-dioxazaborocane-4,8-dione (0.587 g, 3.43 mmol) were combined in 15 ml of 1,4-dioxane and 2 ml of H₂O. The solution was sparged with N₂ before adding dicyclohexyl(2′,4′,6′-triisopropylbiphenyl-2-yl)phosphine (0.082 g, 0.17 mmol), Pd(dba)₂ (0.049 g, 0.086 mmol) and heating to a gentle reflux for 12 h. An additional amount of potassium phosphate (1.822 g, 8.59 mmol), 2,6-dimethyl-1,3,6,2-dioxazaborocane-4,8-dione (0.587 g, 3.43 mmol), pd(dba)₂ (0.049 g, 0.086 mmol), and dicyclohexyl(2′,4′,6′-triisopropylbiphenyl-2-yl)phosphine (0.082 g, 0.172 mmol) were added with continued heating at a gentle reflux for 5 h. More of the 2,6-dimethyl-1,3,6,2-dioxazaborocane-4,8-dione (0.300 g, 1.755 mmol) was added at this time. After 1 h the solution was cooled to rt. and then diluted with DCM and H₂O. The layers were partitioned and the organic layer was concentrated under vacuum to give an orange oil. The oil obtained was chromatographed over silica gel eluting with a gradient of 2% MeOH/DCM to 10% MeOH/DCM. The fractions containing the product were combined and concentrated under vacuum to provide N-(1-(6-fluoro-8-methyl-4-(pyridin-2-yl)quinolin-3-yl)ethyl)-2-methylpropane-2-sulfinamide as a brownish foam which was carried on without further purification. Mass Spectrum (ESI) m/e=386.0 (M+1).

### 1-(6-fluoro-8-methyl-4-(pyridin-2-yl)quinolin-3-yl)ethanamine

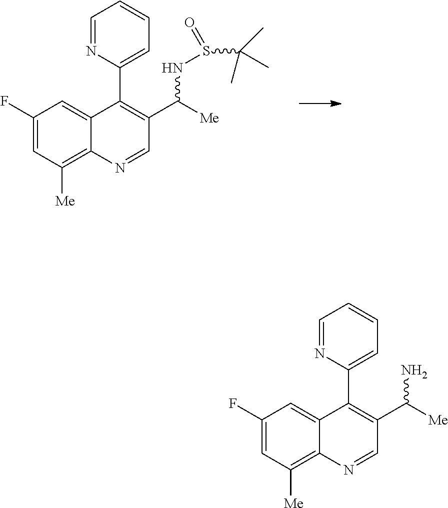 [SMILES: Cc1cc(F)cc2c(-c3ccccn3)c(C(C)NS(=O)C(C)(C)C)cnc12 ; Cc1cc(F)cc2c(-c3ccccn3)c(C(C)N)cnc12]

N-(1-(6-fluoro-8-methyl-4-(pyridin-2-yl)quinolin-3-yl)ethyl)-2-methylpropane-2-sulfinamide (0.298 g, 0.773 mmol) was dissolved in 7 mL of THF, and to this was added 1 ml of conc. HCl. The solution was stirred at rt for 10 min. The pH was adjusted to ˜9 with sat. NaHCO₃, and the product extracted with DCM. The organic layer was dried over MgSO₄ and concentrated under vacuum, to provide brownish oil. The oil obtained was chromatographed with silica gel eluting with a gradient of 2% MeOH/0.2% NH₄OH (˜28% in water)/DCM to 10% MeOH/1.0% NH₄OH (˜28% in water)/DCM. The fractions containing the product were combined and concentrated under vacuum to provide 1-(6-fluoro-8-methyl-4-(pyridin-2-yl)quinolin-3-yl)ethanamine as a light brownish oil which was carried on without further purification. Mass Spectrum (ESI) m/e=282.1 (M+1).

### 4-amino-6-((1-(6-fluoro-8-methyl-4-(pyridin-2-yl)quinolin-3-yl)ethyl)amino)-pyrimidine-5-carbonitrile

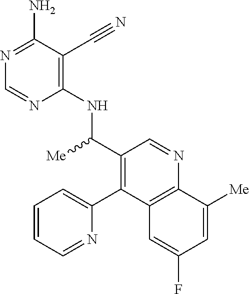 [SMILES: Cc1cc(F)cc2c(-c3ccccn3)c(C(C)Nc3ncnc(N)c3C#N)cnc12]

4-amino-6-((1-(6-fluoro-8-methyl-4-(pyridin-2-yl)quinolin-3-yl)ethyl)amino)-pyrimidine-5-carbonitrile (off white solid, 121 mg) was prepared according to the methods described in General Method A4 from 1-(6-fluoro-8-methyl-4-(pyridin-2-yl)quinolin-3-yl)ethanamine. A mixture of isomers was observed in the ¹H NMR trace. ¹H NMR (400 MHz, DMSO-d₆) δ ppm 9.19 (1H, s), 8.79 (1H, d, J=3.7 Hz), 6.99-8.11 (8H, m), 6.69 (1H, d, J=9.8 Hz), 4.93-5.50 (1H, m), 2.76 (3H, s), 1.21-1.67 (3H, m); Mass Spectrum (ESI) m/e=400.0 (M+1).

### Example 55

### 4-Amino-6-((1-(8-chloro-6-fluoro-4-phenylquinolin-3-yl)ethyl)amino)pyrimidine-5-carbonitrile

### 1-(8-chloro-6-fluoro-4-phenylquinolin-3-yl)ethanone

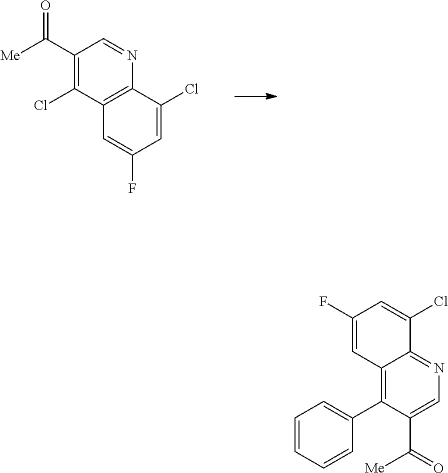 [SMILES: CC(=O)c1cnc2c(Cl)cc(F)cc2c1Cl ; CC(=O)c1cnc2c(Cl)cc(F)cc2c1-c1ccccc1]

1-(4,8-dichloro-6-fluoroquinolin-3-yl)ethanone (0.310 g, 1.20 mmol), phenylboronic acid (0.220 g, 1.80 mmol), and potassium carbonate (0.498 g, 3.60 mmol) were combined in DMF (4.80 mL). The suspension was briefly sparged with N₂ before adding PdCl₂(dppf)CH₂Cl₂ (0.098 g, 0.120 mmol). The suspension was then heated at 90° C. overnight. The next day the suspension was cooled to rt and diluted with ethyl acetate and water. The suspension was filtered through filter paper and then the filtrates were partitioned. The aqueous layer was washed with ethyl acetate and the combined organic layers were dried over MgSO₄, filtered, and concentrated under vacuum. The residue obtained was chromatographed over silica gel eluting with a gradient of 5% acetone/hexane to 15% acetone/hexane. The fractions containing the product were combined and concentrated under vacuum to provide 1-(8-chloro-6-fluoro-4-phenylquinolin-3-yl)ethanone, which was carried on without further purification. Mass Spectrum (ESI) m/e=300.0 (M+1).

### 1-(8-chloro-6-fluoro-4-phenylquinolin-3-yl)ethanamine

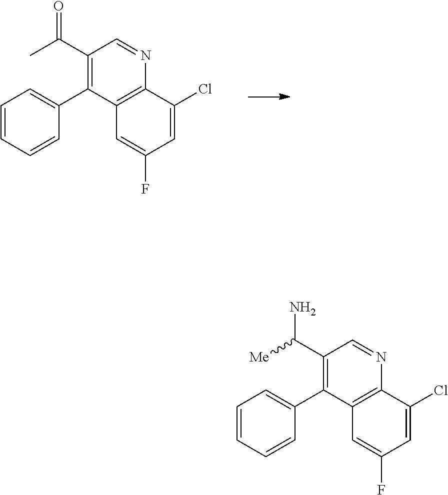 [SMILES: CC(=O)c1cnc2c(Cl)cc(F)cc2c1-c1ccccc1 ; CC(N)c1cnc2c(Cl)cc(F)cc2c1-c1ccccc1]

1-(8-chloro-6-fluoro-4-phenylquinolin-3-yl)ethanamine was prepared according to the methods described in General Method A10 from 1-(8-chloro-6-fluoro-4-phenylquinolin-3-yl)ethanone. Mass Spectrum (ESI) m/e=301.0 (M+1).

### 4-amino-6-((1-(8-chloro-6-fluoro-4-phenylquinolin-3-yl)ethyl)amino)-pyrimidine-5-carbonitrile

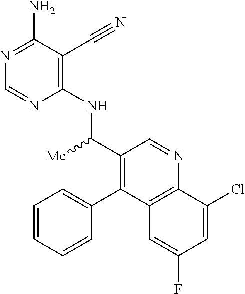 [SMILES: CC(Nc1ncnc(N)c1C#N)c1cnc2c(Cl)cc(F)cc2c1-c1ccccc1]

4-Amino-6-((1-(8-chloro-6-fluoro-4-phenylquinolin-3-yl)ethyl)amino)pyrimidine-5-carbonitrile (white solid) was prepared according to the methods described in General Method A4 from 1-(8-chloro-6-fluoro-4-phenylquinolin-3-yl)ethanamine. ¹H NMR (500 MHz, DMSO-d₆) δ ppm 9.27 (1H, s), 8.00 (1H, dd, J=8.3, 2.7 Hz), 7.93 (1H, d, J=7.1 Hz), 7.86 (1H, s), 7.51-7.65 (4H, m), 7.33 (1H, d, J=7.1 Hz), 7.22 (2H, br. s.), 6.83 (1H, dd, J=9.8, 2.7 Hz), 5.08 (1H, quintet, J=7.2 Hz), 1.47 (3H, d, J=7.1 Hz); Mass Spectrum (ESI) m/e=419.0 (M+1).

### Biological Assays

### Recombinant Expression of PI3Ks

Full length p110 subunits of PI3k α, β and δ, N-terminally labeled with polyHis tag, were coexpressed with p85 with Baculo virus expression vectors in sf9 insect cells. P110/p85 heterodimers were purified by sequential Ni-NTA, Q-HP, Superdex-100 chromatography. Purified α, β and δ isozymes were stored at −20° C. in 20 mM Tris, pH 8, 0.2M NaCl, 50% glycerol, 5 mM DTT, 2 mM Na cholate. Truncated PI3Kγ, residues 114-1102, N-terminally labeled with polyHis tag, was expressed with Baculo virus in Hi5 insect cells. The γ isozyme was purified by sequential Ni-NTA, Superdex-200, Q-HP chromatography. The γ isozyme was stored frozen at −80° C. in NaH₂PO₄, pH 8, 0.2M NaCl, 1% ethylene glycol, 2 mM β-mercaptoethanol.

| | Alpha | | Beta | | Delta | | gamma | |
|---|---|---|---|---|---|---|---|---|
| 50 mM Tris | pH 8 | | pH 7.5 | | pH 7.5 | | pH 8 | |
| MgC12 | 15 | mM | 10 | mM | 10 | mM | 15 | mM |
| Na cholate | 2 | mM | 1 | mM | 0.5 | mM | 2 | mM |
| DTT | 2 | mM | 1 | mM | 1 | mM | 2 | mM |
| ATP | 1 | uM | 0.5 | uM | 0.5 | uM | 1 | uM |
| PIP2 | none | | 2.5 | uM | 2.5 | uM | none | |
| Time | 1 | h | 2 | h | 2 | h | 1 | h |
| [Enzyme] | 15 | nM | 40 | nM | 15 | nM | 50 | nM |

### In Vitro PI3K Enzyme Assays

A PI3K Alphascreen® assay (PerkinElmer, Waltham, Mass.) was used to measure the activity of a panel of four phosphoinositide 3-kinases: PI3Kα, PI3Kβ, PI3Kγ, and PI3Kδ. Enzyme reaction buffer was prepared using sterile water (Baxter, Deerfield, Ill.) and 50 mM Tris HCl pH 7, 14 mM MgCl₂, 2 mM sodium cholate, and 100 mM NaCl. 2 mM DTT was added fresh the day of the experiment. The Alphascreen buffer was made using sterile water and 10 mM Tris HCl pH 7.5, 150 mM NaCl, 0.10% Tween 20, and 30 mM EDTA. 1 mM DTT was added fresh the day of the experiment. Compound source plates used for this assay were 384-well Greiner clear polypropylene plates containing test compounds at 5 mM and diluted 1:2 over 22 concentrations. Columns 23 and 24 contained only DMSO as these wells comprised the positive and negative controls, respectively. Source plates were replicated by transferring 0.5 uL per well into 384-well Optiplates (PerkinElmer, Waltham, Mass.).

Each PI3K isoform was diluted in enzyme reaction buffer to 2× working stocks. PI3Ka was diluted to 1.6 nM, PI3Kβ was diluted to 0.8 nM, PI3Kγ was diluted to 15 nM, and PI3Kδ was diluted to 1.6 nM. PI(4,5)P2 (Echelon Biosciences, Salt Lake City, Utah) was diluted to 10 μM and ATP was diluted to 20 μM. This 2× stock was used in the assays for PI3Kα and PI3Kβ. For assay of PI3Kγ and PI3Kδ, PI(4,5)P2 was diluted to 10 μM and ATP was diluted to 8 μM to prepare a similar 2× working stock. Alphascreen reaction solutions were made using beads from the anti-GST Alphascreen kit (PerkinElmer, Waltham, Mass.). Two 4× working stocks of the Alphascreen reagents were made in Alphascreen reaction buffer. In one stock, biotinylated-IP₄ (Echelon Biosciences, Salt Lake City, Utah) was diluted to 40 nM and streptavadin-donor beads were diluted to 80 μm/mL. In the second stock, PIP₃-binding protein (Echelon Biosciences, Salt Lake City, Utah) was diluted to 40 nM and anti-GST-acceptor beads were diluted to 80 m/mL. As a negative control, a reference inhibitor at a concentration >>Ki (40 uM) was included in column 24 as a negative (100% inhibition) control.

Using a 384-well Multidrop (Titertek, Huntsville, Ala.), 10 μL/well of 2× enzyme stock was added to columns 1-24 of the assay plates for each isoform. 10 μL/well of the appropriate substrate 2× stock (containing 20 μM ATP for the PI3Kα and β assays and containing 8 μM ATP for the PI3Kγ and δ assays) was then added to Columns 1-24 of all plates. Plates were then incubated at room temperature for 20 minutes. In the dark, 10 μL/well of the donor bead solution was added to columns 1-24 of the plates to quench the enzyme reaction. The plates were incubated at room temperature for 30 minutes. Still in the dark, 10 μL/well of the acceptor bead solution was added to columns 1-24 of the plates. The plates were then incubated in the dark for 1.5 h. The plates were read on an Envision multimode Plate Reader (PerkinElmer, Waltham, Mass.) using a 680 nm excitation filter and a 520-620 nm emission filter.

### Alternative In Vitro Enzyme Assays.

Assays were performed in 25 μL with the above final concentrations of components in white polyproplyene plates (Costar 3355). Phospatidyl inositol phosphoacceptor, PtdIns(4,5)P2 P4508, was from Echelon Biosciences. The ATPase activity of the alpha and gamma isozymes was not greatly stimulated by PtdIns(4,5)P2 under these conditions and was therefore omitted from the assay of these isozymes. Test compounds were dissolved in dimethyl sulfoxide and diluted with three-fold serial dilutions. The compound in DMSO (1 μL) was added per test well, and the inhibition relative to reactions containing no compound, with and without enzyme was determined. After assay incubation at rt, the reaction was stopped and residual ATP determined by addition of an equal volume of a commercial ATP bioluminescence kit (Perkin Elmer EasyLite) according to the manufacturer's instructions, and detected using a AnalystGT luminometer.

### Human B Cells Proliferation Stimulate by Anti-IgM

### Isolate Human B Cells:

Isolate PBMCs from Leukopac or from human fresh blood. Isolate human B cells by using Miltenyi protocol and B cell isolation kit II-human B cells were Purified by using AutoMacs™ column.

### Activation of Human B Cells

Use 96 well Flat bottom plate, plate 50000/well purified B cells in B cell proliferation medium (DMEM+5% FCS, 10 mM Hepes, 50 μM 2-mercaptoethanol); 150 μL medium contain 250 ng/mL CD40L-LZ recombinant protein (Amgen) and 2 μg/mL anti-Human IgM antibody (Jackson ImmunoReseach Lab. #109-006-129), mixed with 50 μL B cell medium containing PI3K inhibitors and incubate 72 h at 37° C. incubator. After 72 h, pulse labeling B cells with 0.5-1 uCi/well ³H thymidine for overnight ˜18 h, and harvest cell using TOM harvester.

### Human B Cells Proliferation Stimulate by IL-4

### Isolate Human B Cells:

Isolate human PBMCs from Leukopac or from human fresh blood. Isolate human B cells using Miltenyi protocol-B cell isolation kit. Human B cells were purified by AutoMacs. column.

### Activation of Human B Cells

Use 96-well flat bottom plate, plate 50000/well purified B cells in B cell proliferation medium (DMEM+5% FCS, 50 μM 2-mercaptoethanol, 10 mM Hepes). The medium (150 μL) contain 250 ng/mL CD40L-LZ recombinant protein (Amgen) and 10 ng/mL IL-4 (R&D system #204-IL-025), mixed with 50 150 μL B cell medium containing compounds and incubate 72 h at 37° C. incubator. After 72 h, pulse labeling B cells with 0.5-1 uCi/well 3H thymidine for overnight ˜18 h, and harvest cell using TOM harvester.

### Specific T Antigen (Tetanus Toxoid) Induced Human PBMC Proliferation Assays

Human PBMC are prepared from frozen stocks or they are purified from fresh human blood using a Ficoll gradient. Use 96 well round-bottom plate and plate 2×10⁵ PBMC/well with culture medium (RPMI1640+10% FCS, 50 uM 2-Mercaptoethanol, 10 mM Hepes). For IC₅₀ determinations, PI3K inhibitors was tested from 10 μM to 0.001 μM, in half log increments and in triplicate. Tetanus toxoid, T cell specific antigen (University of Massachusetts Lab) was added at 1 μg/mL and incubated 6 days at 37° C. incubator. Supernatants are collected after 6 days for IL2 ELISA assay, then cells are pulsed with ³H-thymidine for ˜18 h to measure proliferation.

### GFP Assays for Detecting Inhibition of Class Ia and Class III PI3K

AKT1 (PKBa) is regulated by Class Ia PI3K activated by mitogenic factors (IGF-1, PDGF, insulin, thrombin, NGF, etc.). In response to mitogenic stimuli, AKT1 translocates from the cytosol to the plasma membrane Forkhead (FKHRL1) is a substrate for AKT1. It is cytoplasmic when phosphorylated by AKT (survival/growth). Inhibition of AKT (stasis/apoptosis)-forkhead translocation to the nucleus FYVE domains bind to PI(3)P. The majority is generated by constitutive action of PI3K Class III

### AKT Membrane Ruffling Assay (CHO-IR-AKT1-EGFP Cells/GE Healthcare)

Wash cells with assay buffer. Treat with compounds in assay buffer 1 h. Add 10 ng/mL insulin. Fix after 10 min at room temp and image

### Forkhead Translocation Assay (MDA MB468 Forkhead-DiversaGFP Cells)

Treat cells with compound in growth medium 1 h. Fix and image.

### Class III PI(3)P Assay (U2OS EGFP-2×FYVE Cells/GE Healthcare)

Wash cells with assay buffer. Treat with compounds in assay buffer 1 h. Fix and image.

### Control for all 3 Assays is 10 uM Wortmannin:

AKT is cytoplasmic
Forkhead is nuclear
PI(3)P depleted from endosomes

### Biomarker Assay: B-Cell Receptor Stimulation of CD69 or B7.2 (CD86) Expression

Heparinized human whole blood was stimulated with 10 μg/mL anti-IgD (Southern Biotech, #9030-01). 90 μL of the stimulated blood was then aliquoted per well of a 96-well plate and treated with 10 μL of various concentrations of blocking compound (from 10-0.0003 μM) diluted in IMDM+10% FBS (Gibco). Samples were incubated together for 4 h (for CD69 expression) to 6 h (for B7.2 expression) at 37° C. Treated blood (50 μL) was transferred to a 96-well, deep well plate (Nunc) for antibody staining with 10 μL each of CD45-PerCP (BD Biosciences, #347464), CD19-FITC (BD Biosciences, #340719), and CD69-PE (BD Biosciences, #341652). The second 50 μL of the treated blood was transferred to a second 96-well, deep well plate for antibody staining with 10 μL each of CD19-FITC (BD Biosciences, #340719) and CD86-PeCy5 (BD Biosciences, #555666). All stains were performed for 15-30 min in the dark at rt. The blood was then lysed and fixed using 450 μL of FACS lysing solution (BD Biosciences, #349202) for 15 min at rt. Samples were then washed 2× in PBS+2% FBS before FACS analysis. Samples were gated on either CD45/CD19 double positive cells for CD69 staining, or CD19 positive cells for CD86 staining.

### Gamma Counterscreen: Stimulation of Human Monocytes for Phospho-AKT Expression

A human monocyte cell line, THP-1, was maintained in RPMI+10% FBS (Gibco). One day before stimulation, cells were counted using trypan blue exclusion on a hemocytometer and suspended at a concentration of 1×10⁶ cells per mL of media. 100 μL of cells plus media (1×10⁵ cells) was then aliquoted per well of 4-96-well, deep well dishes (Nunc) to test eight different compounds. Cells were rested overnight before treatment with various concentrations (from 10-0.0003 μM) of blocking compound. The compound diluted in media (12 μL) was added to the cells for 10 min at 37° C. Human MCP-1 (12 μL, R&D Diagnostics, #279-MC) was diluted in media and added to each well at a final concentration of 50 ng/mL. Stimulation lasted for 2 min at rt. Pre-warmed FACS Phosflow Lyse/Fix buffer (1 mL of 37° C.) (BD Biosciences, #558049) was added to each well. Plates were then incubated at 37° C. for an additional 10-15 min. Plates were spun at 1500 rpm for 10 min, supernatant was aspirated off, and 1 mL of ice cold 90% MeOH was added to each well with vigorous shaking Plates were then incubated either overnight at −70° C. or on ice for 30 min before antibody staining Plates were spun and washed 2× in PBS+2% FBS (Gibco). Wash was aspirated and cells were suspended in remaining buffer. Rabbit pAKT (50 μL, Cell Signaling, #4058L) at 1:100, was added to each sample for 1 h at rt with shaking Cells were washed and spun at 1500 rpm for 10 min. Supernatant was aspirated and cells were suspended in remaining buffer. Secondary antibody, goat anti-rabbit Alexa 647 (50 μL, Invitrogen, #A21245) at 1:500, was added for 30 min at rt with shaking Cells were then washed 1× in buffer and suspended in 150 μL of buffer for FACS analysis. Cells need to be dispersed very well by pipetting before running on flow cytometer. Cells were run on an LSR II (Becton Dickinson) and gated on forward and side scatter to determine expression levels of pAKT in the monocyte population.

### Gamma Counterscreen Stimulation of Monocytes for Phospho-AKT Expression in Mouse Bone Marrow

Mouse femurs were dissected from five female BALB/c mice (Charles River Labs.) and collected into RPMI+10% FBS media (Gibco). Mouse bone marrow was removed by cutting the ends of the femur and by flushing with 1 mL of media using a 25 gauge needle. Bone marrow was then dispersed in media using a 21 gauge needle. Media volume was increased to 20 mL and cells were counted using trypan blue exclusion on a hemocytometer. The cell suspension was then increased to 7.5×10⁶ cells per 1 mL of media and 100 μL (7.5×10⁵ cells) was aliquoted per well into 4-96-well, deep well dishes (Nunc) to test eight different compounds. Cells were rested at 37° C. for 2 h before treatment with various concentrations (from 10-0.0003 μM) of blocking compound. Compound diluted in media (12 μL) was added to bone marrow cells for 10 min at 37° C. Mouse MCP-1 (12 μL, R&D Diagnostics, #479-JE) was diluted in media and added to each well at a final concentration of 50 ng/mL. Stimulation lasted for 2 min at rt. 1 mL of 37° C. pre-warmed FACS Phosflow Lyse/Fix buffer (BD Biosciences, #558049) was added to each well. Plates were then incubated at 37° C. for an additional 10-15 min. Plates were spun at 1500 rpm for 10 min. Supernatant was aspirated off and 1 mL of ice cold 90% MeOH was added to each well with vigorous shaking Plates were then incubated either overnight at −70° C. or on ice for 30 min before antibody staining Plates were spun and washed 2× in PBS+2% FBS (Gibco). Wash was aspirated and cells were suspended in remaining buffer. Fc block (2 μL, BD Pharmingen, #553140) was then added per well for 10 min at rt. After block, 50 μL of primary antibodies diluted in buffer; CD11b-Alexa488 (BD Biosciences, #557672) at 1:50, CD64-PE (BD Biosciences, #558455) at 1:50, and rabbit pAKT (Cell Signaling, #4058L) at 1:100, were added to each sample for 1 h at rt with shaking Wash buffer was added to cells and spun at 1500 rpm for 10 min. Supernatant was aspirated and cells were suspended in remaining buffer. Secondary antibody; goat anti-rabbit Alexa 647 (50 μL, Invitrogen, #A21245) at 1:500, was added for 30 min at rt with shaking Cells were then washed 1× in buffer and suspended in 100 μL of buffer for FACS analysis. Cells were run on an LSR II (Becton Dickinson) and gated on CD11b/CD64 double positive cells to determine expression levels of pAKT in the monocyte population.

pAKT In Vivo Assay

Vehicle and compounds are administered p.o. (0.2 mL) by gavage (Oral Gavage Needles Popper & Sons, New Hyde Park, N.Y.) to mice (Transgenic Line 3751, female, 10-12 wks Amgen Inc, Thousand Oaks, Calif.) 15 min prior to the injection i.v (0.2 mLs) of anti-IgM FITC (50 ug/mouse) (Jackson Immuno Research, West Grove, Pa.). After 45 min the mice are sacrificed within a CO₂ chamber. Blood is drawn via cardiac puncture (0.3 mL) (l cc 25 g Syringes, Sherwood, St. Louis, Mo.) and transferred into a 15 mL conical vial (Nalge/Nunc International, Denmark). Blood is immediately fixed with 6.0 mL of BD Phosflow Lyse/Fix Buffer (BD Bioscience, San Jose, Calif.), inverted 3×'s and placed in 37° C. water bath. Half of the spleen is removed and transferred to an eppendorf tube containing 0.5 mL of PBS (Invitrogen Corp, Grand Island, N.Y.). The spleen is crushed using a tissue grinder (Pellet Pestle, Kimble/Kontes, Vineland, N.J.) and immediately fixed with 6.0 mL of BD Phosflow Lyse/Fix buffer, inverted 3×'s and placed in 37° C. water bath. Once tissues have been collected the mouse is cervically-dislocated and carcass to disposed. After 15 min, the 15 mL conical vials are removed from the 37° C. water bath and placed on ice until tissues are further processed. Crushed spleens are filtered through a 70 μm cell strainer (BD Bioscience, Bedford, Mass.) into another 15 mL conical vial and washed with 9 mL of PBS. Splenocytes and blood are spun @ 2,000 rpms for 10 min (cold) and buffer is aspirated. Cells are resuspended in 2.0 mL of cold (−20° C.) 90% MeOH (Mallinckrodt Chemicals, Phillipsburg, N.J.). MeOH is slowly added while conical vial is rapidly vortexed. Tissues are then stored at −20° C. until cells can be stained for FACS analysis.

### Multi-Dose TNP Immunization

Blood was collected by retro-orbital eye bleeds from 7-8 week old BALB/c female mice (Charles River Labs.) at day 0 before immunization. Blood was allowed to clot for 30 min and spun at 10,000 rpm in serum microtainer tubes (Becton Dickinson) for 10 min. Sera were collected, aliquoted in Matrix tubes (Matrix Tech. Corp.) and stored at −70° C. until ELISA was performed. Mice were given compound orally before immunization and at subsequent time periods based on the life of the molecule. Mice were then immunized with either 50 μg of TNP-LPS (Biosearch Tech., #T-5065), 50 μg of TNP-Ficoll (Biosearch Tech., #F-1300), or 100 μg of TNP-KLH (Biosearch Tech., #T-5060) plus 1% alum (Brenntag, #3501) in PBS. TNP-KLH plus alum solution was prepared by gently inverting the mixture 3-5 times every 10 min for 1 h before immunization. On day 5, post-last treatment, mice were CO₂ sacrificed and cardiac punctured. Blood was allowed to clot for 30 min and spun at 10,000 rpm in serum microtainer tubes for 10 min. Sera were collected, aliquoted in Matrix tubes, and stored at −70° C. until further analysis was performed. TNP-specific IgG1, IgG2α, IgG3 and IgM levels in the sera were then measured via ELISA. TNP-BSA (Biosearch Tech., #T-5050) was used to capture the TNP-specific antibodies. TNP-BSA (10 μg/mL) was used to coat 384-well ELISA plates (Corning Costar) overnight. Plates were then washed and blocked for 1 h using 10% BSA ELISA Block solution (KPL). After blocking, ELISA plates were washed and sera samples/standards were serially diluted and allowed to bind to the plates for 1 h. Plates were washed and Ig-HRP conjugated secondary antibodies (goat anti-mouse IgG1, Southern Biotech #1070-05, goat anti-mouse IgG2α, Southern Biotech #1080-05, goat anti-mouse IgM, Southern Biotech #1020-05, goat anti-mouse IgG3, Southern Biotech #1100-05) were diluted at 1:5000 and incubated on the plates for 1 h. TMB peroxidase solution (SureBlue Reserve TMB from KPL) was used to visualize the antibodies. Plates were washed and samples were allowed to develop in the TMB solution approximately 5-20 min depending on the Ig analyzed. The reaction was stopped with 2M sulfuric acid and plates were read at an OD of 450 nm.

For the treatment of PI3Kδ-mediated-diseases, such as rheumatoid arthritis, ankylosing spondylitis, osteoarthritis, psoriatic arthritis, psoriasis, inflammatory diseases, and autoimmune diseases, the compounds of the present invention may be administered orally, parentally, by inhalation spray, rectally, or topically in dosage unit formulations containing conventional pharmaceutically acceptable carriers, adjuvants, and vehicles. The term parenteral as used herein includes, subcutaneous, intravenous, intramuscular, intrasternal, infusion techniques or intraperitoneally.

Treatment of diseases and disorders herein is intended to also include the prophylactic administration of a compound of the invention, a pharmaceutical salt thereof, or a pharmaceutical composition of either to a subject (i.e., an animal, preferably a mammal, most preferably a human) believed to be in need of preventative treatment, such as, for example, rheumatoid arthritis, ankylosing spondylitis, osteoarthritis, psoriatic arthritis, psoriasis, inflammatory diseases, and autoimmune diseases and the like.

The dosage regimen for treating PI3Kδ-mediated diseases, cancer, and/or hyperglycemia with the compounds of this invention and/or compositions of this invention is based on a variety of factors, including the type of disease, the age, weight, sex, medical condition of the patient, the severity of the condition, the route of administration, and the particular compound employed. Thus, the dosage regimen may vary widely, but can be determined routinely using standard methods. Dosage levels of the order from about 0.01 mg to 30 mg per kilogram of body weight per day, preferably from about 0.1 mg to 10 mg/kg, more preferably from about 0.25 mg to 1 mg/kg are useful for all methods of use disclosed herein.

The pharmaceutically active compounds of this invention can be processed in accordance with conventional methods of pharmacy to produce medicinal agents for administration to patients, including humans and other mammals.

For oral administration, the pharmaceutical composition may be in the form of, for example, a capsule, a tablet, a suspension, or liquid. The pharmaceutical composition is preferably made in the form of a dosage unit containing a given amount of the active ingredient. For example, these may contain an amount of active ingredient from about 1 to 2000 mg, preferably from about 1 to 500 mg, more preferably from about 5 to 150 mg. A suitable daily dose for a human or other mammal may vary widely depending on the condition of the patient and other factors, but, once again, can be determined using routine methods.

The active ingredient may also be administered by injection as a composition with suitable carriers including saline, dextrose, or water. The daily parenteral dosage regimen will be from about 0.1 to about 30 mg/kg of total body weight, preferably from about 0.1 to about 10 mg/kg, and more preferably from about 0.25 mg to 1 mg/kg.

Injectable preparations, such as sterile injectable aq or oleaginous suspensions, may be formulated according to the known are using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

Suppositories for rectal administration of the drug can be prepared by mixing the drug with a suitable non-irritating excipient such as cocoa butter and polyethylene glycols that are solid at ordinary temperatures but liquid at the rectal temperature and will therefore melt in the rectum and release the drug.

A suitable topical dose of active ingredient of a compound of the invention is 0.1 mg to 150 mg administered one to four, preferably one or two times daily. For topical administration, the active ingredient may comprise from 0.001% to 10% w/w, e.g., from 1% to 2% by weight of the formulation, although it may comprise as much as 10% w/w, but preferably not more than 5% w/w, and more preferably from 0.1% to 1% of the formulation.

Formulations suitable for topical administration include liquid or semi-liquid preparations suitable for penetration through the skin (e.g., liniments, lotions, ointments, creams, or pastes) and drops suitable for administration to the eye, ear, or nose.

For administration, the compounds of this invention are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulfuric acids, acacia, gelatin, sodium alginate, polyvinyl-pyrrolidine, and/or polyvinyl alcohol, and tableted or encapsulated for conventional administration. Alternatively, the compounds of this invention may be dissolved in saline, water, polyethylene glycol, propylene glycol, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The pharmaceutical compositions may be made up in a solid form (including granules, powders or suppositories) or in a liquid form (e.g., solutions, suspensions, or emulsions). The pharmaceutical compositions may be subjected to conventional pharmaceutical operations such as sterilization and/or may contain conventional adjuvants, such as preservatives, stabilizers, wetting agents, emulsifiers, buffers etc.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting, sweetening, flavoring, and perfuming agents.

Compounds of the present invention can possess one or more asymmetric carbon atoms and are thus capable of existing in the form of optical isomers as well as in the form of racemic or non-racemic mixtures thereof. The optical isomers can be obtained by resolution of the racemic mixtures according to conventional processes, e.g., by formation of diastereoisomeric salts, by treatment with an optically active acid or base. Examples of appropriate acids are tartaric, diacetyltartaric, dibenzoyltartaric, ditoluoyltartaric, and camphorsulfonic acid and then separation of the mixture of diastereoisomers by crystallization followed by liberation of the optically active bases from these salts. A different process for separation of optical isomers involves the use of a chiral chromatography column optimally chosen to maximize the separation of the enantiomers. Still another available method involves synthesis of covalent diastereoisomeric molecules by reacting compounds of the invention with an optically pure acid in an activated form or an optically pure isocyanate. The synthesized diastereoisomers can be separated by conventional means such as chromatography, distillation, crystallization or sublimation, and then hydrolyzed to deliver the enantiomerically pure compound. The optically active compounds of the invention can likewise be obtained by using active starting materials. These isomers may be in the form of a free acid, a free base, an ester or a salt.

Likewise, the compounds of this invention may exist as isomers, that is compounds of the same molecular formula but in which the atoms, relative to one another, are arranged differently. In particular, the alkylene substituents of the compounds of this invention, are normally and preferably arranged and inserted into the molecules as indicated in the definitions for each of these groups, being read from left to right. However, in certain cases, one skilled in the art will appreciate that it is possible to prepare compounds of this invention in which these substituents are reversed in orientation relative to the other atoms in the molecule. That is, the substituent to be inserted may be the same as that noted above except that it is inserted into the molecule in the reverse orientation. One skilled in the art will appreciate that these isomeric forms of the compounds of this invention are to be construed as encompassed within the scope of the present invention.

The compounds of the present invention can be used in the form of salts derived from inorganic or organic acids. The salts include, but are not limited to, the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methansulfonate, nicotinate, 2-naphthalenesulfonate, oxalate, palmoate, pectinate, persulfate, 2-phenylpropionate, picrate, pivalate, propionate, succinate, tartrate, thiocyanate, tosylate, mesylate, and undecanoate. Also, the basic nitrogen-containing groups can be quaternized with such agents as lower alkyl halides, such as methyl, ethyl, propyl, and butyl chloride, bromides and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained.

Examples of acids that may be employed to from pharmaceutically acceptable acid addition salts include such inorganic acids as hydrochloric acid, sulfuric acid and phosphoric acid and such organic acids as oxalic acid, maleic acid, succinic acid and citric acid. Other examples include salts with alkali metals or alkaline earth metals, such as sodium, potassium, calcium or magnesium or with organic bases.

Also encompassed in the scope of the present invention are pharmaceutically acceptable esters of a carboxylic acid or hydroxyl containing group, including a metabolically labile ester or a prodrug form of a compound of this invention. A metabolically labile ester is one which may produce, for example, an increase in blood levels and prolong the efficacy of the corresponding non-esterified form of the compound. A prodrug form is one which is not in an active form of the molecule as administered but which becomes therapeutically active after some in vivo activity or biotransformation, such as metabolism, for example, enzymatic or hydrolytic cleavage. For a general discussion of prodrugs involving esters see Svensson and Tunek Drug Metabolism Reviews 165 (1988) and Bundgaard Design of Prodrugs, Elsevier (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bungaard J. Med. Chem. 2503 (1989)). Also, drugs containing an acidic NH group, such as imidazole, imide, indole and the like, have been masked with N-acyloxymethyl groups (Bundgaard Design of Prodrugs, Elsevier (1985)). Hydroxy groups have been masked as esters and ethers. EP 039,051 (Sloan and Little, Apr. 11, 1981) discloses Mannich-base hydroxamic acid prodrugs, their preparation and use. Esters of a compound of this invention, may include, for example, the methyl, ethyl, propyl, and butyl esters, as well as other suitable esters formed between an acidic moiety and a hydroxyl containing moiety. Metabolically labile esters, may include, for example, methoxymethyl, ethoxymethyl, iso-propoxymethyl, α-methoxyethyl, groups such as α-((C₁-C₄)-alkyloxy)ethyl, for example, methoxyethyl, ethoxyethyl, propoxyethyl, iso-propoxyethyl, etc.; 2-oxo-1,3-dioxolen-4-ylmethyl groups, such as 5-methyl-2-oxo-1,3,dioxolen-4-ylmethyl, etc.; C₁-C₃ alkylthiomethyl groups, for example, methylthiomethyl, ethylthiomethyl, isopropylthiomethyl, etc.; acyloxymethyl groups, for example, pivaloyloxymethyl, α-acetoxymethyl, etc.; ethoxycarbonyl-1-methyl; or α-acyloxy-α-substituted methyl groups, for example α-acetoxyethyl.

Further, the compounds of the invention may exist as crystalline solids which can be crystallized from common solvents such as ethanol, N,N-dimethyl-formamide, water, or the like. Thus, crystalline forms of the compounds of the invention may exist as polymorphs, solvates and/or hydrates of the parent compounds or their pharmaceutically acceptable salts. All of such forms likewise are to be construed as falling within the scope of the invention.

While the compounds of the invention can be administered as the sole active pharmaceutical agent, they can also be used in combination with one or more compounds of the invention or other agents. When administered as a combination, the therapeutic agents can be formulated as separate compositions that are given at the same time or different times, or the therapeutic agents can be given as a single composition.

The foregoing is merely illustrative of the invention and is not intended to limit the invention to the disclosed compounds. Variations and changes which are obvious to one skilled in the art are intended to be within the scope and nature of the invention which are defined in the appended claims.

From the foregoing description, one skilled in the art can easily ascertain the essential characteristics of this invention, and without departing from the spirit and scope thereof, can make various changes and modifications of the invention to adapt it to various usages and conditions.

We claim:

1. A compound having the structure: 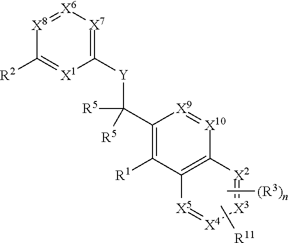
or any pharmaceutically-acceptable salt thereof, wherein:
X¹ is C(R¹⁰) or N;
X² is C or N;
X³ is C or N;
X⁴ is C or N;
X⁵ is C or N; wherein at least two of X², X³, X⁴ and X⁵ are C;
X⁶ is C(R⁶) or N;
X⁷ is C(R⁷) or N;
X⁸ is C(R¹⁰) or N; wherein no more than two of X¹, X⁶, X⁷ and X⁸ are N;
X⁹ is C(R⁴) or N;
X¹⁰ is C(R⁴) or N;
Y is N(R⁸), O or S;
n is 0, 1, 2 or 3;
R¹ is selected from H, halo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a}, —NR^{a}C₂₋₆alkOR^{a}, —NR^{a}C₂₋₆alkCO₂R^{a}, —NR^{a}C₂₋₆alkSO₂R^{b}, —CH₂C(═O)R^{a}, —CH₂C(═O)OR^{a}, —CH₂C(═O)NR^{a}R^{a}, —CH₂C(═NR^{a})NR^{a}R^{a}, —CH₂OR^{a}, —CH₂C(═O)R^{a}, —CH₂C(═O)NR^{a}R^{a}, —CH₂C(═O)N(R^{a})S(═O)₂R^{a}, —CH₂OC₂₋₆alkNR^{a}R^{a}, —CH₂OC₂₋₆alkOR^{a}, —CH₂SR^{a}, —CH₂S(═O)R^{a}, —CH₂S(═O)₂R^{b}, —CH₂S(═O)₂NR^{a}R^{a}, —CH₂S(═O)₂N(R^{a})C(═O)R^{a}, —CH₂S(═O)₂N(R^{a})C(═O)OR^{a}, —CH₂S(═O)₂N(R^{a})C(═O)NR^{a}R^{a}, —CH₂NR^{a}R^{a}, —CH₂N(R^{a})C(═O)R^{a}, —CH₂N(R^{a})C(═O)OR^{a}, —CH₂N(R^{a})C(═O)NR^{a}R^{a}, —CH₂N(R^{a})C(═NR^{a})NR^{a}R^{a}, —CH₂N(R^{a})S(═O)₂R^{a}, —CH₂N(R^{a})S(═O)₂NR^{a}R^{a}, —CH₂NR^{a}C₂₋₆alkNR^{a}R^{a}, —CH₂NR^{a}C₂₋₆alkOR^{a}, —CH₂NR^{a}C₂₋₆alkCO₂R^{a} and —CH₂NR^{a}C₂₋₆alkSO₂R^{b}; or R¹ is a direct-bonded, C₁₋₄alk-linked, OC₁₋₂alk-linked, C₁₋₂alkO-linked, N(R^{a})-linked or O-linked saturated, partially-saturated or unsaturated 3-, 4-, 5-, 6- or 7-membered monocyclic or 8-, 9-, 10- or 11-membered bicyclic ring containing 0, 1, 2, 3 or 4 atoms selected from N, O and S, but containing no more than one O or S atom, substituted by 0, 1, 2 or 3 substituents independently selected from halo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, OC(═O)R^{a}, —OC(═O)NR^{a}R^{a}, OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}, wherein the available carbon atoms of the ring are additionally substituted by 0, 1 or 2 oxo or thioxo groups, and wherein the ring is additionally substituted by 0 or 1 directly bonded, SO₂ linked, C(═O) linked or CH₂ linked group selected from phenyl, pyridyl, pyrimidyl, morpholino, piperazinyl, piperadinyl, pyrrolidinyl, cyclopentyl, cyclohexyl all of which are further substituted by 0, 1, 2 or 3 groups selected from halo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —SR^{a}, —S(═O)R^{a}, —S(═O)₂R^{a}, —S(═O)₂NR^{a}R^{a}, —NR^{a}R^{a}, and —N(R^{a})C(═O)R^{a};
R² is selected from H, halo, C₁₋₆alk, C₁₋₄haloalk, cyano, nitro, OR^{a}, NR^{a}R^{a}, —C(═O)R^{a}, —C(═O)OR^{a}, —C(═O)NR^{a}R^{a}, —C(═NR^{a})NR^{a}R^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a} and —S(═O)₂N(R^{a})C(═O)NR^{a}R^{a};
R³ is, independently, in each instance, H, halo, nitro, cyano, C₁₋₄alk, OC₁₋₄alk, OC₁₋₄haloalk, NHC₁₋₄alk, N(C₁₋₄alk)C₁₋₄alk or C₁₋₄haloalk;
R⁴ is, independently, in each instance, H, halo, nitro, cyano, C₁₋₄alk, OC₁₋₄alk, OC₁₋₄haloalk, NHC₁₋₄alk, N(C₁₋₄alk)C₁₋₄alk, C₁₋₄haloalk or an unsaturated 5-, 6- or 7-membered monocyclic ring containing 0, 1, 2, 3 or 4 atoms selected from N, O and S, but containing no more than one O or S, the ring being substituted by 0, 1, 2 or 3 substituents selected from halo, C₁₋₄alk, C₁₋₃haloalk, —OC₁₋₄alk, —NH₂, —NHC₁₋₄alk, —N(C₁₋₄alk)C₁₋₄alk;
R⁵ is, independently, in each instance, H, halo, C₁₋₆alk, C₁₋₄haloalk, or C₁₋₆alk substituted by 1, 2 or 3 substituents selected from halo, cyano, OH, OC₁₋₄alk, C₁₋₄alk, C₁₋₃haloalk, OC₁₋₄alk, NH₂, NHC₁₋₄alk and N(C₁₋₄alk)C₁₋₄alk;
or both R⁵ groups together form a C₃₋₆spiroalk substituted by 0, 1, 2 or 3 substituents selected from halo, cyano, OH, OC₁₋₄alk, C₁₋₄alk, C₁₋₃haloalk, OC₁₋₄alk, NH₂, NHC₁₋₄alk and N(C₁₋₄alk)C₁₋₄alk;
R⁶ is H, halo, NHR⁹ or OH, cyano, OC₁₋₄alk, C₁₋₄alk, C₁₋₃haloalk, OC₁₋₄alk, —C(═O)OR^{a}, —C(═O)N(R^{a})R^{a} or —N(R^{a})C(═O)R^{b};
R⁷ is selected from H, halo, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a}, —NR^{a}C₂₋₆alkOR^{a} and C₁₋₆alk, wherein the C₁₋₆alk is substituted by 0, 1, 2 or 3 substituents selected from halo, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}, and the C₁₋₆alk is additionally substituted by 0 or 1 saturated, partially-saturated or unsaturated 5-, 6- or 7-membered monocyclic rings containing 0, 1, 2, 3 or 4 atoms selected from N, O and S, but containing no more than one O or S, wherein the available carbon atoms of the ring are substituted by 0, 1 or 2 oxo or thioxo groups, wherein the ring is substituted by 0, 1, 2 or 3 substituents independently selected from halo, nitro, cyano, C₁₋₄alk, OC₁₋₄alk, OC₁₋₄haloalk, NHC₁₋₄alk, N(C₁₋₄alk)C₁₋₄alk and C₁₋₄haloalk; or R⁷ and R⁸ together form a —C═N— bridge wherein the carbon atom is substituted by H, halo, cyano, or a saturated, partially-saturated or unsaturated 5-, 6- or 7-membered monocyclic ring containing 0, 1, 2, 3 or 4 atoms selected from N, O and S, but containing no more than one O or S, wherein the available carbon atoms of the ring are substituted by 0, 1 or 2 oxo or thioxo groups, wherein the ring is substituted by 0, 1, 2, 3 or 4 substituents selected from halo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, OC(═O)R^{a}, OC(═O)NR^{a}R^{a}, OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a} and —NR^{a}C₂₋₆alkOR^{a}; or R⁷ and R⁹ together form a —N═C— bridge wherein the carbon atom is substituted by H, halo, C₁₋₆alk, C₁₋₄haloalk, cyano, nitro, OR^{a}, NR^{a}R^{a}, —C(═O)R^{a}, —C(═O)OR^{a}, —C(═O)NR^{a}R^{a}, —C(═NR^{a})NR^{a}R^{a}, —S(═O)R^{a}, —S(═O)₂R^{a} or —S(═O)₂NR^{a}R^{a};
R⁸ is H, C₁₋₆alk, C(═O)N(R^{a})R^{a}, C(═O)R^{b} or C₁₋₄haloalk;
R⁹ is H, C₁₋₆alk or C₁₋₄haloalk;
R¹⁰ is in each instance H, halo, C₁₋₃alk, C₁₋₃haloalk or cyano;
R¹¹ is selected from H, halo, C₁₋₆alk, C₁₋₄haloalk, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkNR^{a}R^{a}, —OC₂₋₆alkOR^{a}, —SR^{a}, —S(═O)R^{a}, —S(═O)₂R^{b}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkNR^{a}R^{a}, —NR^{a}C₂₋₆alkOR^{a}, —NR^{a}C₂₋₆alkCO₂R^{a}, —NR^{a}C₂₋₆alkSO₂R^{b}, —CH₂C(═O)R^{a}, —CH₂C(═O)OR^{a}, —CH₂C(═O)NR^{a}R^{a}, —CH₂C(═NR^{a})NR^{a}R^{a}, —CH₂OR^{a}, —CH₂C(═O)R^{a}, —CH₂C(═O)NR^{a}R^{a}, —CH₂C(═O)N(R^{a})S(═O)₂R^{a}, —CH₂OC₂₋₆alkNR^{a}R^{a}, —CH₂OC₂₋₆alkOR^{a}, —CH₂SR^{a}, —CH₂S(═O)R^{a}, —CH₂S(═O)₂R^{b}, —CH₂S(═O)₂NR^{a}R^{a}, —CH₂S(═O)₂N(R^{a})C(═O)R^{a}, —CH₂S(═O)₂N(R^{a})C(═O)OR^{a}, —CH₂S(═O)₂N(R^{a})C(═O)NR^{a}R^{a}, —CH₂NR^{a}R^{a}, —CH₂N(R^{a})C(═O)R^{a}, —CH₂N(R^{a})C(═O)OR^{a}, —CH₂N(R^{a})C(═O)NR^{a}R^{a}, —CH₂N(R^{a})C(═NR^{a})NR^{a}R^{a}, —CH₂N(R^{a})S(═O)₂R^{a}, —CH₂N(R^{a})S(═O)₂NR^{a}R^{a}, —CH₂NR^{a}C₂₋₆alkNR^{a}R^{a}, —CH₂NR^{a}C₂₋₆alkOR^{a}, —CH₂NR^{a}C₂₋₆alkCO₂R^{a}, —CH₂NR^{a}C₂₋₆alkSO₂R^{b}, —CH₂R^{c}, —C(═O)R^{c} and —C(═O)N(R^{a})R^{c};
R^{a} is independently, at each instance, H or R^{b};
R^{b} is independently, at each instance, phenyl, benzyl or C₁₋₆alk, the phenyl, benzyl and C₁₋₆alk being substituted by 0, 1, 2 or 3 substituents selected from halo, C₁₋₄alk, C₁₋₃haloalk, —OH, —OC₁₋₄alk, —NH₂, —NHC₁₋₄alk and —N(C₁₋₄alk)C₁₋₄alk; and
R^{c} is a saturated or partially-saturated 4-, 5- or 6-membered ring containing 1, 2 or 3 heteroatoms selected from N, O and S, the ring being substituted by 0, 1, 2 or 3 substituents selected from halo, C₁₋₄alk, C₁₋₃haloalk, —OC₁₋₄alk, —NH₂, —NHC₁₋₄alk and —N(C₁₋₄alk)C₁₋₄alk.

2. A compound according to claim 1, wherein the compound is:
3-(1-(((6-amino-5-cyano-4-pyrimidinyl)amino)ethyl)-4-(2-pyridinyl)-8-quinolinecarbonitrile;
4-amino-6-(((1R)-1-(4-(2-pyridinyl)-3-cinnolinyl)ethyl)amino)-5-pyrimidinecarbonitrile;
4-amino-6-(((1R)-1-(4-(2-pyridinyl)-3-quinolinyl)ethyl)amino)-5-pyrimidinecarbonitrile;
4-amino-6-(((1R)-1-(4-(3,5-difluorophenyl)-3-cinnolinyl)ethyl)amino)-5-pyrimidinecarbonitrile;
4-amino-6-(((1R)-1-(4-(3,5-difluorophenyl)-3-quinolinyl)ethyl)amino)-5-pyrimidinecarbonitrile;
4-amino-6-(((1R)-1-(4-(3,5-difluorophenyl)-6-fluoro-3-quinolinyl)ethyl)amino)-5-pyrimidinecarbonitrile;
4-amino-6-(((1R)-1-(4-(3,5-difluorophenyl)-8-fluoro-3-quinolinyl)ethyl)amino)-5-pyrimidinecarbonitrile;
4-amino-6-(((1R)-1-(4-(3-cyanophenyl)-6-fluoro-3-quinolinyl)ethyl)amino)-5-pyrimidinecarbonitrile;
4-amino-6-(((1R)-1-(4-(3-fluorophenyl)-3-cinnolinyl)ethyl)amino)-5-pyrimidinecarbonitrile;
4-amino-6-(((1R)-1-(4-(4-fluorophenyl)-3-quinolinyl)ethyl)amino)-5-pyrimidinecarbonitrile;
4-amino-6-(((1R)-1-(4-cyclopropyl-6-fluoro-3-quinolinyl)ethyl)amino)-5-pyrimidinecarbonitrile;
4-amino-6-(((1R)-1-(4-phenyl-3-cinnolinyl)ethyl)amino)-5-pyrimidinecarbonitrile;
4-amino-6-(((1R)-1-(4-phenyl-3-isoquinolinyl)ethyl)amino)-5-pyrimidinecarbonitrile;
4-amino-6-(((1R)-1-(4-phenyl-3-quinolinyl)ethyl)amino)-5-pyrimidinecarbonitrile;
4-amino-6-(((1R)-1-(5-fluoro-4-phenyl-3-quinolinyl)ethyl)amino)-5-pyrimidinecarbonitrile;
4-amino-6-(((1R)-1-(6-chloro-4-(2-pyridinyl)-3-quinolinyl)ethyl)amino)-5-pyrimidinecarbonitrile;
4-amino-6-(((1R)-1-(6-fluoro-4-(2-pyridinyl)-3-cinnolinyl)ethyl)amino)-5-pyrimidinecarbonitrile;
4-amino-6-(((1R)-1-(6-fluoro-4-(2-pyridinyl)-3-quinolinyl)ethyl)amino)-5-pyrimidinecarbonitrile;
4-amino-6-(((1R)-1-(6-fluoro-4-(3-fluorophenyl)-3-quinolinyl)ethyl)amino)-5-pyrimidinecarbonitrile;
4-amino-6-(((1R)-1-(6-fluoro-4-phenyl-3-cinnolinyl)ethyl)amino)-5-pyrimidinecarbonitrile;
4-amino-6-(((1R)-1-(6-fluoro-4-phenyl-3-quinolinyl)ethyl)amino)-5-pyrimidinecarbonitrile;
4-amino-6-(((1R)-1-(7-chloro-4-(2-pyridinyl)-3-quinolinyl)ethyl)amino)-5-pyrimidinecarbonitrile;
4-amino-6-(((1R)-1-(7-fluoro-4-(2-pyridinyl)-3-quinolinyl)ethyl)amino)-5-pyrimidinecarbonitrile;
4-amino-6-(((1R)-1-(8-chloro-4-(2-pyridinyl)-3-quinolinyl)ethyl)amino)-5-pyrimidinecarbonitrile;
4-amino-6-(((1R)-1-(8-chloro-4-(3-fluorophenyl)-3-quinolinyl)ethyl)amino)-5-pyrimidinecarbonitrile;
4-amino-6-(((1R)-1-(8-chloro-6-fluoro-4-(2-pyridinyl)-3-quinolinyl)ethyl)amino)-5-pyrimidinecarbonitrile;
4-amino-6-(((1R)-1-(8-chloro-6-fluoro-4-(2-pyridinyl)-3-quinolinyl)ethyl)amino)-5-pyrimidinecarbonitrile;
4-amino-6-(((1R)-1-(8-fluoro-4-(2-pyridinyl)-3-quinolinyl)ethyl)amino)-5-pyrimidinecarbonitrile;
4-amino-6-(((1R)-1-(8-fluoro-4-(3-fluorophenyl)-3-quinolinyl)ethyl)amino)-5-pyrimidinecarbonitrile;
4-amino-6-(((1R)-1-(8-fluoro-4-phenyl-3-quinolinyl)ethyl)amino)-5-pyrimidinecarbonitrile;
4-amino-6-(((1S)-1-(4-(2-pyridinyl)-3-cinnolinyl)ethyl)amino)-5-pyrimidinecarbonitrile;
4-amino-6-(((1S)-1-(4-(2-pyridinyl)-3-quinolinyl)ethyl)amino)-5-pyrimidinecarbonitrile;
4-amino-6-(((1S)-1-(4-(3,5-difluorophenyl)-3-cinnolinyl)ethyl)amino)-5-pyrimidinecarbonitrile;
4-amino-6-(((1S)-1-(4-(3,5-difluorophenyl)-3-quinolinyl)ethyl)amino)-5-pyrimidinecarbonitrile;
4-amino-6-(((1S)-1-(4-(3,5-difluorophenyl)-6-fluoro-3-quinolinyl)ethyl)amino)-5-pyrimidinecarbonitrile;
4-amino-6-(((1S)-1-(4-(3,5-difluorophenyl)-8-fluoro-3-quinolinyl)ethyl)amino)-5-pyrimidinecarbonitrile;
4-amino-6-(((1S)-1-(4-(3-cyanophenyl)-6-fluoro-3-quinolinyl)ethyl)amino)-5-pyrimidinecarbonitrile;
4-amino-6-(((1S)-1-(4-(3-fluorophenyl)-3-cinnolinyl)ethyl)amino)-5-pyrimidinecarbonitrile;
4-amino-6-(((1S)-1-(4-(4-fluorophenyl)-3-quinolinyl)ethyl)amino)-5-pyrimidinecarbonitrile;
4-amino-6-(((1S)-1-(4-cyclopropyl-6-fluoro-3-quinolinyl)ethyl)amino)-5-pyrimidinecarbonitrile;
4-amino-6-(((1S)-1-(4-phenyl-3-cinnolinyl)ethyl)amino)-5-pyrimidinecarbonitrile;
4-amino-6-(((1S)-1-(4-phenyl-3-isoquinolinyl)ethyl)amino)-5-pyrimidinecarbonitrile;
4-amino-6-(((1S)-1-(4-phenyl-3-quinolinyl)ethyl)amino)-5-pyrimidinecarbonitrile;
4-amino-6-(((1S)-1-(5-fluoro-4-phenyl-3-quinolinyl)ethyl)amino)-5-pyrimidinecarbonitrile;
4-amino-6-(((1S)-1-(6-chloro-4-(2-pyridinyl)-3-quinolinyl)ethyl)amino)-5-pyrimidinecarbonitrile;
4-amino-6-(((1S)-1-(6-fluoro-4-(2-pyridinyl)-3-cinnolinyl)ethyl)amino)-5-pyrimidinecarbonitrile;
4-amino-6-(((1S)-1-(6-fluoro-4-(2-pyridinyl)-3-quinolinyl)ethyl)amino)-5-pyrimidinecarbonitrile;
4-amino-6-(((1S)-1-(6-fluoro-4-(3-fluorophenyl)-3-quinolinyl)ethyl)amino)-5-pyrimidinecarbonitrile;
4-amino-6-(((1S)-1-(6-fluoro-4-phenyl-3-cinnolinyl)ethyl)amino)-5-pyrimidinecarbonitrile;
4-amino-6-(((1S)-1-(6-fluoro-4-phenyl-3-quinolinyl)ethyl)amino)-5-pyrimidinecarbonitrile;
4-amino-6-(((1S)-1-(7-chloro-4-(2-pyridinyl)-3-quinolinyl)ethyl)amino)-5-pyrimidinecarbonitrile;
4-amino-6-(((1S)-1-(7-fluoro-4-(2-pyridinyl)-3-quinolinyl)ethyl)amino)-5-pyrimidinecarbonitrile;
4-amino-6-(((1S)-1-(8-chloro-4-(2-pyridinyl)-3-quinolinyl)ethyl)amino)-5-pyrimidinecarbonitrile;
4-amino-6-(((1S)-1-(8-chloro-4-(3-fluorophenyl)-3-quinolinyl)ethyl)amino)-5-pyrimidinecarbonitrile;
4-amino-6-(((1S)-1-(8-chloro-6-fluoro-4-(2-pyridinyl)-3-quinolinyl)ethyl)amino)-5-pyrimidinecarbonitrile;
4-amino-6-(((1S)-1-(8-chloro-6-fluoro-4-(2-pyridinyl)-3-quinolinyl)ethyl)amino)-5-pyrimidinecarbonitrile;
4-amino-6-(((1S)-1-(8-fluoro-4-(2-pyridinyl)-3-quinolinyl)ethyl)amino)-5-pyrimidinecarbonitrile;
4-amino-6-(((1S)-1-(8-fluoro-4-(3-fluorophenyl)-3-quinolinyl)ethyl)amino)-5-pyrimidinecarbonitrile;
4-amino-6-(((1S)-1-(8-fluoro-4-phenyl-3-quinolinyl)ethyl)amino)-5-pyrimidinecarbonitrile;
4-amino-6-((1-(1-(3,5-difluorophenyl)-2-naphthalenyl)ethyl)amino)-5-pyrimidinecarbonitrile;
4-amino-6-((1-(4-(2-pyridinyl)-3-cinnolinyl)ethyl)amino)-5-pyrimidinecarbonitrile;
4-amino-6-((1-(4-(2-pyridinyl)-3-quinolinyl)ethyl)amino)-5-pyrimidinecarbonitrile;
4-amino-6-((1-(4-(3-(methylsulfonyl)phenyl)-3-cinnolinyl)ethyl)amino)-5-pyrimidinecarbonitrile;
4-amino-6-((1-(4-(3,5-difluorophenyl)-3-cinnolinyl)ethyl)amino)-5-pyrimidinecarbonitrile;
4-amino-6-((1-(4-(3,5-difluorophenyl)-3-quinolinyl)ethyl)amino)-5-pyrimidinecarbonitrile;
4-amino-6-((1-(4-(3,5-difluorophenyl)-6-fluoro-3-quinolinyl)ethyl)amino)-5-pyrimidinecarbonitrile;
4-amino-6-((1-(4-(3,5-difluorophenyl)-8-fluoro-3-quinolinyl)ethyl)amino)-5-pyrimidinecarbonitrile;
4-amino-6-((1-(4-(3-cyanophenyl)-6-fluoro-3-quinolinyl)ethyl)amino)-5-pyrimidinecarbonitrile;
4-amino-6-((1-(4-(3-fluorophenyl)-3-cinnolinyl)ethyl)amino)-5-pyrimidinecarbonitrile;
4-amino-6-((1-(4-(4-(methylsulfonyl)phenyl)-3-cinnolinyl)ethyl)amino)-5-pyrimidinecarbonitrile;
4-amino-6-((1-(4-(4-cyanophenyl)-6-fluoro-3-quinolinyl)ethyl)amino)-5-pyrimidinecarbonitrile;
4-amino-6-((1-(4-(4-fluorophenyl)-3-quinolinyl)ethyl)amino)-5-pyrimidinecarbonitrile;
4-amino-6-((1-(4-cyclopropyl-6-fluoro-3-quinolinyl)ethyl)amino)-5-pyrimidinecarbonitrile;
4-amino-6-((1-(4-phenyl-3-cinnolinyl)ethyl)amino)-5-pyrimidinecarbonitrile;
4-amino-6-((1-(4-phenyl-3-isoquinolinyl)ethyl)amino)-5-pyrimidinecarbonitrile;
4-amino-6-((1-(4-phenyl-3-quinolinyl)ethyl)amino)-5-pyrimidinecarbonitrile;
4-amino-6-((1-(5-fluoro-4-phenyl-3-quinolinyl)ethyl)amino)-5-pyrimidinecarbonitrile;
4-amino-6-((1-(6-chloro-4-(2-pyridinyl)-3-quinolinyl)ethyl)amino)-5-pyrimidinecarbonitrile;
4-amino-6-((1-(6-fluoro-4-(2-pyrazinyl)-3-quinolinyl)ethyl)amino)-5-pyrimidinecarbonitrile;
4-amino-6-((1-(6-fluoro-4-(2-pyridinyl)-3-cinnolinyl)ethyl)amino)-5-pyrimidinecarbonitrile;
4-amino-6-((1-(6-fluoro-4-(2-pyridinyl)-3-quinolinyl)ethyl)amino)-5-pyrimidinecarbonitrile;
4-amino-6-((1-(6-fluoro-4-(3-fluorophenyl)-3-cinnolinyl)ethyl)amino)-5-pyrimidinecarbonitrile;
4-amino-6-((1-(6-fluoro-4-(3-fluorophenyl)-3-quinolinyl)ethyl)amino)-5-pyrimidinecarbonitrile;
4-amino-6-((1-(6-fluoro-4-phenyl-3-cinnolinyl)ethyl)amino)-5-pyrimidinecarbonitrile;
4-amino-6-((1-(6-fluoro-4-phenyl-3-quinolinyl)ethyl)amino)-5-pyrimidinecarbonitrile;
4-amino-6-((1-(7-chloro-4-(2-pyridinyl)-3-quinolinyl)ethyl)amino)-5-pyrimidinecarbonitrile;
4-amino-6-((1-(7-fluoro-4-(2-pyrazinyl)-3-quinolinyl)ethyl)amino)-5-pyrimidinecarbonitrile;
4-amino-6-((1-(7-fluoro-4-(2-pyridinyl)-3-quinolinyl)ethyl)amino)-5-pyrimidinecarbonitrile;
4-amino-6-((1-(8-(3,5-difluorophenyl)-7-quinolinyl)ethyl)amino)-5-pyrimidinecarbonitrile;
4-amino-6-((1-(8-chloro-4-(1H-pyrazol-5-yl)-3-quinolinyl)ethyl)amino)-5-pyrimidinecarbonitrile;
4-amino-6-((1-(8-chloro-4-(2-pyridinyl)-3-quinolinyl)ethyl)amino)-5-pyrimidinecarbonitrile;
4-amino-6-((1-(8-chloro-4-(3-fluorophenyl)-3-quinolinyl)ethyl)amino)-5-pyrimidinecarbonitrile;
4-amino-6-((1-(8-chloro-6-fluoro-4-(2-pyridinyl)-3-quinolinyl)ethyl)amino)-5-pyrimidinecarbonitrile;
4-amino-6-((1-(8-chloro-6-fluoro-4-(2-pyridinyl)-3-quinolinyl)ethyl)amino)-5-pyrimidinecarbonitrile;
4-amino-6-((1-(8-fluoro-4-(2-pyridinyl)-3-quinolinyl)ethyl)amino)-5-pyrimidinecarbonitrile;
4-amino-6-((1-(8-fluoro-4-(3-fluorophenyl)-3-quinolinyl)ethyl)amino)-5-pyrimidinecarbonitrile;
4-amino-6-((1-(8-fluoro-4-phenyl-3-quinolinyl)ethyl)amino)-5-pyrimidinecarbonitrile;
4-amino-6-((1R)-1-(4-(2-pyridinyl)-3-quinolinyl)ethoxy)-5-pyrimidinecarbonitrile;
4-amino-6-((1S)-1-(4-(2-pyridinyl)-3-quinolinyl)ethoxy)-5-pyrimidinecarbonitrile;
4-amino-6-(1-(4-(2-pyridinyl)-3-quinolinyl)ethoxy)-5-pyrimidinecarbonitrile;
N-((1R)-1-(4-phenyl-3-cinnolinyl)ethyl)-9H-purin-6-amine;
N-((1R)-1-(6-fluoro-4-(3-fluorophenyl)-3-quinolinyl)ethyl)-9H-purin-6-amine;
N-((1R)-1-(6-fluoro-4-phenyl-3-quinolinyl)ethyl)-9H-purin-6-amine;
N-((1S)-1-(4-phenyl-3-cinnolinyl)ethyl)-9H-purin-6-amine;
N-((1S)-1-(6-fluoro-4-(3-fluorophenyl)-3-quinolinyl)ethyl)-9H-purin-6-amine;
N-((1S)-1-(6-fluoro-4-phenyl-3-quinolinyl)ethyl)-9H-purin-6-amine;
N-((1-(4-phenyl-3-cinnolinyl)ethyl)-9H-purin-6-amine;
N-(1-(6-fluoro-4-(2-pyridinyl)-3-cinnolinyl)ethyl)-9H-purin-6-amine;
N-(1-(6-fluoro-4-(2-pyridinyl)-3-quinolinyl)ethyl)-9H-purin-6-amine;
N-(1-(6-fluoro-4-(3-fluorophenyl)-3-quinolinyl)ethyl)-9H-purin-6-amine;
N-(1-(6-fluoro-4-phenyl-3-quinolinyl)ethyl)-9H-purin-6-amine;
N-(1-(7-fluoro-4-(2-pyridinyl)-3-quinolinyl)ethyl)-9H-purin-6-amine; and
N-(1-(8-(3,5-difluorophenyl)-7-quinolinyl)ethyl)-9H-purin-6-amine; or any pharmaceutically-acceptable salt thereof.

3. A method of treating rheumatoid arthritis, ankylosing spondylitis, osteoarthritis, psoriatic arthritis, psoriasis, inflammatory diseases and autoimmune diseases, inflammatory bowel disorders, inflammatory eye disorders, inflammatory or unstable bladder disorders, skin complaints with inflammatory components, chronic inflammatory conditions, autoimmune diseases, systemic lupus erythematosis (SLE), myestenia gravis, rheumatoid arthritis, acute disseminated encephalomyelitis, idiopathic thrombocytopenic purpura, multiples sclerosis, Sjoegren's syndrome and autoimmune hemolytic anemia, allergic conditions and hypersensitivity, comprising the step of administering a compound according to claim 1.

4. A method of treating cancers, which are mediated, dependent on or associated with p110δ activity, comprising the step of administering a compound according to claim 1.

5. A pharmaceutical composition comprising a compound according to claim 1 and a pharmaceutically-acceptable diluent or carrier.
